(19) United States
(12) Patent Application Publication
(10) Pub. No.: US 2015/0150893 A1
(43) Pub. Date: Jun. 4, 2015

(54) ANDROGRAPHOLIDE ANALOGS AND THEIR USE FOR MEDICATION

(71) Applicant: LIAONING LIFENG SCIENTIFIC & TECHNOLOGY DEVELOPMENT COMPANY LTD., Shenyang, Liaoning (CN)

(72) Inventor: Lifeng XU, San Diego, CA (US)

(21) Appl. No.: 14/406,206

(22) PCT Filed: May 31, 2013
(86) PCT No.: PCT/CN2013/076521

§ 371 Date: Dec. 6, 2014

(30) Foreign Application Priority Data
Jun. 18, 2012 (CN) 201210200037.X

(51) Int. Cl. A61K 31/704; A61K 31/365; A61K 45/06; C07D 405/14; A61K 31/4439; C07D 405/12; A61K 31/4178; A61K 31/506; C07D 473/18; A61K 31/5377; C07H 15/26; A61K 31/7034; C07F 9/655; A61K 31/683; C07D 487/18; A61K 31/53; C07F 9/6568; A61K 31/665; C07D 307/60

(52) U.S. Cl. CPC A61K 31/704; C07D 307/60; A61K 31/365; A61K 45/06; C07D 405/14; A61K 31/4439; C07D 405/12; A61K 31/4178; A61K 31/506; C07D 473/18; A61K 31/5377; C07H 15/26; A61K 31/7034; C07F 9/65515; A61K 31/683; C07D 487/18; A61K 31/53; C07F 9/65686; A61K 31/665

(57) ABSTRACT

This invention relates to novel andrographolide analogs of formula I that are useful for the treatment, prevention and/or amelioration of human diseases of viruses and cancers. This invention also relates with their pharmaceutical compositions, preparative methods and applications.

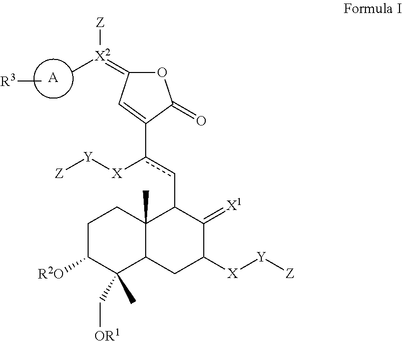

### THE FIELD OF THE INVENTION

This invention relates to novel andrographolide analogs that are useful for the treatment, prevention and/or amelioration of human diseases of viruses and cancers, the pharmaceutical compositions containing these compounds and the methods for their preparation.

### BACKGROUND OF THE INVENTION

Andrographolide is the main active component of the Herba Andrographitis which is widely used in many countries of Asia for the treatment of viral infections, diabetes, rheumatic arthritis, pharyngolaryngitis and diarrhea (Puri et al., J. Nat. Prod. 1993, 56, 995-999; Zhang and Tan, Clin. Exp. Pharmacol. Physiol. 1996, 23, 675-678; Zhang and Tan, Clin. Exp. Pharmacol. Physiol. 2000, 27, 358-363). Recent report indicated that andrographolide exhibited good cancer therapeutic effects (Satyanarayana et al. Science 2003, 299, 363-370). Significant attention has been paid by several research groups on andrographolide and analogs recent years due to its antitumorogenic and anti-viral activities for treating or preventing pathogenecity of diseases such as AIDS, Alzheimer's disease and hepatitis WO96/17605, U.S. Pat. No. 6,486,196B, US20060106098A1, US6576, 662B2, US6486, 196B2, US2006/0106098A1, US2011/0077295A1, US2002/0016363A1, US2002/0016324A1, US7625945B2 and US20020032229A1 disclosed anti-cancer activity of andrographolide analogs; US20110077295A1 reported the medicaments of andrographolide derivatives for treatment of cancers, diabetes, inflammantion, bacterials and viral infections; US2005/0215628A1 and US2012/0015923A1 disclosed the medicaments of andrographolide derivatives for treatment of inflammation; US2006/0223785 reported the medicaments of andrographolide derivatives for treatment of virus diseases. To date there has been no report related with structural modification of andrographolide with the introduction of substituents to form andrographolide analogs at both sites of C₇ and C₁₅ positions, nor studies of antiviral activity and anticancer activity, nor structure-activity relationship studies of antiviral activity and anticancer activity by the andrographolide analogs modified at both sites of C₇ and C₁₅ positions from all literature reviewed.

### DETAILED DESCRIPTION OF THE INVENTION

The purpose of the present invention is to provide a novel andrographolide analogs to their use as antivirus and anticancer agents, to pharmaceutical compositions containing these compounds and to the methods for their preparation, which have the general formula I

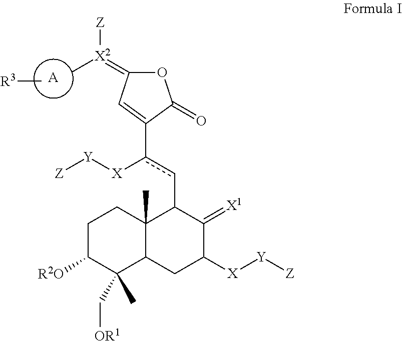

or stereoisomers, tautoers, prodrug, pharmaceutically acceptable salts, complex salts or solvates thereof, wherein: the dotted lines [symbol] are absent or selected but is not limited from: —C—C—, —C═C—, —C-heterobond,

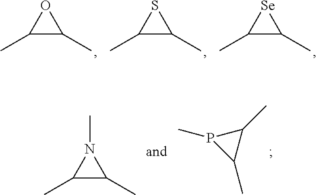

X, X¹ and X² is absent or independently selected but is not limited from: hydrogen, halogen, —C—, —S—, —O—, —NH—, —NR—, —NRR, —NHC(O)—, —NRC(O)—, —NHSO₂—, —NRSO₂—, —SO₂NH—, —SO₂NR—, —C(O)—, —C(O)NH—, —C(O)NR—, —C(O)R—, —CO₂—, —C(O)H, —C(O)NH₂—, —CO₂H, —C(NH)NH—, —C(NH)NR—, —C(S)—, —C(S)NH—, —C(S)NR—, —C(S)R—, —C(NH)NH—, —C(NH)NR—, ═CH—, ═CH₂, ═CH—O—, ═CH—S—, ═CH—Se—, ═CH—NR—, ═CH—NH—, ═CH—PR—, wherein R is selected but is not limited from: —C₁₋₆ alkyl, —CO₂H, —CO₂C₁₋₆alkyl, COC₁₋₆alkyl, phenyl, —CH₂phenyl, heteroalkyl and heteroaryl;

Y is absent or selected but is not limited from: —CH₂—, —CH₂—CH₂—, —CH₂—CH₂—CH₂—, —CHF—, and —CF₂—, wherein each CH₂ and CHF is unsubstituted or substituted with 1 or 2 substituents selected from R^{a};

Z is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, aryl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —C(O)OC₁₋₆alkyl, —C(O)NHC₁₋₆alkyl, P(O)(OH)2, P(O)(OR)2, —(CH₂)ₘP(O)(OH)₂, —(CH₂)ₘP(O)(OC₁₋₆alkyl)₂, —(CH₂)ₘP(O)(NR^{b}C(R^{c}))₂, C₃₋₈ amino acid, C₃₋₁₀(OH)₀₋₁₀polyhydroxide;

each R¹, R² and R³ is absent or independently selected but is not limited from: hydrogen, halogen, —C₁₋₁₀alkyl, —C₂₋₆alkenyl, —C₂₋₆lkynyl, —(CH₂)ₚC₃₋₁₀ cycloalkyl, —(CH₂)ₚC₃₋₇cycloalkylaryl, —(CH₂)ₚC₃₋₇cycloalkylheteroaryl, —(CH₂)ₚC₄₋₁₀cycloalkenyl, —(CH₂)ₚC₄₋₇ cycloalkenylaryl, —(CH₂)ₚC₄₋₇cycloalkenylheteroaryl, —(CH₂)ₚheteroaryl, —C₂₋₆alkenylalkyl, —C₂₋₆alkenylaryl, —C₂₋₆alkenyl-heteroaryl, —C₂₋₆alkenyl-C₃₋₇cycloalkyl, —C₂₋₆alkynylalkyl, —C₂₋₆alkynylaryl, —C₂₋₆alkynyheteroaryl, —C₂₋₆alkynyl-C₃₋₇cycloalkyl, —C₂₋₆alkynyl-C₃₋₇cycloalkenyl, —C₂₋₆alkynyl-C₂₋₇cycloheteroalkyl, —C₂₋₆alkynyl-C₂₋₇cycloheteroalkenyl, —C(O)(CH₂)₀₋₃phenyl, —(CH₂)ₚC(O)phenyl, —C(NH) (CH₂)₀₋₃phenyl, —(CH₂)ₘP(O)(NR^{b}C(R^{c}))₂, C₃₋₈ amino acid, C₃₋₁₀(OH)₀₋₁₀polyhydroxide, aryl, biphenyl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, —NHC(O)C₁₋₆alkyl, guanidine, amidine —SC₁₋₆alkyl, —SOC₁₋₆alkyl, —SO₂C₁₋₆alkyl, —NHSO₂C₁₋₆alkyl, —SO₂NHC₁₋₆alkyl, —C(O)OH, —C(O)OC₁₋₆alkyl, —C(O)NHC₁₋₆alkyl, P(O)(OH)2, P(O)(OR)2, —(CH₂)ₘP(O)(OH)₂, —(CH₂)ₘP(O)(OC₁₋₆alkyl)₂, —(CH₂)ₘP(O)(NR^{b}C(R^{c}))₂, C₃₋₈ amino acid, C₃₋₁₀(OH)₀₋₁₀polyhydroxide;

wherein each CH₂ is unsubstituted or substituted with 1 or 2 substituents selected from: halogen, —CF₃, —OH, —NH₂, —C₁₋₆alkyl, —OC₁₋₅alkyl, —NHC₁₋₆alkyl and —N(C₁₋₆alkyl)₂, each alkyl, alkenyl and alkynyl is unsubstituted or substituted with 1, 2 or 3 substituents selected from: halogen, CF₃, —OH, —NH₂, —C₁₋₆alkyl, —OC₁₋₆alkyl, —NHC₁₋₆alkyl and —N(C₁₋₆alkyl)₂, and each cycloalkyl, cycloalkenyl, cycloheteroalkyl, cycloheteroalkenyl, phenyl, aryl and heteroaryl is unsubstituted or substituted with 1, 2, 3 or 4 substituents independently selected from R^{a};

A ring is selected from: —C₃₋₁₄alkylcycle, —C₃₋₁₄arylcycle, —C₃₋₁₄heterocycle and —C₃₋₁₄heteroaryl;

said —C₃₋₁₄alkylcycle, arylcycle, heterocycle and —C₃₋₁₄heteroaryl are selected but are not limited from acridinyl, azetidinyl, acridinyl, azocinyl, azepanyl, azepinyl, aziridinyl, azirinyl, azete, benzothiazole, benzofuranyl, benzimidazolyl, benzofuranyl, benzothranyl, benzothiofuranyl, benzthiazolyl, benztetrazolyl, benzisoxazolyl, benzisothiazolyl, benzimidazolinyl, benzopyrazolyl, benzotriazolyl, benzothiophenyl, benzoxazolyl, benz-1H-tetrazolyl, benz-2H-tetrazolyl, benz-3H-tetrazolyl, benz-4H-tetrazolyl, benz-5H-tetrazolyl, benzothienyl, benzofurazanyl, benzodiazepinyl, carbazolyl, carbolinyl, cinnolinyl, carbazolyl, carbolinyl, chromanyl, chromenyl, coumarinyl, decahydroquinolinyl, 4aH-carbazolyl, 1,4-dioxanyl, 2H, 6H-1,5,2-dithiazinyl, dihydrofuro[2,3-b]tetrahydrothran, furazanyl, hexahydroazepinyl, imidazole, imidazolyl, indolinyl, indolazinyl, indazolyl, isoindolyl isoquinolyl, imidazolidinyl, imidazolinyl, indolyl, indolazinyl, indazolyl, 1H-indazolyl, indolenyl, indolinyl, indolizinyl, 3H-indolyl, isobenzofuranyl, isoquinolyl, 1,2-Isoxazole, 1,3-Isoxazole, isoxazolyl, isochromanyl, isoindolinyl, isothiazolyl, isoxazoline, isoquinolinyl, methylenedioxybenzoyl, methylenedioxyphenyl, morpholinyl, naphthpyridinyl, N-oxides oxetanyl, oxadiazolyl, oxazolyl, oxazolinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxolanyl, oxirenyl, oxete, oxiranyl, oxanyl, oxetanyl, oxepanyl, oxepinyl, oxazolinyl, pyrazole, 2(1H)pyrimidinone, piperidine, thiiranyl, phenanthridinyl, phenanthrolinyl, phenazinyl, phenothiazinyl, phenoxathiinyl, phenoxazinyl, phthalazinyl, piperidinyl, 2H-pyrrolyl, pyridin-2-one, piperazinyl, piperidonyl, 4-piperizinyl, piperonyl, pteridinyl, purinyl, pyrazinyl, pyrazolidinyl, pyridazine, pyrazolinyl, pyridazinyl, pyridoimidazole, pyridothiazole, pyridyl, pyrimidinyl, pyridopyridinyl, pyrrazolyl, pyrrolinyl, pyrazolyl, pyrrolyl, pyranyl, pyrazine, pyridinyl, pyrrolidinyl, quinazolinyl, quinolyl, quinoxalinyl, 4H-quinolizinyl, quinuclidinyl, quinoxaline-2(1H)one, quinolinyl, thiadiazolyl, thranyl, 6H-1,2,5-thiadiazinyl, 1,2,3-thiadiazolyl, 1,2,4-thiadiazinyl, 1,2,5-thiadiazinyl, 1,3,4-trizzolyl, 1,3,4-thiadiazinyl, thiomorpholinyl, thienyl, thiomorpholinyl, triazolyl, thiirenyl, thietanyl, thiete, thiolanyl, thiophenyl, thianyl, thiopyranyl, thiepanyl, thiepinyl, thiazole, thionaphthenyl, purinyl, 2,4,6-trihydroxypurinyl, 1,2-thiazole, 1,3-thiazole, xanthenyl;
wherein A ring includes the above said heteroaryls, as well as dihydro and tetrathydro analogs and is, unsubstituted or optionally substituted with 1, 2 or 3 same or different substituents and each substituent selected but are not limited from: hydrogen, halogen, —OH, —OR, —SH—, —SR—, —O—, —NO₂, —NH₂, —NH—, —NR—, —NRR, —CF₃, —CN, —C(O)—, —NHC(O)—, —NRC(O)—, —C(O)NH—, —C(O)NR—, —NHSO₂—, —NRSO₂—, —SO₂NH—, —SO₂NR—, —C(O)R—, —CO₂—, —C(NH)NH—, —C(NH)NR—, wherein R is selected from: —C₁ alkyl, —CH₂F, —CHF₂, —CF₃, —CO₂H, —CO₂C₁₋₆alkyl, COC₁₋₆alkyl, phenyl, —CH₂phenyl, heteroalkyl and heteroaryl oxo, —(CH₂)₀₋₃OH, —CN, —NH₂, —NH(C₁₋₆alkyl), —N(C₁₋₆alkyl)2, —C₁₋₆alkyl, —OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂ heteroaryl;

R^{a}, R^{b} and R^{c} are same or different selected but is not limited from hydrogen, halogen, —C—, —S—, —O—, —NH—, —NR—, —NRR, —NHC(O)—, —NRC(O)—, —NHSO₂—, —NRSO₂—, —SO₂NH—, —SO₂NR—, —C(O)—, —C(O)NH—, —C(O)NR—, —C(O)R—, —CO₂—, —C(O)H, —C(O)NH₂—, —CO₂H, —C(NH)NH—, —C(NH)NR—, —C(S)—, —C(S)NH—, —C(S)NR—, —C(S)R—, —C(NH)NH—, —C(NH)NR—, wherein R is selected from: —C₁₋₆ alkyl, —CO₂H, —CO₂C₁₋₆alkyl, COC₁₋₆alkyl, phenyl, —CH₂phenyl, heteroalkyl and heteroaryl; m is 0, 1, 2, 3 or 4; p is 0, 1, 2, or 3;

The invention provides that a compound of andrographolide derivatives and analogs is selected but is not limited from the exemplified examples or stereoisomers, tautomers, pharmaceutically acceptable salts, inorganic acid salt, organic acid salt, organic basic salt, complex salt, prodrug or solvates thereof in association with a pharmaceutically acceptable excipient or carrier.

The invention provides that a process for the manufacture of a compound of formula I to form andrographolide derivatives and analogs is obtained by modification at 7-, 12- or 15-position with a bond of C—C, C—O, C—S, C—N or C—P under catalysis at −78° C. to 90° C., by a solvent selected from THF, 1,4-dioxane, N,N-dimethylformamide amide, N, N-dimethylacetamide, toluene, ethanol, or methanol, at room temperature to 180° C., and a catalyst selected from organic base, inorganic base, molecular sieves or alumina;

The invention provides that a compound of andrographolide derivatives and analogs for treating, preventing or slowing the progression of virus, cancer, bacteria, fungi and other infections, including inflammation, inflammatory diseases and immune system disease associated with viruses, cancer, bacterial and fungi, alone or with the following drugs known to be used in conjunction dose of 0.02 mg/kg-2.0 g/kg (intravenous, intramuscular, oral, topical and other routes of administration); means of various methods of treatment and therapy, where the virus are selected but are not limited from: adenovirus, herpes simplex type 1, herpes simplex type 2, varicellazoster virus, epsteinbarr virus, human cytomegalovirus, human herpesvirus, type 8 human papillomavirus, BK virus, JC virus, smallpox, human bocavirus, parvovirus, B19 human astrovirus, norwalk virus, coxsackievirus, hepatitis A virus, hepatitis B virus, hepatitis C virus, poliovirus, rhinovirus, severe acute respiratory syndrome virus, yellow fever virus, dengue virus, west nile virus, rubella virus Hepatitis E virus, human immunodeficiency virus, influenza virus, guanarito virus, junin virus, lassa virus, machupo virus, Sabiá virus, Crimean-Congo hemorrhagic fever virus, ebola virus, marburg virus, measles virus, mumps virus, parainfluenza virus, respiratory syncytial virus, human metapneumovirus, rabies virus, hepatitis D rotavirus.

The invention provides that a compound of andrographolide derivatives and analogs for treating, preventing or slowing the progression of virus, bacteria, fungi and other infections associated with inflammation and inflammatory diseases, immune system complications of viral infection by respiratory tract, urinary tract, skin and soft tissue, sepsis, bone and joint, abdominal, pelvic viridans and endocarditis selected, but not limited from: aids, cutaneous lesion associated with AIDS, AIDS related malignant tumours, alphaviruses causing encephalitis, arenavirus infection, arthropodborne viral encephalitis, avian influenza, bolivian hemorrhagic fever, coxsackievirus infection, crimeancongo hemorrhagic fever, cytomegalovirus infection, dengue, eastern equine encephalitis, ebola virus infection, echovirus infection, epsteinbarr virus infection, epsteinbarr virusrelated malignant tumours, fifth disease, filovirus infection, flavivirus infection, german measles, hemorrhagic fever with renal syndrome, herpes virus infection, herpes simplex virus infection, herpes zoster virus infection, human papilloma virus associated epidermal lesions, human papilloma virus in cervical cancer, Japanese encephalitis, kaposi sarcoma, korean hemorrhagic fever, kyasanur forest disease, lassa fever, lymphocytic choriomeningitis, molluscum contagiosum, murray valley encephalitis, norwalk virus related diarrhea, omsk hemorrhagic fever, orthomyxoviruses, parainfluenza virus infection, paramyxovirus, parvovirus B19 infection, picornavirus, rotavirus diarrhea, poxviruses, rabies, respiratory syncytial virus infection, rubeola, smallpox, St. Louis encephalitis, tickborne encephalitis, variola, venezuelan equine encephalitis, viral hemorrhagic fevers, viruses in leukemia and lymphoma, western equine encephalitis, west Nile virus disease septicemia, endocarditis infection, adenovirus serotype 14, T-cell leukemia/lymphoma, alastrim, andes viral infection, argentine hemorrhagic fever, astrovirus infection, avian encephalomyelitis viral infection, avian nephritis viral infection, avian orthoreoviral infection, avian pneumoviral infection, borna disease, bornholm disease, bovine adenoviral infection, bovine coronaviral infection, bovine ephemeral fever, bovine herpesviral infection 4, bovine parvoviral infection, bovine viral diarrhea, brazilian hemorrhagic fever, bronchiolitis, bundibugyo ebolaviral infection, bundibugyo viral infection, catflu, cervical intraepithelial neoplasia, chandipura viral infection, channel catfish viral infection, chicken anaemia viral infection, chickenpox, chikungunya outbreaks, common cold, cowpox, coxsackie viral infection, cricket paralysis viral infection, cuevaviral infection, cytomegaloviral infection, cytomegalovirus colitis, cytomegalovirus retinitis, derzsy's disease, downie bodies, dukes' disease, ebola viral infection, ebolaviral infection, elephant endotheliotropic herpesviral infection, epidemic polyarthritis, epidermodysplasia verruciformis, epsteinbarr virus infection, feline leukemia viral infection, filoviridae, foot-and-mouth disease, genital wart, hantaviral infection, henipaviral infection, hepatitis A, hepatitis B, hepatitis C, hepatoviral infection, herpes genitalis, herpes simplex, herpes zoster, herpesviral encephalitis, herpesviral meningitis, herpetic keratoconjunctivitis, HPV-positive oropharyngeal cancer, human bocaviral infection, human cytomegaloviral infection, human respiratory syncytial viral infection, body rhinitis, infectious mononucleosis, infectious pancreatic necrosis, koi herpes viral infection, kunjin viral infection, labrea fever, laryngeal papillomatosis, leucosis, liebermeister's rule, lloviu cuevaviral infection, lloviu viral infection, lujo viral infection, marburg marburgviral infection, marburg virus disease, mayaro virus disease, menangle viral infection, monkeypox, mononegavirales infection, mononucleosis, mumps, encephalitis viral infection, myxomatosis, nephropathia epidemica, oropouche fever, parvoviral B19, phytoreoviral infection, plantar wart, pogosta disease, porcine adenoviral infection, central nervous system lymphoma, retinal necrosis, rubella panencephalitis, qalyub viral infection, rabbit haemorrhagic disease, ramsay hunt syndrome type II, ravn viral infection, reston ebolaviral infection, reston viral infection, rhinoviral infection, rocio viral encephalitis, roseoloviral infection, ross river fever, rotaviral infection, shope papilloma viral infection, simian foamy viral infection, sudan ebolaviral infection, sudan viral infection, swine vesicular disease, taï forest ebolaviral infection, tropical spastic paraparesis, turkey coronaviral infection, turkey viral hepatitis, turkeypox, varicella zoster viral infection, venezuelan hemorrhagic fever, verruca plana, viral infection rthritis, viral arthritis, viral hemorrhagic septicemia, viral systemic infection, virus sin nombre, woodchuck hepatitis viral infection, yellow fever, zika fever, template, zoonotic viral infection.

The invention provides that a compound of andrographolide derivatives and analogs or pharmaceutically acceptable salts is administered together with at least one known antiviral agents, antifungal agents or antiinflammatory agents selected but is not limited from a cytidine analog, an uridine analog, an adenosine analog, a guanosine analog, a thymidine analog or an inosine analog comprising: deoxycytidine; 2′,3′-dideoxycytidine; 2′,3′-didehydrocytidine carbocyclic, 2′,3′-didehydro-2′,3′-dideoxycytidine, 2′,3′-didehydro-2′,3′-dideoxy-5-methylcytidine, fluoro-2′,3′-dideoxycytidine, 3-(4-hydroxy-1′,2′-butadienyl)cytosine, 3′-azido-2′,3′-dideoxy-5-methylcytosine, 3′-azido-2′,3′-dideoxy-5-methylcytosine, 3′-azido-2′,3′-dideoxy-5-methylcytosine-N4-OH, 3′-azido-2′,3′-dideoxy-5-methylcytosine-N4Me, 3′-azido-2′,3′-dideoxycytosine, 3′-azido-2′,3′-dideoxy-5-fluorocytosine, 2′,3′-dideoxy-2′,3′-didehydrocytidine, beta-L-5-fluoro-2′,3′-dideoxy-2′,3′-didehydro-lamivudine, racivir, elvucitabine, apricitabine, emtricitabine, apricit abine, deoxyuridine, 5-Methyluridine, 3′-azido-2′,3′-dideoxy-5-chlorouridine, 3′-azido-2′,3′-dideoxy-5-ethyluridine, 3′-azido-2′,3′-dideox-yuridine, 3′-fluoro-2′,3′-dideoxy-5-bromouridine, 3′-fluoro-2′,3′-dideoxy-5-ethyluridine, 3′-azido-2′,3′-dideoxy-5-bromouridine, 3′-azido-2′,3′-dide-oxyuridine, 3′-fluoro-2′,3′-dideoxy-5-chlorouridine, 3′-fluoro-2′,3′-dideox-yuridine, 2′,3′-dideoxy-3′-azidouridine, 2,3′-dideoxy-3′-3′-fluoro-5-chlorouridine, deoxyadenosine, 2,3′-dideoxyadenosine, 2′,3′-dideoxy-2′-fluoro-ara-adenosine, 2-chiorodeoxyadenosine, 9-(4-hydroxy-1′,2′-butadienyl)adenine, 9-(2-phosphonomethoxyethyl) adenine, 2′,3′-didehydro-2′,3′-dideoxyadenosine, dideoxyadenosine, 5-methyl-2′,3′-dideoxyadenosine, 3′-fluoro-2′,3′-dideoxyarabinothranosyladenine, 3′-fluoro-2′,3′-dideoxyadenosine, 2,3′-dideoxy-2′,3′-didehydro-N6-(O-methylbenzyl)adenosine, 2′,3′-dideoxy-2′,3′-didehydro-N6-(2-methylpropyl)adenoma, 2′,3′-dideoxy-3′-fluo-roadenosine, 2,3′-dideoxyguanosine, 2′,3′-didehydroguanosine; 3′-azido-3′-deoxyguanosine, 3′-fluoro-2′,3′-dideoxy-guanosine, dideoxyguanosine, 3′-azideo-2′,3′-dideoxyguanosine, 3′-fluoro-2′,3′-dideoxyguanosine, 2′,3′-dideoxy-3′-azidoguanosine, 3′-deoxythy-midine; 2′,3′-dideoxythymidine, 2′,3′-didehydrothymidine, 3′-azido-3′-deoxythymidine; 3′-fluoro-3′-deoxythymidine, 3′-fluoro-2′,3′-dideoxythy-midine, 3′-deoxy-2′,3′-didehydrothymidine, 2′,3′-didehydro-2′,3′-dideoxythymidine, 2′,3′-dideoxyinosine, 2,6-diaminopurine-2′,3′-dideoxyriboside; 2,6-diaminopurine-3′-azido-2′,3′-dideoxyri-boside; 2,6-di-aminopurine-3′-fluoro-2′,3′-dideoxyriboside, 3-phosphonomethoxyethyl-2,6-diaminopurine, 2,6-di-aminopurine-2′,3′-dideoxyriboside, 3′-azido-2′,3′-dideoxy-diaminopurine, 3′-fluoro-2′,3′-dideoxy diaminopurine, 2′,3′-di-deoxy-3′-fluoro-2,6-diaminopurineriboside, abacavir, acyclovir aciclovir, adefovir, alovudine, amantadine, ampligen, amprenavir, arbidol, atazanavir, atripla, boceprevir, cidofovir, combivir, cytarabine, darunavir, delavirdine, didanosine, desciclovir, didanosine, disoproxil, docosanol, edoxudine, efavirenz, enfuvirtide, entecavir, entry inhibitors, elvucitabine, emtricitabine, famciclovir, fomivirsen, fosamprenavir, foscarnet, fosfonet, fusion inhibitor, ganciclovir, ibacitabine, imunovir, idoxuridine, imiquimod, indinavir, inosine, oseltamivir, penciclovir, peramivir, rimantadine, ribavirin, ritonavir, saquinavir, stavudine, tenofovir, tenofovir, fiacitabine, Fialuridine, doxuridine, Foscamet, Lobucavir, Sorivudine, trifluridine, tromantadine, ribavirine, stavudine, tipranavir, trizivir, truvada, valaciclovir, valganciclovir, vicriviroc, idarabine, viramidine, zalcitabine, zan amivir, zidovudine, synergistic enhancer, integrase inhibitor, interferon type III, interferon type II, interferon type I, interferon, lopinavir, loviride, maraviroc, moroxydine, methisazone, nelfinavir, nevirapine, nexavir, peginterferon alfa-2a, pleconaril, protease inhibitor, raltegravir, reverse transcriptase inhibitor.

The invention provides that a method for treating cancer, comprising: administration to a compound of the andrographolide, derivatives and analogs, a pharmaceutically acceptable salt or prodrug from thereof; a cancer is selected but is not limited from the multiple myeloma, leukemia, lymphoma, acute leukemia, chronic leukemia, acute lymphocytic leukemia, acute nonlym phocytic leukemia, chronic lymphocytic leukemia, chronic myelogenous leukemia, acute myeloid leukemia, hairy cell leukemia, Hodgkin's lymphoma, non-Hodgkin's lymphoma, multiple myeloma, hematologic cancer is of low, intermediate, or high grade, brain cancer, cancers of the head and neck, lung cancer, breast cancer, cancers of the reproductive system, cancers of the digestive system, pancreatic cancer, and cancers of the urinary system, cancer of the upper digestive tract or colorectal cancer, bladder cancer, renal cell carcinoma, prostate cancer, cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancers may be selected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bronchial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genitourinary organs; thyroid cancer or other endocrine cancer; and cutaneous T-cell lymphoma, both granulocytic and monocytic, adenocarcinoma, angiosarcoma, astrocytoma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendotheliosarcoma, medullary thyroid carcinoma, medulloblastoma, meningioma mesothelioma, myelomas, myxosarcoma neuroblastoma, neurofibrosarcoma, ohgodendroglioma, osteogenic sarcoma, epithelial ovarian cancer, papillary carcinoma, papillary adenocarcinomas, parathyroid tumors, pheochromocytoma, pinealoma, plasmacytomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, Wilm's tumor, ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pan creatic duct; bladder cancer such as a transitional cell carcinoma inurinary bladder, urothelial carcinomas, tumors in the urothelial cells that line the bladder, squamous cell carcinomas, adenocarcinomas, small cell cancers; myelodysplasia, myeloproliferative disorders; bone cancer; lung cancer such as non-small cell lung cancer, squamous cell carcinomas, adenocarcinomas, large cell undifferentiated carcinomas, small cell lung cancer; skin cancer, basal cell carcinoma, melanoma, squamous cell carcinoma actinic keratosis, eye retinoblastoma; cutaneous or intraocular melanoma; primary liver cancer; kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus, hepatitis C virus, and hepa tocellular carcinoma; human lymphotropic virus-type 1 and adult T-cell leukemia/lymphoma; and human papilloma virus and cervical cancer; central nervous system cancers, primary brain tumors, gliomas, Oligodendroglioma, Ependymoma, Meningioma, Lymphoma, Schannoma, Medulloblastoma; peripheral nervous system cancers, acoustic neuromas, malignant peripheral nerve sheath tumor including neurofi bromas and schwannomas, malignant fibrous cytoma, malig nant fibrous histiocytoma, malignant meningioma, malignant mesothelioma, and malignant mixed Müllerian tumor; oral cavity and oropharyngeal cancer such as, hypopharyngeal cancer, laryngeal cancer, nasopharyngeal cancer, and oropha ryngeal cancer; stomach cancer such as lymphomas, gastric stromal tumors, and carcinoid tumors; testicular cancer such as germ cell tumors, which include seminomas and nonseminomas, and gonadal stromal tumors, which include Leydig cell tumors and Sertoli cell tumors; thymus cancer such as to thymomas, thymic carcinomas, carcinoids or carcinoid tumors; rectal cancer; and colon cancer.

The invention provides that said compound of andrographolide derivatives and analogs is administered together with at least one known cancer, chemotherapeutic and immune agent selected but is not limited from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, podophyllotoxin, tamoxifen, doxazosin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoro-methyl ornithine, fenretinide, N-4-carboxyphenyl retinamide, genistein, ara-C, CB-64D, CB-184, ILX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, Roscovitine, Olonoucine, Valecoxib.

The invention provides that a compound of andrographolide derivatives and analogs is, independently at each occurrence, selected but is not limited from the example 1 to 440 and see as described in claim 9.

The invention provides that the administration of a compound of andrographolide derivatives and analogs may be by oral route, parenteral, subcutaneous, intravenous, intramuscular, intra-peritoneal, transdermal, buccal, intrathecal, intracranial, intranasal or topical routes.

The compositions of the invention are useful for inhibiting viral replication, the treatment of viral infections which are caused by DNA viruses, such as herpes simplex virus, the cytomegalovirus, papovavirus, the varicella zoster virus or Epstein-Barr virus; the treatment of infections which are caused by RNA viruses, such as togaviruses or retroviruses, the treatment of infections which are caused by HTLV-I and II, the treatment of infections which are caused by lentiviruses; In some embodiments, and the treatment of infections which are caused by HIV-1 and 2.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane and the like; bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, parabromophenylsulfonic acid, propionic acid, p-toluenesulfonic acid, salicylic acid, stearic acid, succinic acid, taimic acid, tartaric acid, thioglycolic acid, toluenesulfonic acid, uric acid, and the like; salts of polyprotic acids, such as sodium phosphate, disodium hydrogen phosphate, and sodium dihydrogen phosphate can also be used; when the base is a salt, the cation can be any pharmaceutically acceptable cation, such as ammonium, alkali metals, alkaline earth metals, and the like, example may include sodium, potassium, lithium, magnesium, calcium and ammonium; suitable acids are pharmaceutically acceptable organic or inorganic acids, examples of inorganic acids include hydrochloric acid, hydrobromic acid, hydriodic acid, sulfuric acid, nitric acid, boric acid, phosphoric acid, and the like.

### Chemical Synthesis

The following reaction schemes illustrate methods which may be employed for the synthesis of the compounds of structural formula I described in this invention. All substituents are as defined above unless indicated otherwise. Several strategies based upon synthetic transformations known in the literature of organic synthesis may be employed for the preparation of the title compounds of general formula I.

In Scheme 1, andrographolide in toluene was reacted by the reflux in the presence of Al₂O₃ catalyst to afford dehydration andrographolide, a key intermediate A. In Scheme II, compound B in THF was reacted with a reagent containing aldehyde group in the presence of base catalyst to afford compound C with the formation of double bond at C15 position. In Scheme III, compound D was reacted with an peroxide reagent to afford compound E by epoxidation at double bond(C8-C17) site position.

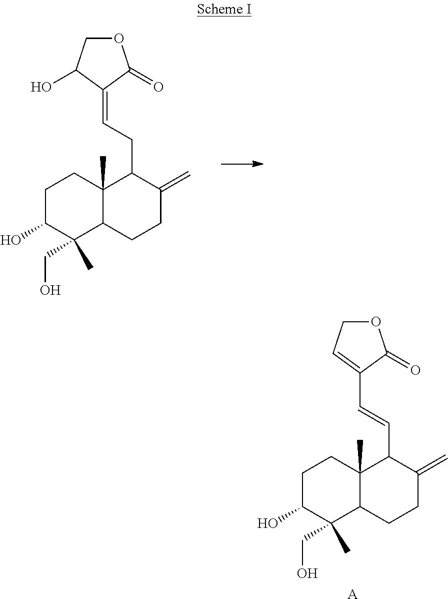

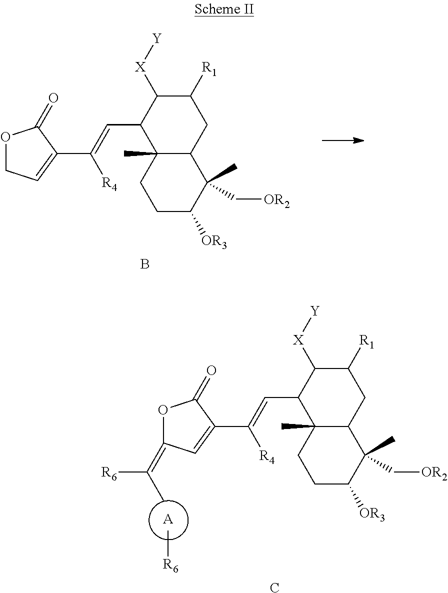

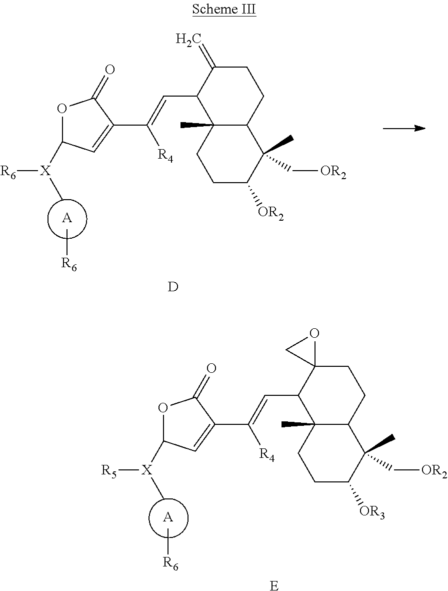

In Scheme IV, compound F was reacted with an nucleophilic reagent to afford compound G by formation of C-hetero bond at C12 positions. In Scheme V, compound H was reacted with an nucleophilic reagent to afford compound I by formation of C-hetero bond at C7 positions. In Scheme VI, compound J was reacted with acylchloride or halogenated reagent in the presence of DMAP and triethylamine to afford compound K by formation of ester or ether bond at C3 or C19 positions.

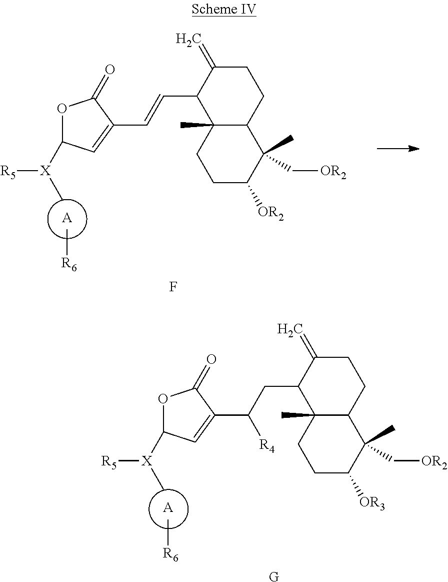

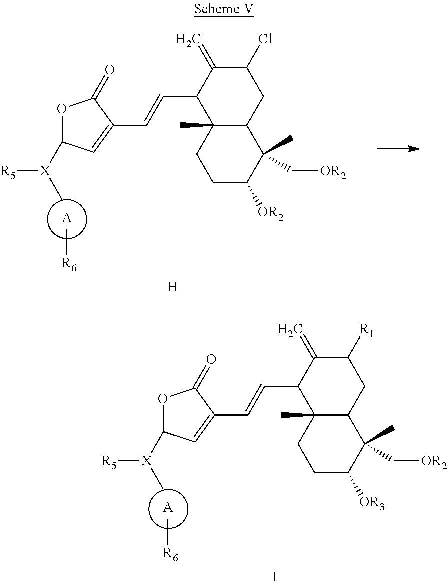

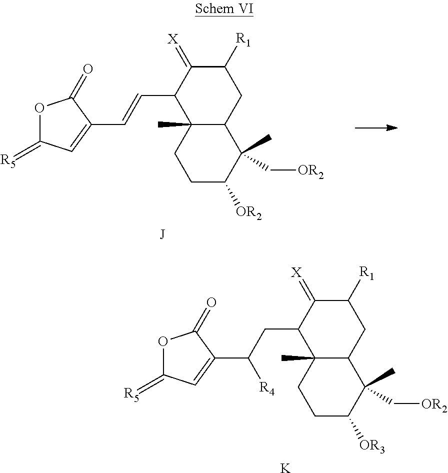

### Synthesis and Preparative Example

### Implementation of the Case and its Structure 1-436 Table 1

### Examples

The following examples illustrate the present invention. If no mentioned otherwise, the reactions take place at room temperature. Andrographolide was purchased from Huatai Biotechnology Co., China.

### General Method A (Hydroxy Esterification)

To a mixture of andrographolide 3.00 g (10 mmol) and DMAP 1.2 g (10 mmol), triethylamine 1.5 g (15 mmol) in 20 mL methylene chloride were added 4-O-glucosylbenzoyl chloride 4.8 g (15 mmol). The mixture was stirred until the reaction was complete. The reaction solution was filtered. The crude was separated by silica gel column chromatography to give the title compound.

### General Method B (Hydroxy Deprotection)

To a mixture of acetyled andrographolide 4.30 g (10 mmol) in EtOH 20 mL and H₂O 5 mL were added and K₂CO₃ 1.50 g. The mixture was refluxed for 2 h. The reaction solution was filtered. The crude was separated by silica gel column chromatography to give the title compound.

### General Method C (C═C Bond Formation at 15 Position)

To a mixture of acetyled andrographolide 2.1 g (6 mmol) in MeOH 20 mL were added 2-formyl furan 0.80 g (8.34 mmol) and Na₂CO₃ 0.50 g (4.72 mmol). The mixture was reacted for 4 h, 50° C. The reaction solution was extracted by EtOAC. The crude was separated by silica gel column chromatography to give the title compound.

### General Method D (C—C Bond Formation at 12 Position)

To a mixture of acetyled andrographolide 2.1 g (6 mmol) in MeOH 10 mL were added nitromethane 4 mL and NaOCH₃ 1.04 g (18.27 mmol). The mixture was reacted for 4 h, 50° C. The reaction solution was extracted by EtOAC. The crude was separated by silica gel column chromatography to give the title compound.

### General Method E (C-Hetero Bond Formation at 7 Position)

To a mixture of acetyled-7-chloroandrographolide 2.2 g (6 mmol) in THF 10 mL were added morpholine 4 mL and NaOCH₃ 5.0204 g (9.14 mmol). The mixture was reacted for 4 h, 50° C. The reaction solution was extracted by EtOAC. The crude was separated by silica gel column chromatography to give the title compound.

### Example 1

### Preparation of 11,12-dedihydro-14-deoxyandrographolide

To a mixture of andrographolide 3.00 g (8.6 mmol) in pyridine 20 mL were added Al₂O₃ 6.00 g (5.9 mmol). The mixture was refluxed for 5 h. The reaction solution was extracted by EtOAC. The crude was separated by silica gel column chromatography to give the title compound; IR(KBr,cm⁻):3295, 3081, 2969, 2934, 2851, 1744, 1637, 1451, 1389, 1350, 1273, 1086, 1026, 997, 890, 884 ¹H-NMR (600 MHz, DMSO-d₆): δ 7.65 (s, 1H), 6.74 (m, 1H), 6.12 (d, J=15.6 Hz, 1H), 5.02 (d, J=4.8 Hz, 1H), 4.89 (d, J=1.2 Hz, 2H), 4.73 (d, J=1.2 Hz, 1H), 4.42 (d, J=1.2 Hz, 1H), 4.13 (m, 1H), 3.85 (dd, J=3.0 Hz, J=3.0 Hz, 1H), 3.29 (m, 1H), 3.23 (m, 1H), 2.36 (d, J=10.8 Hz, 2H), 1.98 (m, 1H), 1.72 (dd, J=2.4 Hz, J=2.4 Hz, 1H), 1.41 (m, 1H), 1.33 (m, 1H), 1.19 (m, 1H), 1.14 (m, 1H), 1.09 (s, 3H), 0.76 (s, 3H).

### Example 2

### Preparation of 3,14,19-triacetylandrographolide

To a mixture of andrographolide 3.00 g (8.6 mmol) in acetic anhydride 20 mL were added zinc chloride 2.0 g (0.3 mmol). The mixture was reacted for 5 h, 80° C. The reaction solution was extracted by EtOAC. The crude was separated by silica gel column chromatography to give the title compound; IR(KBr, cm⁻¹):3436, 3078, 2983, 2935, 2872, 1753, 1736, 1728, 1642, 1440, 1376,1347, 1247, 1132, 1095, 1077; ¹H-NMR (300 MHz, CDCl₃): δ6.89 (t, 1H), 5.93 (d, J=6 Hz, 1H), 4.74 (br s, 2H), 4.56 (m, 2H), 4.33 (d, J=11.7 Hz, 1H), 4.25 (dd, J=1.8 Hz, J=1.8 Hz, 1H), 4.17 (d, J=11.7 Hz, 1H), 3.04 (d, J=6.3 Hz, 1H), 2.94 (d, J=7.8 Hz, 1H), 2.25 (s, 3H), 2.12 (s, 3H), 2.08 (s, 3H), 2.06 (s, 1H), 2.04 (s, 1H), 1.87 (d, J=2.4 Hz, 2H), 1.85 (d, J=3.6 Hz, 2H), 1.54 (d, J=3.9 Hz, 1H), 1.36 (dd, J=2.1 Hz, J=1.8 Hz, 2H), 1.18 (s, 3H), 1.08 (s, 3H).

### Example 3

### Preparation of 7-((2-((4H-imidazol-2-yl)amino)-1-oxopropan-2-yl)amino)-12-(2-(pyridine-2-yl)amino)-14-deoxy-(E)-15-(4-(dimethylamino)benzylidene)-8,17-epoxyandrographolide

Analogously to General Method C, the title compound was prepared from 7-((4H-imidazol-2-yl)amino)-1-oxopropan-2-yl)amino)-8-epoxyethanyl-12-(pyridin-2-yl)-amino-14-deoxyandrographolide and 2-(4-(dimethylamino)phenyl)-2-oxyacetic acid; ¹H NMR: δ 7.65 (s, 1H), 7.74 (m, 2H), 7.58 (d, J=9.0 Hz, 2H), 7.04 (m, 3H), 6.31 (m, 1H), 6.06 (d, J=15.6 Hz, 1H), 5.80 (s, 1H), 5.02 (s, 1H), 4.85 (d, J=1.8 Hz, 2H), 4.15 (m, 1H), 3.85 (m, 1H), 3.74 (m, 1H), 3.33 (m, 2H), 3.31 (m, 1H), 2.98 (s, 6H), 2.90 (m, 1H), 2.63 (d, J=3.0 Hz, 1H), 2.16 (d, J=10.2 Hz, 1H), 1.74 (m, 3H), 1.58 (m, 2H), 1.52 (dd, J=3.6 Hz, 4.2 Hz, 1H), 1.49 (m, 2H), 1.32 (m, 2H), 1.28 (s, 3H), 1.08 (s, 3H), 1.07 (m, 2H), 0.89 (s, 3H).

### Example 4

### Preparation of 3,19-diacetyl-11,12-dedihydro-14-deoxyandrographolide

To a mixture of 14-deoxy-11,12-dedihydroandrographolide 3.00 g (10.0 mmol) in CH₂Cl₂ 20 mL were added acetyl chloride 3.50 g (45.2 mmol) and triethylamine 3 mL. The mixture was reacted for 3 h, rt. The reaction solution was extracted by EtOAC. The crude was separated by silica gel column chromatography to give the title compound; IR(KBr, cm⁻¹):3436,3078, 2983, 2935, 2872, 2849, 1748, 1728, 1642, 1440, 1376, 1347, 1247, 1132, 1095, 1077, 1037,1000, 989, 892; ¹H-NMR (300 MHz, CDCl₃): δ7.00 (d, J=1.8 Hz, 1H), 6.69 (d, J=0.9 Hz, 1H), 6.16 (dd, J=1.2 Hz, 1H), 4.88 (s, 1H), 4.61 (m, 1H), 4.41 (t, J=13.0 Hz, 1H)4.12 (d, J=12.0 Hz, 1H), 2.49 (t, 1H), 2.41 (t, 1H), 2.05 (s, 6H), 1.87 (d, J=2.4 Hz, 2H), 1.85 (d, J=3.6 Hz, 2H), 1.54 (d, J=3.9 Hz, 1H), 1.36 (dd, J=2.1 Hz, J=1.8 Hz, 2H), 1.04 (s, 3H), 0.78 (s, 3H).

### Example 5

### Preparation of 11,12-dedihydro-14-deoxy-(E)-15-(4-hydroxy-3-metho-xy-5-nitrobenzylidene)andrographolide

Analogously to General Method C, the title compound was prepared from 14-deoxy-11,12-dedihydroandrographolide and 3-nitro-4-hydroxy-5-methoxybenzaldehyde; ¹H NMR: δ7.62 (s, 1H), 7.69 (s, 1H), 7.55 (s, 1H), 6.72 (m, 1H), 6.21 (s, 1H), 6.19 (s, 1H), 5.03 (br, 1H), 4.73 (m, 1H), 4.43 (m, 1H), 4.13 (br, 1H), 3.84 (d, J=9.6 Hz, 1H), 3.84 (s, 3H), 3.27 (m, 1H), 3.22 (m, 1H), 2.38 (t, J=10.8 Hz, 2H), 1.97 (m, 1H), 1.72 (d, J=13.2 Hz, 1H), 1.58 (m, 2H), 1.40 (d, J=4.2 Hz, 3.6 Hz, 1H), 1.34 (d, J=13.2 Hz, 1H), 1.17 (m, 2H), 1.08 (s, 3H), 0.77 (s, 3H).

### Example 6

### Preparation of (E)-2-(1,2-dihydroxyethyl)-4-(6-hydroxy-5-(hydroxyl-methyl)-5,8a-dimethyl-2-methylenedecahydronaphthalen-1-yl)but-2-enehydrazide

To a mixture of andrographolide 3.00 g (8.6 mmol) in THF 20 mL were added hydrazine 2 mL (40%). The mixture was reacted for 2 h, rt. The reaction solution was extracted by EtOAC. The crude was separated by silica gel column chromatography to give the title compound; IR(KBr, cm⁻¹): 3398-3325, 3092, 2978, 2957, 2849, 1727, 1674, 1649, 1456, 1366, 1221, 1074.

### Example 7

### Preparation of 7-((2-((4H-imidazol-2-yl)amino)-1-oxopropan-2-yl)amino)-11,12-dedihydro-14-deoxy-(Z)-15-(1-(4-(dimethylamino)phenyl)-2-ethoxy-2-oxoethylid-ene)-andrographolide

Analogously to General Method C, the title compound was prepared from 7-((2-((4-1H-imidazol-2-yl)amino)-1-oxopropan-2-yl)amino)-11,12-dedihydro-14-deoxyandrographolide and 2-(4-(dimethylamino)phenyl)-2-oxyacetic acid; ¹H NMR: δ11.0 (s, 1H), 7.61 (s, 1H), 7.21 (d, J=9.0 Hz, 2H), 7.02 (d, J=7.0 Hz, 2H), 6.75 (d, J=9.0 Hz, 2H), 6.31 (m, 1H), 6.24 (s, 1H), 6.16 (d, J=15.6 Hz, 1H), 5.20 (m, 2H), 5.05 (s, 1H), 4.73 (m, 1H), 4.43 (m, 1H), 4.15 (m, 1H), 3.84 (d, J=10.8 Hz, 1H), 3.74 (m, 3H), 3.29 (m, 1H), 3.27-3.22 (m, 2H), 3.06 (s, 6H), 2.42-2.35 (m, 2H), 1.60-1.56 (m, 2H), 1.39 (q, J=4.2 Hz, 1H), 1.33 (d, J=13.8 Hz, 1H), 1.27 (s, 3H), 1.08 (s, 3H), 0.77 (s, 3H).

### Example 8

### Preparation of (E)-4-hydroxy-3-(2-(3,3,6a,10b-tetramethyl-8-methylenedecahydro-1H-naphtho[2,1-d][1,3]dioxin-7-yl)ethylidene)dihydrofuran-2(3H)-one

To a mixture of andrographolide 3.00 g (10.0 mmol) in benzene 300 mL and DMSO 40 mL were added p-toluenesulfonic acid 0.7 g (4.07 mmol) and 2,2-dimethoxypropane 40 g (384.61 mmol). The mixture was reacted for 12 h, 80° C. The reaction solution was extracted by EtOAC. The crude was separated by silica gel column chromatography to give the title compound; IR(KBr,cm⁻¹):3351-3076, 2970, 1760, 1640, 1199, 1077, 1047, 1034, 913; ¹H NMR (600 MHz, DMSO-d₆): δ6.60 (m, 1H), 5.72 (d, J=6.0 Hz, 1H), 5.06 (d, J=4.8 Hz, 1H), 4.89 (d, J=12 Hz, 2H), 4.73 (d, J=1.2 Hz, 1H), 4.60 (m, 1H), 4.42 (d, J=1.2 Hz, 1H), 4.37 (m, 1H), 4.13 (m, 1H), 3.85 (d, J=3.0 Hz, 1H), 3.29 (m, 1H), 3.23 (m, 1H), 2.36 (d, J=10.8 Hz, 2H), 1.98 (m, 1H), 1.72 (d, J=2.4 Hz, 1H), 1.54 (s, 3H), 1.53 (s, 3H), 1.41 (m, 1H), 1.33 (m, 1H), 1.19 (m, 1H), 1.14 (m, 1H), 1.12 (s, 3H), 1.01 (s, 3H).

### Example 9

### Preparation of 7-((2-((4H-imidazol-2-yl)amino)-1-oxopropan-2-yl)amino)-11,12-dedihydro-14-deoxy-8,17-epoxyandrographolide

Analogously to General Method E, the title compound was prepared from 8-epoxyethanyl-11,12-dedihydro-14-deoxyandrographolide and 2-((4H-imidazol-2-yl)amino)-1-oxopropan-2-amine; ¹H NMR: δ7.65 (m, 1H), 7.04 (m, 3H), 6.31 (m, 1H), 6.06 (d, J=15.6 Hz, 1H), 5.02 (s, 1H), 4.89 (d, J=12 Hz, 2H), 4.15 (m, 1H), 3.96 (br, 1H), 3.74 (m, 1H), 3.33 (m, 2H), 3.31 (d, J=10.2 Hz, 1H), 2.90 (m, 1H), 2.63 (d, J=3.0 Hz, 1H), 3.86 (s, 3H), 2.16 (d, J=10.2 Hz, 1H), 1.74 (m, 2H), 1.52 (dd, J=3.6 Hz, 4.2 Hz, 1H), 1.43-1.30 (m, 2H), 1.32 (m, 2H), 1.08 (s, 3H), 1.07 (m, 2H), 0.89 (s, 3H).

### Example 10

### Preparation of (E)-3-(2-(3,3,6a,10b-tetramethyl-8-methylene-decahydro-1H-naphtho[2,1-d][1,3]dioxin-7-yl)vinyl)furan-2(5H)one

To a mixture of 14-deoxy-11,12-dedihydroandrographolide 3.00 g (10.0 mmol) in benzene 300 mL and DMSO 40 mL were added p-toluenesulfonic acid 0.7 g (4.07 mmol) and 2,2-dimethoxypropane 4.0 g (38.4 mmol). The mixture was reacted for 7 h, 80° C. The reaction solution was extracted by EtOAC. The crude was separated by silica gel column chromatography to give the title compound; IR(KBr,cm⁻¹): 3068-2996, 1760, 1637, 1248, 1093, 1076,1042; ¹H NMR: δ7.12 (m, 1H), 6.91 (m, 1H), 6.12 (d, J=15.6 Hz, 1H), 4.80 (d, J=12 Hz, 2H), 4.73 (d, J=1.2 Hz, 1H), 4.55 (m, 1H), 4.09 (d, J=12 Hz, 1H), 3.47 (d, J=3.0 Hz, 1H), 3.24 (m, 1H), 2.40 (m, 2H), 2.05 (m, 2H), 1.74 (m, 2H), 1.53 (m, 1H), 1.44 (s, 3H), 1.38 (s, 3H), 1.35 (m, 2H), 1.28 (m, 1H), 1.22 (m, 2H), 1.13 (m, 1H), 1.01 (s, 3H).

### Example 11

### Preparation of 12-nitromethyl-14-deoxyandrographolide

Analogously to method D, the title compound was prepared from andrographolide and nitromethane; IR(KBr, cm⁻¹): 3418-3018, 2927, 1770, 1643, 1556, 1260, 1165, 1082, 1036; ¹H NMR: δ7.65 (m, 1H), 4.87 (m, 2H), 4.82 (m, 2H), 4.75 (m, 2H), 4.58 (m, 1H), 3.78 (d, J=10.8 Hz, 1H), 3.22 (m, 1H), 3.19 (m, 1H), 3.17 (t, J=6 Hz, 1H), 2.29 (d, J=12.6 Hz, 1H), 1.75 (m, 3H), 1.57 (m, 4H), 1.39 (d, J=9.6 Hz, 1H), 1.28 (m, 1H), 1.06 (d, J=1.8 Hz, 1H), 1.03 (s, 3H), 0.56 (s, 3H).

### Example 12

### Preparation of 7-((2-((4H-imidazol-2-yl)amino)-1-oxopropan-2-yl)amino)-11,12-dedihydro-14-deoxy-(E)-15-((2-(4-methylpiperazin-1-yl)pyrimidin-5-yl)methylene)-andrographolide

Analogously to method C, the title compound was prepared from 7-((2-((4H-imidazol-2-yl)amino)-1-oxopropan-2-yl)amino)-11,12-dedihydro-14-deoxyandrographolide and 2-(4-methylpiperazin-1-yl)pyrimidine-5-carbaldehyde; ¹H NMR: δ8.05 (s, 2H), 7.61 (s, 1H), 7.02 (d, J=7.0 Hz, 2H), 6.31 (m, 1H), 6.24 (s, 1H), 6.16 (d, J=15.6 Hz, 1H), 5.20 (m, 2H), 5.05 (s, 1H), 4.73 (m, 1H), 4.43 (m, 1H), 4.15 (m, 1H), 3.84 (d, J=10.8 Hz, 1H), 3.74 (m, 3H), 3.29 (m, 1H), 3.27-3.22 (m, 2H), 3.21 (m, 4H), 2.50 (m, 4H), 2.42-2.35 (m, 2H), 2.27 (s, 3H), 1.60-1.56 (m, 2H), 1.39 (q, J=4.2 Hz, 1H), 1.33 (d, J=13.8 Hz, 1H), 1.27 (s, 3H), 1.08 (s, 3H), 0.77 (s, 3H).

### Example 13 Preparation of 11,12-dedihydro-14-deoxy-8,17-epoxyandrographolide

To a mixture of 14-deoxy-11,12-dedihydroandrographolide 1.50 g (5.0 mmol) in CHCl₃ 30 mL was added 3-chloroperoxy-benzoic acid 15 mL and 2,2-dimethoxypropane 2.0 g (19.2 mmol). The mixture was reacted for 3 h, rt. The reaction solution was extracted by EtOAC. The crude was separated by silica gel column chromatography to give the title compound; IR(KBr,cm⁻¹):3369-3079, 2969, 1753, 1350, 1273, 1086; ¹H NMR: δ7.61 (m, 1H), 6.30 (m, 1H), 6.06 (d, J=15.6 Hz, 1H), 5.73 (s, 1H), 5.02 (br, 1H), 4.85 (d, J=1.8 Hz, 2H), 4.15 (m, 1H), 3.85 (m, 1H), 3.33 (m, 2H), 3.31 (d, J=10.2 Hz, 1H), 2.63 (d, J=3.0 Hz, 1H), 2.16 (d, J=10.2 Hz, 1H), 1.74 (m, 2H), 1.58 (m, 2H), 1.52 (dd, J=3.6 Hz, 4.2 Hz, 1H), 1.32 (m, 2H), 1.08 (s, 3H), 1.07 (m, 1H), 0.89 (s, 3H).

### Example 14

### Preparation of 3,19-diacetyl-7-oxo-11,12-dedihydro-14-deoxyandrographolide

To a mixture of 3,19-diacetyl-14-deoxy-11,12-dedihydroandrographolide 2.0 g (4.81 mmol) in CH₂Cl₂ 20 mL was added tertbutyl hydroperoxide 0.87 g (9.67 mmol) SeO₂ 0.53 g (4.81 mmol), 3-chloroperoxy-benzoic acid 15 mL and 2,2-dimethoxypropane 2.0 g (19.2 mmol). The mixture was reacted for 3 h, rt. The reaction solution was extracted by EtOAC. The crude was separated by silica gel column chromatography to give the title compound; IR(KBr,cm⁻¹):3469-3080, 2947, 2876, 1756, 1732, 1649, 1443, 1374, 1245, 1083, 1034, 990, 899; ¹H NMR (300 MHz, CDCl₃): δ7.00 (m, 1H), 6.65 (m, 1H)6.16 (d, J=12 Hz, 1H), 4.88 (m, 1H), 4.61 (m, 1H), 4.45 (m, 1H), 4.38 (d, J=11.7 Hz, 1H), 4.41 (t, J=13.0 Hz, 1H), 4.12 (d, J=12.0 Hz, 1H), 2.45 (m, 3H), 2.05 (s, 6H), 1.87 (m, 2H), 1.85 (m, 2H), 1.79 (m, 1H), 1.54 (d, J=3.9 Hz, 1H), 1.36 (m, 2H), 1.04 (s, 3H), 0.78 (s, 3H).

### Example 15

### Preparation of 7-((2-((4H-imidazol-2-yl)amino)-1-oxopropan-2-yl)amino)-12-(2-(pyridine-2-yl)amino)-14-deoxy-(E)-15-((2-(4-methylpiperazin-1-yl)pyrimidin-5-yl)methylene)-8,17-epoxyandrographolide

Analogously to method C, the title compound was prepared from 7-((2-((4H-imidazol-2-yl)amino)-1-oxopropan-2-yl)amino-8-epoxyethanyl-12-((pyridin-2-yl)amino)-14-deoxyandrographolide and 2-(4-methylpiperazin-1-yl)pyrimidine-5-carbaldehyde; ¹H NMR: 8.07 (m, 3H), 7.74 (m, 2H), 7.02 (m, 3H), 6.75 (m, 2H), 6.22 (s, 1H), 4.73 (m, 1H), 4.43 (m, 1H), 4.15 (m, 1H), 3.84 (d, J=10.8 Hz, 1H), 3.74 (m, 2H), 2.41 (m, 1H), 3.27-3.22 (m, 7H), 2.52-2.35 (m, 8H), 2.20 (s, 3H), 1.60-1.50 (m, 4H), 1.39 (q, J=4.2 Hz, 1H), 1.33 (d, J=13.8 Hz, 1H), 1.27 (s, 3H), 1.17 (m, 2H), 1.08 (s, 3H), 0.77 (s, 3H).

### Example 16

### Preparation of 7-hydroxyl-11,12-dedihydro-14-deoxyandrographolide

Analogously to method E, the title compound was prepared from 14-deoxy-11,12-dedihydro-andrographolide; IR(KBr,cm⁻¹):3443-3080, 2947, 1753, 1649, 1443, 1374, 1245, 1083, 1034.

### Example 17

### Preparation of (E)-4-hydroxy-3-(2-(9-hydroxy-3,3,6a,10b-tetramethyl-8-methylenedecahydro-1H-naphtho[2,1-d][1,3]dioxin-7-yl)ethylidene)dihydrofuran-2(3H)-one

Analogously to method E, the title compound was prepared from (E)-4-hydroxy-3-(2-(3,3,6-a,10b-tetramethyl-8-methylenedecahydro-1H-naphtho[2,1-d][1,3]dioxin-7-yl)ethylidene)dihydrofuran-2(3H)-one; IR(KBr,cm⁻): 3346-3079, 2972, 2931, 2868, 1753, 1640, 1440, 1383, 1350, 1199, 1076, 1047, 1021, 913.

### Example 18

### Preparation of (E)-4-hydroxy-3-(2-(3,3,6a,10b tetramethyldecahy drospiro[naphtho[2,1-d][1,3]dioxine-8,2′-oxiran]-7-yl)ethylidene)dihydrofuran-2(3H)-one

To a mixture of (E)-4-hydroxy-3-(2-(3,3,6a,10b-tetramethyl-8-methylenedecahydro-1H-naphtho[2,1-d][1,3]dioxin-7-yl)ethylidene)dihydrofuran-2(3H)-one, 1.50 g (3.85 mmol) in CHCl₃ 20 mL was added 3-chloroperoxybenzoic acid 0.8 g (4.64 mmol). The mixture was reacted for 3 h, rt. The reaction solution was extracted by EtOAC. The crude was separated by silica gel columnchromatography to give the title compound; IR(KBr,cm⁻): 3369-3079, 2972, 2931, 2855, 1760, 1640, 1440, 1384, 1350, 1220, 1202, 1199, 1077, 1047, 1034, 913.

### Example 19

### Preparation of 3,19-diacetyl-7-chloro-11,12-dedihydro-14-deoxyandrographolide

To a mixture of 3,19-diacetyl-14-deoxy-11,12-dedihydroandrographolide 4 g (9.26 mmol) in CHCl₃ 40 mL and pyridine 3.7 g (46.85 mmol) was added thionylchloride 5.5 g (46.22 mmol). The mixture was reacted for 3 h, rt. The reaction solution was extracted b EtOAC. The crude was separated by silica gel column chromatography to give the title compound; IR(KBr,cm⁻):3436-3078, 2983, 1748, 1728, 1642, 1220, 1132, 1095, 1077; ¹H NMR: δ7.54 (m, 1H), 6.74 (dd, J=10.0 Hz, 15.8 Hz, 1H), 6.11 (d, J=15.8 Hz, 1H), 5.05 (d, J=1.5 Hz, 1H), 4.93 (d, J=1.5 Hz, 1H), 4.22 (d, J=11.1 Hz, 1H), 3.50 (dd, J=11.4 Hz, J=4.7 Hz, 1H), 3.35 (d, J=11.0 Hz, 1H), 2.47 (m, 1H), 2.33 (d, J=9.8 Hz, 1H), 2.05 (m, 1H), 2.01 (s, 3H), 1.93 (s, 3H), 1.82 (m, 1H), 1.78 (m, 2H), 1.53 (m, 3H), 1.34 (m, 1H), 1.26 (s, 3H), 1.20 (m, 1H), 0.82 (s, 3H).

### Example 20

### Preparation of 7-((2-((4H-imidazol-2-yl)amino)-1-oxopropan-2-yl)amino)-11,12-dedihydro-14-deoxy-15-(4-(dimethylamino)benzylidene)andrographolide

Analogously to General Method C, the title compound was prepared from 7-((2-((4H-imidazol-2-yl)amino)-1-oxopropan-2-ylamino)-14-deoxy-11,12-dedihydroandrographolide and 4-(dimethylamino)benzaldehyde; ¹H NMR: δ7.61 (s, 1H), 7.58 (d, J=9.0 Hz, 2H), 7.02 (d, J=7.0 Hz, 2H), 6.75 (d, J=9.0 Hz, 2H), 6.31 (m, 1H), 6.24 (s, 1H), 6.16 (d, J=15.6 Hz, 1H), 5.20 (m, 2H), 5.05 (s, 1H), 4.73 (m, 1H), 4.43 (m, 1H), 4.15 (m, 1H), 3.84 (d, J=10.8 Hz, 1H), 3.74 (m, 3H), 3.29 (m, 1H), 3.27-3.22 (m, 2H), 3.06 (s, 6H), 2.42-2.35 (m, 2H), 1.60-1.56 (m, 2H), 1.39 (q, J=4.2 Hz, 1H), 1.33 (d, J=13.8 Hz, 1H), 1.27 (s, 3H), 1.08 (s, 3H), 0.77 (s, 3H).

### Example 21

### Preparation of 3,19-diacetyl-7-oxo-11,12-dedihydro-14-deoxyandrographolide

To a mixture of 3,19-diacetyl-14-deoxy-11,12-dedihydroandrographolide 1.50 g (3.9 mmol) in DMF 15 mL was added PDC 0.75 g (3.47 mmol). The mixture was reacted for 3 h, 60° C. The reaction solution was extracted by EtOAC. The crude was separated by silica gel column chromatography to give the title compound; IR(KBr,cm⁻¹):3453-2944, 1755, 1736, 1688, 1247, 1083, 1040; ¹H NMR: δ6.88 (m, 1H), 6.58 (m, 1H), 6.18 (d, J=15.6, 1H), 4.81 (m, 2H), 4.62 (dd, J=4.8 Hz, 4.2 Hz, 1H), 4.43 (d, J=12.0 Hz, 1H), 4.29 (d, J=12.0 Hz, 1H), 2.80 (dd, J=1.8 Hz, 3.0 Hz, 1H), 2.54 (m, 2H), 2.09 (s, 3H), 2.06 (s, 3H), 1.87 (m, 1H), 1.72 (m, 3H), 1.49 (m, 1H), 1.36 (m, 1H), 1.25 (m, 1H), 1.04 (s, 3H), 0.91 (s, 3H).

### Example 22

### Preparation of 3,19-diacetyl-7-(4-morpholino)-11,12-dedihydro-14-deoxyandrographolide

Analogously to General Method E, the title compound was prepared from 3,19-diacetyl-7-chloro-14-deoxy-11,12-dedihydroandrographolide and morpholine; IR(KBr,cm⁻):3430-3078, 1748, 1728, 1642, 1347, 1272, 1222, 1132, 1083, 1036; ¹H NMR: δ 7.65 (m, 1H), 7.59 (m, 1H), 7.09 (s, 1H), 5.12 (d, J=15.6 Hz, 1H), 5.02 (d, J=4.8 Hz, 1H), 4.42 (m, 1H), 3.67 (m, 4H), 3.29 (m, 1H), 3.23 (m, 1H), 2.86 (m, 4H), 2.36 (d, J=10.8 Hz, 2H), 3.09 (m, 3H), 2.06 (s, 6H), 1.98 (m, 1H), 1.72 (d, J=2.4 Hz, 1H), 1.41 (m, 2H), 1.33 (m, 1H), 1.14 (m, 1H), 1.09 (s, 3H), 0.76 (s, 3H).

### Example 23

### Preparation of 7-((2-((4H-imidazol-2-yl)amino)-1-oxopropan-2-yl)amino)-11,12-dedihydro-14-deoxy-(E)-15-((2-(4-methylpiperazin-1-yl)pyrimidin-5-yl)methylene)-8,17-epoxyandrographolide

Analogously to General Method C, the title compound was prepared from 7-((2-((4H-imidazol-2-yl)amino)-1-oxopropan-2-yl)amino)-8-epoxyethanyl-14-deoxyandrographolide and 4-(methylpyrazinyl)benzaldehyde; ¹H NMR: 8.07 (s, 2H), 7.64 (s, 1H), 7.02 (s, 2H), 6.75 (m, 1H), 6.22 (s, 1H), 6.16 (d, J=15.6 Hz, 1H), 4.73 (s, 1H), 4.43 (m, 1H), 4.15 (m, 1H), 3.84 (d, J=10.8 Hz, 1H), 3.74 (m, 3H), 2.41 (m, 1H), 3.27-3.22 (m, 6H), 2.52-2.35 (m, 8H), 2.20 (s, 3H), 1.60-1.56 (m, 2H), 1.39 (q, J=4.2 Hz, 1H), 1.33 (d, J=13.8 Hz, 1H), 1.27 (s, 3H), 1.17 (m, 2H), 1.08 (s, 3H), 0.77 (s, 3H).

### Example 24

### Preparation of 11,12-dedihydro-14-deoxy-(E)-15-(4-(dimethylamino)benzylidene)-8,17-epoxyandrographolide

Analogously to General Method C, the title compound was prepared from 14-deoxy-11,12-dedihydroandrographolide and 2-(4-(dimethylamino)phenyl)-2-oxyacetic acid; IR(KBr,cm⁻¹): 3430-3087, 2931, 1744, 1640, 1599, 1559, 1525, 1445, 1384, 1367, 1311, 1187, 1167, 1128, 1082, 1038; ¹H NMR: δ7.61 (s, 1H), 7.58 (d, J=9.0 Hz, 2H), 6.75 (d, J=9.0 Hz, 2H), 6.31 (m, 1H), 6.24 (s, 1H), 6.16 (d, J=15.6 Hz, 1H), 5.01 (d, J=4.8 Hz, 1H), 4.14 (q, J=2.4 Hz, 1H), 3.85 (d, J=2.4 Hz, 1H), 3.21 (m, 1H), 2.98 (s, 6H), 2.97 (s, 1H), 2.67 (d, J=2.4 Hz, 1H), 2.51 (d, J=4.2 Hz, 1H), 2.21 (d, J=9.6 Hz, 1H), 1.78 (m, 2H), 1.62 (m, 2H), 1.33 (m, 2H), 1.09 (s, 3H), 0.91 (s, 3H).

### Example 25

### Preparation of 12-(4-morpholino)-14-deoxyandrographolide

Analogously to General Method C, the title compound was prepared from andrographolide and morpholine; IR(KBr,cm⁻¹): 3399-2926, 1755, 1711, 1635, 1607, 1509, 1448, 1385, 1273, 1247,1171, 1115, 1077, 1038; ¹H NMR: δ7.67 (m, 1H), 4.84 (s, 2H), 4.43 (d, J=12.0 Hz, 1H), 4.37 (d, J=11.4 Hz, 1H), 3.94 (m, 1H), 3.75 (m, 1H), 3.45 (m, 4H), 3.32 (m, 4H), 3.21 (m, 1H), 3.16 (d, J=3.0 Hz, 2H), 2.86 (t, J=9.6 Hz, 4H), 2.23 (m, 1H), 2.05 (m, 2H), 1.71-1.52 (m, 4H), 1.32 (m, 2H), 1.21 (s, 3H), 0.62 (s, 3H).

### Example 26

### Preparation of (E)-5-oxo-4-(2-(3,3,6a,10b-tetramethyl-8-methylene-decahydro-1H-naphtho[2,1-d][1,3]dioxin-7-yl)ethylidene)tetrahydrofuran-3-yl methanesulfonate

To a mixture of (E)-4-hydroxy-3-(2-(3,3,6a,10b-tetramethyl-8-methylenedecahydro-1H-naphtho[2,1-d][1,3]dioxin-7-yl)ethylidene)dihydrofuran-2(3H)-one 1.50 g (3.85 mmol) in CHCl₃20 mL was added methanesulfonyl chloride 0.60 g (5.24 mmol) and triethylamine 1.30 g (12.82 mmol). The mixture was reacted for 2 h, rt. The reaction solution was used for next reaction.

### Example 27

### Preparation of 12-((5-amino-4H-1,2,4-triazol-3-yl)thio)-14-deoxy-andrographolide

Analogously to General Method E, the title compound was prepared from andrographolide and 3-amino-5-thio-1,2,4-triazole; IR(KBr,cm⁻¹): 3432-3362, 3079, 2947, 2927, 2875, 1753, 1643, 1593, 1497, 1452, 1383, 1260, 1165, 1082, 1036; 1H NMR: ¹H NMR (600M Hz, DMSO-d₆): δ 6.54 (m, 1H), 5.71 (m, 1H), 5.05 (br, 1H), 4.83 (s, 1H), 4.62 (m, 1H), 4.41 (q, J=4.2 Hz, 1H), 4.06 (d, J=8.4 Hz, 1H), 3.85 (d, J=5.4 Hz, 1H), 3.26 (m, 2H), 2.50 (m, 2H), 2.34 (m, 1H), 1.94 (m, 1H), 1.76-1.63 (m, 4H), 1.34 (m, 1H), 1.23 (m, 2H), 1.09 (s, 3H), 0.66 (s, 3H).

### Example 28

### Preparation of 12-((2-((4H-imidazol-2-yl)amino)-1-oxopropan-2-yl)amino))-14-deoxy-15-(4-(dimethylamino)benzylidene)-8,17-epoxyandrographolide

Analogously to General Method C, the title compound was prepared from 8-epoxyethanyl-12-((2-((4H-imidazol-2-yl)amino)-1-oxopropan-2-yl)amino))-14-deoxyandrographolide and 4-dimethylaminobenzaldehyde; ¹H NMR: δ7.61 (s, 1H), 7.58 (d, J=9.0 Hz, 2H), 7.03 (d, J=7.0 Hz, 2H), 6.75 (d, J=9.0 Hz, 2H), 5.01 (s, 1H), 4.14 (q, J=2.4 Hz, 1H), 3.85 (d, J=2.4 Hz, 1H), 3.21 (m, 1H), 3.75 (m, 4H), 2.98 (s, 6H), 2.97 (s, 1H), 2.67 (m, 3H), 2.51 (d, J=4.2 Hz, 1H), 2.21 (m, 3H), 1.78 (m, 3H), 1.62 (m, 2H), 1.33 (m, 5H), 1.09 (s, 3H), 0.91 (s, 3H).

### Example 29

### Preparation of 12-((4,6-dimethylpyrimidin-2-yl)thio)-14-deoxyandrographolide

Analogously to General Method E, the title compound was prepared from andrographolide and 2,4-dimethy-6-thiopyrimidin; IR(KBr,cm⁻¹):3436-3080, 2927, 2857, 1754, 1644, 1603, 1514, 1452, 1384, 1260, 1173, 1151, 1079, 1038, 967, 919; ¹H NMR: δ7.56 (m, 1H), 6.96 (m, 1H), 5.02 (m, 1H), 4.98 (m, 1H), 4.90 (m, 1H), 4.61 (m, 1H), 4.46 (m, 1H), 4.26 (m, 1H), 4.04 (d, J=11.2 Hz, 1H), 3.48 (m, 1H), 3.41 (d, J=11.2 Hz, 1H), 2.57 (m, 1H), 2.42 (m, 1H), 2.27 (m, 1H), 1.88-1.78 (m, 3H), 1.65-1.75 (m, 3H), 1.37 (s, 3H), 1.35 (m, 1H), 1.29 (s, 3H), 1.28 (s, 3H), 1.25-1.18 (m, 1H), 0.87 (m, 1H), 0.81 (s, 3H).

### Example 30

### Preparation of 7-(4-morpholino)-12-((2-((4H-imidazol-2-yl)amino)-1-oxopropan-2-yl)amino)-14-deoxyandrographolide

Analogously to General Method D, the title compound was prepared from 7-morpholino-8-epoxyethanyl-14-deoxy-11,12-dedihydroandrographolide and (2((4H-imidazol-2-yl)amino)-1-oxopropan-2-yl)amino)2-amine; ¹H NMR: δ7.65 (m, 1H), 7.59 (m, 1H), 7.09 (s, 1H), 7.02 (d, J=7.0 Hz, 2H), 5.12 (d, J=15.6 Hz, 1H), 5.02 (d, J=4.8 Hz, 1H), 4.42 (m, 1H), 3.74 (m, 1H), 3.67 (m, 5H), 3.29 (m, 2H), 3.23 (m, 3H), 2.86 (m, 4H), 2.36 (d, J=10.8 Hz, 2H), 3.09 (s, 3H), 1.98 (m, 1H), 1.72 (q, J=2.4 Hz, 1H), 1.41 (m, 3H), 1.33 (m, 1H), 1.28 (s, 3H), 1.14 (m, 1H), 1.09 (s, 3H), 0.76 (s, 3H).

### Example 31

### Preparation of 12-((2-aminophenyl)thio)andrographolide

Analogously to General Method E, the title compound was prepared from andrographolide and 2-aminothio-phenol; IR(KBr,cm⁻¹):3436-3362, 3076, 2926, 2854, 1749, 1642, 1609, 1478, 1446, 1384, 1201, 1080, 1036; ¹H NMR: δ8.24 (m, 2H), 7.86 (m, 1H), 7.70 (t, J=8.1 Hz, 1H), 7.55 (s, 1H), 6.0 (s, 1H), 4.83 (m, 3H), 4.66 (m, 1H), 4.31 (m, 1H), 4.23 (d, J=11.4 Hz, 1H), 3.63 (d, J=12 Hz, 1H), 3.49 (d, J=11.4 Hz, 1H), 3.44 (m, 2H), 2.49 (m, 1H), 2.36 (m, 1H), 1.96-1.85 (m, 3H), 1.78-1.69 (m, 2H), 1.63 (m, 1H), 1.57 (d, J=9.6 Hz, 1H), 1.35 (m, 3H), 1.28-1.08 (m, 4H), 0.75 (s, 3H).

### Example 32

### Preparation of 11,12-dedihydro-14-deoxy-15-(propan-2-ylidene) andrographolide

Analogously to General Method C, the title compound was prepared from 14-deoxy-11,12-dedihydroandrographolide and acetone; IR(KBr,cm⁻¹):3337-3080, 2928, 2855, 1746, 1642,1445, 1175, 1073, 1034; ¹H NMR: δ7.26 (m, 1H), 6.83 (dd, J=10.0 Hz, 15.8 Hz, 1H), 6.15 (d, J=15.8 Hz, 1H), 4.78 (d, J=1.5 Hz, 1H), 4.54 (d, J=1.48 Hz, 1H), 4.22 (d, J=11.1 Hz, 1H), 3.50 (dd, J=11.4 Hz, 4.7 Hz, 1H), 3.35 (d, J=11.0 Hz, 1H), 2.47 (m, 1H), 2.33 (d, J=9.8 Hz, 1H), 2.05 (m, 1H), 2.01 (s, 3H), 1.93 (s, 3H), 1.82 (m, 1H)1.78 (m, 2H), 1.53 (m, 1H), 1.34 (m, 1H), 1.26 (s, 3H), 1.25 (m, 1H), 1.20 (m, 1H), 0.82 (s, 3H).

### Example 33

### Preparation of 11,12-dedihydro-14-deoxy-(E)-15-(1,3-dihydroxypropan-2-ylidene)andrographolide

Analogously to General Method C, the title compound was prepared from 14-deoxy-11,12-dedihydroandrographolide and paracarboxaldehyde; IR(KBr,cm⁻¹): 3394-2934, 2873, 1753, 1654, 1451, 1388, 1106, 1080, 1035; ¹H NMR: δ7.35 (m, 1H), 6.90 (dd J=10.8 Hz, 15.8 Hz, 1H), 6.15 (d, J=15.8 Hz, 1H), 4.76 (d, J=1.7 Hz, 1H), 4.66 (d, J=4.5 Hz, 1H), 4.51 (d, J=1.7 Hz, 1H), 4.30 (br, 1H), 4.13 (m, 1H), 3.80 (m, 4H), 3.42 (m, 1H), 3.33 (m, 1H), 3.0 (m, 1H), 2.41 (m, 2H), 2.06 (m, 1H), 1.80 (m, 1H), 1.73 (m, 2H), 1.47 (m, 1H), 1.45 (m, 1H), 1.31 (dd, J=2.0 Hz, 12.3 Hz, 1H), 1.23 (s, 3H), 1.25 (m, 1H), 1.20 (m, 1H), 0.83 (s, 3H)

### Example 34

### Preparation of 7-(4-morpholino)-11,12-dedihydro-14-deoxy-(E)-15-((2-amino-9-methyl-6-oxo-6,9-dihydro-1H-purin-8-yl)methylene)-8,17-epoxyandrographolide

Analogously to General Method C, the title compound was prepared from 7-morpholino-8-epoxyethanyl-14-deoxy-11,12-dedihydroandrographolid and 2-amino-9-methyl-6-oxo-6,9-dihydro-1H-purine-8-carbaldehyde IR(KBr,cm⁻¹):3440-3078, 2983, 2935, 1745, 1724, 1642, 1450, 1347, 1222, 1122, 1083, 1009; ¹H NMR: δ8.90 (s, 1H), 6.72 (m, 1H), 6.31 (m, 2H), 6.06 (d, J=15.6 Hz, 1H), 4.73 (s, 1H), 4.43 (m, 1H), 4.13 (br, 1H), 3.84 (d, J=9.6 Hz, 1H), 3.67 (m, 4H), 3.22 (m, 1H), 3.70 (s, 3H), 3.31 (d, J=10.2 Hz, 1H), 3.22 (m, 1H), 2.86 (m, 4H), 2.16 (d, J=10.2 Hz, 1H), 2.38 (t, J=10.8 Hz, 2H), 1.97 (m, 1H), 1.72 (d, J=13.2 Hz, 1H), 1.40 (dd, J=4.2 Hz, 3.6 Hz, 1H), 1.34 (d, J=13.2 Hz, 1H), 1.17 (m, 2H), 1.09 (s, 3H), 0.76 (s, 3H).

### Example 35

### Preparation of 11,12-dedihydro-14-deoxy-(E)-15-(2,4-dichlorobenzylidene)andrographolide

Analogously to General Method C, the title compound was prepared from 14-deoxy-11,12-dedihydroandrographolide and 2,4-dichlorobenzaldehyde; IR(KBr,cm⁻¹):3390-3080, 2923, 2857, 1750, 1646, 1601, 1508, 1221, 1201, 1173, 1037; ¹H NMR: δ8.03 (d, J=8.4 Hz, 1H), 7.83 (s, 1H), 7.69 (s, 1H), 7.53 (d, J=8.4 Hz, 1H), 6.83 (m, 1H), 6.48 (s, 1H), 6.26 (d, J=15.6 Hz, 1H), 5.03 (s, 1H), 4.73 (m, 1H), 4.42 (m, 1H), 4.13 (d, J=4.8 Hz, 1H), 3.84 (d, J=10.8 Hz, 1H), 3.28 (d, J=7.2 Hz, 1H), 3.21 (d, J=9.6 Hz, 1H), 3.16 (d, J=3.0 Hz, 1H), 2.43 (d, J=10.2 Hz, 1H), 2.36 (d, J=13.2 Hz, 1H), 1.98 (d, J=10.2 Hz, 1H), 1.71 (d, J=12.0 Hz 1H), 1.60 (m, 1H), 1.57 (d, J=11.4 Hz, 1H), 1.39 (dd, J=3.0 Hz, 3.6 Hz, 1H), 1.32 (d, J=13.2 Hz, 1H), 1.16 (m, 3H), 1.08 (s, 3H), 0.77 (m, 1H).

### Example 36

### Preparation of 11,12-dedihydro-14-deoxy-(E)-15-(2,4-difluorobenzylidene)andrographolide

Analogously to General Method C, the title compound was prepared from 14-deoxy-11,12-dedihydroandrographolide and 2,4-difluorobenzaldehyde; IR(KBr,cm⁻¹):3390-3080, 2923, 1750, 1646, 1605, 1555, 1224, 1201, 1173, 1053; ¹H NMR: δ8.03 (d, J=8.4 Hz, 1H), 7.83 (s, 1H), 7.69 (s, 1H), 7.53 (d, J=8.4 Hz, 1H), 6.83 (m, 1H), 6.48 (s, 1H), 6.26 (d, J=15.6 Hz, 1H), 5.03 (s, 1H), 4.73 (m, 1H), 4.42 (m, 1H), 4.13 (d, J=4.8 Hz, 1H), 3.84 (d, J=10.8 Hz, 1H), 3.28 (d, J=7.2 Hz, 1H), 3.21 (d, J=9.6 Hz, 1H), 3.16 (d, J=3.0 Hz, 1H), 2.43 (d, J=10.2 Hz, 1H), 2.36 (d, J=13.2 Hz, 1H), 1.98 (d, J=10.2 Hz, 1H), 1.71 (d, J=12.0 Hz, 1H), 1.60 (m, 1H), 1.57 (d, J=11.4 Hz, 1H), 1.39 (dd, J=3.0 Hz, 3.6 Hz, 1H), 1.32 (d, J=13.2 Hz, 1H), 1.16 (m, 3H), 1.08 (s, 3H), 0.77 (m, 1H).

### Example 37

### Preparation of 11,12-dedihydro-14-deoxy-15-(4-(dimethylamino)benzylidene)andrographolide

Analogously to General Method C, the title compound was prepared from 14-deoxy-11,12-dedihydroandrographolide and 4-(dimethylamino)benzaldehyde; IR(KBr,cm⁻¹): 3446-3080, 2927, 1750, 1646, 1384, 1201, 1173, 1053; ¹H NMR: δ7.62 (m, 1H), 7.59 (d, J=9.0 Hz, 2H), 6.75 (d, J=9.0 Hz, 2H), 6.72 (m, 1H), 6.21 (s, 1H), 6.15 (d, J=15.8 Hz, 1H), 5.03 (br, 1H), 4.73 (m, 1H), 4.43 (m, 1H), 4.13 (br, 1H), 3.84 (d, J=9.6 Hz, 1H), 3.27 (m, 1H), 3.22 (m, 1H), 2.98 (s, 6H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 38

### Preparation of 11,12-dedihydro-14-deoxy-15-(2-amino-5-oxygen-3H-pyrimidine-5-ylmethylene)-8,17-epoxyandrographolide

Analogously to General Method C, the title compound was prepared from 8-epoxyethanyl-14-deoxy-11,12-dedihydroandrographolide and 2-amino-7-methyl-7H-pyrrolo[2,3-d]pyrimidine-5-carboxaldehyde; ¹H NMR: δ8.42 (br, 2H), 7.62 (s, 1H), 6.72 (m, 1H), 6.21 (m, 1H), 6.06 (d, J=15.6 Hz, 1H), 4.85 (d, J=1.8 Hz, 2H), 4.15 (m, 1H), 3.85 (m, 1H), 3.33 (m, 2H), 3.22 (m, 1H), 2.63 (d, J=3.0 Hz, 1H), 2.16 (d, J=10.2 Hz, 1H), 1.97 (m, 1H), 1.74 (m, 2H), 1.58 (m, 2H), 1.52 (dd, J=3.6 Hz, 4.2 Hz, 1H),1.32 (m, 2H), 1.08 (s, 3H), 1.07 (m, 2H), 0.89 (s, 3H).

### Example 39

### Preparation of 11,12-dedihydro-14-deoxy-(E)-15-(furan-3-ylmethylene) andrographolide

Analogously to General Method C, the title compound was prepared from 14-deoxy-11,12-dedihydroandrographolide and furan-2-carboxaldehyde; IR(KBr,cm⁻¹): 3390-3076, 2923, 2857, 1750, 1646, 1555, 1037, 1017, 952, 880, 803, 753; ¹H NMR: δ7.77 (d, J=4.2 Hz, 1H), 7.59 (s, 1H), 6.86 (d, J=3.0 Hz, 1H), 6.77 (d, J=3.0 Hz, 1H), 6.74 (m, 1H),6.21 (d, J=3.6 Hz, 1H), 6.12 (d, J=15.6 Hz, 1H), 4.91 (m, 2H), 4.38 (m, 1H), 3.23 (m, 1H), 2.36 (m, 2H), 1.94 (m, 1H), 1.68 (d, J=10.8 Hz, 1H), 1.55 (m, 2H), 1.32 (m, 3H), 1.14 (m, 4H), 1.05 (s, 3H), 0.75 (s, 3H).

### Example 40

### Preparation of 11,12-dedihydro-14-deoxy-15-(4-((3,4,5-trihydroxy-6-(hydroxymethyl)tetrahydro-2H-pyran-2-yl)oxy)benzylidene)andrographolide

Analogously to General Method C, the title compound was prepared from 14-deoxy-11,12-dedihydroandrographolide and 4-((3,4,5-trihydroxy-6-hydroxymethyl)tetrahydro-2H-pyran-2-yl)oxy)benzaldehyde; IR(KBr,cm⁻¹): 3428-3079, 2923, 2857, 1750, 1646, 1601, 1511, 1447, 1384, 1228, 1088, 1037, 1017, 952; ¹H NMR: δ7.70 (d, J=9.0 Hz, 2H), 7.07 (d, J=9.0 Hz, 2H), 6.81 (m, 1H), 6.28 (s, 1H), 6.24 (d, J=15.6 Hz, 1H), 5.16 (d, J=8.4 Hz, 1H), 5.03-4.96 (m, 2H), 4.74 (m, 1H), 4.66 (m, 1H), 4.49 (m, 1H), 4.44 (m, 1H), 4.13 (m, 1H), 3.92 (m, 1H), 3.84 (d, J=10.8 Hz, 1H), 3.72-3.65 (m, 2H), 3.30-3.21 (m, 3H), 3.28 (m, 2H), 3.16 (m, 1H), 2.41 (m, 2H), 1.98 (m, 1H), 1.72 (d, J=12.6 Hz, 1H), 1.65-1.56 (m, 2H), 1.45-1.30 (m, 1H), 1.18 (m, 3H), 1.11 (s, 3H), 0.77 (s, 3H).

### Example 41

### Preparation of 11,12-dedihydro-14-deoxy-(E)-15-(4-hydroxy-3-methoxy-5-nitrobenzylidene)-8,17-epoxyandrographolide

Analogously to General Method C, the title compound was prepared from 8-epoxyethanyl-14-deoxy-11,12-dedihydroandrographolide and 4-hydroxy-3-methoxy-5-nitrobenzaldehyde; ¹H NMR: δ7.61 (s, 1H), 7.65 (s, 1H), 7.55 (s, 1H), 6.31 (m, 1H), 6.24 (s, 1H), 6.16 (d, J=15.6 Hz, 1H), 5.01 (d, J=4.8 Hz, 1H), 4.14 (q, J=2.4 Hz, 1H), 3.85 (d, J=2.4 Hz, 1H), 3.80 (s, 3H), 3.21 (m, 2H), 2.97 (s, 1H), 2.67 (d, J=2.4 Hz, 1H), 2.51 (d, J=4.2 Hz, 1H), 2.21 (d, J=9.6 Hz, 1H), 1.78 (m, 2H), 1.62 (m, 2H), 1.52 (m, 1H), 1.33 (m, 2H), 1.09 (s, 3H), 0.94 (d, J=4.2 Hz, 1H), 0.91 (s, 3H).

### Example 42

### Preparation of 3-(4-((3,4,5-trihydroxy-6-(hydroxymethyl)tetrahydro-2H-pyran-2-yl)oxy)benzoyl)-11,12-dedihydro-14-deoxyandrographolide

Analogously to General Method A, the title compound was prepared from 14-deoxy-11,12-dedihydroandrographolide and 4-((3,4,5-triacetoxy-6-(hydroxymethyl)tetrahydro-2H-pyran-2-yl)oxy)benzoic acid; IR(KBr,cm⁻¹): 3414-2929, 1741, 1667, 1232, 1076, 1040; ¹H NMR: δ 7.90 (d, J=8.4 Hz, 2H), 7.67 (m, 1H), 7.11 (d, J=8.4 Hz, 2H), 6.15 (m, 1H), 5.88 (m, 1H), 5.22 (d, J=7.8 Hz, 1H), 5.10 (s, 1H), 5.02 (s, 1H), 4.74 (m, 1H), 4.66 (d, J=7.2 Hz, 1H), 4.49 (m, 1H), 4.44 (m, 1H), 4.13 (m, 1H), 3.92 (m, 1H), 3.84 (d, J=10.8 Hz, 1H), 3.69 (m, 2H), 3.43 (m, 3H), 3.28 (m, 2H), 3.11 (d, J=15.0 Hz, 1H), 2.41 (d, J=9.6 Hz, 1H), 2.37 (d, J=13.2 Hz, 1H), 1.98 (m, 1H), 1.72 (d, J=12.6 Hz, 1H), 1.58 (m, 2H), 1.41 (d, J=9.6 Hz, 1H), 1.34 (d, J=13.2 Hz, 1H), 1.18 (m, 3H), 1.11 (s, 3H), 0.77 (s, 3H).

### Example 43

### Preparation of 3-(4-((3,4,5-trihydroxy-6-(hydroxymethyl)tetrahydro-2H-pyran-2-yl)oxy)benzoyl)-12-(4-morpholino)-14-deoxyandrographolide

Analogously to General Method A, the title compound was prepared from 14-deoxy-12-morpholine-11,12-dedihydroandrographolide and 4-((3,4,5-trihydroxy-6-hydroxymethyl)tetrahydro-2H-pyran-2-yl)oxy)benzoic acid; IR(KBr,cm⁻¹):3430-3079, 2926, 2853, 1741, 1667, 1614, 1513, 1474, 1384, 1304, 1271, 1232, 1076, 1036, 891; ¹H NMR: δ7.91 (d, J=9.0 Hz, 2H), 7.56 (m, 1H), 7.11 (d, J=9.0 Hz, 2H), 5.88 (m, 1H), 5.22 (m, 2H), 5.15 (s, 1H), 5.08 (s, 1H), 4.84 (m, 2H), 4.43 (d, J=12.0 Hz, 2H), 4.37 (d, J=11.4 Hz, 2H), 3.94 (m, 1H), 3.75 (m, 1H), 3.72 (q, J=7.8 Hz, 1H), 3.67 (d, J=10.8 Hz, 1H), 3.6-3.44 (m, 6H), 3.32 (m, 4H), 3.22 (m, 2H), 2.37 (d, J=11.4 Hz, 1H), 2.32 (d, J=3.0 Hz, 2H), 2.05 (m, 1H), 2.02 (m, 1H), 1.71-1.52 (m, 5H), 1.23 (m, 2H), 1.16 (s, 3H), 1.08 (m, 1H), 0.76 (s, 3H).

### Example 44

### Preparation of 11,12-dedihydro-14-deoxy-(E)-15-(4-(dimethylamino)-2-hydroxybenzylidene))andrographolide

Analogously to General Method C, the title compound was prepared from 14-deoxy-11,12-dedihydroandrographolide and 4-(dimethylamino)-2-hydroxybenzaldehyde; ¹H NMR: δ7.62 (s, 1H), 7.09 (d, J=7.5 Hz, 1H), 6.27 (d, J=9.0 Hz, 2H), 6.72 (m, 1H), 6.21 (s, 1H), 6.19 (s, 1H), 5.03 (br, 1H), 4.73 (m, 1H), 4.43 (m, 1H), 4.13 (br, 1H), 3.84 (d, J=9.6 Hz, 1H), 3.27 (m, 1H), 3.22 (m, 1H), 3.06 (s, 6H), 2.38 (t, J=10.8 Hz, 2H), 1.97 (m, 1H), 1.72 (d, J=13.2 Hz, 1H), 1.62 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 45

### Preparation of (E)-3-(2-(6a,10b-dimethyl-8-methylene-3-(4-((3,4,5-trihydroxy-6-(hydroxymethyl)tetrahydro-2H-pyran-2-yl)oxy)phenyl)decahydro-1H-naphtho[2,1-d][1,3]dioxin-7-yl)ethylidene)-4-hydroxydihydrofuran-2(3H)-one

To a mixture of andrographolide 1.0 g (3 mmol) in toluene 20 mL were added 4-((3,4,5-triethoxy-6-(ethoxy-methyl)tetrahydro-2H-pyran-2-yl)oxy)benzaldehyde 1.3 g (3 mmol). The mixture was refluxed for 7 h. The reaction solution was extracted by EtOAC. The crude was separated by silica gel column chromato-graphy to give the title compound; IR(KBr,cm⁻¹): 3428-2929, 1741, 1667, 1615, 1232, 1076, 1040; ¹H NMR: δ7.33 (d, J=8.4 Hz, 2H), 6.97 (d, J=8.4 Hz, 2H), 6.82 (t, J=6.6 Hz, 1H), 5.77 (m, 1H), 5.48 (m, 1H), 5.05 (m, 1H), 4.93 (d, J=7.2 Hz, 2H), 4.62 (d, J=7.2 Hz, 1H), 4.43 (m, 2H), 4.27 (d, J=11.4 Hz, 1H), 4.05 (dd, J=2.4 Hz, 4.2 Hz, 1H), 3.92 (m, 1H), 3.66 (m, 2H), 3.55 (m, 2H), 3.44 (m, 4H), 3.17 (m, 1H), 2.02 (m, 3H), 1.73 (m, 1H), 1.61 (m, 2H), 1.55 (m, 1H), 1.37 (m, 1H), 1.34 (s, 3H), 1.20 (m, 2H), 1.07 (m, 1H), 0.86 (s, 3H).

### Example 46

### Preparation of 11,12-dedihydro-14-deoxy-(E)-15-((2-amino-7-methyl-7H-pyrrolo[2,3-d]pyrimidin-5-yl)methylene)andrographolide

Analogously to General Method C, the title compound was prepared from 14-deoxy-11,12-dedihydroandrographolide and 2-amino-7-methyl-7H-pyrrolo[2,3-d]pyrimidin-5-carbaldehyde; ¹H NMR: δ8.90 (s, 1H), 7.62 (s, 1H), 7.08 (s, 1H), 7.0 (br, 2H), 6.72 (m, 1H), 6.21 (s, 1H), 6.19 (s, 1H), 5.03 (br, 1H), 4.73 (s, 1H), 4.43 (m, 1H), 4.13 (br, 1H), 3.84 (d, J=9.6 Hz, 1H), 3.22 (m, 1H), 3.60 (s, 3H), 3.22 (m, 1H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 47

### Preparation of 3-(4-((3,4,5-trihydroxy-6-(hydroxymethyl)tetrahydro-2H-pyran-2-yl)oxy)benzoyl)-12-nitromethyl-14-deoxyandrographolide

Analogously to General Method A, the title compound was prepared from 12-nitromethyl-14-deoxyandrographolide and 4-((3,4,5-triacetoxy-6-(acetoxymethyl)tetrahydro-2H-pyran-2-yl)oxy)benzoic acid; IR(KBr,cm⁻¹): 3430-3081, 2924, 1754, 1690, 1607, 1554, 1511, 1427, 1374, 1228, 1088, 1045; ¹H NMR: δ7.99 (d, J=9.0 Hz, 2H), 7.41 (m, 1H), 7.12 (d, J=9.0 Hz, 2H), 5.68 (m, 1H), 5.22 (m, 1H), 4.75 (m, 1H), 4.52 (d, J=1.2 Hz, 1H), 4.13 (m, 1H), 3.85 (d, J=3.0 Hz, 1H), 3.44 (m, 4H), 3.29 (m, 1H), 3.23 (m, 1H), 2.72 (d, J=9.6 Hz, 2H), 2.49 (d, J=10.8 Hz, 2H), 2.36 (d, J=10.8 Hz, 2H), 2.27 (m, 4H), 2.26 (s, 3H), 1.98 (m, 1H), 1.72 (d, J=2.4 Hz, 1H), 1.52 (m, 3H), 1.41 (m, 1H), 1.33 (m, 1H), 1.23 (m, 1H), 1.16 (s, 3H), 0.76 (s, 3H).

### Example 48

### Preparation of 3-(4-(4-methylpiperazin-1-yl)-4-oxobutanoyl)-12-nitromethyl-14-deoxyandrographolide

Analogously to General Method A, the title compound was prepared from 12-nitromethyl-14-deoxyandrographolide and 4-(4-methylpiperazin-1-yl)-4-oxobutanoic acid; IR(KBr,cm⁻¹): 3430-3078, 2928, 2850, 1795, 1754, 1739, 1627, 1445, 1385, 1291, 1223, 1174, 1144, 1086, 1050, 1002; ¹H NMR: δ7.65 (m, 1H), 4.87 (m, 2H), 4.82 (m, 2H), 4.75 (m, 2H), 4.58 (m, 1H), 3.78 (d, J=10.8 Hz, 1H), 3.44 (m, 4H), 3.22 (m, 1H), 3.19 (m, 1H), 3.17 (t, J=6 Hz, 1H), 2.72 (d, J=9.6 Hz, 2H), 2.49 (d, J=10.8 Hz, 2H), 2.29 (d, J=12.6 Hz, 1H), 2.27 (m, 4H), 2.16 (s, 3H), 1.75 (m, 3H), 1.57 (m, 4H), 1.39 (d, J=9.6 Hz, 1H), 1.28 (m, 1H), 1.06 (m, 1H), 1.03 (s, 3H), 0.87 (m, 1H), 0.56 (s, 3H).

### Example 49

### Preparation of 3-(4-((3,4,5-trihydroxy-6-(hydroxymethyl)tetrahydro-2H-pyran-2-yl)oxy)benzoyl)-12-((2-aminophenyl)thio)-14-deoxyandrographolide

Analogously to General Method A, the title compound was prepared from 12-((2-aminophenyl)thio)-14-deoxyandrographolide and 4-((3,4,5-triacetoxy-6-(acetoxymethyl)tetrahydro-2H-pyran-2-yl)oxy)benzoic acid; IR(KBr,cm⁻¹):3436-3362, 3079, 2929, 2846, 1741, 1667, 1614, 1513, 1473, 1384, 1304, 1265, 1130, 1076, 1040; ¹H NMR: δ9.56 (s, 1H), 7.64 (s, 1H), 7.53 (d, J=9.0 Hz, 2H), 7.09 (d, J=9.0 Hz, 2H), 7.04 (s, 1H), 4.95-5.20 (br, 5H), 4.75 (m, 1H), 4.71 (m, 1H), 4.51 (m, 1H), 4.41 (m, 1H), 4.19 (m, 1H), 4.13 (m, 1H), 3.93 (m, 2H), 3.69 (m, 5H), 2.50 (m, 3H), 1.99-1.23 (m, 10H), 1.18-0.87 (m, 7H), 0.45 (s, 3H).

### Example 50

### Preparation of 11,12-dedihydro-14-deoxy-15-(2-amino-4-oxo-3H-pyrimidine-5-ylmethylene)andrographolide

Analogously to General Method C, the title compound was prepared from 14-deoxy-11,12-dedihydroandrographolide and 2-amino-6-oxo-1,6-dihydropyrimidin-5-carbaldehyde; ¹H NMR: δ8.42 (br, 2H), 7.62 (s, 1H), 6.72 (m, 1H), 6.26 (d, J=15.6 Hz, 1H), 6.21 (s, 1H), 6.19 (s, 1H), 5.03 (br, 1H), 4.73 (m, 1H), 4.43 (m, 1H), 4.13 (br, 1H), 3.84 (d, J=9.6 Hz, 1H), 3.27 (m, 1H), 3.22 (m, 1H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 51

### Preparation of 12-(diethoxyphosphoryl)-14-deoxyandrographolide

Analogously to General Method E, the title compound was prepared from 14-deoxyandrographolide and diethyl phosphate; IR(KBr,cm⁻¹):3436-3085, 2951, 2851, 1734, 1644, 1445, 1349, 1296, 1219, 1069, 1056, 1011, 990, 837; ¹H NMR: δ6.65 (m, 1H), 4.97 (s, 1H), 4.94 (m, 1H), 4.89 (m, 1H), 4.54 (m, 1H), 4.10 (d, J=7.2 Hz, 1H), 4.07 (m, 4H), 3.80 (d, J=6 Hz, 1H), 3.22 (d, J=4.8 Hz, 1H), 3.17 (d, J=5.4 Hz, Hz, 1H), 2.33 (d, J=13.2 Hz, 1H), 1.96 (m, 1H), 1.76 (m, 3H), 1.61 (m, 3H), 1.48 (d, J=10.8 Hz, 1H), 1.31 (m, 2H), 1.29 (s, 3H), 1.18 (s, 3H), 1.08 (m, 2H), 1.06 (s, 3H), 0.61 (s, 3H).

### Example 52

### Preparation of (E)-3-(2-(6a,10b-dimethyl-8-methylene-3-(3-nitrophenyl) decahydro-1H-naphtho[2,1-d][1,3]dioxin-7-yl)ethylidene)-4-hydroxydihydrofuran-2(3H)-one

To a mixture of andrographolide 1.50 g (4.7 mmol) in toluene 20 mL was added p-toluene-sulfonic acid 0.27 g (1.4 mmol) and m-nitrobenzaldehyde 4.31 g (28.5 mmol). The mixture was reacted for 10 h, 60° C. The reaction solution was extracted by EtOAC. The crude was separated by silica gel column chromatography to give the title compound; IR(KBr,cm⁻¹):3449-3080, 2922, 2855, 1755, 1732, 1670, 1640, 1527, 1448, 1382, 1352, 1217, 1112, 1019, 998; ¹H NMR: δ 8.24 (d, J=7.8 Hz, 2H), 7.87 (d, J=7.8 Hz, 1H), 6.70 (t, J=7.2 Hz, 1H), 6.66 (s, 1H), 6.02 (s, 1H), 4.95 (s, 1H), 4.87 (s, 1H), 4.68 (m, 1H), 4.41 (t, J=6.6 Hz, 1H), 4.27 (d, J=10.8 Hz, 1H), 4.06 (d, J=9.6 Hz, 1H), 3.65 (m, 2H), 2.37 (d, J=12.6 Hz, 1H), 2.01 (m, 1H), 1.94 (d, J=9.6 Hz, 2H), 1.85 (d, J=12.6 Hz, 1H), 1.78 (d, J=10.2 Hz, 2H), 1.34 (s, 3H), 1.28 (m, 5H), 0.86 (s, 3H).

### Example 53

### Preparation of 12-((3-chlorophenyl)amino)-14-deoxyandrographolide

Analogously to General Method E, the title compound was prepared from (E)-5-oxo-4-(2-(3,3,6a,10b-tetramethyl-8-methylenedecahydro-1H-naphtho[2,1-d][1,3]dioxin-7-yl)ethylidene)tetrahydrofuran-3-yl methanesulfonate and m-chloroaniline; IR(KBr,cm⁻¹):3394-3080, 2970, 2931, 2868, 1760,1640, 1450, 1384, 1350, 1221, 1199, 1077, 1047; ¹H NMR: δ7.65 (m, 1H), 7.01 (t, J=8.1 Hz, 1H), 6.51 (d, J=2.4 Hz, 1H), 6.50 (d, J=8.4 Hz, 1H), 6.44 (d, J=8.4 Hz, 1H), 4.97 (s, 1H), 4.94 (s, 1H), 4.89 (m, 1H), 4.54 (m, 1H), 4.10 (d, J=7.2 Hz, 1H), 3.80 (d, J=6 Hz, 1H), 3.22 (d, J=4.8 Hz, 1H), 3.17 (d, J=5.4 Hz, 1H), 2.33 (d, J=13.2 Hz, 1H), 1.96 (m, 1H), 1.76 (m, 3H), 1.61 (m, 3H), 1.48 (d, J=10.8 Hz, 1H), 1.31 (m, 2H), 1.08 (m, 2H), 1.06 (s, 3H), 0.61 (s, 3H).

### Example 54

### Preparation of 7-((2-((4H-imidazol-2-yl)amino)-1-oxopropan-2-yl)amino)-11,12-dedihydro-14-deoxy-(Z)-15-(carboxyl(1-methyl-1H-benzo[d]imidazol-5-yl)methylene)-8,17-epoxyandrographolide

Analogously to General Method E, the title compound was prepared from 8,17-epoxy-7-((1H-imidazol-2-ylamino)-1-oxopropan-2-yl)amino)-14-deoxy-11,12-dedihydroandrographolide and 2-(1-methyl-1H-benzo[d]imidazol-5-yl)-2-oxoacetic acid; ¹H NMR: δ 13.2 (br, 1H), 11.1 (s, 1H), 9.17 (br, 1H), 8.05 (s, 1H), 7.46 (s, 1H), 7.41 (m, 1H), 7.31 (s, 1H), 7.04 (m, 3H), 6.31 (m, 1H), 6.06 (d, J=15.6 Hz, 1H), 5.02 (s, 1H), 4.15 (m, 1H), 3.96 (br, 3H), 3.74 (m, 1H), 3.33 (m, 2H), 3.31 (d, J=10.2 Hz, 1H), 2.90 (m, 1H), 2.63 (d, J=3.0 Hz, 1H), 3.86 (s, 3H), 2.16 (d, J=10.2 Hz, 1H), 1.74 (m, 2H), 1.52 (dd, J=3.6 Hz, 4.2 Hz, 1H), 1.43-1.30 (m, 2H), 1.32 (m, 2H), 1.08 (s, 3H), 1.07 (m, 2H), 0.89 (s, 3H).

### Example 55

### Preparation of 12-((1-(((1r,3s,5R,7S)-3-hydroxyadamantan-1-yl)amino)-1-oxopropan-2-yl)amino)-14-deoxyandrographolide

Analogously to General Method E, the title compound was prepared from (E)-5-oxo-4-(2-(3,3,6a,10b-tetramethyl-8-methylenedecahydro-1H-naphtho[2,1-d][1,3]dioxin-7-yl)ethylidene)tetrahydrofuran-3-yl methanesulfonate and 3-amino-1-adamantanol; IR(KBr,cm⁻¹): 3432-2947, 2875, 1753, 1643, 1452, 1383, 1260, 1165, 1082, 1036, 1021, 917; ¹H NMR: δ7.70 (m, 1H), 6.81 (m, 1H), 5.15 (m, 1H), 5.10 (m, 1H), 5.05 (m, 1H), 4.96 (s, 1H), 4.74 (m, 1H), 4.66 (d, J=6.6 Hz, 1H), 4.49 (m, 1H), 4.43 (m, 1H), 4.13 (m, 1H), 3.92 (m, 1H), 3.85 (d, J=10.8 Hz, 1H), 3.72-3.64 (m, 2H), 3.43 (m, 4H), 3.29 (m, 2H), 3.22 (d, J=10.2 Hz, 1H), 3.16 (m, 1H), 2.43 (d, J=3.0 Hz, 1H), 2.37 (d, J=13.2 Hz, 1H), 1.97 (m, 1H), 1.72 (d, J=12.6 Hz, 1H), 1.60 (m, 2H), 1.41 (d, J=3.6 Hz, 1H), 1.34 (d, J=13.2 Hz, 2H), 1.25-1.12 (m, 3H), 1.08 (s, 3H), 0.78 (s, 3H).

### Example 56

### Preparation of 12-((2-((4H-imidazol-2-yl)amino)-1-oxopropan-2-yl)amino))-14-deoxy-15-(4-(dimethylamino)benzylidene)andrographolide

Analogously to General Method C, the title compound was prepared from 14-deoxy-12-((1-((4H-imidazol-2-yl)amino)-1-oxopropan-2-yl)amino))andrographolid and 4-(dimethylamino)benzaldehyde; ¹H NMR: 7.62 (s, 1H), 7.59 (d, J=9.0 Hz, 2H), 7.04 (d, J=7.5 Hz, 2H), 6.75 (d, J=9.0 Hz, 2H), 6.72 (m, 1H), 6.21 (s, 1H), 6.19 (s, 1H), 5.03 (br, 1H), 4.73 (s, 1H), 4.43 (m, 1H), 4.13 (br, 1H), 3.84 (d, J=9.6 Hz, 1H), 3.27 (m, 1H), 3.74 (m, 1H), 3.22 (m, 1H), 2.98 (m, 7H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 5H), 1.08 (s, 3H), 0.77 (s, 3H).

### Example 57

### Preparation of 12-((1s,3s,5s)-1,3,5-triazaadamantan-7-ylamino)-14-deoxyandrographolide

Analogously to General Method C, the title compound was prepared from (E)-5-oxo-4-(2-(3,3,6a,10b-tetramethyl-8-methylenedecahydro-1H-naphtho[2,1-d][1,3]dioxin-7-yl)ethylidene)tetrahydrofuran-3-yl methanesulfonate and 7-amino-1,3,5-triazaamantadine; IR(KBr, cm⁻¹):3428-2933, 1754, 1644, 1173, 1151, 1079, 1038; ¹H NMR: δ7.75 (m, 1H), 4.93 (m, 1H), 4.85 (m, 1H), 4.79 (m, 1H), 4.51 (d, J=10.8 Hz, 1H), 4.13 (d, J=4.8 Hz, 1H), 3.81 (m, 2H), 3.21 (m, 4H), 3.09 (m, 3H), 3.01 (m, 1H), 2.95 (m, 2H), 2.31 (s, 3H), 1.97 (m, 3H), 1.75-1.60 (m, 6H), 1.33 (m, 2H), 1.19 (m, 1H), 1.16 (m, 1H), 1.12 (s, 3H), 1.09 (s, 3H), 1.03 (m, 1H).

### Example 58

### Preparation of 12-((1-((((1r,3s,5R,7S)-3-hydroxyadamantan-1-yl)amino)-1-oxopropan-2-yl)amino)-14-deoxyandrographolide

Analogously to General Method E, the title compound was prepared from (E)-5-oxo-4-(2-(3,3,6a,10b-tetramethyl-8-methylenedecahydro-1H-naphtho[2,1-d][1,3]dioxin-7-yl)ethylidene)tetrahydrofuran-3-ylmethanesulfonate and 2-amino-N-((1r, 3s, 5R, 7S)-3-hydroxyadamantan-1-yl)propanamide; IR(KBr,cm⁻¹):3436-3421, 2933, 2871, 1754, 1677, 1647, 1450, 1384, 1331, 1173, 1151, 1034 cm⁻¹; ¹H NMR: δ7.87 (m, 1H), 6.74 (s, 1H), 6.12 (d, J=10.8 Hz, 1H), 4.93 (m, 2H), 4.78 (d, J=12.6 Hz, 1H), 4.73 (d, J=10.8 Hz, 1H), 3.82 (m, 2H), 3.60 (d, J=6.6 Hz, 2H), 3.36 (m, 3H), 3.10 (m, 4H), 2.36 (m, 4H), 1.97-1.21 (m, 16H), 1.07 (s, 3H), 0.93 (m, 1H), 0.81 (m, 1H), 0.77 (m, 1H), 0.61 (s, 3H).

### Example 59

### Preparation of 11,12-dedihydro-14-deoxy-(E)-15-((2-amino-7-methyl-7H-pyrrolo[2,3-d]pyrimidin-5-yl)methylene)-8,17-epoxyandrographolide

Analogously to General Method C, the title compound was prepared from 8-epoxyethanyl-14-deoxy-11,12-dedihydroan-drographolide and 2-amino-7-methyl-7H-pyrrolo[2,3-d]pyrimidin-5-carboxaldehyd; ¹HNM R: δ 8.90 (s, 1H), 7.62 (s, 1H), 7.08 (s, 1H), 7.0 (m, 1H), 6.72 (m, 1H), 6.06 (d, J=15.6 Hz, 1H), 5.03 (br, 1H), 4.73 (m, 1H), 4.43 (m, 1H), 4.13 (br, 1H), 3.84 (d, J=9.6 Hz, 1H), 3.22 (m, 1H), 3.60 (s, 3H), 3.31 (d, J=10.2 Hz, 1H), 3.22 (m, 1H), 2.16 (d, J=10.2 Hz, 1H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 60

### Preparation of 11,12-dedihydro-14-deoxy-(E)-15-(4-(diethylamino)-2-hydroxybenzylidene))andrographolide

Analogously to General Method C, the title compound was prepared from 14-deoxy-11,12-dedihydroandrographolide and 4-(diethylamino) salicylaldehyde; R(KBr,cm⁻):3426-2969, 2931, 2871, 1721, 1603, 1557, 1525, 1452, 1410, 1376, 1355, 1272, 1230, 1147, 1104, 1078, 1035, 939; ¹H NMR: δ9.86 (s, 1H), 7.77 (d, J=9.0 Hz, 1H), 7.62 (s, 1H), 6.67 (m, 1H), 6.45 (s, 1H), 6.33 (dd, J=2.4 Hz, 1.8 Hz, 1H), 6.17 (s, 1H), 6.14 (s, 1H), 6.12 (d, J=15.6 Hz, 1H), 5.04 (d, J=4.8 Hz, 1H), 4.72 (m, 1H), 4.43 (m, 1H), 4.15 (d, J=5.4 Hz, 1H), 3.85 (d, J=9.6 Hz, 1H), 3.32 (m, 4H), 3.27 (m, 1H), 3.22 (m, 1H), 2.36 (t, J=9.6 Hz, 2H), 1.97 (m, 1H), 1.71 (d, J=13.2 Hz, 1H), 1.52 (m, 2H), 1.38 (dd, J=3.6 Hz, 4.2 Hz, 1H), 1.34 (d, J=13.8 Hz, 1H), 1.17 (d, J=13.8 Hz, 1H), 1.09 (m, 6H), 0.75 (s, 3H).

### Example 61

### Preparation of 11,12-dedihydro-14-deoxy-(E)-15-(4-(4-methylpiperazin-1-yl)benzylidene)andrographolide

Analogously to General Method C, the title compound was prepared from 14-deoxy-11,12-dedihydroandrographolide and 4-(methylpyrazinyl)benzaldehyde; IR(KBr,cm⁻¹): 3411-3096, 2936, 2849, 1748, 1641, 1577, 1515, 1448, 1379, 1344, 1246, 1188, 1145, 1038; ¹H NMR: δ7.64 (s, 1H), 7.59 (d, J=4.5 Hz, 2H), 6.97 (d, J=9.0 Hz, 2H), 6.75 (m, 1H), 6.22 (s, 1H), 6.20 (d, J=15.6 Hz, 1H), 5.05 (m, 2H), 4.73 (m, 1H), 4.43 (m, 1H), 4.15 (m, 1H), 3.84 (d, J=10.8 Hz, 1H), 3.29-3.22 (m, 6H), 2.42-2.35 (m, 6H), 2.20 (s, 3H), 1.96 (d, J=8.4 Hz, 1H), 1.72 (d, J=13.2 Hz, 1H), 1.60-1.56 (m, 1H), 1.39 (d, J=4.2 Hz, 1H), 1.33 (d, J=13.8 Hz, 1H), 1.17 (m, 2H), 1.08 (s, 3H), 0.77 (s, 3H).

### Example 62

### Preparation of 12-(2-(pyridine-2-yl)amino)-14-deoxy-(Z)-15-(1-(4-(dimethylamino)phenyl)-2-ethoxy-2-oxoethylidene)andrographolide

Analogously to General Method C, the title compound was prepared from 14-deoxy-11,12-dedihydroandrographolide and 2-(4-(dimethylamino)phenyl)-2-oxyacetic acid; IR(KBr,cm⁻¹):3546-3210, 2923, 1750, 1705, 1647 1385, 1205, 1176, 1053, 1037; ¹H NMR: δ10.22 (br, 1H)7.22 (s, 1H), 7.28 (d, J=9.0 Hz, 2H) 6.72 (m, 1H), 6.26 (d, J=15.6 Hz, 1H), 6.19 (s, 1H), 5.03 (br, 1H), 4.85 (d, J=1.8 Hz, 2H), 3.85 (m, 1H), 3.33 (m, 1H), 3.27 (m, H), 3.22 (m, 1H), 3.02 (s, 6H), 2.63 (d, J=3.0 Hz, 1H), 2.16 (d, J=10.2 Hz, 1H), 1.74 (m, 2H), 1.58 (m, 2H), 1.52 (q, J=3.6 Hz, 1H), 1.32 (m, 2H), 1.08 (s, 3H), 0.89 (s, 3H).

### Example 63

### Preparation of 11,12-dedihydro-14-deoxy-(E)-15-((1-methyl-2-oxo-1,2,3,8a-tetrahydroimidazo[1,2-a]pyridin-6-yl)methylene)andrographolide

Analogously to General Method C, the title compound was prepared from 14-deoxy-11,12-dedihydroandrographolide and 1-methyl-2-oxo-1,2,3,8a-tetrahydroimidazo[1,2-a]pyridin-6-carboxaldehyde; IR(KBr,cm⁻¹):3435-3067, 2923, 1740, 1705, 1660, 1647, 1385, 1205, 1176, 1053, 1037; ¹H NMR: δ7.62 (s, 1H), 7.22 (s, 1H), 6.72 (m, 1H), 6.41 (d, J=11 Hz, 1H), 6.26 (d, J=15.6 Hz, 1H), 6.19 (s, 1H), 5.78 (d, J=11 Hz, 1H), 4.85 (m, 2H), 4.73 (m, 1H), 4.43 (m, 1H), 4.13 (br, 1H), 3.84 (d, J=9.6 Hz, 1H), 3.27 (m, 1H), 3.51 (s, 3H), 3.42 (m, 2H), 3.22 (m, 1H), 2.38 (t, J=10.8 Hz, 2H), 1.97 (m, 1H), 1.72 (d, J=13.2 Hz, 1H), 1.58 (m, 2H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 64

### Preparation of 7-(4-morpholino)-11,12-dedihydro-14-deoxy-(E)-15-((2-(4-methylpiperazin-1-yl)pyrimidin-5-yl)methylene)-8,17-epoxyandrographolide

Analogously to General Method C, the title compound was prepared from 7-morpholino-8-epoxyethanyl-14-deoxy-11,12-dedihydroandrographolide and 4-(methylpyrazinyl)oxy]benzaldehyde; ¹H NMR: 8.07 (s, 2H), 7.64 (s, 1H), 6.75 (m, 1H), 6.31 (m, 1H), 6.06 (d, J=15.6 Hz, 1H), 4.73 (m, 1H), 4.43 (m, 1H), 4.15 (m, 1H), 3.84 (d, J=10.8 Hz, 1H), 3.67 (m, 3H), 2.41 (m, 1H), 3.27-3.22 (m, 6H), 2.86 (m, 4H), 2.52-2.35 (m, 8H), 2.20 (s, 3H), 1.60-1.56 (m, 2H), 1.39 (q, J=4.2 Hz, 1H), 1.33 (d, J=13.8 Hz, 1H), 1.17 (m, 2H), 1.08 (s, 3H), 0.77 (s, 3H).

### Example 65

### Preparation of 11,12-dedihydro-14-deoxy-(E)-15-((2-(2-(hydroxymethyl) pyrrolidin-1-yl)pyrimidin-5-yl)methylene)andrographolide

Analogously to General Method C, the title compound was prepared from 14-deoxy-11,12-dedihydroandrographolide and 2-(2-(hydroxymethyl)pyrrolidin-1-yl)pyrimidin-5-carboxaldehyde; IR(KBr,cm⁻¹):3392, 2934, 1754, 1654, 1558, 1451, 1376, 1357, 1211, 1106, 1080, 1035, 1043; ¹H NMR: δ7.62 (s, 1H), 8.09 (s, 2H), 6.72 (m, 1H), 6.26 (d, J=15.6 Hz, 1H), 6.19 (s, 1H), 5.03 (br, 1H), 4.73 (m, 1H), 4.43 (m, 1H), 4.13 (br, 1H), 3.84 (d, J=9.6 Hz, 1H), 3.45 (m, 2H), 3.27 (m, 1H), 3.22 (m, 1H), 2.98 (s, 6H), 2.85 (m, 2H), 2.38 (t, J=10.8 Hz, 2H), 1.97 (m, 1H), 1.72 (d, J=13.2 Hz, 1H), 1.58 (m, 6H), 1.40 (dd, J=4.2 Hz, 3.6 Hz, 1H), 1.34 (d, J=13.2 Hz, 1H), 1.17 (m, 2H), 1.08 (s, 3H), 0.77 (s, 3H).

### Example 66

### Preparation of 11,12-dedihydro-14-deoxy-(E)-15-(4-(diethylamino)-2-hydroxybenzylidene))-8,17-epoxyandrographolide

Analogously to General Method C, the title compound was prepared from 8-epoxyethanyl-14-deoxy-11,12-dedihydroandrographolide and 4-(diethylamino)-2-hydroxybenzaldehyde; IR(KBr,cm⁻¹):3322-2934, 1654, 1558, 1451, 1350, 1273, 1100, 1060, 1035; ¹H NMR: δ9.86 (s, 1H), 7.77 (d, J=9.0 Hz, 1H), 7.62 (s, 1H), 6.67 (m, 1H), 6.45 (s, 1H), 6.33 (m, 1H), 6.20 (d, J=15.6 Hz, 1H), 6.17 (s, 1H), 6.14 (s, 1H), 4.43 (m, 1H), 4.15 (d, J=5.4 Hz, 1H), 3.85 (d, J=9.6 Hz, 1H), 3.32 (m, 5H), 3.27 (m, 1H), 2.63 (m, 1H), 2.36 (t, J=9.6 Hz, 2H), 1.71 (d, J=13.2 Hz, 2H), 1.52 (m, 3H), 1.38 (dd, J=3.6 Hz, 4.2 Hz, 1H), 1.34 (d, J=13.8 Hz, 1H), 1.17 (d, J=13.8 Hz, 1H), 1.09 (m, 9H), 0.75 (s, 3H).

### Example 67

### Preparation of 7-(4-morpholino)-11,12-dedihydro-14-deoxy-(E)-15-(4-(dimethylamino)-2-hydroxybenzylidene))-8,17-epoxyandrographolide

Analogously to General Method C, the title compound was prepared from 7-morpholino-8-epoxyethanyl-14-deoxy-11,12-dedihydroandrographolide and 4-(dimethylamino)-2-hydroxybenzaldehyde; ¹H NMR: δ7.61 (s, 1H), 7.08 (d, J=7.2 Hz, 1H), 6.31 (m, 1H), 6.28 (d, J=7.2 Hz, 1H), 6.24 (s, 1H), 6.21 (s, 1H), 6.16 (d, J=15.6 Hz, 1H), 5.01 (d, J=4.8 Hz, 1H), 4.14 (q, J=2.4 Hz, 1H), 3.85 (d, J=2.4 Hz, 1H), 3.67 (m, 4H), 3.21 (m, 2H), 2.98 (s, 6H), 2.97 (s, 1H), 2.88 (m, 1H), 2.86 (m, 4H), 2.67 (d, J=2.4 Hz, 1H), 2.51 (d, J=4.2 Hz, 1H), 2.21 (d, J=9.6 Hz, 1H), 1.78 (m, 2H), 1.62 (m, 1H), 1.33 (m, 2H), 1.09 (s, 3H), 0.94 (d, J=4.2 Hz, 1H), 0.91 (s, 3H).

### Example 68

### Preparation of 11,12-dedihydro-14-deoxy-(E)-15-((1-methyl-2-oxo-1,2,3,8a-tetrahydroimidazo[1,2-a]pyridin-6-yl)methylene)-8,17-epoxyandrographolide

Analogously to General Method C, the title compound was prepared from 8-epoxyethanyl-14-deoxy-11,12-dedihydroandrographolide and 1-methyl-2-oxo-1,2,3,8a-tetrahydroimidazo[1,2-a]pyridin-6-carboxaldehyde; IR(KBr, cm⁻¹):3435-3067, 2923, 1740, 1660, 1647, 1385, 1205, 1176, 1105, 1037; ¹H NMR: δ7.22 (s, 1H), 6.42 (m, 1H), 6.41 (d, J=11 Hz, 1H), 6.31 (m, 1H), 6.06 (d, J=15.6 Hz, 1H), 6.90 (m, 1H), 5.78 (d, J=11 Hz, 1H), 5.02 (br, 1H), 4.15 (m, 1H), 3.85 (m, 1H), 3.48 (s, 3H), 3.41 (m, 4H), 3.31 (d, J=10.2 Hz, 1H), 2.63 (d, J=3.0 Hz, 1H), 2.16 (d, J=10.2 Hz, 1H), 1.74 (m, 2H), 1.58 (m, 2H), 1.52 (dd, J=3.6 Hz, 4.2 Hz, 1H), 1.32 (m, 2H), 1.08 (s, 3H), 1.07 (m, 2H), 0.89 (s, 3H).

### Example 69

### Preparation of 11,12-dedihydro-14-deoxy-(E)-15-((2-(2-(hydroxymethyl) pyrrolidin-1-yl)pyrimidin-5-yl)methylene)-8,17-epoxyandrographolide

Analogously to General Method C, the title compound was prepared from 8-epoxyethanyl-14-deoxy-11,12-dedihydroandrographolide and 2-(2-(hydroxymethyl)pyrrolidin-1-yl)pyrimidin-5-carboxaldehyde; IR(KBr,cm⁻¹): 3392,2934, 1754, 1644, 1558, 1451, 1376, 1357, 1211, 1116, 1080, 1043; ¹H NMR: δ8.05 (s, 2H), 7.61 (s, 1H), 6.72 (m, 1H), 6.21 (m, 1H), 6.19 (s, 1H), 6.06 (d, J=15.6, 5.02 (br, 1H), 4.85 (d, J=1.8 Hz, 2H), 4.15 (m, 1H), 3.85 (m, 1H), 3.33 (m, 2H), 3.45 (m, 2H), 3.31 (d, J=10.2 Hz, 1H), 2.85 (m, 2H), 2.63 (d, J=3.0 Hz, 1H), 38 (t, J=10.8 Hz, 2H), 2.20 (m, 1H), 2.16 (d, J=10.2 Hz, 1H), 1.74 (m, 2H), 1.58 (m, 6H), 1.52 (dd, J=3.6 Hz, 4.2 Hz, 1H), 1.40 (dd, J=4.2 Hz, 3.6 Hz, 1H), 1.34 (m, 1H), 1.08 (s, 3H), 1.07 (m, 2H), 0.89 (s, 3H).

### Example 70

### Preparation of 12-(2-(pyridine-2-yl)amino)-14-deoxy-(Z)-15-(1-(4-(dimethylamino)phenyl)-2-ethoxy-2-oxoethylidene)-8,17-epoxyandrographolide

Analogously to General Method C, the title compound was prepared from 8-epoxyethanyl-14-deoxy-11,12-dedihydroandrographolide and 2-(4-(dimethylamino)phenyl)-2-oxoacetic acid; IR(KBr,cm⁻) 3650-2900, 2968-2810, 1753, 1720, 1350, 1273, 1086; ¹H NMR: δ10.22 (br, 1H), 7.28 (d, J=9.0 Hz, 2H), 7.22 (s, 1H), 6.80 (d, J=9.0 Hz, 2H), 6.21 (m, 1H), 6.19 (d, J=15.6 Hz, 1H), 5.03 (br, 1H), 4.85 (d, J=1.8 Hz, 2H), 4.15 (br, 1H), 3.85 (m, 1H), 3.33 (m, 2H), 3.27 (m, 1H), 3.22 (m, 1H), 3.02 (s, 6H), 2.63 (d, J=3.0 Hz, 1H), 2.38 (t, J=10.2 Hz, 2H), 2.16 (d, J=10.2 Hz, 1H), 1.97 (m, 1H), 1.52 (dd, J=3.6 Hz, 4.2 Hz, 1H), 1.08 (s, 3H), 1.07 (m, 2H), 0.89 (s, 3H).

### Example 71

### Preparation of 11,12-dedihydro-14-deoxy-(E)-15-(4-(4-methylpiperazin-1-yl)benzylidene)-8,17-epoxyandrographolide

Analogously to General Method C, the title compound was prepared from 8-epoxyethanyl-14-deoxy-11,12-dedihydroandrographolide and 4-(methylpyrazin)benzaldehyde; IR(KBr,cm⁻¹):3417-3083, 2925, 1745, 1600, 1452, 1380, 1324, 1296, 1242, 1185, 1145, 1078, 1038, 1008, 976, 923, 815; ¹H NMR: δ7.63 (s, 1H), 7.60 (d, J=9.0 Hz, 2H), 6.98 (d, J=9.0 Hz, 2H), 6.33 (m, 1H), 6.20 (s, 1H), 6.17 (d, J=15.6 Hz, 1H), 5.01 (d, J=4.8 Hz, 1H), 4.14 (q, J=3.0 Hz, 1H), 3.85 (q, J=2.4 Hz, 1H), 3.21 (m, 4H), 2.67 (d, J=4.2 Hz, 1H), 2.50 (m, 4H), 2.27-2.20 (m, 4H), 1.76 (m, 2H), 1.59 (m, 3H), 1.53 (m, 2H), 1.33 (dd, J=3.6 Hz, 2.4 Hz, 3H), 1.14 (s, 1H), 1.09 (s, 3H), 0.92 (s, 3H).

### Example 72

### Preparation of 7-(4-morpholino)-11,12-dedihydro-14-deoxy-8,17-epoxyandrographolide

Analogously to dehydrolysis reaction, the title compound was prepared from 7-morpholino-8-epoxyethanyl-14-deoxy-11,12-dedihydroandrographolide; IR(KBr,cm⁻¹): 3369, 3079, 2968-2810, 1753, 1454, 1350, 1336, 1273, 1120, 1086, 1009; ¹H NMR: δ7.61 (m, 1H), 6.31 (m, 1H), 6.06 (d, J=15.6 Hz, 1H), 5.02 (s, 1H), 4.85 (d, J=1.8 Hz, 2H), 4.15 (m, 1H), 3.85 (m, 1H), 3.67 (m, 4H), 3.33 (m, 2H), 3.31 (d, J=10.2 Hz, 1H), 2.63 (d, J=3.0 Hz, 1H), 2.86 (m, 5H), 2.16 (d, J=10.2 Hz, 1H), 1.74 (m, 2H), 1.58 (m, 1H), 1.52 (dd, J=3.6 Hz, 4.2 Hz, 1H), 1.32 (m, 1H), 1.08 (s, 3H), 1.07 (m, 2H), 0.89 (s, 3H).

### Example 73

### Preparation of 7-(4-morpholino)-11,12-dedihydro-14-deoxy-(E)-15-((2-(2-(hydroxymethyl)pyrrolidin-1-yl)pyrimidin-5-yl)methylene)-8,17-epoxyandrographolide

Analogously to General Method C, the title compound was prepared from 7-morpholino-8-epoxyethanyl-14-deoxy-11,12-dedihydroandrographolide and 2-(2-(hydroxymethyl)pyrrolidin-1-yl) pyrimidin-5-carboxaldehyde; ¹H NMR: δ8.08 (s, 2H), 7.61 (m, 1H), 6.31 (m, 1H), 6.06 (d, J=15.6 Hz, 1H), 5.81 (s, 1H), 5.02 (s, 1H), 4.85 (d, J=1.8 Hz, 2H), 4.15 (m, 1H), 3.85 (m, 1H), 3.67 (m, 4H), 3.33 (m, 2H), 3.31 (d, J=10.2 Hz, 1H), 2.63 (d, J=3.0 Hz, 1H), 2.86-2.7 (m, 8H), 2.16 (d, J=10.2 Hz, 1H), 1.74-1.63 (m, 4H), 1.58-1.42 (m, 3H), 1.52 (dd, J=3.6 Hz, 4.2 Hz, 1H), 1.32 (m, 1H), 1.08 (s, 3H), 1.07 (m, 2H), 0.89 (s, 3H).

### Example 74

### Preparation of 7-(4-morpholino)-11,12-dedihydro-14-deoxy-(Z)-15-(1-(4-(dimethylamino)phenyl)-2-ethoxy-2-oxoethylidene)-8,17-epoxyandrographolide

Analogously to General Method C, the title compound was prepared from 7-morpholino-8-epoxyethanyl-14-deoxy-11,12-dedihydroandrographolide and 2-(4-(dimethylamino)phenyl)-2-oxoacetic acid; IR (KBr,cm⁻¹):3650-2900, 2968-2810, 1753, 1720, 1454, 1350, 1283, 1273, 1120, 1086, 1009; ¹H NMR: δ10.22 (br, 1H), 7.22 (s, 1H), 7.28 (d, J=9.0 Hz, 2H), 6.80 (d, J=9.0 Hz, 2H), 6.21 (m, 1H), 6.19 (d, J=15.6 Hz, 1H), 5.03 (br, 1H), 4.85 (d, J=1.8 Hz, 2H), 3.85 (m, 1H), 3.67 (m, 4H), 3.33 (m, 2H), 3.27 (m, 1H), 3.22 (m, 1H), 3.02 (s, 6H), 2.90 (m, 1H), 2.86 (m, 4H), 2.63 (d, J=3.0 Hz, 1H), 2.16 (d, J=10.2 Hz, 1H), 1.74 (m, 2H), 1.52 (dd, J=3.6 Hz, 4.2 Hz, 1H), 1.32 (m, 2H), 1.08 (s, 3H), 0.89 (s, 3H).

### Example 75

### Preparation of 7-(4-morpholino)-11,12-dedihydro-14-deoxy-(E)-15-(4-(N-(2-(dimethylamino)ethyl)carbamimidoyl)benzylidene)-8,17-epoxyandrographolide

Analogously to General Method C, the title compound was prepared from 7-morpholino-8-epoxyethanyl-14-deoxy-11,12-dedihydroandrographolide and N-(2-(dimethylamino)ethyl)-4-formylbenzi-imidamide; IR(KBr,cm⁻¹)3450-2910, 2968-2810, 2230, 1753, 1680, 1454, 1350, 1283, 1273, 1120, 1086, 1009; ¹H NMR: δ10.22 (br, 1H), 7.22 (s, 1H), 7.28 (d, J=9.0 Hz, 2H), 6.80 (d, J=9.0 Hz, 2H), 6.21 (m, 1H), 6.19 (m, 1H), 6.19 (d, J=15.6 Hz, 1H), 5.81 (m, 1H), 5.03 (br, 1H), 4.85 (d, J=1.8 Hz, 2H), 3.85 (m, 1H), 3.67 (m, 4H), 3.33 (m, 2H), 3.27 (m, 1H), 3.22 (m, 1H), 3.02 (s, 6H), 2.98 (t, J=7.2 Hz, 2H), 2.90 (m, 1H), 2.86 (m, 4H), 2.63 (d, J=3.0 Hz, 1H), 2.51 (t, J=7.2 Hz, 2H), 2.16 (d, J=10.2 Hz, 1H), 1.74 (m, 2H), 1.52 (dd, J=3.6 Hz, 4.2 Hz, 1H), 1.32 (m, 2H), 1.08 (s, 3H), 0.89 (s, 3H).

### Example 76

### Preparation of 7-(4-morpholino)-11,12-dedihydro-14-deoxy-15-(4-(dimethylamino)benzylidene)andrographolide

Analogously to General Method C, the title compound was prepared from 7-morpholino-8-epoxyethanyl-14-deoxy-11,12-dedihydroandrographolide and 4-dimethylaminobenzaldehyde; IR(KBr, cm⁻¹):3100-2750, 1750, 1646, 1454, 1384, 1283, 1201, 1173, 1120, 1053, 1009; ¹H NMR: δ7.62 (s, 1H), 7.59 (d, J=9.0 Hz, 2H), 6.75 (d, J=9.0 Hz, 2H), 6.72 (m, 1H), 6.31 (m, 1H), 6.19 (d, J=15.6 Hz, 1H), 5.02 (s, 1H), 4.85 (d, J=1.8 Hz, 2H), 4.15 (m, 1H), 3.85 (m, 1H), 3.67 (m, 4H), 3.33 (m, 2H), 3.31 (d, J=10.2 Hz, 1H), 3.02 (d, 6H), 2.63 (d, J=3.0 Hz, 1H), 2.86 (m, 5H), 2.16 (d, J=10.2 Hz, 1H), 1.74 (m, 2H), 1.58 (m, 1H), 1.52 (dd, J=3.6 Hz, 4.2 Hz, 1H), 1.32 (m, 1H), 1.08 (s, 3H), 0.89 (s, 3H).

### Example 77

### Preparation of 11,12-dedihydro-14-deoxy-(E)-15-(4-(1H-imidazol-1-yl)benzylidene)-8,17-epoxyandrographolide

Analogously to General Method C, the title compound was prepared from 8-epoxyethanyl-14-deoxy-11,12-dedihydroandrographolide and 4-(1H-imidazol-1-yl)benzaldehyde; IR(KBr,cm⁻¹):3700-2800, 1753, 1640, 1520, 1450, 1350, 1233, 1086; ¹H NMR: δ7.61 (s, 1H), 7.57 (d, J=9.0 Hz, 2H), 7.47 (d, J=7.0 Hz, 1H), 7.32 (d, J=9.0 Hz, 2H), 7.17 (d, J=7.0 Hz, 1H), 7.15 (s, 1H), 6.31 (m, 1H), 6.24 (s, 1H), 6.16 (d, J=15.6 Hz, 1H), 5.01 (d, J=4.8 Hz, 1H), 4.14 (q, J=2.4 Hz, 1H), 3.85 (d, J=2.4 Hz, 1H), 3.21 (m, 1H), 2.97 (s, 1H), 2.67 (d, J=2.4 Hz, 1H), 2.51 (d, J=4.2 Hz, 1H), 2.21 (d, J=9.6 Hz, 1H), 1.78 (m, 2H), 1.62 (m, 2H), 1.52 (m, 1H), 1.33 (m, 2H), 1.09 (s, 3H), 0.94 (d, J=4.2 Hz, 1H), 0.91 (s, 3H).

### Example 78

### Preparation of 11,12-dedihydro-14-deoxy-15-(4-(dimethylamino)-2-hydroxybenzylidene))-8,17-epoxyandrographolide

Analogously to General Method C, the title compound was prepared from 8-epoxyethanyl-14-deoxy-11,12-dedihydroandrographolide and 4-dime-thylamino-2-hydroxybenzaldehyde; IR(KBr,cm⁻¹):3369-3079, 3021, 2969, 2831, 1753, 1350,1273, 1604, 1086; ¹H NMR: δ7.61 (s, 1H), 7.08 (d, J=7.2 Hz, 1H), 6.31 (m, 1H), 6.28 (d, J=7.2 Hz, 1H), 6.24 (s, 1H), 6.21 (s, 1H), 6.16 (d, J=15.6 Hz, 1H), 5.01 (d, J=4.8 Hz, 1H), 4.14 (q, J=2.4 Hz, 1H), 3.85 (d, J=2.4 Hz, 1H), 3.21 (m, 3H), 2.98 (s, 6H), 2.97 (s, 1H), 2.67 (d, J=2.4 Hz, 1H), 2.51 (d, J=4.2 Hz, 1H), 2.21 (d, J=9.6 Hz, 1H), 1.78 (m, 2H), 1.62 (m, 2H), 1.52 (m, 1H), 1.33 (m, 2H), 1.09 (s, 3H), 0.94 (d, J=4.2 Hz, 1H), 0.91 (s, 3H).

### Example 79

### Preparation 11,12-dedihydro-14-deoxy-(E)-15-(4-(1H-imidazol-1-yl)benzylidene)andrographolide

Analogously to General Method C, the title compound was prepared from 14-deoxy-11,12-dedihydroandrographolide and 4-(1H-imidazol-1-yl)benzaldehyde; IR(KBr,cm⁻¹):3414, 3082, 1763, 1640, 1606, 1262, 1187, 1116, 1080, 1057; ¹H NMR: δ7.59 (d, J=8.4 Hz, 1H), 7.81 (s, 1H), 7.76 (d, J=8.4 Hz, 1H), 7.71 (m, 2H), 7.65 (m, 2H), 7.12 (s, 1H), 6.85 (m, 1H), 6.28 (d, J=15.6 Hz, 1H), 5.08 (s, 1H), 4.47 (s, 1H), 4.43 (s, 1H), 4.20 (s, 1H), 3.84 (d, J=10.8 Hz, 1H), 1.96 (d, J=10.8 Hz, 1H), 1.98 (s, 1H), 1.70 (s, 1H), 1.63 (m, 2H), 1.56 (s, 2H), 1.36 (m, 2H), 1.21 (m, 2H), 1.09 (s, 3H), 1.06 (s, 1H), 0.92 (s, 1H), 0.90 (s, 3H).

TABLE 1
Example 1-436

| Example | Chemical Structure | M⁺/e |
|---|---|---|
| 1 | 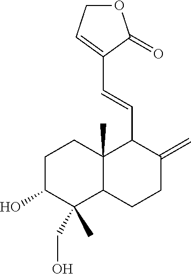 [SMILES: C=C1CCC2[C@](C)(CC[C@@H](O)[C@@]2(C)CO)C1/C=C/C1=CCOC1=O] | 332.20 |
| 2 | 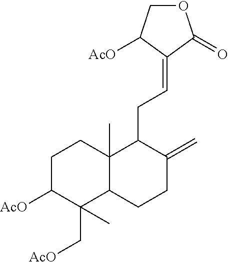 [SMILES: C=C1CCC2C(C)(COC(C)=O)C(OC(C)=O)CCC2(C)C1C/C=C1/C(=O)OCC1OC(C)=O] | 476.24 |
| 3 | 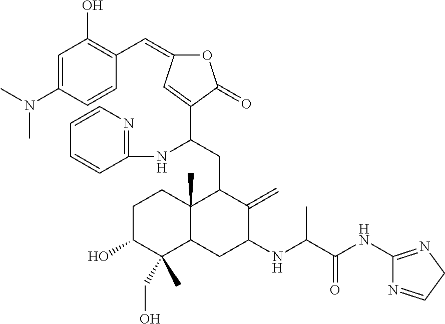 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2ccc(N(C)C)cc2O)OC1=O] | 725.39 |
| 4 | 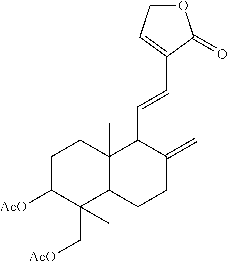 [SMILES: C=C1CCC2C(C)(COC(C)=O)C(OC(C)=O)CCC2(C)C1/C=C/C1=CCOC1=O] | 416.22 |
| 5 | 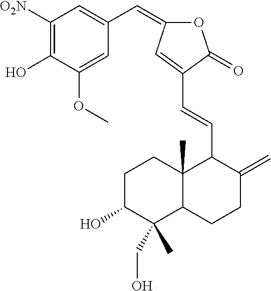 [SMILES: C=C1CCC2[C@](C)(CC[C@@H](O)[C@@]2(C)CO)C1/C=C/C1=CC(=C\c2cc(OC)c(O)c([N+](=O)[O-])c2)/OC1=O] | 511.22 |
| 6 | 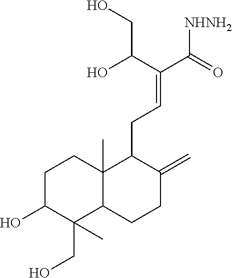 [SMILES: C=C1CCC2C(C)(CO)C(O)CCC2(C)C1C/C=C(/C(=O)NN)C(O)CO] | 382.25 |
| 7 | 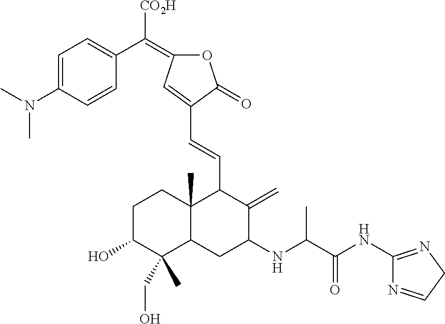 [SMILES: C=C1C(NC(C)C(=O)NC2=NCC=N2)CC2[C@](C)(CC[C@@H](O)[C@@]2(C)CO)C1/C=C/C1=CC(=C(/C(=O)O)c2ccc(N(C)C)cc2)/OC1=O] | 659.33 |
| 8 | 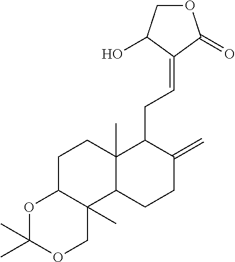 [SMILES: C=C1CCC2C3(C)COC(C)(C)OC3CCC2(C)C1C/C=C1/C(=O)OCC1O] | 390.24 |
| 9 | 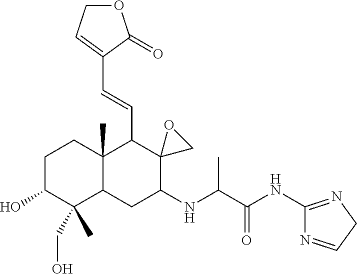 [SMILES: CC(NC1CC2[C@](C)(CC[C@@H](O)[C@@]2(C)CO)C(/C=C/C2=CCOC2=O)C12CO2)C(=O)NC1=NCC=N1] | 500.26 |
| 10 | 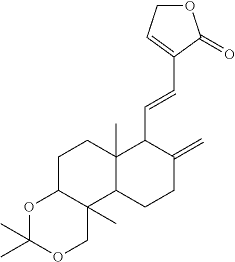 [SMILES: C=C1CCC2C3(C)COC(C)(C)OC3CCC2(C)C1/C=C/C1=CCOC1=O] | 372.23 |
| 11 | 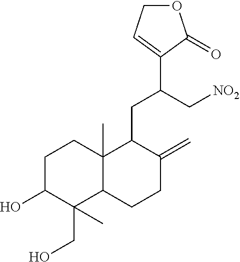 [SMILES: C=C1CCC2C(C)(CO)C(O)CCC2(C)C1CC(C[N+](=O)[O-])C1=CCOC1=O] | 393.22 |
| 12 | 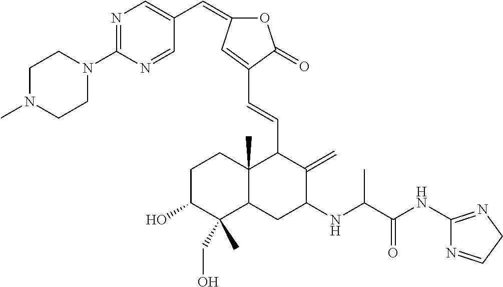 [SMILES: C=C1C(NC(C)C(=O)NC2=NCC=N2)CC2[C@](C)(CC[C@@H](O)[C@@]2(C)CO)C1/C=C/C1=CC(=C\c2cnc(N3CCN(C)CC3)nc2)/OC1=O] | 672.37 |
| 13 | 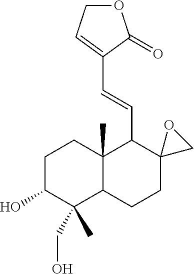 [SMILES: C[C@]12CC[C@@H](O)[C@@](C)(CO)C1CCC1(CO1)C2/C=C/C1=CCOC1=O] | 348.19 |
| 14 | 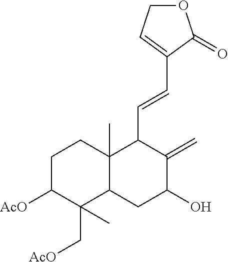 [SMILES: C=C1C(O)CC2C(C)(COC(C)=O)C(OC(C)=O)CCC2(C)C1/C=C/C1=CCOC1=O] | 432.21 |
| 15 | 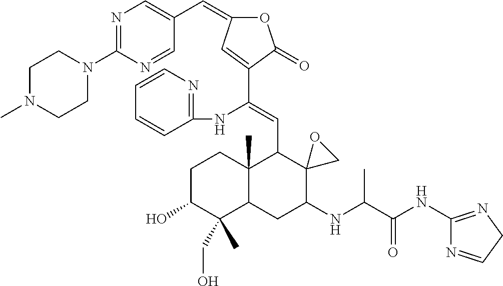 [SMILES: CC(NC1CC2[C@](C)(CC[C@@H](O)[C@@]2(C)CO)C(/C=C(\Nc2ccccn2)C2=C/C(=C\c3cnc(N4CCN(C)CC4)nc3)OC2=O)C12CO2)C(=O)NC1=NCC=N1] | 782.42 |
| 16 | 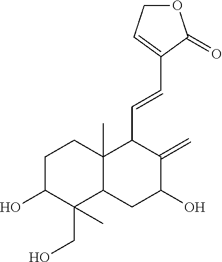 [SMILES: C=C1C(O)CC2C(C)(CO)C(O)CCC2(C)C1/C=C/C1=CCOC1=O] | 348.19 |
| 17 | 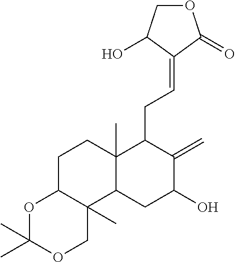 [SMILES: C=C1C(O)CC2C3(C)COC(C)(C)OC3CCC2(C)C1C/C=C1/C(=O)OCC1O] | 406.24 |
| 18 | 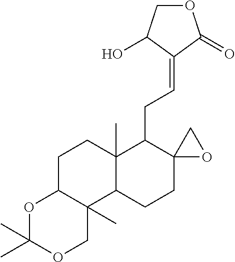 [SMILES: CC1(C)OCC2(C)C(CCC3(C)C(C/C=C4/C(=O)OCC4O)C4(CCC23)CO4)O1] | 406.24 |
| 19 | 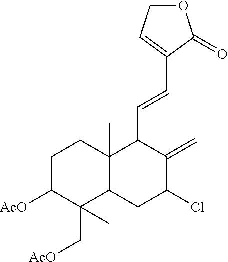 [SMILES: C=C1C(Cl)CC2C(C)(COC(C)=O)C(OC(C)=O)CCC2(C)C1/C=C/C1=CCOC1=O] | 450.18 |
| 20 | 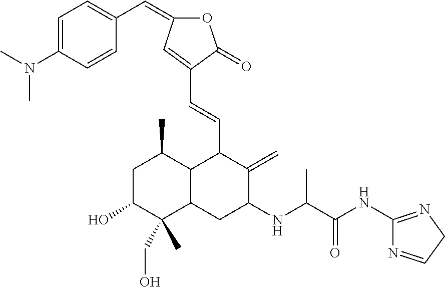 [SMILES: C=C1C(NC(C)C(=O)NC2=NCC=N2)CC2C(C1/C=C/C1=CC(=C\c3ccc(N(C)C)cc3)/OC1=O)[C@H](C)C[C@@H](O)[C@@]2(C)CO] | 615.34 |
| 21 | 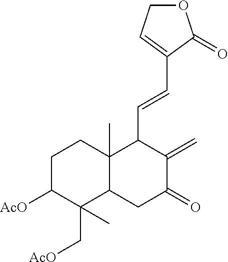 [SMILES: C=C1C(=O)CC2C(C)(COC(C)=O)C(OC(C)=O)CCC2(C)C1/C=C/C1=CCOC1=O] | 430.20 |
| 22 | 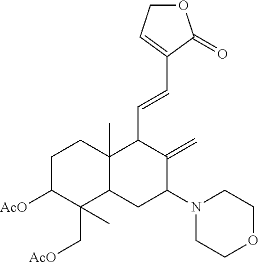 [SMILES: C=C1C(N2CCOCC2)CC2C(C)(COC(C)=O)C(OC(C)=O)CCC2(C)C1/C=C/C1=CCOC1=O] | 501.27 |
| 23 | 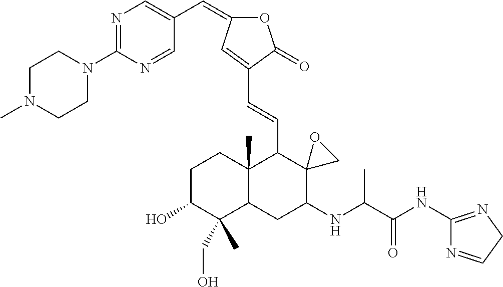 [SMILES: CC(NC1CC2[C@](C)(CC[C@@H](O)[C@@]2(C)CO)C(/C=C/C2=CC(=C\c3cnc(N4CCN(C)CC4)nc3)/OC2=O)C12CO2)C(=O)NC1=NCC=N1] | 688.37 |
| 24 | 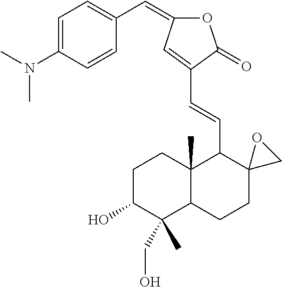 [SMILES: CN(C)c1ccc(/C=C2C=C(/C=C/C3C4(CCC5[C@]3(C)CC[C@@H](O)[C@@]5(C)CO)CO4)C(=O)O\2)cc1] | 479.27 |
| 25 | 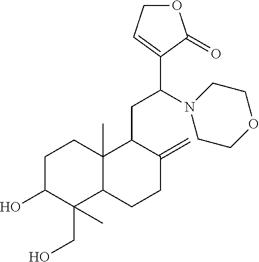 [SMILES: C=C1CCC2C(C)(CO)C(O)CCC2(C)C1CC(C1=CCOC1=O)N1CCOCC1] | 419.27 |
| 26 | 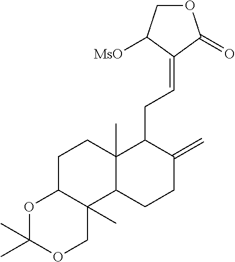 [SMILES: C=C1CCC2C3(C)COC(C)(C)OC3CCC2(C)C1C/C=C1/C(=O)OCC1OS(C)(=O)=O] | 468.22 |
| 27 | 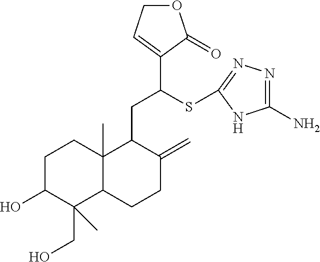 [SMILES: C=C1CCC2C(C)(CO)C(O)CCC2(C)C1CC(Sc1nnc(N)[nH]1)C1=CCOC1=O] | 448.21 |
| 28 | 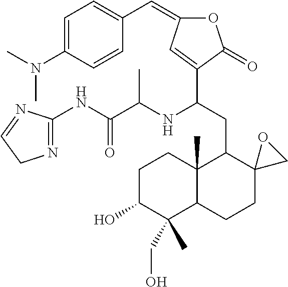 [SMILES: CC(NC(CC1C2(CCC3[C@]1(C)CC[C@@H](O)[C@@]3(C)CO)CO2)C1=C/C(=C\c2ccc(N(C)C)cc2)OC1=O)C(=O)NC1=NCC=N1] | 633.35 |
| 29 | 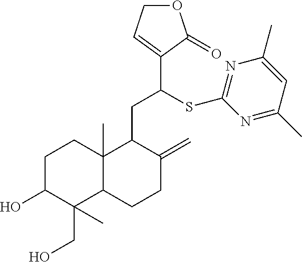 [SMILES: C=C1CCC2C(C)(CO)C(O)CCC2(C)C1CC(Sc1nc(C)cc(C)n1)C1=CCOC1=O] | 472.24 |
| 30 | 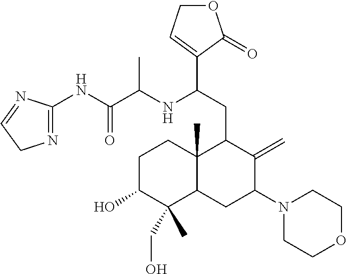 [SMILES: C=C1C(N2CCOCC2)CC2[C@](C)(CC[C@@H](O)[C@@]2(C)CO)C1CC(NC(C)C(=O)NC1=NCC=N1)C1=CCOC1=O] | 571.34 |
| 31 | 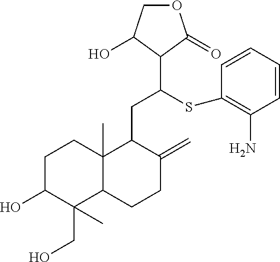 [SMILES: C=C1CCC2C(C)(CO)C(O)CCC2(C)C1CC(Sc1ccccc1N)C1C(=O)OCC1O] | 475.24 |
| 32 | 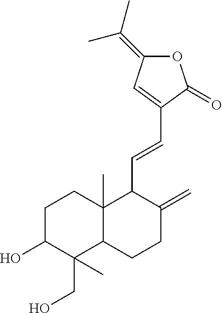 [SMILES: C=C1CCC2C(C)(CO)C(O)CCC2(C)C1/C=C/C1=CC(=C(C)C)OC1=O] | 372.23 |
| 33 | 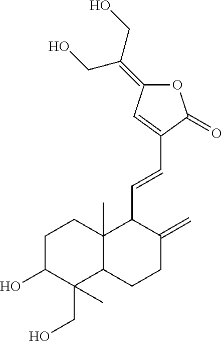 [SMILES: C=C1CCC2C(C)(CO)C(O)CCC2(C)C1/C=C/C1=CC(=C(CO)CO)OC1=O] | 404.22 |
| 34 | 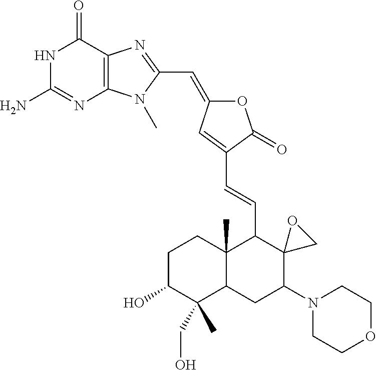 [SMILES: Cn1c(/C=C2C=C(/C=C/C3C4(CO4)C(N4CCOCC4)CC4[C@]3(C)CC[C@@H](O)[C@@]4(C)CO)C(=O)O\2)nc2c(=O)[nH]c(N)nc21] | 608.30 |
| 35 | 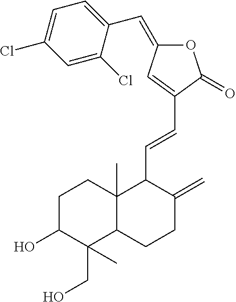 [SMILES: C=C1CCC2C(C)(CO)C(O)CCC2(C)C1/C=C/C1=CC(=C\c2ccc(Cl)cc2Cl)/OC1=O] | 488.15 |
| 36 | 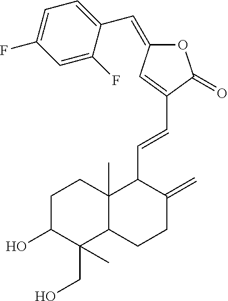 [SMILES: C=C1CCC2C(C)(CO)C(O)CCC2(C)C1/C=C/C1=CC(=C\c2ccc(F)cc2F)/OC1=O] | 456.21 |
| 37 | 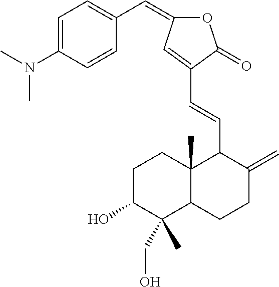 | 463.27 |
| 38 | 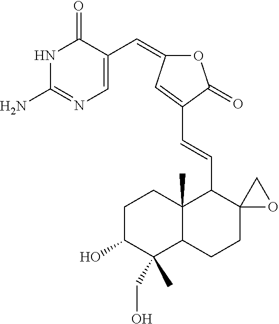 [SMILES: C[C@]12CC[C@@H](O)[C@@](C)(CO)C1CCC1(CO1)C2/C=C/C1=CC(=C\c2cnc(N)[nH]c2=O)/OC1=O] | 469.22 |
| 39 | 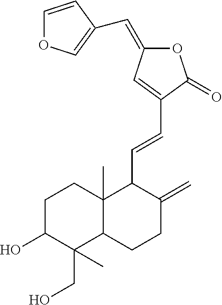 [SMILES: C=C1CCC2C(C)(CO)C(O)CCC2(C)C1/C=C/C1=CC(=C\c2ccoc2)/OC1=O] | 410.21 |
| 40 | 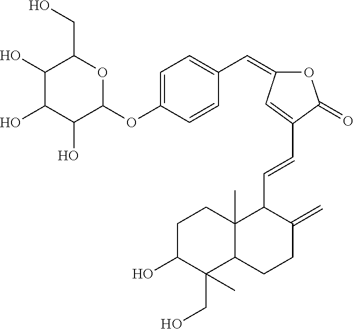 [SMILES: C=C1CCC2C(C)(CO)C(O)CCC2(C)C1/C=C/C1=CC(=C\c2ccc(OC3OC(CO)C(O)C(O)C3O)cc2)/OC1=O] | 598.28 |
| 41 | 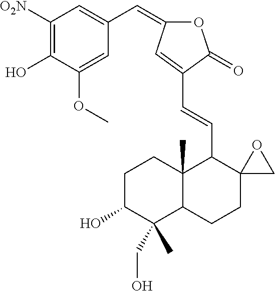 [SMILES: COc1cc(/C=C2C=C(/C=C/C3C4(CCC5[C@]3(C)CC[C@@H](O)[C@@]5(C)CO)CO4)C(=O)O\2)cc([N+](=O)[O-])c1O] | 527.22 |
| 42 | 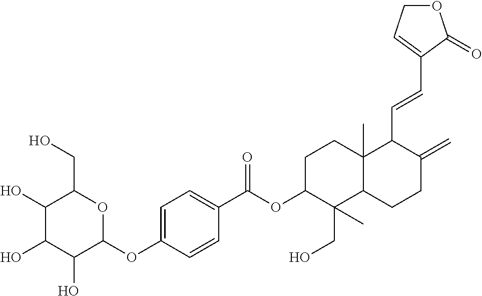 [SMILES: C=C1CCC2C(C)(CO)C(OC(=O)c3ccc(OC4OC(CO)C(O)C(O)C4O)cc3)CCC2(C)C1/C=C/C1=CCOC1=O] | 614.27 |
| 43 | 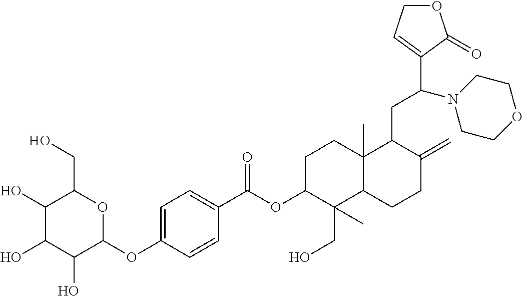 [SMILES: C=C1CCC2C(C)(CO)C(OC(=O)c3ccc(OC4OC(CO)C(O)C(O)C4O)cc3)CCC2(C)C1CC(C1=CCOC1=O)N1CCOCC1] | 701.34 |
| 44 | 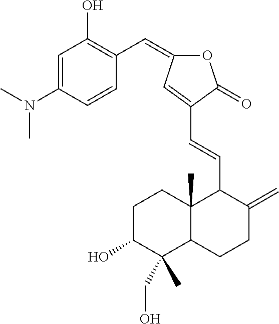 | 479.27 |
| 45 | 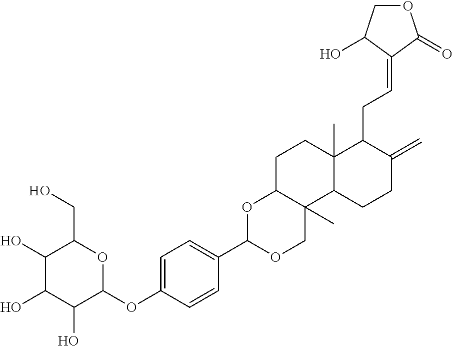 [SMILES: C=C1CCC2C3(C)COC(c4ccc(OC5OC(CO)C(O)C(O)C5O)cc4)OC3CCC2(C)C1C/C=C1/C(=O)OCC1O] | 616.29 |
| 46 | 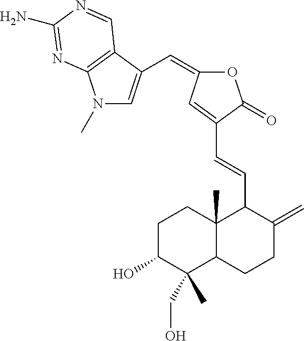 [SMILES: C=C1CCC2[C@](C)(CC[C@@H](O)[C@@]2(C)CO)C1/C=C/C1=CC(=C\c2cn(C)c3nc(N)ncc23)/OC1=O] | 490.26 |
| 47 | 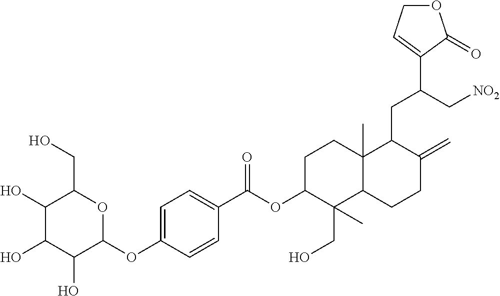 [SMILES: C=C1CCC2C(C)(CO)C(OC(=O)c3ccc(OC4OC(CO)C(O)C(O)C4O)cc3)CCC2(C)C1CC(C[N+](=O)[O-])C1=CCOC1=O] | 675.29 |
| 48 | 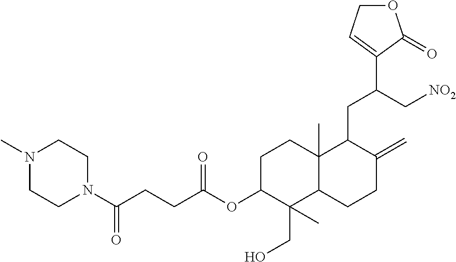 [SMILES: C=C1CCC2C(C)(CO)C(OC(=O)CCC(=O)N3CCN(C)CC3)CCC2(C)C1CC(C[N+](=O)[O-])C1=CCOC1=O] | 575.32 |
| 49 | 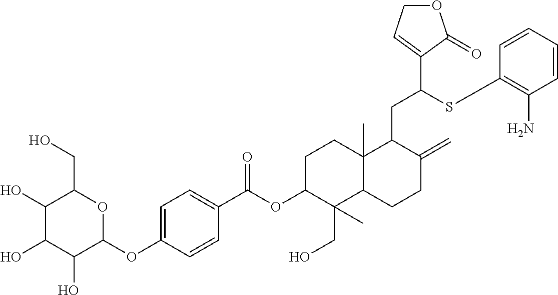 [SMILES: C=C1CCC2C(C)(CO)C(OC(=O)c3ccc(OC4OC(CO)C(O)C(O)C4O)cc3)CCC2(C)C1CC(Sc1ccccc1N)C1=CCOC1=O] | 739.30 |
| 50 | 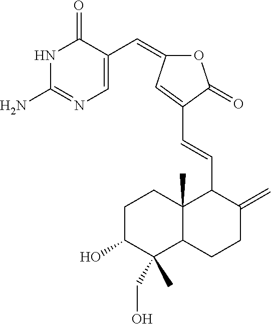 [SMILES: C=C1CCC2[C@](C)(CC[C@@H](O)[C@@]2(C)CO)C1/C=C/C1=CC(=C\c2cnc(N)[nH]c2=O)/OC1=O] | 453.23 |
| 51 | 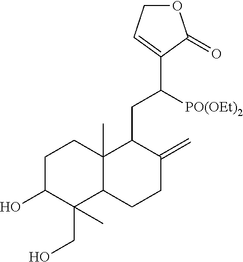 | 470.24 |
| 52 | 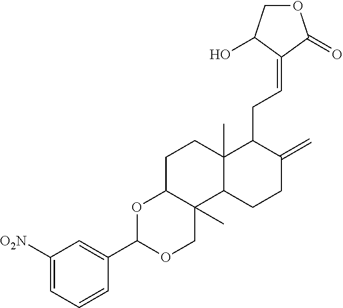 [SMILES: C=C1CCC2C3(C)COC(c4cccc([N+](=O)[O-])c4)OC3CCC2(C)C1C/C=C1/C(=O)OCC1O] | 483.23 |
| 53 | 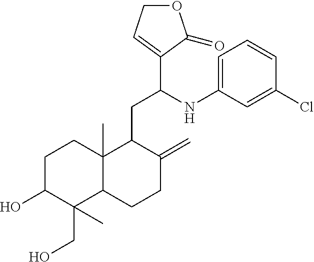 [SMILES: C=C1CCC2C(C)(CO)C(O)CCC2(C)C1CC(Nc1cccc(Cl)c1)C1=CCOC1=O] | 459.22 |
| 54 | 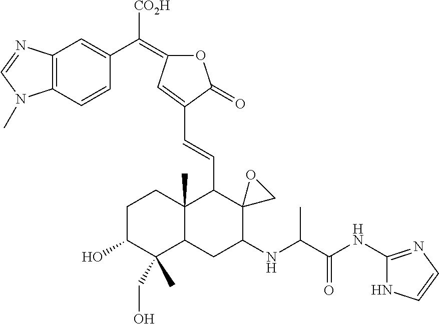 [SMILES: CC(NC1CC2[C@](C)(CC[C@@H](O)[C@@]2(C)CO)C(/C=C/C2=CC(=C(/C(=O)O)c3ccc4c(c3)ncn4C)/OC2=O)C12CO2)C(=O)Nc1ncc[nH]1] | 686.31 |
| 55 | 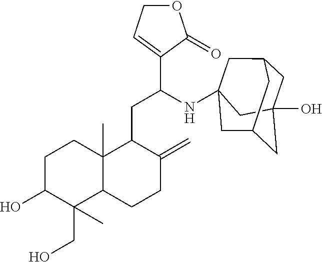 [SMILES: C=C1CCC2C(C)(CO)C(O)CCC2(C)C1CC(NC12CC3CC(CC(O)(C3)C1)C2)C1=CCOC1=O] | 499.33 |
| 56 | 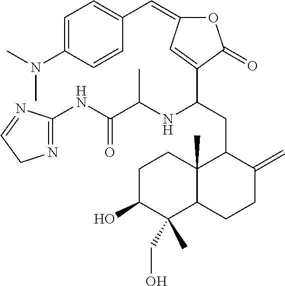 [SMILES: C=C1CCC2[C@](C)(CC[C@H](O)[C@@]2(C)CO)C1CC(NC(C)C(=O)NC1=NCC=N1)C1=C/C(=C\c2ccc(N(C)C)cc2)OC1=O] | 617.36 |
| 57 | 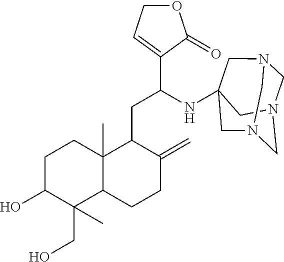 [SMILES: C=C1CCC2C(C)(CO)C(O)CCC2(C)C1CC(NC12CN3CN(CN(C3)C1)C2)C1=CCOC1=O] | 486.32 |
| 58 | 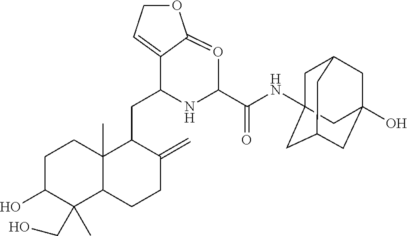 | 570.37 |
| 59 | 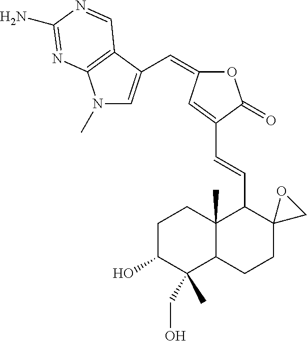 [SMILES: Cn1cc(/C=C2C=C(/C=C/C3C4(CCC5[C@]3(C)CC[C@@H](O)[C@@]5(C)CO)CO4)C(=O)O\2)c2cnc(N)nc21] | 506.25 |
| 60 | 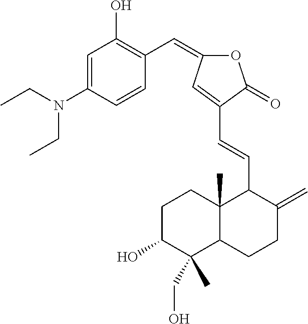 [SMILES: C=C1CCC2[C@](C)(CC[C@@H](O)[C@@]2(C)CO)C1/C=C/C1=CC(=C\c2ccc(N(CC)CC)cc2O)/OC1=O] | 507.30 |
| 61 | 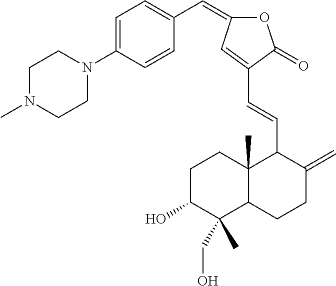 | 518.31 |
| 62 | 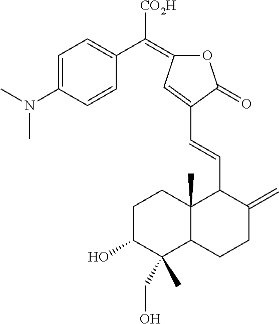 | 507.26 |
| 63 | 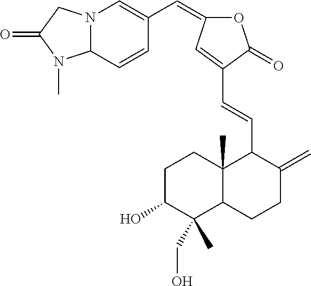 [SMILES: C=C1CCC2[C@](C)(CC[C@@H](O)[C@@]2(C)CO)C1/C=C/C1=CC(=C\C2=CN3CC(=O)N(C)C3C=C2)/OC1=O] | 492.26 |
| 64 | 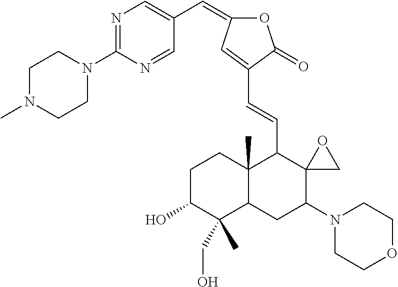 [SMILES: CN1CCN(c2ncc(/C=C3C=C(/C=C/C4C5(CO5)C(N5CCOCC5)CC5[C@]4(C)CC[C@@H](O)[C@@]5(C)CO)C(=O)O\3)cn2)CC1] | 621.35 |
| 65 | 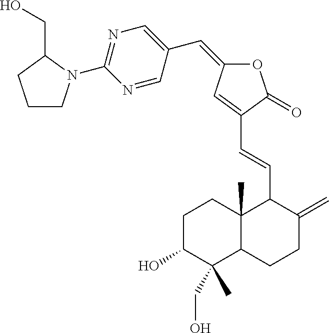 [SMILES: C=C1CCC2[C@](C)(CC[C@@H](O)[C@@]2(C)CO)C1/C=C/C1=CC(=C\c2cnc(N3CCCC3CO)nc2)/OC1=O] | 521.29 |
| 66 | 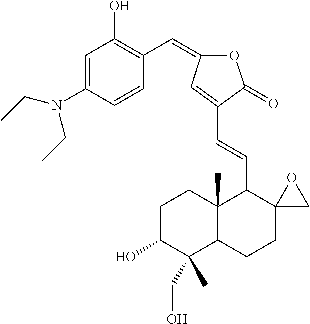 [SMILES: CCN(CC)c1ccc(/C=C2C=C(/C=C/C3C4(CCC5[C@]3(C)CC[C@@H](O)[C@@]5(C)CO)CO4)C(=O)O\2)c(O)c1] | 523.29 |
| 67 | 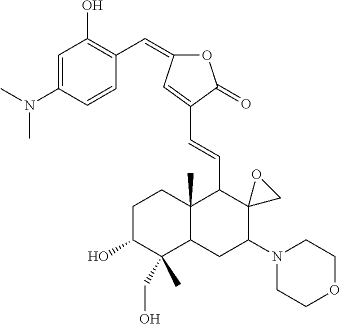 [SMILES: CN(C)c1ccc(/C=C2C=C(/C=C/C3C4(CO4)C(N4CCOCC4)CC4[C@]3(C)CC[C@@H](O)[C@@]4(C)CO)C(=O)O\2)c(O)c1] | 580.31 |
| 68 | 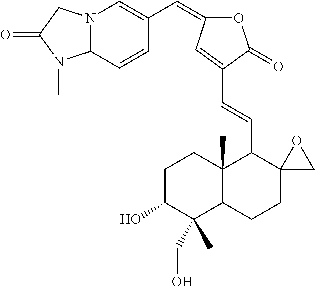 [SMILES: CN1C(=O)CN2C=C(/C=C3C=C(/C=C/C4C5(CCC6[C@]4(C)CC[C@@H](O)[C@@]6(C)CO)CO5)C(=O)O\3)C=CC21] | 508.26 |
| 69 | 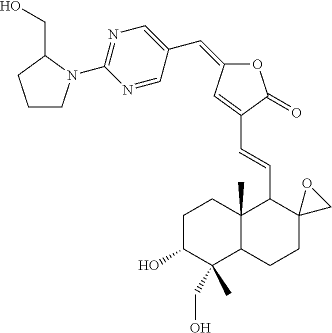 [SMILES: C[C@]12CC[C@@H](O)[C@@](C)(CO)C1CCC1(CO1)C2/C=C/C1=CC(=C\c2cnc(N3CCCC3CO)nc2)/OC1=O] | 537.28 |
| 70 | 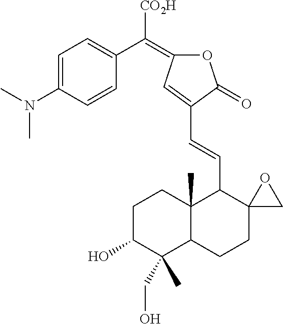 [SMILES: CN(C)c1ccc(/C(C(=O)O)=C2C=C(/C=C/C3C4(CCC5[C@]3(C)CC[C@@H](O)[C@@]5(C)CO)CO4)C(=O)O\2)cc1] | 523.26 |
| 71 | 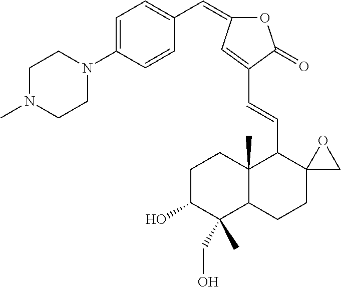 [SMILES: CN1CCN(c2ccc(/C=C3C=C(/C=C/C4C5(CCC6[C@]4(C)CC[C@@H](O)[C@@]6(C)CO)CO5)C(=O)O\3)cc2)CC1] | 534.31 |
| 72 | 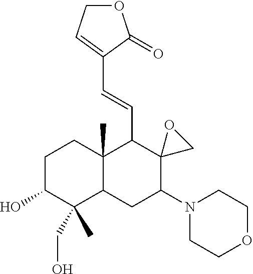 [SMILES: C[C@]12CC[C@@H](O)[C@@](C)(CO)C1CC(N1CCOCC1)C1(CO1)C2/C=C/C1=CCOC1=O] | 433.25 |
| 73 | 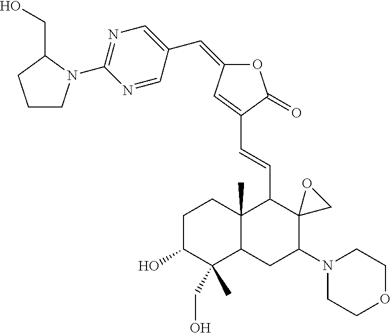 [SMILES: C[C@]12CC[C@@H](O)[C@@](C)(CO)C1CC(N1CCOCC1)C1(CO1)C2/C=C/C1=CC(=C\c2cnc(N3CCCC3CO)nc2)/OC1=O] | 622.34 |
| 74 | 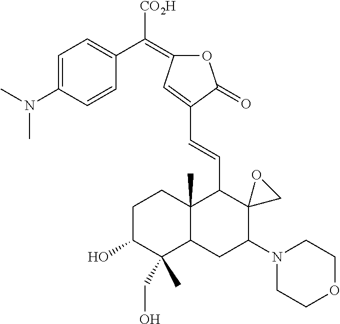 [SMILES: CN(C)c1ccc(/C(C(=O)O)=C2C=C(/C=C/C3C4(CO4)C(N4CCOCC4)CC4[C@]3(C)CC[C@@H](O)[C@@]4(C)CO)C(=O)O\2)cc1] | 608.31 |
| 75 | 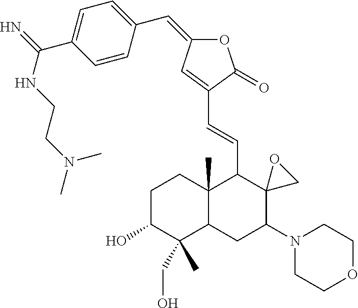 [SMILES: CN(C)CCNC(=N)c1ccc(/C=C2C=C(/C=C/C3C4(CO4)C(N4CCOCC4)CC4[C@]3(C)CC[C@@H](O)[C@@]4(C)CO)C(=O)O\2)cc1] | 633.37 |
| 76 | 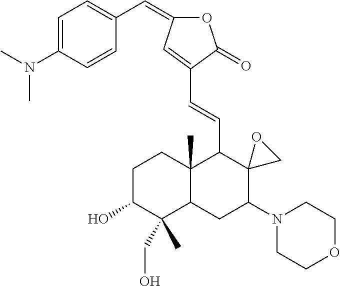 [SMILES: CN(C)c1ccc(/C=C2C=C(/C=C/C3C4(CO4)C(N4CCOCC4)CC4[C@]3(C)CC[C@@H](O)[C@@]4(C)CO)C(=O)O\2)cc1] | 564.32 |
| 77 | 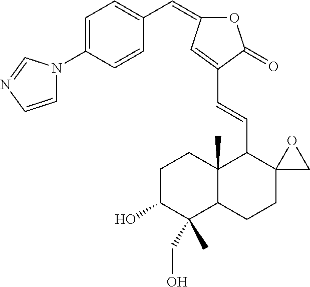 [SMILES: C[C@]12CC[C@@H](O)[C@@](C)(CO)C1CCC1(CO1)C2/C=C/C1=CC(=C\c2ccc(-n3ccnc3)cc2)/OC1=O] | 502.25 |
| 78 | 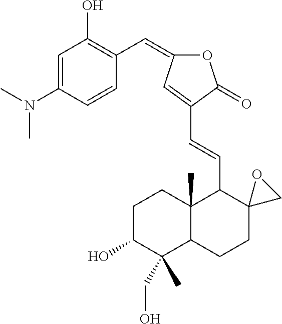 [SMILES: CN(C)c1ccc(/C=C2C=C(/C=C/C3C4(CCC5[C@]3(C)CC[C@@H](O)[C@@]5(C)CO)CO4)C(=O)O\2)c(O)c1] | 495.26 |
| 79 | 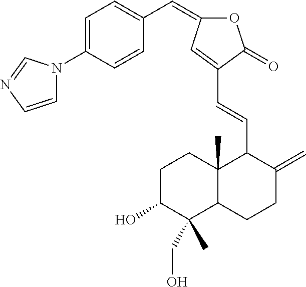 [SMILES: C=C1CCC2[C@](C)(CC[C@@H](O)[C@@]2(C)CO)C1/C=C/C1=CC(=C\c2ccc(-n3ccnc3)cc2)/OC1=O] | 486.25 |
| 80 | 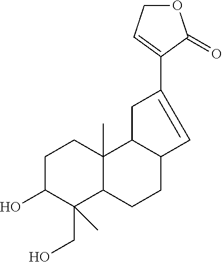 [SMILES: CC1(CO)C(O)CCC2(C)C3CC(C4=CCOC4=O)=CC3CCC12] | 332.20 |
| 81 | 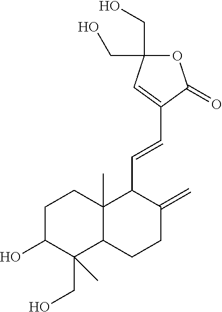 [SMILES: C=C1CCC2C(C)(CO)C(O)CCC2(C)C1/C=C/C1=CC(CO)(CO)OC1=O] | 392.22 |
| 82 | 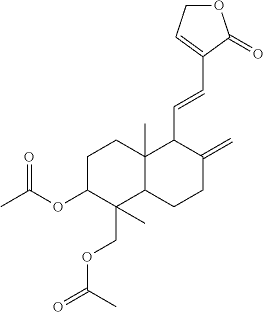 [SMILES: C=C1CCC2C(C)(COC(C)=O)C(OC(C)=O)CCC2(C)C1/C=C/C1=CCOC1=O] | 416.22 |
| 83 | 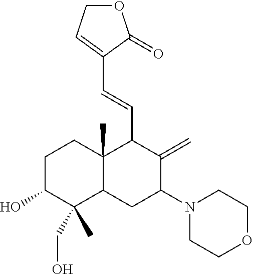 [SMILES: C=C1C(N2CCOCC2)CC2[C@](C)(CC[C@@H](O)[C@@]2(C)CO)C1/C=C/C1=CCOC1=O] | 417.25 |
| 84 | 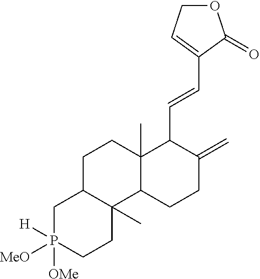 [SMILES: C=C1CCC2C3(C)CC[PH](OC)(OC)CC3CCC2(C)C1/C=C/C1=CCOC1=O] | 420.24 |
| 85 | 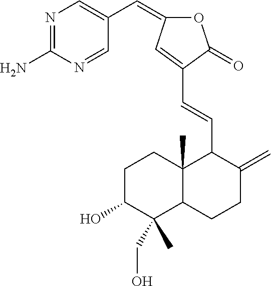 [SMILES: C=C1CCC2[C@](C)(CC[C@@H](O)[C@@]2(C)CO)C1/C=C/C1=CC(=C\c2cnc(N)nc2)/OC1=O] | 437.23 |
| 86 | 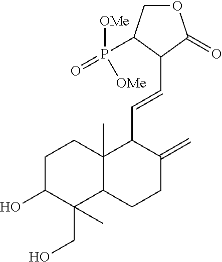 [SMILES: C=C1CCC2C(C)(CO)C(O)CCC2(C)C1/C=C/C1C(=O)OCC1P(=O)(OC)OC] | 442.21 |
| 87 | 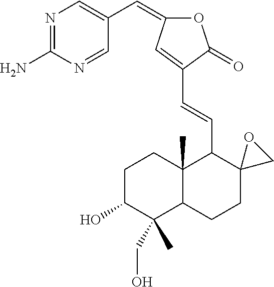 [SMILES: C[C@]12CC[C@@H](O)[C@@](C)(CO)C1CCC1(CO1)C2/C=C/C1=CC(=C\c2cnc(N)nc2)/OC1=O] | 453.23 |
| 88 | 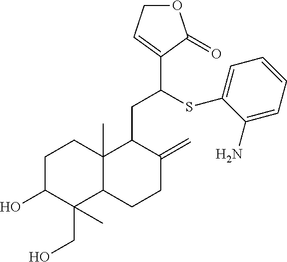 [SMILES: C=C1CCC2C(C)(CO)C(O)CCC2(C)C1CC(Sc1ccccc1N)C1=CCOC1=O] | 457.23 |
| 89 | 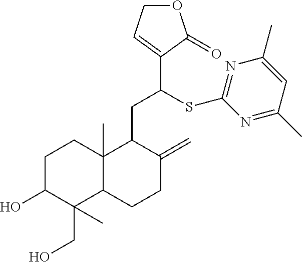 [SMILES: C=C1CCC2C(C)(CO)C(O)CCC2(C)C1CC(Sc1nc(C)cc(C)n1)C1=CCOC1=O] | 472.24 |
| 90 | 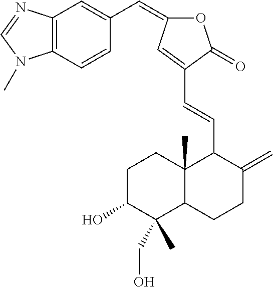 [SMILES: C=C1CCC2[C@](C)(CC[C@@H](O)[C@@]2(C)CO)C1/C=C/C1=CC(=C\c2ccc3c(c2)ncn3C)/OC1=O] | 474.25 |
| 91 | 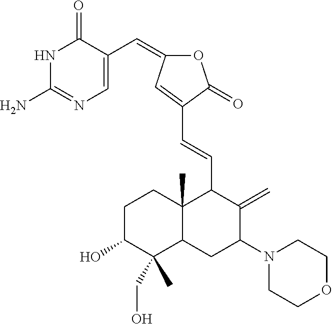 [SMILES: C=C1C(N2CCOCC2)CC2[C@](C)(CC[C@@H](O)[C@@]2(C)CO)C1/C=C/C1=CC(=C\c2cnc(N)[nH]c2=O)/OC1=O] | 474.30 |
| 92 | 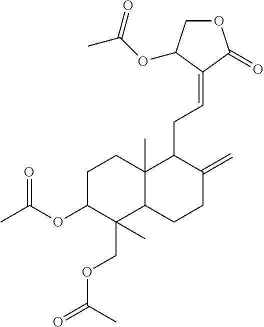 [SMILES: C=C1CCC2C(C)(COC(C)=O)C(OC(C)=O)CCC2(C)C1C/C=C1/C(=O)OCC1OC(C)=O] | 476.24 |
| 93 | 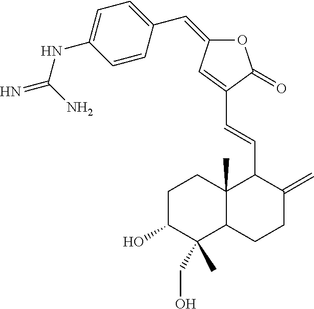 [SMILES: C=C1CCC2[C@](C)(CC[C@@H](O)[C@@]2(C)CO)C1/C=C/C1=CC(=C\c2ccc(NC(=N)N)cc2)/OC1=O] | 477.26 |
| 94 | 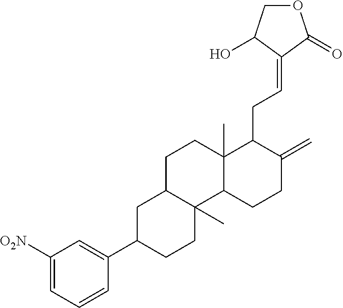 [SMILES: C=C1CCC2C3(C)CCC(c4cccc([N+](=O)[O-])c4)CC3CCC2(C)C1C/C=C1/C(=O)OCC1O] | 479.27 |
| 95 | 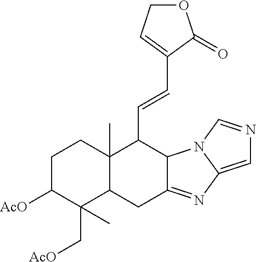 [SMILES: CC(=O)OCC1(C)C(OC(C)=O)CCC2(C)C(/C=C/C3=CCOC3=O)C3C(=Nc4cncn43)CC12] | 481.22 |
| 96 | 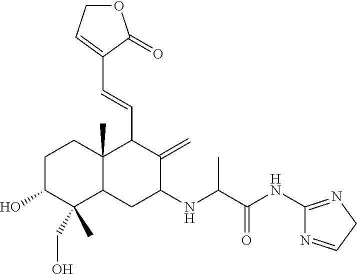 [SMILES: C=C1C(NC(C)C(=O)NC2=NCC=N2)CC2[C@](C)(CC[C@@H](O)[C@@]2(C)CO)C1/C=C/C1=CCOC1=O] | 484.27 |
| 97 | 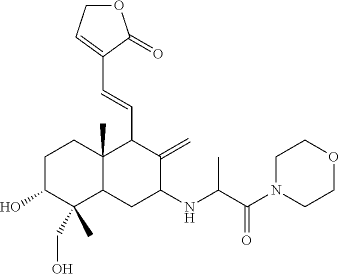 [SMILES: C=C1C(NC(C)C(=O)N2CCOCC2)CC2[C@](C)(CC[C@@H](O)[C@@]2(C)CO)C1/C=C/C1=CCOC1=O] | 488.29 |
| 98 | 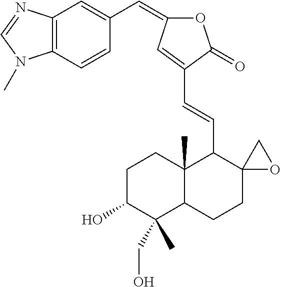 [SMILES: Cn1cnc2cc(/C=C3C=C(/C=C/C4C5(CCC6[C@]4(C)CC[C@@H](O)[C@@]6(C)CO)CO5)C(=O)O\3)ccc21] | 490.25 |
| 99 | 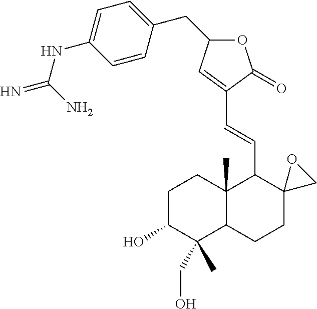 [SMILES: C[C@]12CC[C@@H](O)[C@@](C)(CO)C1CCC1(CO1)C2/C=C/C1=CC(Cc2ccc(NC(=N)N)cc2)OC1=O] | 493.26 |
| 100 | 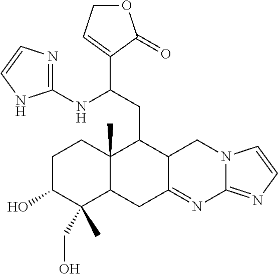 [SMILES: C[C@]12CC[C@@H](O)[C@@](C)(CO)C1CC1=Nc3nccn3CC1C2CC(Nc1ncc[nH]1)C1=CCOC1=O] | 494.26 |
| 101 | 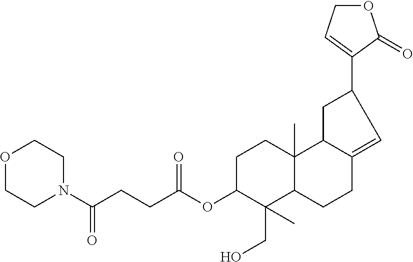 [SMILES: CC1(CO)C(OC(=O)CCC(=O)N2CCOCC2)CCC2(C)C3CC(C4=CCOC4=O)C=C3CCC12] | 501.27 |
| 102 | 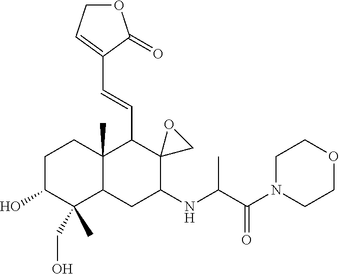 [SMILES: CC(NC1CC2[C@](C)(CC[C@@H](O)[C@@]2(C)CO)C(/C=C/C2=CCOC2=O)C12CO2)C(=O)N1CCOCC1] | 504.28 |
| 103 | 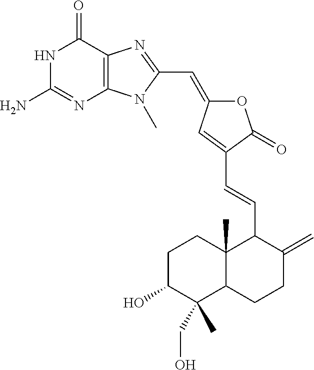 [SMILES: C=C1CCC2[C@](C)(CC[C@@H](O)[C@@]2(C)CO)C1/C=C/C1=CC(=C\c2nc3c(=O)[nH]c(N)nc3n2C)/OC1=O] | 507.25 |
| 104 | 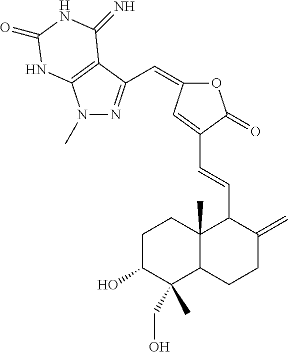 [SMILES: C=C1CCC2[C@](C)(CC[C@@H](O)[C@@]2(C)CO)C1/C=C/C1=CC(=C\c2nn(C)c3[nH]c(=O)[nH]c(=N)c23)/OC1=O] | 507.25 |
| 105 | 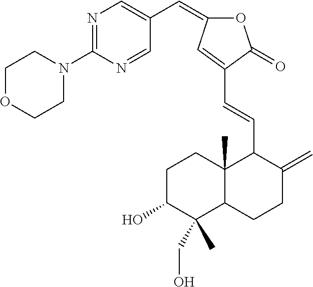 [SMILES: C=C1CCC2[C@](C)(CC[C@@H](O)[C@@]2(C)CO)C1/C=C/C1=CC(=C\c2cnc(N3CCOCC3)nc2)/OC1=O] | 507.27 |
| 106 | 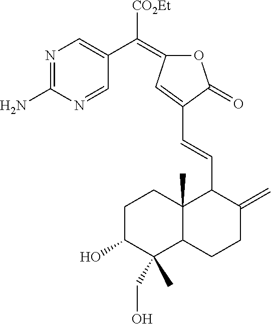 [SMILES: C=C1CCC2[C@](C)(CC[C@@H](O)[C@@]2(C)CO)C1/C=C/C1=CC(=C(/C(=O)OCC)c2cnc(N)nc2)/OC1=O] | 509.25 |
| 107 | 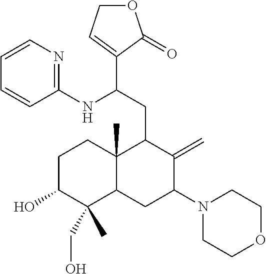 [SMILES: C=C1C(N2CCOCC2)CC2[C@](C)(CC[C@@H](O)[C@@]2(C)CO)C1CC(Nc1ccccn1)C1=CCOC1=O] | 511.30 |
| 108 | 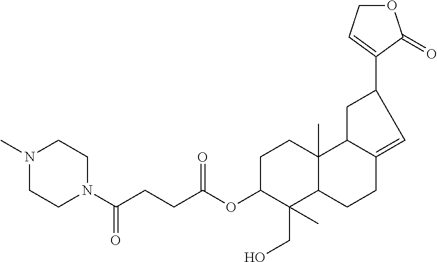 [SMILES: CN1CCN(C(=O)CCC(=O)OC2CCC3(C)C4CC(C5=CCOC5=O)C=C4CCC3C2(C)CO)CC1] | 514.30 |
| 109 | 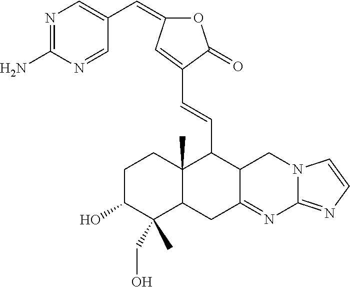 [SMILES: C[C@]12CC[C@@H](O)[C@@](C)(CO)C1CC1=Nc3nccn3CC1C2/C=C/C1=CC(=C\c2cnc(N)nc2)/OC1=O] | 516.25 |
| 110 | 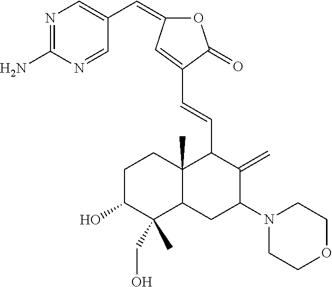 [SMILES: C=C1C(N2CCOCC2)CC2[C@](C)(CC[C@@H](O)[C@@]2(C)CO)C1/C=C/C1=CC(=C\c2cnc(N)nc2)/OC1=O] | 522.28 |
| 111 | 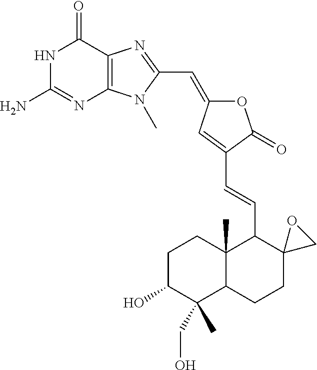 [SMILES: Cn1c(/C=C2C=C(/C=C/C3C4(CCC5[C@]3(C)CC[C@@H](O)[C@@]5(C)CO)CO4)C(=O)O\2)nc2c(=O)[nH]c(N)nc21] | 523.24 |
| 112 | 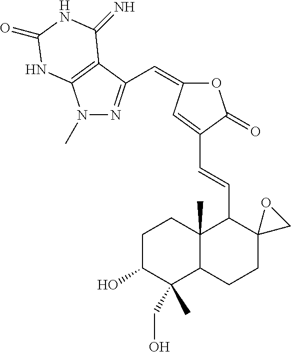 [SMILES: Cn1nc(/C=C2C=C(/C=C/C3C4(CCC5[C@]3(C)CC[C@@H](O)[C@@]5(C)CO)CO4)C(=O)O\2)c2c(=N)[nH]c(=O)[nH]c21] | 523.24 |
| 113 | 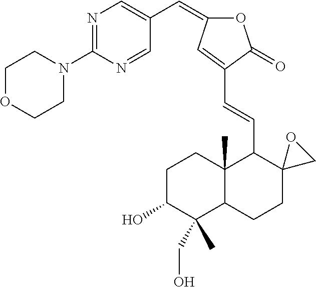 [SMILES: C[C@]12CC[C@@H](O)[C@@](C)(CO)C1CCC1(CO1)C2/C=C/C1=CC(=C\c2cnc(N3CCOCC3)nc2)/OC1=O] | 523.27 |
| 114 | 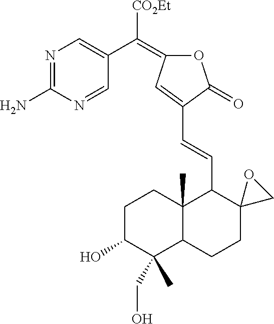 [SMILES: CCOC(=O)/C(=C1C=C(/C=C/C2C3(CCC4[C@]2(C)CC[C@@H](O)[C@@]4(C)CO)CO3)C(=O)O/1)c1cnc(N)nc1] | 525.25 |
| 115 | 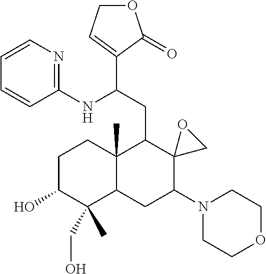 [SMILES: C[C@]12CC[C@@H](O)[C@@](C)(CO)C1CC(N1CCOCC1)C1(CO1)C2CC(Nc1ccccn1)C1=CCOC1=O] | 527.30 |
| 116 | 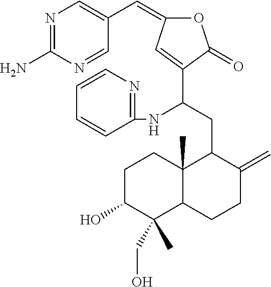 [SMILES: C=C1CCC2[C@](C)(CC[C@@H](O)[C@@]2(C)CO)C1CC(Nc1ccccn1)C1=C/C(=C\c2cnc(N)nc2)OC1=O] | 531.28 |
| 117 | 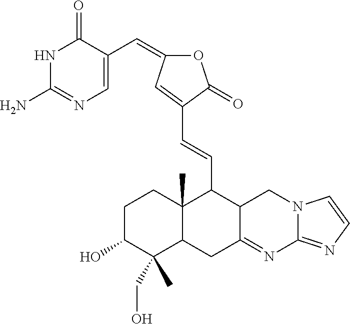 [SMILES: C[C@]12CC[C@@H](O)[C@@](C)(CO)C1CC1=Nc3nccn3CC1C2/C=C/C1=CC(=C\c2cnc(N)[nH]c2=O)/OC1=O] | 532.24 |
| 118 | 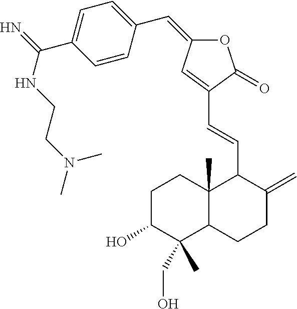 [SMILES: C=C1CCC2[C@](C)(CC[C@@H](O)[C@@]2(C)CO)C1/C=C/C1=CC(=C\c2ccc(C(=N)NCCN(C)C)cc2)/OC1=O] | 533.33 |
| 119 | 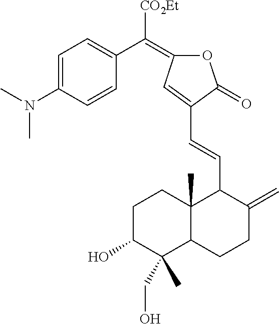 | 535.29 |
| 120 | 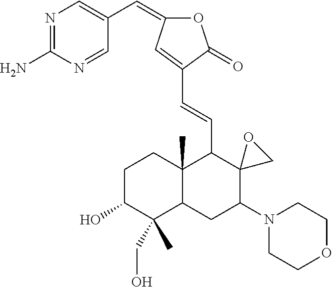 [SMILES: C[C@]12CC[C@@H](O)[C@@](C)(CO)C1CC(N1CCOCC1)C1(CO1)C2/C=C/C1=CC(=C\c2cnc(N)nc2)/OC1=O] | 538.28 |
| 121 | 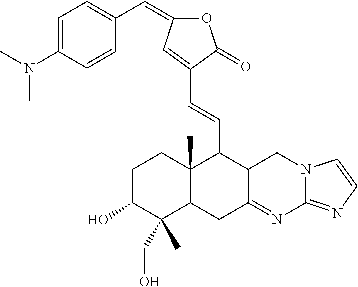 [SMILES: CN(C)c1ccc(/C=C2C=C(/C=C/C3C4Cn5ccnc5N=C4CC4[C@]3(C)CC[C@@H](O)[C@@]4(C)CO)C(=O)O\2)cc1] | 542.29 |
| 122 | 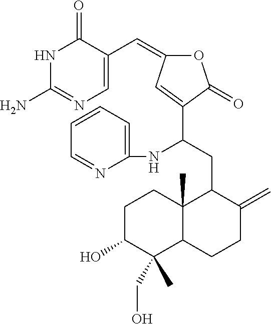 [SMILES: C=C1CCC2[C@](C)(CC[C@@H](O)[C@@]2(C)CO)C1CC(Nc1ccccn1)C1=C/C(=C\c2cnc(N)[nH]c2=O)OC1=O] | 547.28 |
| 123 | 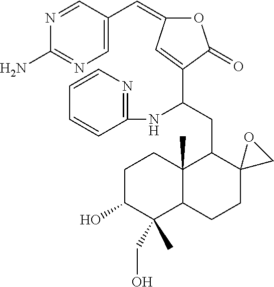 [SMILES: C[C@]12CC[C@@H](O)[C@@](C)(CO)C1CCC1(CO1)C2CC(Nc1ccccn1)C1=C/C(=C\c2cnc(N)nc2)OC1=O] | 547.28 |
| 124 | 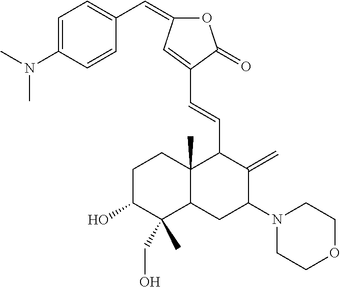 [SMILES: C=C1C(N2CCOCC2)CC2[C@](C)(CC[C@@H](O)[C@@]2(C)CO)C1/C=C/C1=CC(=C\c2ccc(N(C)C)cc2)/OC1=O] | 548.33 |
| 125 | 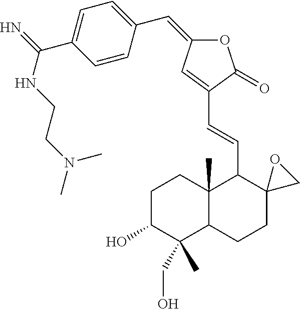 [SMILES: CN(C)CCNC(=N)c1ccc(/C=C2C=C(/C=C/C3C4(CCC5[C@]3(C)CC[C@@H](O)[C@@]5(C)CO)CO4)C(=O)O\2)cc1] | 549.32 |
| 126 | 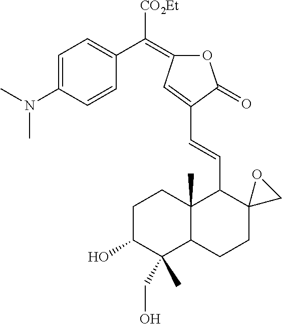 [SMILES: CCOC(=O)/C(=C1C=C(/C=C/C2C3(CCC4[C@]2(C)CC[C@@H](O)[C@@]4(C)CO)CO3)C(=O)O/1)c1ccc(N(C)C)cc1] | 551.29 |
| 127 | 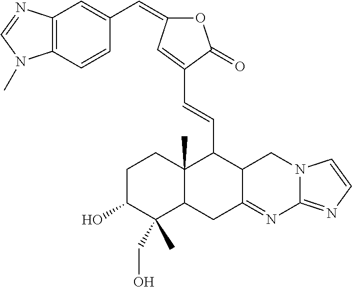 [SMILES: Cn1cnc2cc(/C=C3C=C(/C=C/C4C5Cn6ccnc6N=C5CC5[C@]4(C)CC[C@@H](O)[C@@]5(C)CO)C(=O)O\3)ccc21] | 553.27 |
| 128 | 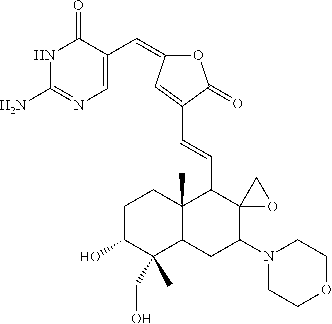 [SMILES: C[C@]12CC[C@@H](O)[C@@](C)(CO)C1CC(N1CCOCC1)C1(CO1)C2/C=C/C1=CC(=C\c2cnc(N)[nH]c2=O)/OC1=O] | 554.27 |
| 129 | 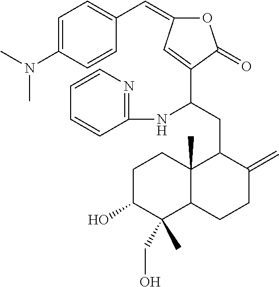 | 557.33 |
| 130 | 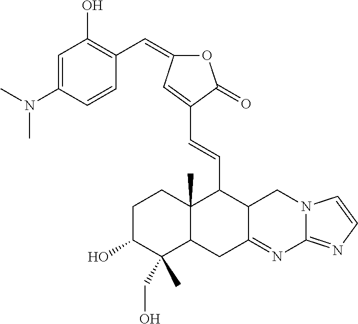 [SMILES: CN(C)c1ccc(/C=C2C=C(/C=C/C3C4Cn5ccnc5N=C4CC4[C@]3(C)CC[C@@H](O)[C@@]4(C)CO)C(=O)O\2)c(O)c1] | 558.28 |
| 131 | 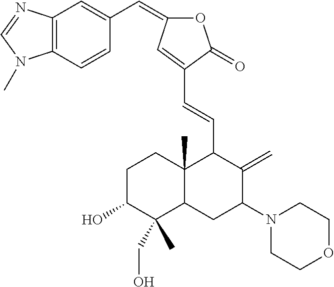 [SMILES: C=C1C(N2CCOCC2)CC2[C@](C)(CC[C@@H](O)[C@@]2(C)CO)C1/C=C/C1=CC(=C\c2ccc3c(c2)ncn3C)/OC1=O] | 559.30 |
| 132 | 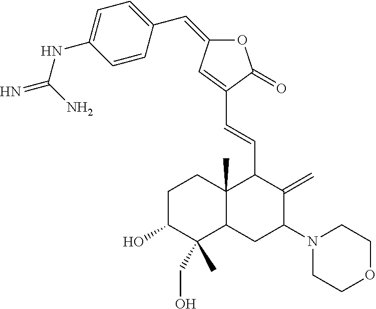 [SMILES: C=C1C(N2CCOCC2)CC2[C@](C)(CC[C@@H](O)[C@@]2(C)CO)C1/C=C/C1=CC(=C\c2ccc(NC(=N)N)cc2)/OC1=O] | 562.32 |
| 133 | 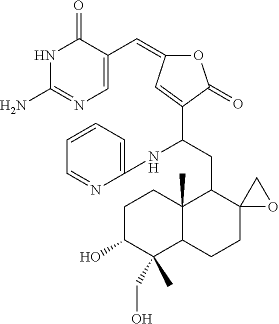 [SMILES: C[C@]12CC[C@@H](O)[C@@](C)(CO)C1CCC1(CO1)C2CC(Nc1ccccn1)C1=C/C(=C\c2cnc(N)[nH]c2=O)OC1=O] | 563.27 |
| 134 | 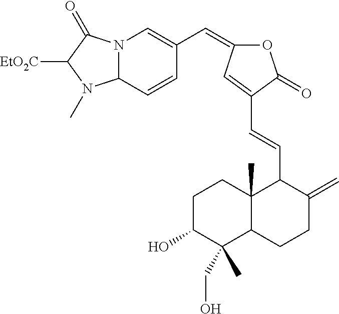 [SMILES: C=C1CCC2[C@](C)(CC[C@@H](O)[C@@]2(C)CO)C1/C=C/C1=CC(=C\C2=CN3C(=O)C(C(=O)OCC)N(C)C3C=C2)/OC1=O] | 564.28 |
| 135 | 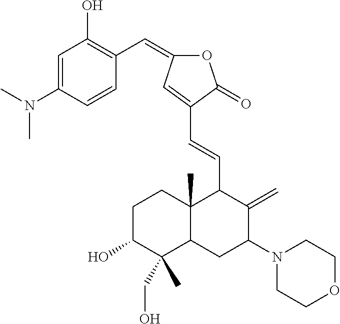 [SMILES: C=C1C(N2CCOCC2)CC2[C@](C)(CC[C@@H](O)[C@@]2(C)CO)C1/C=C/C1=CC(=C\c2ccc(N(C)C)cc2O)/OC1=O] | 564.32 |
| 136 | 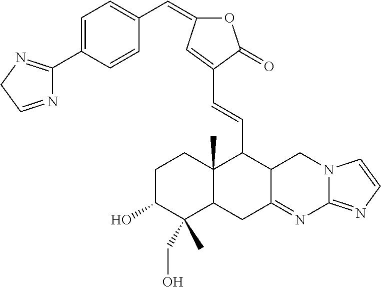 [SMILES: C[C@]12CC[C@@H](O)[C@@](C)(CO)C1CC1=Nc3nccn3CC1C2/C=C/C1=CC(=C\c2ccc(C3=NCC=N3)cc2)/OC1=O] | 565.27 |
| 137 | 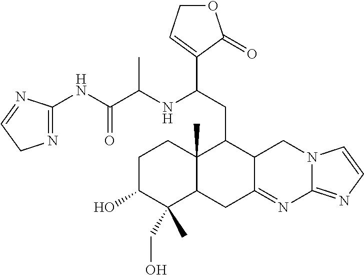 [SMILES: CC(NC(CC1C2Cn3ccnc3N=C2CC2[C@]1(C)CC[C@@H](O)[C@@]2(C)CO)C1=CCOC1=O)C(=O)NC1=NCC=N1] | 565.30 |
| 138 | 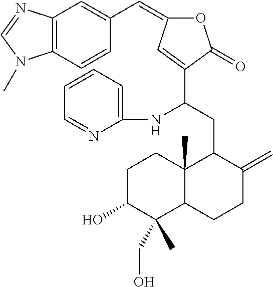 [SMILES: C=C1CCC2[C@](C)(CC[C@@H](O)[C@@]2(C)CO)C1CC(Nc1ccccn1)C1=C/C(=C\c2ccc3c(c2)ncn3C)OC1=O] | 568.30 |
| 139 | 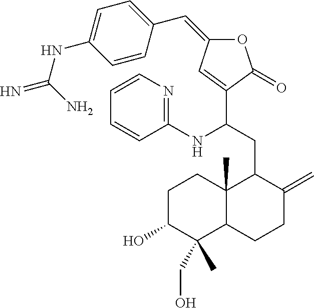 [SMILES: C=C1CCC2[C@](C)(CC[C@@H](O)[C@@]2(C)CO)C1CC(Nc1ccccn1)C1=C/C(=C\c2ccc(NC(=N)N)cc2)OC1=O] | 571.32 |
| 140 | 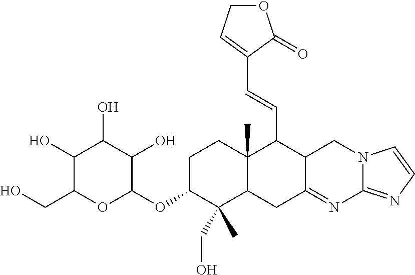 [SMILES: C[C@]12CC[C@@H](OC3OC(CO)C(O)C(O)C3O)[C@@](C)(CO)C1CC1=Nc3nccn3CC1C2/C=C/C1=CCOC1=O] | 573.27 |
| 141 | 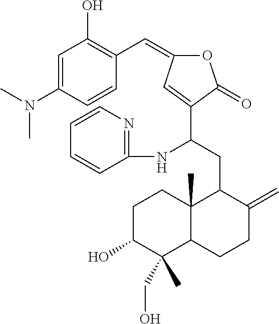 | 573.32 |
| 142 | 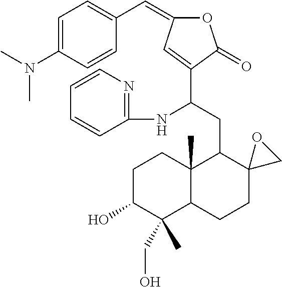 [SMILES: CN(C)c1ccc(/C=C2\C=C(C(CC3C4(CCC5[C@]3(C)CC[C@@H](O)[C@@]5(C)CO)CO4)Nc3ccccn3)C(=O)O2)cc1] | 573.32 |
| 143 | 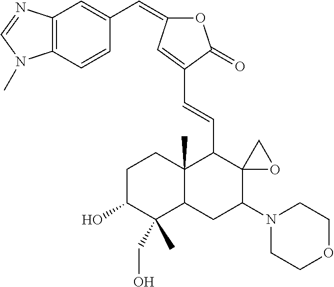 [SMILES: Cn1cnc2cc(/C=C3C=C(/C=C/C4C5(CO5)C(N5CCOCC5)CC5[C@]4(C)CC[C@@H](O)[C@@]5(C)CO)C(=O)O\3)ccc21] | 575.30 |
| 144 | 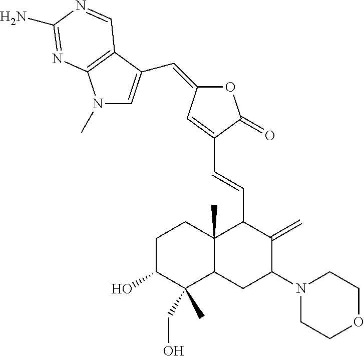 [SMILES: C=C1C(N2CCOCC2)CC2[C@](C)(CC[C@@H](O)[C@@]2(C)CO)C1/C=C/C1=CC(=C\c2cn(C)c3nc(N)ncc23)/OC1=O] | 575.31 |
| 145 | 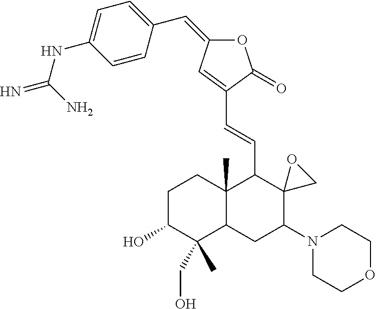 [SMILES: C[C@]12CC[C@@H](O)[C@@](C)(CO)C1CC(N1CCOCC1)C1(CO1)C2/C=C/C1=CC(=C\c2ccc(NC(=N)N)cc2)/OC1=O] | 578.31 |
| 146 | 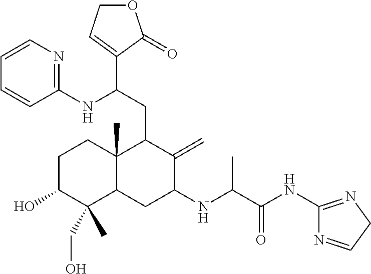 [SMILES: C=C1C(NC(C)C(=O)NC2=NCC=N2)CC2[C@](C)(CC[C@@H](O)[C@@]2(C)CO)C1CC(Nc1ccccn1)C1=CCOC1=O] | 578.32 |
| 147 | 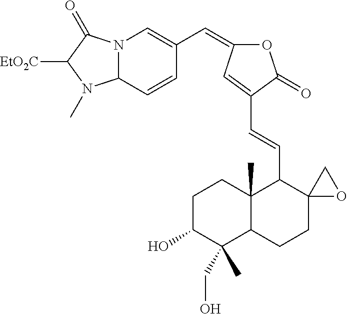 [SMILES: CCOC(=O)C1C(=O)N2C=C(/C=C3C=C(/C=C/C4C5(CCC6[C@]4(C)CC[C@@H](O)[C@@]6(C)CO)CO5)C(=O)O\3)C=CC2N1C] | 580.28 |
| 148 | 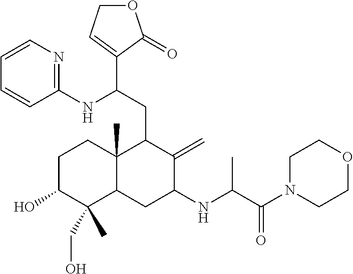 [SMILES: C=C1C(NC(C)C(=O)N2CCOCC2)CC2[C@](C)(CC[C@@H](O)[C@@]2(C)CO)C1CC(Nc1ccccn1)C1=CCOC1=O] | 582.34 |
| 149 | 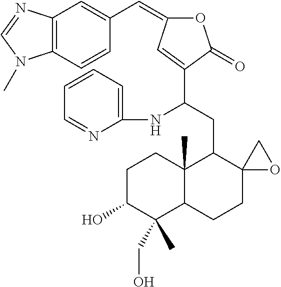 [SMILES: Cn1cnc2cc(/C=C3\C=C(C(CC4C5(CCC6[C@]4(C)CC[C@@H](O)[C@@]6(C)CO)CO5)Nc4ccccn4)C(=O)O3)ccc21] | 584.30 |
| 150 | 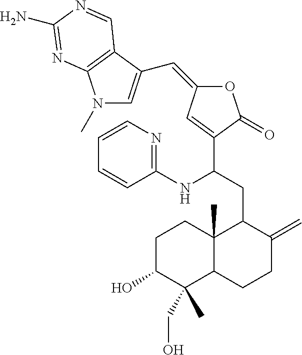 [SMILES: C=C1CCC2[C@](C)(CC[C@@H](O)[C@@]2(C)CO)C1CC(Nc1ccccn1)C1=C/C(=C\c2cn(C)c3nc(N)ncc23)OC1=O] | 584.31 |
| 151 | 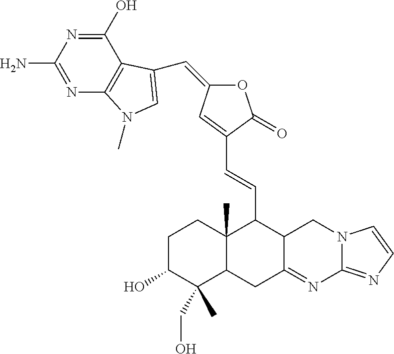 [SMILES: Cn1cc(/C=C2C=C(/C=C/C3C4Cn5ccnc5N=C4CC4[C@]3(C)CC[C@@H](O)[C@@]4(C)CO)C(=O)O\2)c2c(O)nc(N)nc21] | 585.27 |
| 152 | 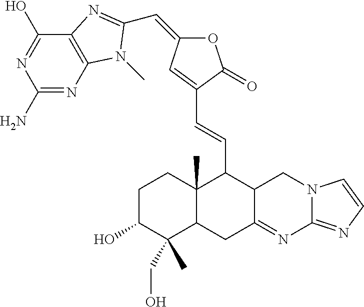 [SMILES: Cn1c(/C=C2C=C(/C=C/C3C4Cn5ccnc5N=C4CC4[C@]3(C)CC[C@@H](O)[C@@]4(C)CO)C(=O)O\2)nc2c(O)nc(N)nc21] | 586.27 |
| 153 | 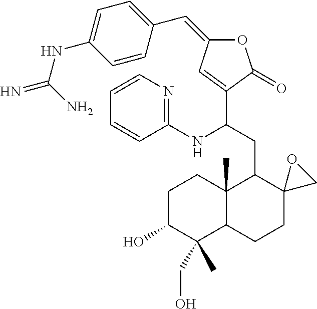 [SMILES: C[C@]12CC[C@@H](O)[C@@](C)(CO)C1CCC1(CO1)C2CC(Nc1ccccn1)C1=C/C(=C\c2ccc(NC(=N)N)cc2)OC1=O] | 587.31 |
| 154 | 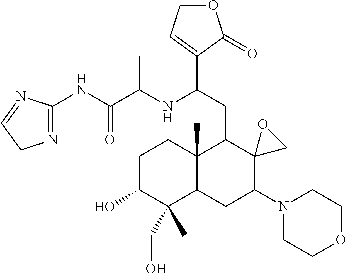 [SMILES: CC(NC(CC1C2(CO2)C(N2CCOCC2)CC2[C@]1(C)CC[C@@H](O)[C@@]2(C)CO)C1=CCOC1=O)C(=O)NC1=NCC=N1] | 587.33 |
| 155 | 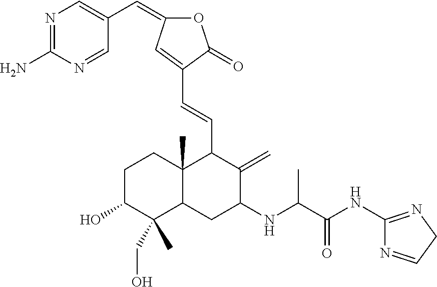 [SMILES: C=C1C(NC(C)C(=O)NC2=NCC=N2)CC2[C@](C)(CC[C@@H](O)[C@@]2(C)CO)C1/C=C/C1=CC(=C\c2cnc(N)nc2)/OC1=O] | 589.30 |
| 156 | 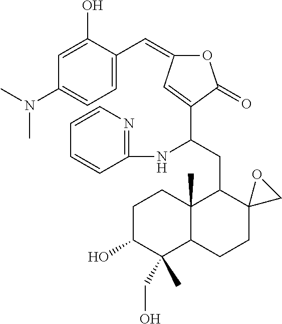 [SMILES: CN(C)c1ccc(/C=C2\C=C(C(CC3C4(CCC5[C@]3(C)CC[C@@H](O)[C@@]5(C)CO)CO4)Nc3ccccn3)C(=O)O2)c(O)c1] | 589.32 |
| 157 | 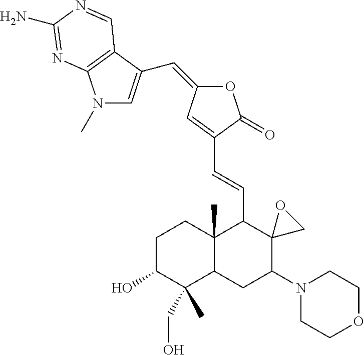 [SMILES: Cn1cc(/C=C2C=C(/C=C/C3C4(CO4)C(N4CCOCC4)CC4[C@]3(C)CC[C@@H](O)[C@@]4(C)CO)C(=O)O\2)c2cnc(N)nc21] | 591.31 |
| 158 | 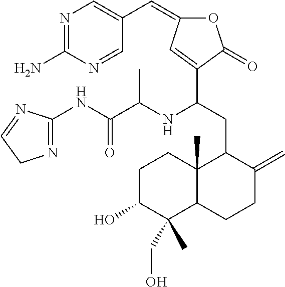 [SMILES: C=C1CCC2[C@](C)(CC[C@@H](O)[C@@]2(C)CO)C1CC(NC(C)C(=O)NC1=NCC=N1)C1=C/C(=C\c2cnc(N)nc2)OC1=O] | 591.32 |
| 159 | 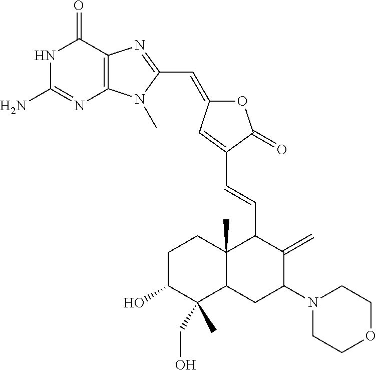 [SMILES: C=C1C(N2CCOCC2)CC2[C@](C)(CC[C@@H](O)[C@@]2(C)CO)C1/C=C/C1=CC(=C\c2nc3c(=O)[nH]c(N)nc3n2C)/OC1=O] | 592.30 |
| 160 | 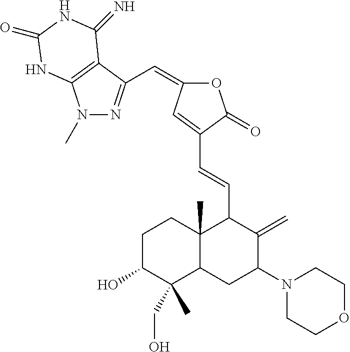 [SMILES: C=C1C(N2CCOCC2)CC2[C@](C)(CC[C@@H](O)[C@@]2(C)CO)C1/C=C/C1=CC(=C\c2nn(C)c3[nH]c(=O)[nH]c(=N)c23)/OC1=O] | 592.30 |
| 161 | 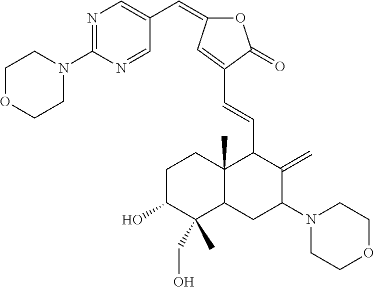 [SMILES: C=C1C(N2CCOCC2)CC2[C@](C)(CC[C@@H](O)[C@@]2(C)CO)C1/C=C/C1=CC(=C\c2cnc(N3CCOCC3)nc2)/OC1=O] | 592.33 |
| 162 | 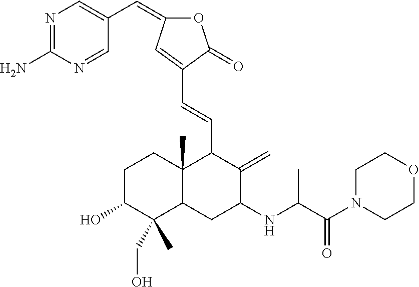 [SMILES: C=C1C(NC(C)C(=O)N2CCOCC2)CC2[C@](C)(CC[C@@H](O)[C@@]2(C)CO)C1/C=C/C1=CC(=C\c2cnc(N)nc2)/OC1=O] | 593.32 |
| 163 | 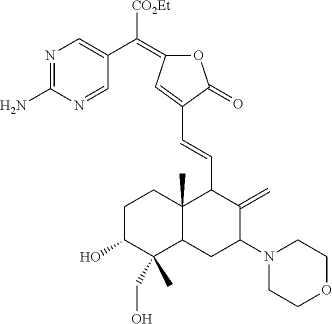 [SMILES: C=C1C(N2CCOCC2)CC2[C@](C)(CC[C@@H](O)[C@@]2(C)CO)C1/C=C/C1=CC(=C(/C(=O)OCC)c2cnc(N)nc2)/OC1=O] | 594.31 |
| 164 | 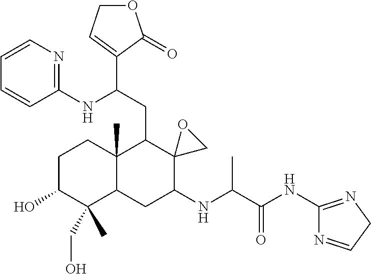 [SMILES: CC(NC1CC2[C@](C)(CC[C@@H](O)[C@@]2(C)CO)C(CC(Nc2ccccn2)C2=CCOC2=O)C12CO2)C(=O)NC1=NCC=N1] | 594.32 |
| 165 | 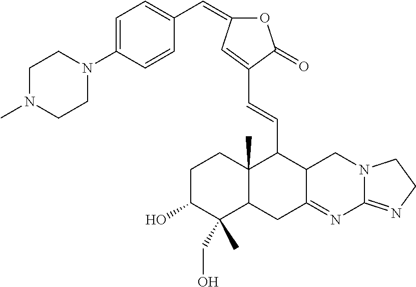 [SMILES: CN1CCN(c2ccc(/C=C3C=C(/C=C/C4C5CN6CCN=C6N=C5CC5[C@]4(C)CC[C@@H](O)[C@@]5(C)CO)C(=O)O\3)cc2)CC1] | 597.33 |
| 166 | 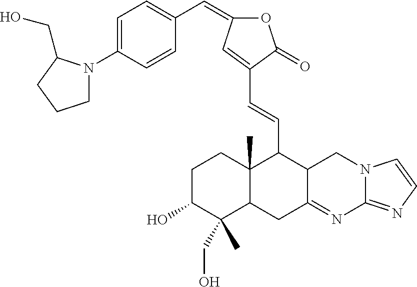 [SMILES: C[C@]12CC[C@@H](O)[C@@](C)(CO)C1CC1=Nc3nccn3CC1C2/C=C/C1=CC(=C\c2ccc(N3CCCC3CO)cc2)/OC1=O] | 598.32 |
| 167 | 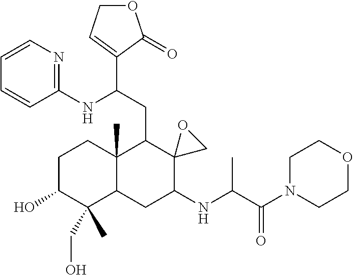 [SMILES: CC(NC1CC2[C@](C)(CC[C@@H](O)[C@@]2(C)CO)C(CC(Nc2ccccn2)C2=CCOC2=O)C12CO2)C(=O)N1CCOCC1] | 598.34 |
| 168 | 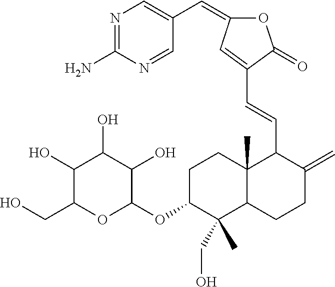 [SMILES: C=C1CCC2[C@](C)(CC[C@@H](OC3OC(CO)C(O)C(O)C3O)[C@@]2(C)CO)C1/C=C/C1=CC(=C\c2cnc(N)nc2)/OC1=O] | 599.28 |
| 169 | 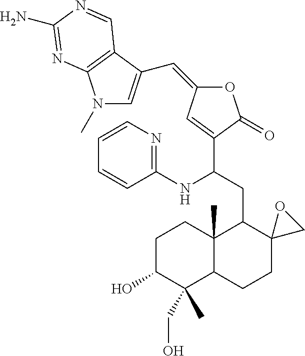 [SMILES: Cn1cc(/C=C2\C=C(C(CC3C4(CCC5[C@]3(C)CC[C@@H](O)[C@@]5(C)CO)CO4)Nc3ccccn3)C(=O)O2)c2cnc(N)nc21] | 600.31 |
| 170 | 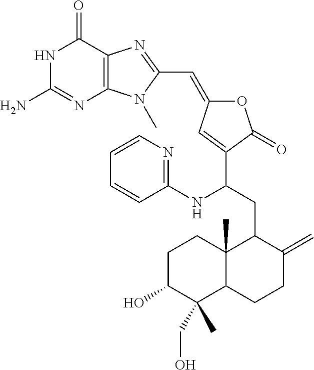 [SMILES: C=C1CCC2[C@](C)(CC[C@@H](O)[C@@]2(C)CO)C1CC(Nc1ccccn1)C1=C/C(=C\c2nc3c(=O)[nH]c(N)nc3n2C)OC1=O] | 601.30 |
| 171 | 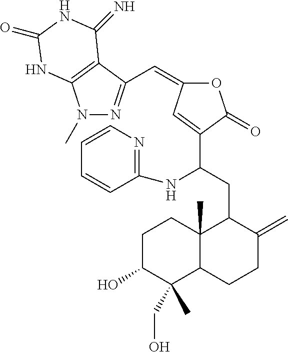 [SMILES: C=C1CCC2[C@](C)(CC[C@@H](O)[C@@]2(C)CO)C1CC(Nc1ccccn1)C1=C/C(=C\c2nn(C)c3[nH]c(=O)[nH]c(=N)c23)OC1=O] | 601.30 |
| 172 | 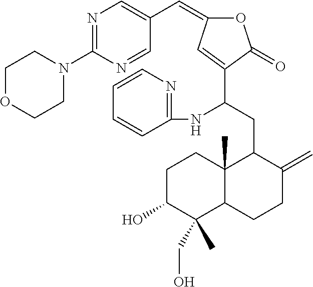 [SMILES: C=C1CCC2[C@](C)(CC[C@@H](O)[C@@]2(C)CO)C1CC(Nc1ccccn1)C1=C/C(=C\c2cnc(N3CCOCC3)nc2)OC1=O] | 601.33 |
| 173 | 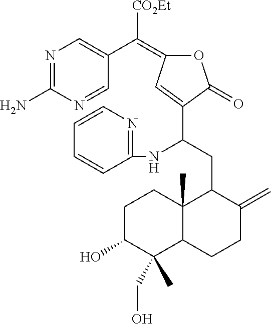 [SMILES: C=C1CCC2[C@](C)(CC[C@@H](O)[C@@]2(C)CO)C1CC(Nc1ccccn1)C1=C/C(=C(/C(=O)OCC)c2cnc(N)nc2)OC1=O] | 603.31 |
| 174 | 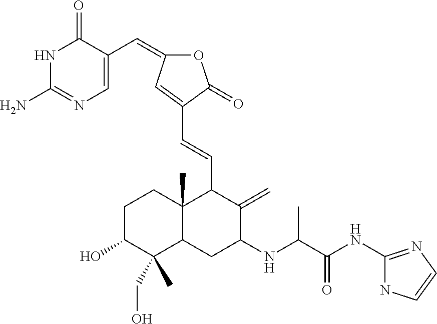 [SMILES: C=C1C(NC(C)C(=O)Nc2ncc[nH]2)CC2[C@](C)(CC[C@@H](O)[C@@]2(C)CO)C1/C=C/C1=CC(=C\c2cnc(N)[nH]c2=O)/OC1=O] | 605.30 |
| 175 | 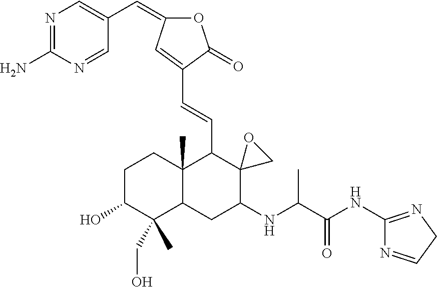 [SMILES: CC(NC1CC2[C@](C)(CC[C@@H](O)[C@@]2(C)CO)C(/C=C/C2=CC(=C\c3cnc(N)nc3)/OC2=O)C12CO2)C(=O)NC1=NCC=N1] | 605.30 |
| 176 | 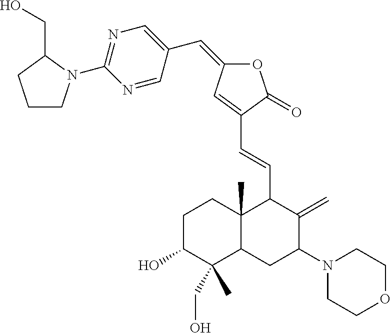 [SMILES: C=C1C(N2CCOCC2)CC2[C@](C)(CC[C@@H](O)[C@@]2(C)CO)C1/C=C/C1=CC(=C\c2cnc(N3CCCC3CO)nc2)/OC1=O] | 606.34 |
| 177 | 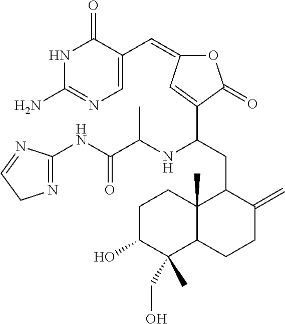 [SMILES: C=C1CCC2[C@](C)(CC[C@@H](O)[C@@]2(C)CO)C1CC(NC(C)C(=O)NC1=NCC=N1)C1=C/C(=C\c2cnc(N)[nH]c2=O)OC1=O] | 607.31 |
| 178 | 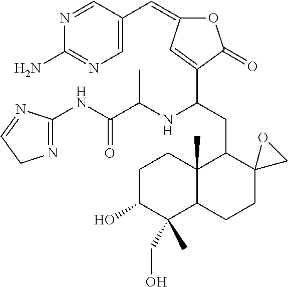 [SMILES: CC(NC(CC1C2(CCC3[C@]1(C)CC[C@@H](O)[C@@]3(C)CO)CO2)C1=C/C(=C\c2cnc(N)nc2)OC1=O)C(=O)NC1=NCC=N1] | 607.31 |
| 179 | 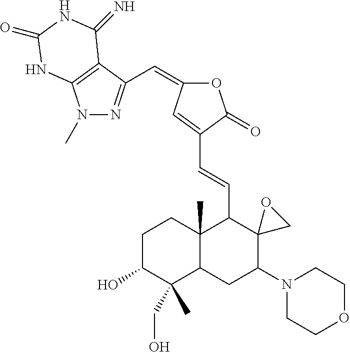 [SMILES: Cn1nc(/C=C2C=C(/C=C/C3C4(CO4)C(N4CCOCC4)CC4[C@]3(C)CC[C@@H](O)[C@@]4(C)CO)C(=O)O\2)c2c(=N)[nH]c(=O)[nH]c21] | 608.30 |
| 180 | 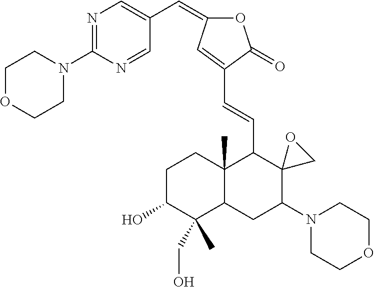 [SMILES: C[C@]12CC[C@@H](O)[C@@](C)(CO)C1CC(N1CCOCC1)C1(CO1)C2/C=C/C1=CC(=C\c2cnc(N3CCOCC3)nc2)/OC1=O] | 608.32 |
| 181 | 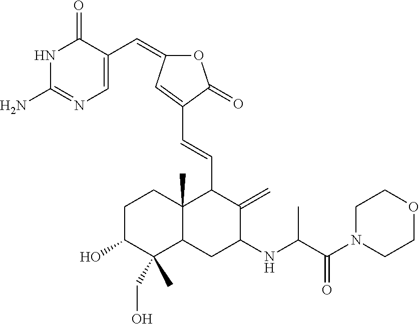 [SMILES: C=C1C(NC(C)C(=O)N2CCOCC2)CC2[C@](C)(CC[C@@H](O)[C@@]2(C)CO)C1/C=C/C1=CC(=C\c2cnc(N)[nH]c2=O)/OC1=O] | 609.32 |
| 182 | 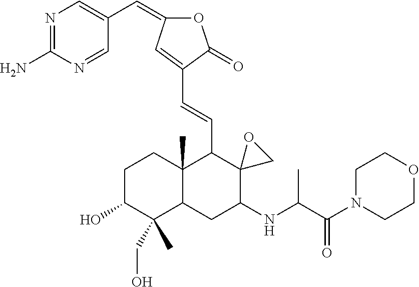 [SMILES: CC(NC1CC2[C@](C)(CC[C@@H](O)[C@@]2(C)CO)C(/C=C/C2=CC(=C\c3cnc(N)nc3)/OC2=O)C12CO2)C(=O)N1CCOCC1] | 609.32 |
| 183 | 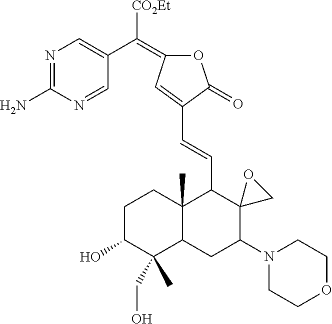 [SMILES: CCOC(=O)/C(=C1C=C(/C=C/C2C3(CO3)C(N3CCOCC3)CC3[C@]2(C)CC[C@@H](O)[C@@]3(C)CO)C(=O)O/1)c1cnc(N)nc1] | 610.30 |
| 184 | 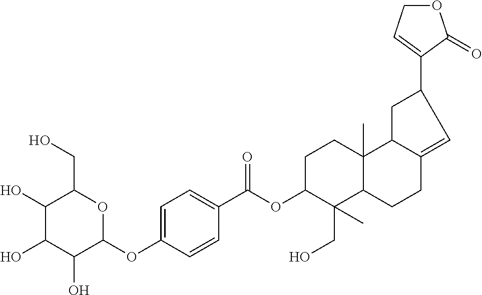 [SMILES: CC1(CO)C(OC(=O)c2ccc(OC3OC(CO)C(O)C(O)C3O)cc2)CCC2(C)C3CC(C4=CCOC4=O)C=C3CCC12] | 614.27 |
| 185 | 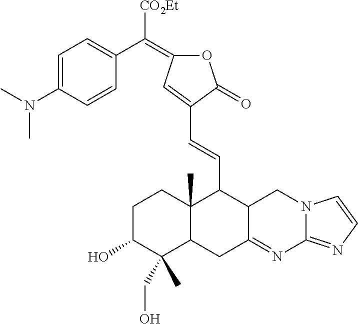 [SMILES: CCOC(=O)/C(=C1C=C(/C=C/C2C3Cn4ccnc4N=C3CC3[C@]2(C)CC[C@@H](O)[C@@]3(C)CO)C(=O)O/1)c1ccc(N(C)C)cc1] | 614.31 |
| 186 | 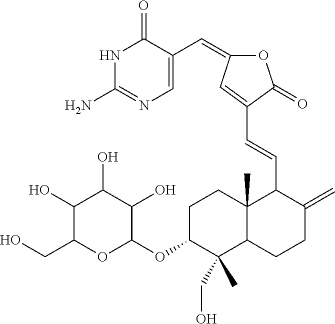 [SMILES: C=C1CCC2[C@](C)(CC[C@@H](OC3OC(CO)C(O)C(O)C3O)[C@@]2(C)CO)C1/C=C/C1=CC(=C\c2cnc(N)[nH]c2=O)/OC1=O] | 615.28 |
| 187 | 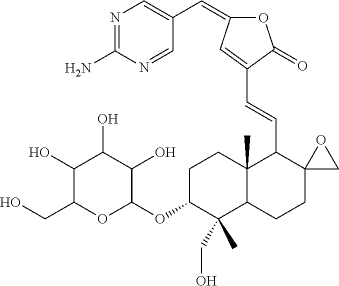 [SMILES: C[C@]12CC[C@@H](OC3OC(CO)C(O)C(O)C3O)[C@@](C)(CO)C1CCC1(CO1)C2/C=C/C1=CC(=C\c2cnc(N)nc2)/OC1=O] | 615.28 |
| 188 | 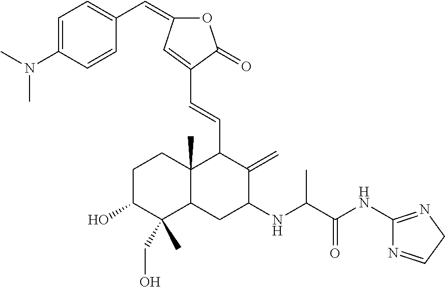 [SMILES: C=C1C(NC(C)C(=O)NC2=NCC=N2)CC2[C@](C)(CC[C@@H](O)[C@@]2(C)CO)C1/C=C/C1=CC(=C\c2ccc(N(C)C)cc2)/OC1=O] | 615.34 |
| 189 | 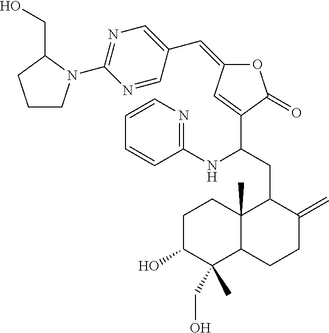 [SMILES: C=C1CCC2[C@](C)(CC[C@@H](O)[C@@]2(C)CO)C1CC(Nc1ccccn1)C1=C/C(=C\c2cnc(N3CCCC3CO)nc2)OC1=O] | 615.34 |
| 190 | 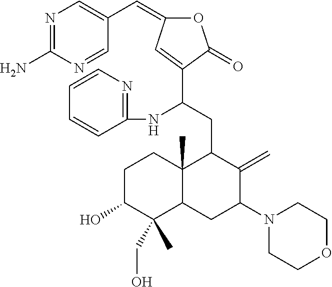 [SMILES: C=C1C(N2CCOCC2)CC2[C@](C)(CC[C@@H](O)[C@@]2(C)CO)C1CC(Nc1ccccn1)C1=C/C(=C\c2cnc(N)nc2)OC1=O] | 616.34 |
| 191 | 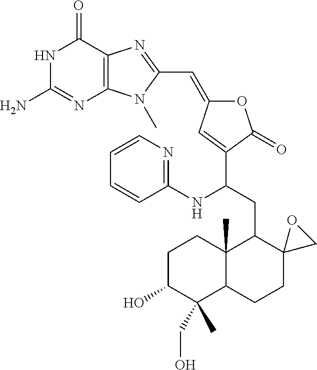 [SMILES: Cn1c(/C=C2\C=C(C(CC3C4(CCC5[C@]3(C)CC[C@@H](O)[C@@]5(C)CO)CO4)Nc3ccccn3)C(=O)O2)nc2c(=O)[nH]c(N)nc21] | 617.30 |
| 192 | 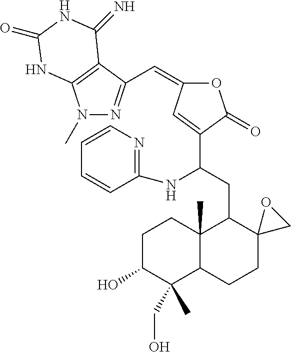 [SMILES: Cn1nc(/C=C2\C=C(C(CC3C4(CCC5[C@]3(C)CC[C@@H](O)[C@@]5(C)CO)CO4)Nc3ccccn3)C(=O)O2)c2c(=N)[nH]c(=O)[nH]c21] | 617.30 |
| 193 | 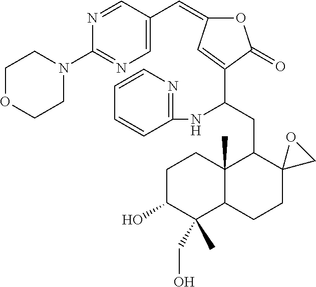 [SMILES: C[C@]12CC[C@@H](O)[C@@](C)(CO)C1CCC1(CO1)C2CC(Nc1ccccn1)C1=C/C(=C\c2cnc(N3CCOCC3)nc2)OC1=O] | 617.32 |
| 194 | 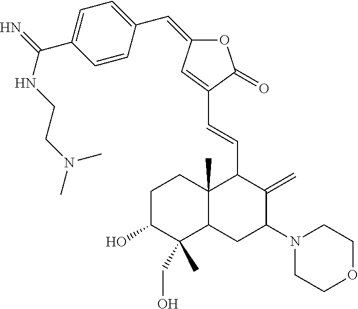 [SMILES: C=C1C(N2CCOCC2)CC2[C@](C)(CC[C@@H](O)[C@@]2(C)CO)C1/C=C/C1=CC(=C\c2ccc(C(=N)NCCN(C)C)cc2)/OC1=O] | 618.38 |
| 195 | 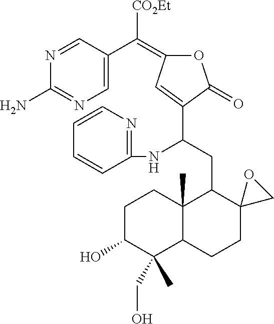 [SMILES: CCOC(=O)/C(=C1/C=C(C(CC2C3(CCC4[C@]2(C)CC[C@@H](O)[C@@]4(C)CO)CO3)Nc2ccccn2)C(=O)O1)c1cnc(N)nc1] | 619.30 |
| 196 | 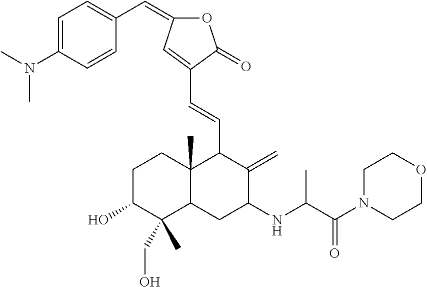 [SMILES: C=C1C(NC(C)C(=O)N2CCOCC2)CC2[C@](C)(CC[C@@H](O)[C@@]2(C)CO)C1/C=C/C1=CC(=C\c2ccc(N(C)C)cc2)/OC1=O] | 619.36 |
| 197 | 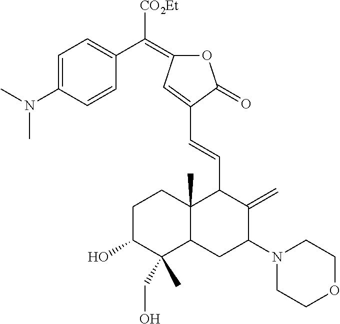 [SMILES: C=C1C(N2CCOCC2)CC2[C@](C)(CC[C@@H](O)[C@@]2(C)CO)C1/C=C/C1=CC(=C(/C(=O)OCC)c2ccc(N(C)C)cc2)/OC1=O] | 620.35 |
| 198 | 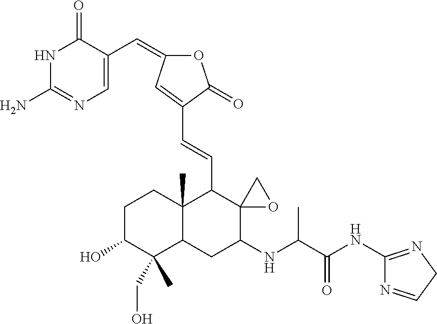 [SMILES: CC(NC1CC2[C@](C)(CC[C@@H](O)[C@@]2(C)CO)C(/C=C/C2=CC(=C\c3cnc(N)[nH]c3=O)/OC2=O)C12CO2)C(=O)NC1=NCC=N1] | 621.29 |
| 199 | 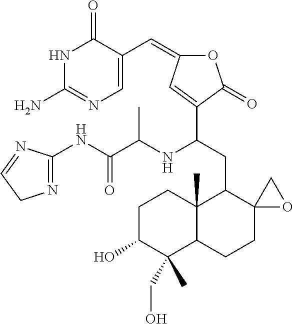 [SMILES: CC(NC(CC1C2(CCC3[C@]1(C)CC[C@@H](O)[C@@]3(C)CO)CO2)C1=C/C(=C\c2cnc(N)[nH]c2=O)OC1=O)C(=O)NC1=NCC=N1] | 623.31 |
| 200 | 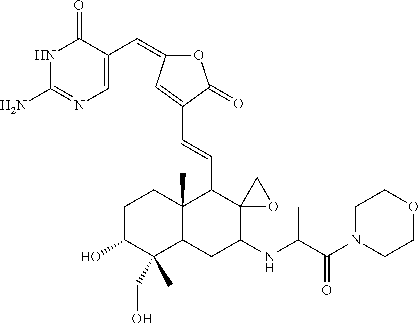 [SMILES: CC(NC1CC2[C@](C)(CC[C@@H](O)[C@@]2(C)CO)C(/C=C/C2=CC(=C\c3cnc(N)[nH]c3=O)/OC2=O)C12CO2)C(=O)N1CCOCC1] | 625.31 |
| 201 | 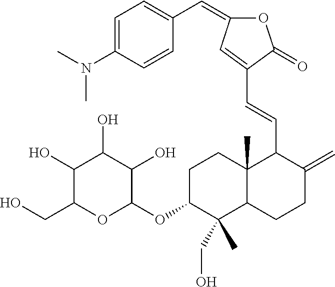 [SMILES: C=C1CCC2[C@](C)(CC[C@@H](OC3OC(CO)C(O)C(O)C3O)[C@@]2(C)CO)C1/C=C/C1=CC(=C\c2ccc(N(C)C)cc2)/OC1=O] | 625.33 |
| 202 | 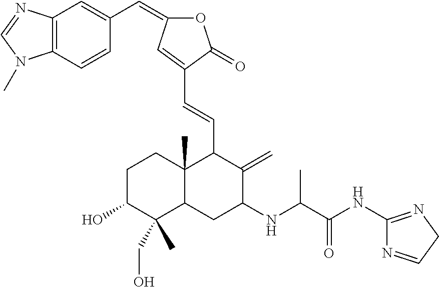 [SMILES: C=C1C(NC(C)C(=O)NC2=NCC=N2)CC2[C@](C)(CC[C@@H](O)[C@@]2(C)CO)C1/C=C/C1=CC(=C\c2ccc3c(c2)ncn3C)/OC1=O] | 626.32 |
| 203 | 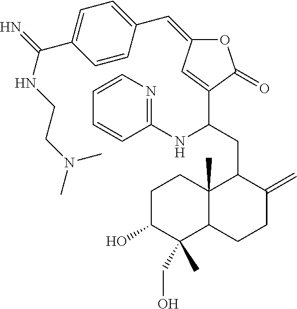 [SMILES: C=C1CCC2[C@](C)(CC[C@@H](O)[C@@]2(C)CO)C1CC(Nc1ccccn1)C1=C/C(=C\c2ccc(C(=N)NCCN(C)C)cc2)OC1=O] | 627.38 |
| 204 | 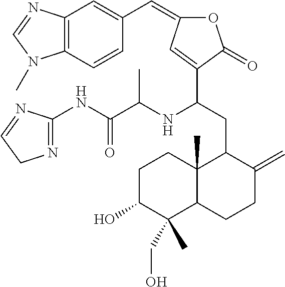 [SMILES: C=C1CCC2[C@](C)(CC[C@@H](O)[C@@]2(C)CO)C1CC(NC(C)C(=O)NC1=NCC=N1)C1=C/C(=C\c2ccc3c(c2)ncn3C)OC1=O] | 628.34 |
| 205 | 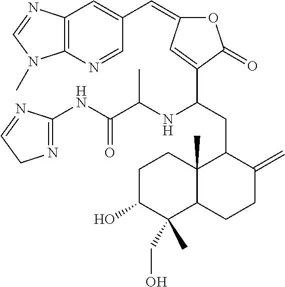 [SMILES: C=C1CCC2[C@](C)(CC[C@@H](O)[C@@]2(C)CO)C1CC(NC(C)C(=O)NC1=NCC=N1)C1=C/C(=C\c2cnc3c(c2)ncn3C)OC1=O] | 629.33 |
| 206 | 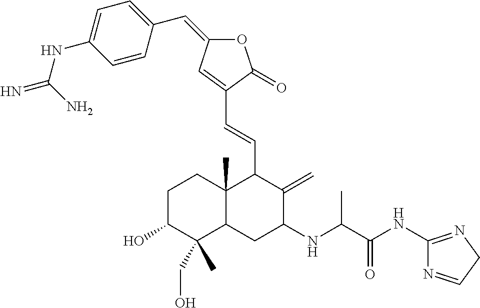 [SMILES: C=C1C(NC(C)C(=O)NC2=NCC=N2)CC2[C@](C)(CC[C@@H](O)[C@@]2(C)CO)C1/C=C/C1=CC(=C\c2ccc(NC(=N)N)cc2)/OC1=O] | 629.33 |
| 207 | 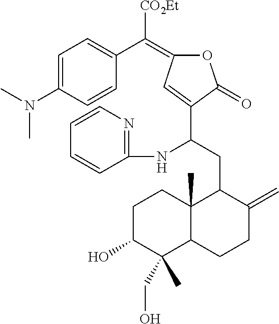 | 629.35 |
| 208 | 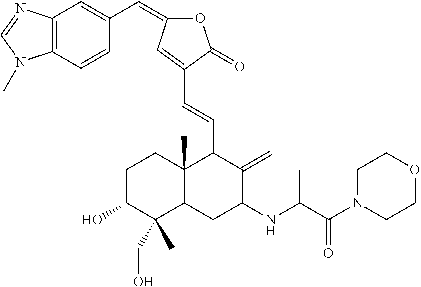 [SMILES: C=C1C(NC(C)C(=O)N2CCOCC2)CC2[C@](C)(CC[C@@H](O)[C@@]2(C)CO)C1/C=C/C1=CC(=C\c2ccc3c(c2)ncn3C)/OC1=O] | 630.34 |
| 209 | 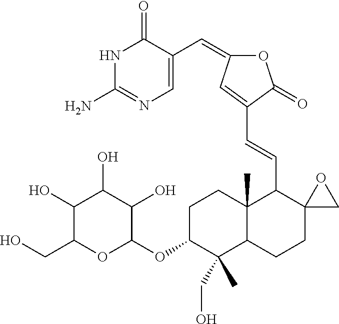 [SMILES: C[C@]12CC[C@@H](OC3OC(CO)C(O)C(O)C3O)[C@@](C)(CO)C1CCC1(CO1)C2/C=C/C1=CC(=C\c2cnc(N)[nH]c2=O)/OC1=O] | 631.27 |
| 210 | 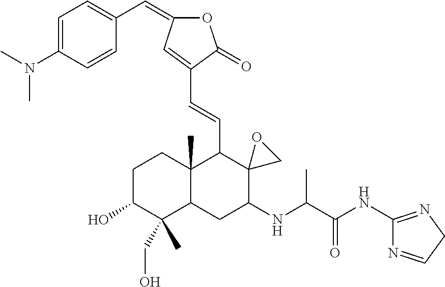 [SMILES: CC(NC1CC2[C@](C)(CC[C@@H](O)[C@@]2(C)CO)C(/C=C/C2=CC(=C\c3ccc(N(C)C)cc3)/OC2=O)C12CO2)C(=O)NC1=NCC=N1] | 631.34 |
| 211 | 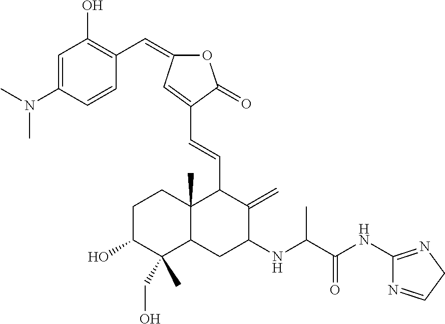 [SMILES: C=C1C(NC(C)C(=O)NC2=NCC=N2)CC2[C@](C)(CC[C@@H](O)[C@@]2(C)CO)C1/C=C/C1=CC(=C\c2ccc(N(C)C)cc2O)/OC1=O] | 631.34 |
| 212 | 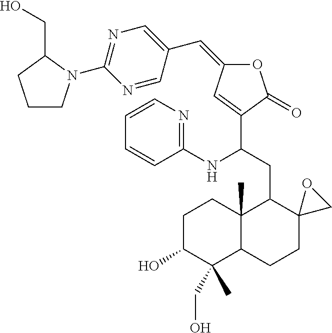 [SMILES: C[C@]12CC[C@@H](O)[C@@](C)(CO)C1CCC1(CO1)C2CC(Nc1ccccn1)C1=C/C(=C\c2cnc(N3CCCC3CO)nc2)OC1=O] | 631.34 |
| 213 | 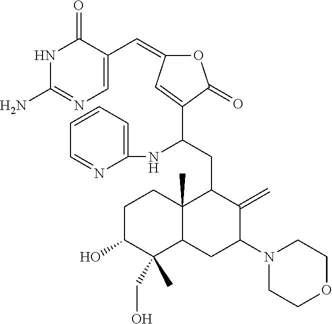 [SMILES: C=C1C(N2CCOCC2)CC2[C@](C)(CC[C@@H](O)[C@@]2(C)CO)C1CC(Nc1ccccn1)C1=C/C(=C\c2cnc(N)[nH]c2=O)OC1=O] | 632.33 |
| 214 | 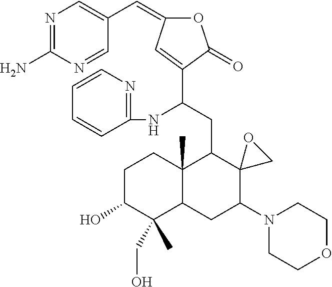 [SMILES: C[C@]12CC[C@@H](O)[C@@](C)(CO)C1CC(N1CCOCC1)C1(CO1)C2CC(Nc1ccccn1)C1=C/C(=C\c2cnc(N)nc2)OC1=O] | 632.33 |
| 215 | 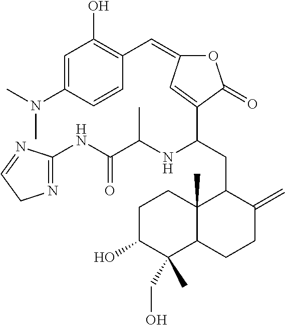 [SMILES: C=C1CCC2[C@](C)(CC[C@@H](O)[C@@]2(C)CO)C1CC(NC(C)C(=O)NC1=NCC=N1)C1=C/C(=C\c2ccc(N(C)C)cc2O)OC1=O] | 633.35 |
| 216 | 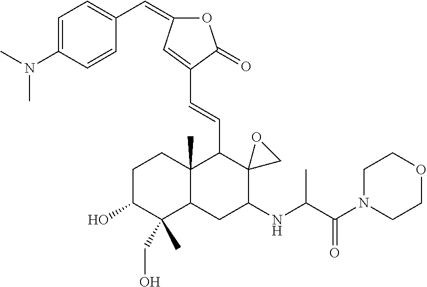 [SMILES: CC(NC1CC2[C@](C)(CC[C@@H](O)[C@@]2(C)CO)C(/C=C/C2=CC(=C\c3ccc(N(C)C)cc3)/OC2=O)C12CO2)C(=O)N1CCOCC1] | 635.36 |
| 217 | 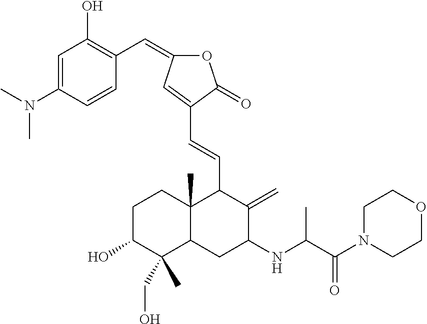 [SMILES: C=C1C(NC(C)C(=O)N2CCOCC2)CC2[C@](C)(CC[C@@H](O)[C@@]2(C)CO)C1/C=C/C1=CC(=C\c2ccc(N(C)C)cc2O)/OC1=O] | 635.36 |
| 218 | 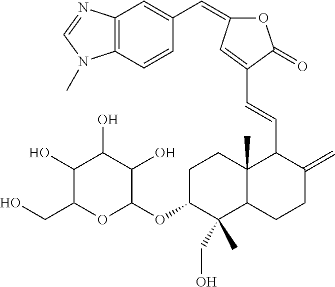 [SMILES: C=C1CCC2[C@](C)(CC[C@@H](OC3OC(CO)C(O)C(O)C3O)[C@@]2(C)CO)C1/C=C/C1=CC(=C\c2ccc3c(c2)ncn3C)/OC1=O] | 636.30 |
| 219 | 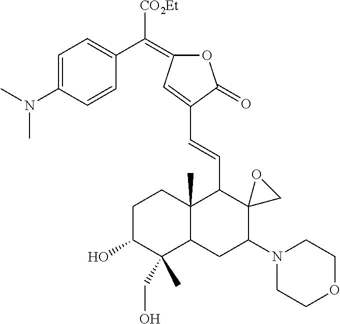 [SMILES: CCOC(=O)/C(=C1C=C(/C=C/C2C3(CO3)C(N3CCOCC3)CC3[C@]2(C)CC[C@@H](O)[C@@]3(C)CO)C(=O)O/1)c1ccc(N(C)C)cc1] | 636.34 |
| 220 | 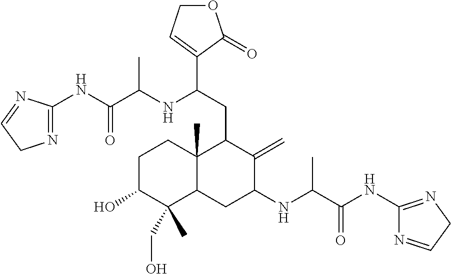 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=CCOC1=O] | 638.35 |
| 221 | 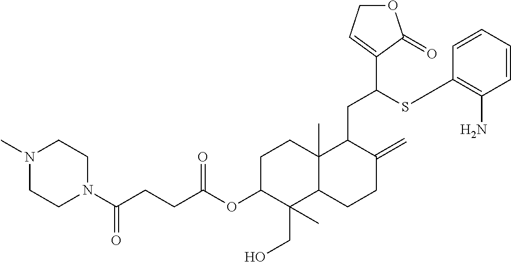 [SMILES: C=C1CCC2C(C)(CO)C(OC(=O)CCC(=O)N3CCN(C)CC3)CCC2(C)C1CC(Sc1ccccc1N)C1=CCOC1=O] | 639.33 |
| 222 | 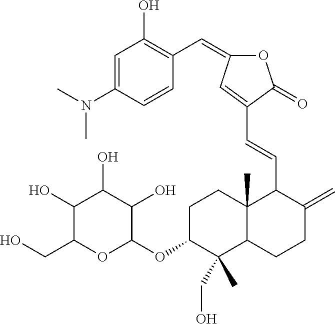 [SMILES: C=C1CCC2[C@](C)(CC[C@@H](OC3OC(CO)C(O)C(O)C3O)[C@@]2(C)CO)C1/C=C/C1=CC(=C\c2ccc(N(C)C)cc2O)/OC1=O] | 641.32 |
| 223 | 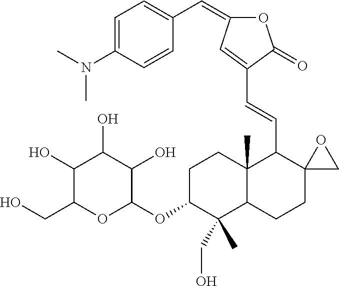 [SMILES: CN(C)c1ccc(/C=C2C=C(/C=C/C3C4(CCC5[C@]3(C)CC[C@@H](OC3OC(CO)C(O)C(O)C3O)[C@@]5(C)CO)CO4)C(=O)O\2)cc1] | 641.32 |
| 224 | 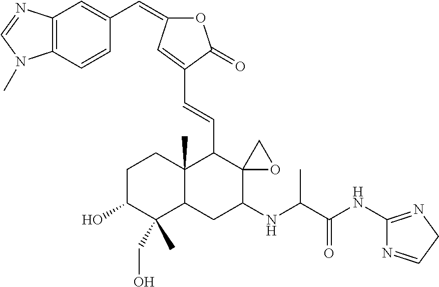 [SMILES: CC(NC1CC2[C@](C)(CC[C@@H](O)[C@@]2(C)CO)C(/C=C/C2=CC(=C\c3ccc4c(c3)ncn4C)/OC2=O)C12CO2)C(=O)NC1=NCC=N1] | 642.32 |
| 225 | 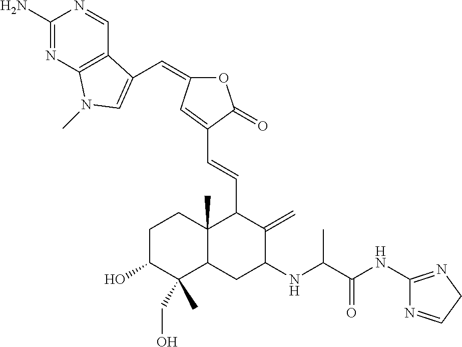 [SMILES: C=C1C(NC(C)C(=O)NC2=NCC=N2)CC2[C@](C)(CC[C@@H](O)[C@@]2(C)CO)C1/C=C/C1=CC(=C\c2cn(C)c3nc(N)ncc23)/OC1=O] | 642.33 |
| 226 | 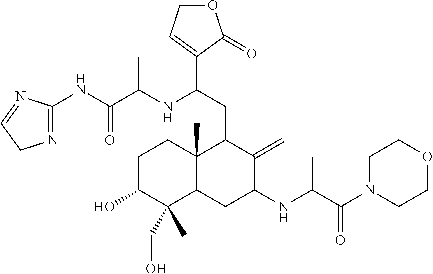 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=CCOC1=O] | 642.37 |
| 227 | 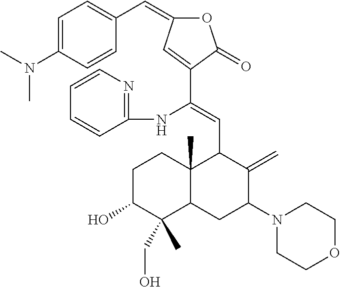 [SMILES: C=C1C(N2CCOCC2)CC2[C@](C)(CC[C@@H](O)[C@@]2(C)CO)C1/C=C(\Nc1ccccn1)C1=C/C(=C\c2ccc(N(C)C)cc2)OC1=O] | 642.38 |
| 228 | 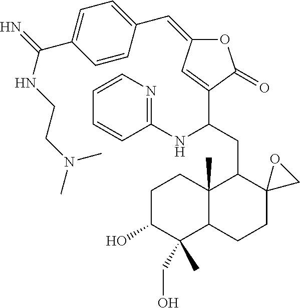 [SMILES: CN(C)CCNC(=N)c1ccc(/C=C2\C=C(C(CC3C4(CCC5[C@]3(C)CC[C@@H](O)[C@@]5(C)CO)CO4)Nc3ccccn3)C(=O)O2)cc1] | 643.37 |
| 229 | 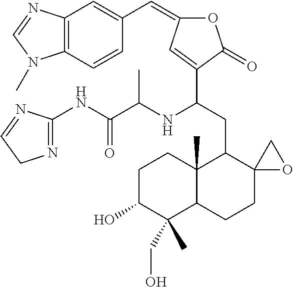 [SMILES: CC(NC(CC1C2(CCC3[C@]1(C)CC[C@@H](O)[C@@]3(C)CO)CO2)C1=C/C(=C\c2ccc3c(c2)ncn3C)OC1=O)C(=O)NC1=NCC=N1] | 644.33 |
| 230 | 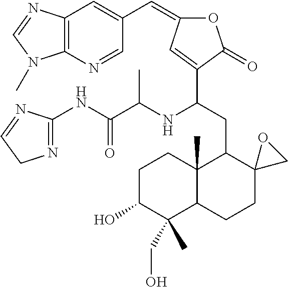 [SMILES: CC(NC(CC1C2(CCC3[C@]1(C)CC[C@@H](O)[C@@]3(C)CO)CO2)C1=C/C(=C\c2cnc3c(c2)ncn3C)OC1=O)C(=O)NC1=NCC=N1] | 645.33 |
| 231 | 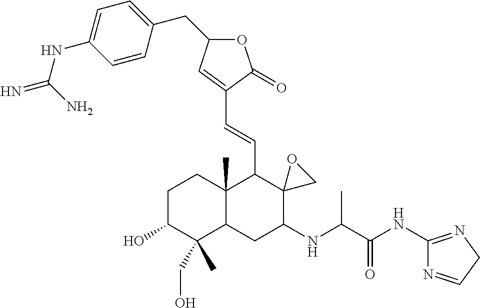 [SMILES: CC(NC1CC2[C@](C)(CC[C@@H](O)[C@@]2(C)CO)C(/C=C/C2=CC(Cc3ccc(NC(=N)N)cc3)OC2=O)C12CO2)C(=O)NC1=NCC=N1] | 645.33 |
| 232 | 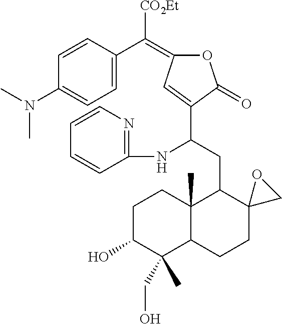 [SMILES: CCOC(=O)/C(=C1/C=C(C(CC2C3(CCC4[C@]2(C)CC[C@@H](O)[C@@]4(C)CO)CO3)Nc2ccccn2)C(=O)O1)c1ccc(N(C)C)cc1] | 645.34 |
| 233 | 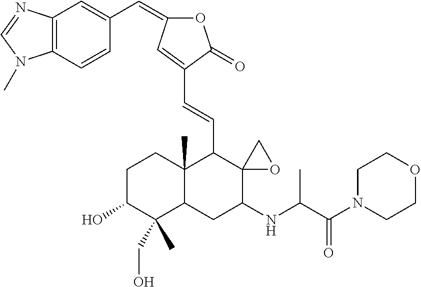 [SMILES: CC(NC1CC2[C@](C)(CC[C@@H](O)[C@@]2(C)CO)C(/C=C/C2=CC(=C\c3ccc4c(c3)ncn4C)/OC2=O)C12CO2)C(=O)N1CCOCC1] | 646.34 |
| 234 | 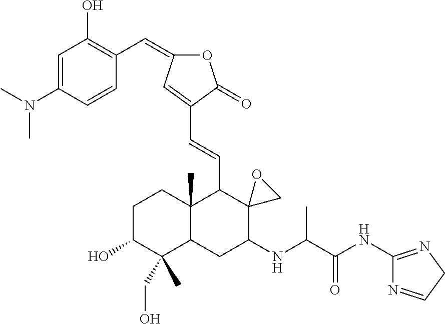 [SMILES: CC(NC1CC2[C@](C)(CC[C@@H](O)[C@@]2(C)CO)C(/C=C/C2=CC(=C\c3ccc(N(C)C)cc3O)/OC2=O)C12CO2)C(=O)NC1=NCC=N1] | 647.33 |
| 235 | 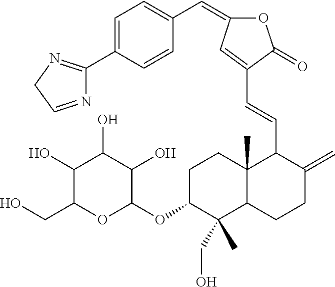 [SMILES: C=C1CCC2[C@](C)(CC[C@@H](OC3OC(CO)C(O)C(O)C3O)[C@@]2(C)CO)C1/C=C/C1=CC(=C\c2ccc(C3=NCC=N3)cc2)/OC1=O] | 648.30 |
| 236 | 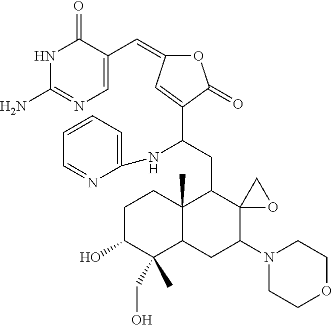 [SMILES: C[C@]12CC[C@@H](O)[C@@](C)(CO)C1CC(N1CCOCC1)C1(CO1)C2CC(Nc1ccccn1)C1=C/C(=C\c2cnc(N)[nH]c2=O)OC1=O] | 648.33 |
| 237 | 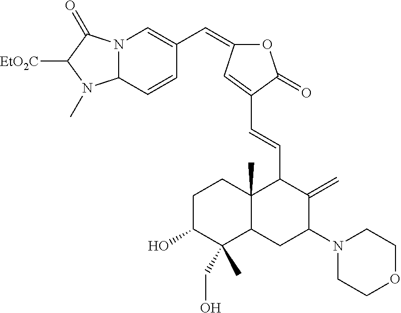 [SMILES: C=C1C(N2CCOCC2)CC2[C@](C)(CC[C@@H](O)[C@@]2(C)CO)C1/C=C/C1=CC(=C\C2=CN3C(=O)C(C(=O)OCC)N(C)C3C=C2)/OC1=O] | 649.34 |
| 238 | 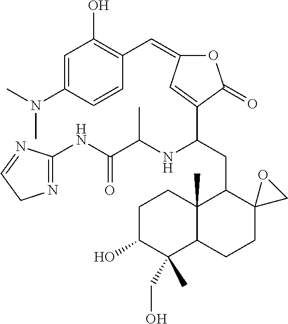 [SMILES: CC(NC(CC1C2(CCC3[C@]1(C)CC[C@@H](O)[C@@]3(C)CO)CO2)C1=C/C(=C\c2ccc(N(C)C)cc2O)OC1=O)C(=O)NC1=NCC=N1] | 649.35 |
| 239 | 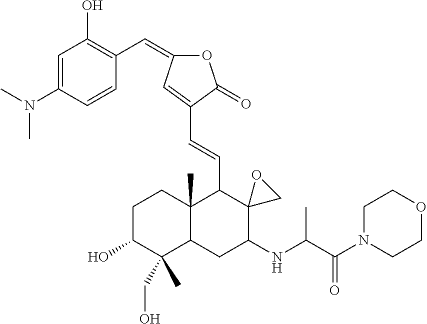 [SMILES: CC(NC1CC2[C@](C)(CC[C@@H](O)[C@@]2(C)CO)C(/C=C/C2=CC(=C\c3ccc(N(C)C)cc3O)/OC2=O)C12CO2)C(=O)N1CCOCC1] | 651.35 |
| 240 | 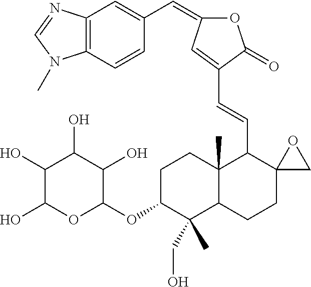 [SMILES: Cn1cnc2cc(/C=C3C=C(/C=C/C4C5(CCC6[C@]4(C)CC[C@@H](OC4OC(O)C(O)C(O)C4O)[C@@]6(C)CO)CO5)C(=O)O\3)ccc21] | 652.30 |
| 241 | 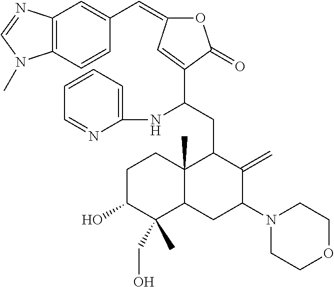 [SMILES: C=C1C(N2CCOCC2)CC2[C@](C)(CC[C@@H](O)[C@@]2(C)CO)C1CC(Nc1ccccn1)C1=C/C(=C\c2ccc3c(c2)ncn3C)OC1=O] | 653.36 |
| 242 | 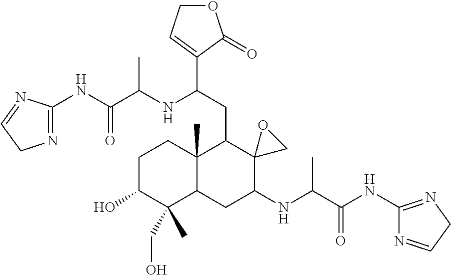 [SMILES: CC(NC(CC1C2(CO2)C(NC(C)C(=O)NC2=NCC=N2)CC2[C@]1(C)CC[C@@H](O)[C@@]2(C)CO)C1=CCOC1=O)C(=O)NC1=NCC=N1] | 654.35 |
| 243 | 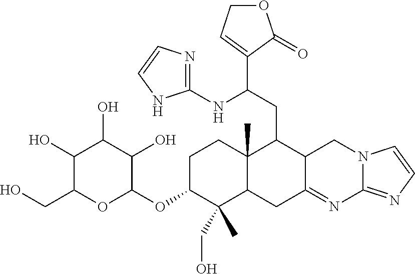 [SMILES: C[C@]12CC[C@@H](OC3OC(CO)C(O)C(O)C3O)[C@@](C)(CO)C1CC1=Nc3nccn3CC1C2CC(Nc1ncc[nH]1)C1=CCOC1=O] | 656.32 |
| 244 | 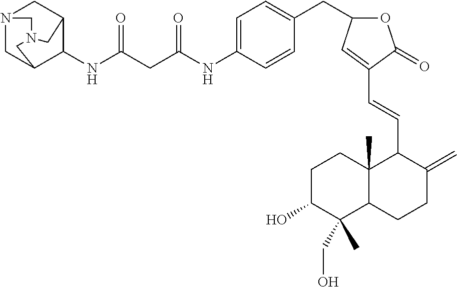 [SMILES: C=C1CCC2[C@](C)(CC[C@@H](O)[C@@]2(C)CO)C1/C=C/C1=CC(Cc2ccc(NC(=O)CC(=O)NC3C4CN5CC3CN(C4)C5)cc2)OC1=O] | 656.36 |
| 245 | 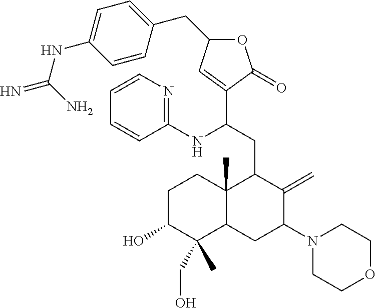 [SMILES: C=C1C(N2CCOCC2)CC2[C@](C)(CC[C@@H](O)[C@@]2(C)CO)C1CC(Nc1ccccn1)C1=CC(Cc2ccc(NC(=N)N)cc2)OC1=O] | 656.37 |
| 246 | 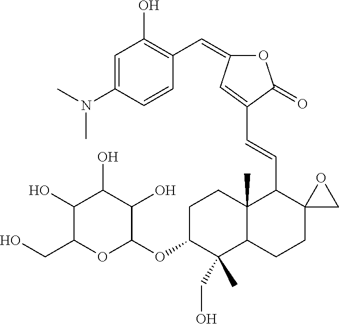 [SMILES: CN(C)c1ccc(/C=C2C=C(/C=C/C3C4(CCC5[C@]3(C)CC[C@@H](OC3OC(CO)C(O)C(O)C3O)[C@@]5(C)CO)CO4)C(=O)O\2)c(O)c1] | 657.31 |
| 247 | 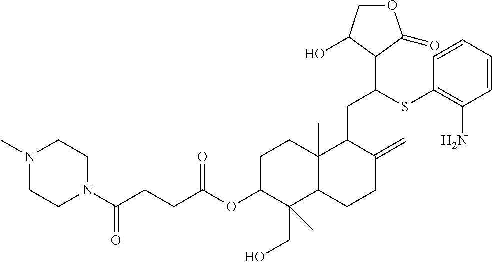 [SMILES: C=C1CCC2C(C)(CO)C(OC(=O)CCC(=O)N3CCN(C)CC3)CCC2(C)C1CC(Sc1ccccc1N)C1C(=O)OCC1O] | 657.34 |
| 248 | 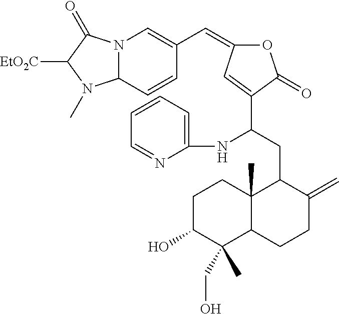 [SMILES: C=C1CCC2[C@](C)(CC[C@@H](O)[C@@]2(C)CO)C1CC(Nc1ccccn1)C1=C/C(=C\C2=CN3C(=O)C(C(=O)OCC)N(C)C3C=C2)OC1=O] | 658.34 |
| 249 | 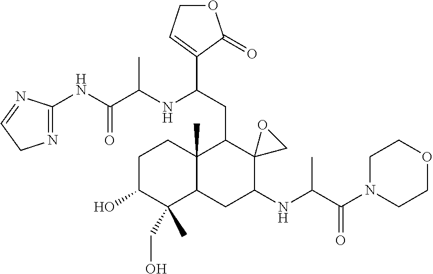 [SMILES: CC(NC(CC1C2(CO2)C(NC(C)C(=O)N2CCOCC2)CC2[C@]1(C)CC[C@@H](O)[C@@]2(C)CO)C1=CCOC1=O)C(=O)NC1=NCC=N1] | 658.37 |
| 250 | 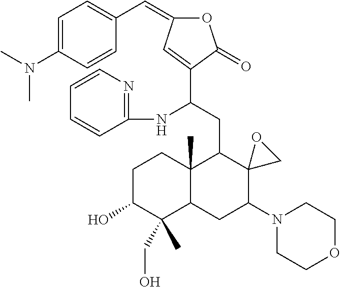 [SMILES: CN(C)c1ccc(/C=C2\C=C(C(CC3C4(CO4)C(N4CCOCC4)CC4[C@]3(C)CC[C@@H](O)[C@@]4(C)CO)Nc3ccccn3)C(=O)O2)cc1] | 658.37 |
| 251 | 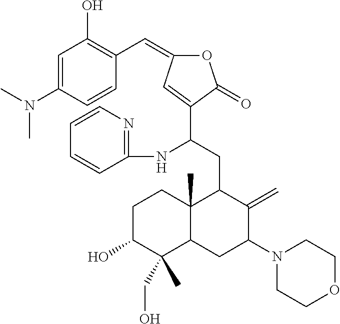 [SMILES: C=C1C(N2CCOCC2)CC2[C@](C)(CC[C@@H](O)[C@@]2(C)CO)C1CC(Nc1ccccn1)C1=C/C(=C\c2ccc(N(C)C)cc2O)OC1=O] | 658.37 |
| 252 | 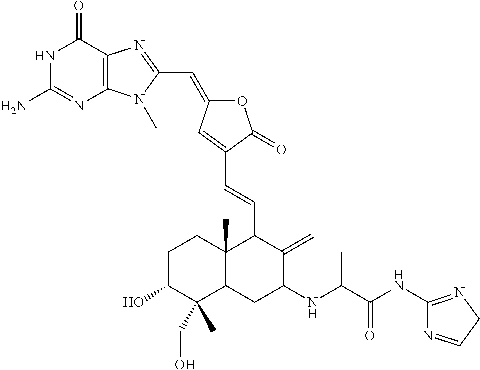 [SMILES: C=C1C(NC(C)C(=O)NC2=NCC=N2)CC2[C@](C)(CC[C@@H](O)[C@@]2(C)CO)C1/C=C/C1=CC(=C\c2nc3c(=O)[nH]c(N)nc3n2C)/OC1=O] | 659.32 |
| 253 | 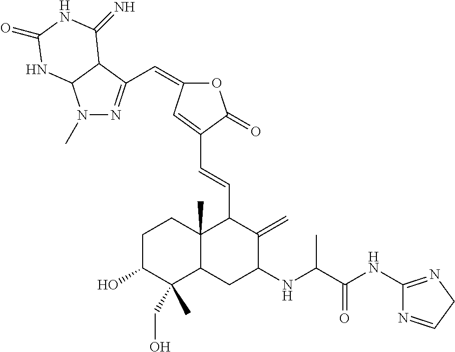 [SMILES: C=C1C(NC(C)C(=O)NC2=NCC=N2)CC2[C@](C)(CC[C@@H](O)[C@@]2(C)CO)C1/C=C/C1=CC(=C\C2=NN(C)C3NC(=O)NC(=N)C23)/OC1=O] | 659.32 |
| 254 | 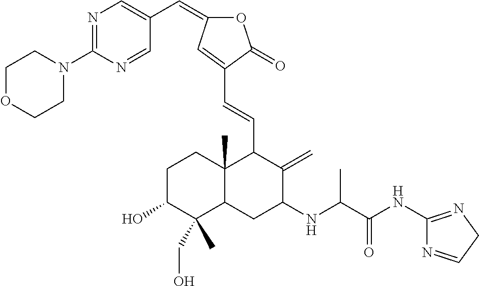 [SMILES: C=C1C(NC(C)C(=O)NC2=NCC=N2)CC2[C@](C)(CC[C@@H](O)[C@@]2(C)CO)C1/C=C/C1=CC(=C\c2cnc(N3CCOCC3)nc2)/OC1=O] | 659.34 |
| 255 | 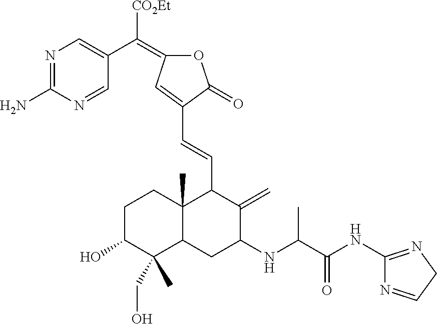 [SMILES: C=C1C(NC(C)C(=O)NC2=NCC=N2)CC2[C@](C)(CC[C@@H](O)[C@@]2(C)CO)C1/C=C/C1=CC(=C(/C(=O)OCC)c2cnc(N)nc2)/OC1=O] | 661.32 |
| 256 | 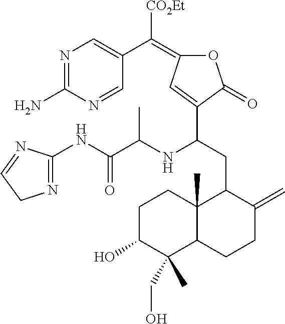 [SMILES: C=C1CCC2[C@](C)(CC[C@@H](O)[C@@]2(C)CO)C1CC(NC(C)C(=O)NC1=NCC=N1)C1=C/C(=C(/C(=O)OCC)c2cnc(N)nc2)OC1=O] | 663.34 |
| 257 | 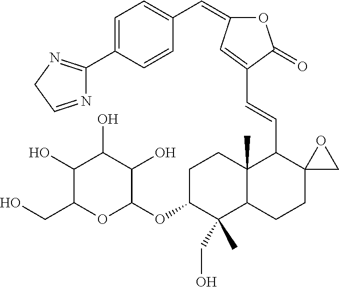 [SMILES: C[C@]12CC[C@@H](OC3OC(CO)C(O)C(O)C3O)[C@@](C)(CO)C1CCC1(CO1)C2/C=C/C1=CC(=C\c2ccc(C3=NCC=N3)cc2)/OC1=O] | 664.30 |
| 258 | 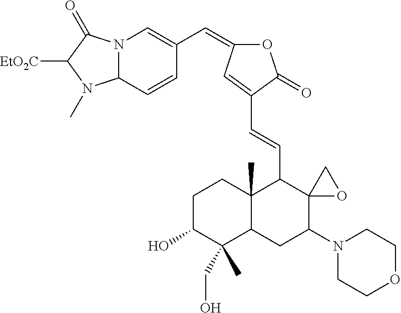 [SMILES: CCOC(=O)C1C(=O)N2C=C(/C=C3C=C(/C=C/C4C5(CO5)C(N5CCOCC5)CC5[C@]4(C)CC[C@@H](O)[C@@]5(C)CO)C(=O)O\3)C=CC2N1C] | 665.33 |
| 259 | 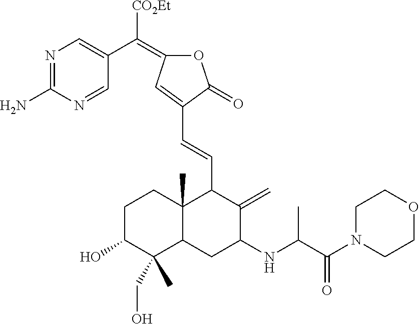 [SMILES: C=C1C(NC(C)C(=O)N2CCOCC2)CC2[C@](C)(CC[C@@H](O)[C@@]2(C)CO)C1/C=C/C1=CC(=C(/C(=O)OCC)c2cnc(N)nc2)/OC1=O] | 665.34 |
| 260 | 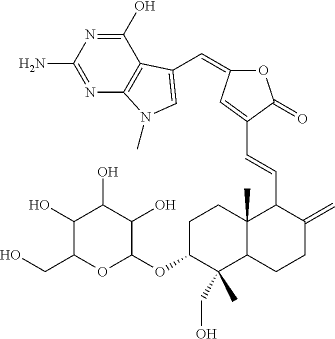 [SMILES: C=C1CCC2[C@](C)(CC[C@@H](OC3OC(CO)C(O)C(O)C3O)[C@@]2(C)CO)C1/C=C/C1=CC(=C\c2cn(C)c3nc(N)nc(O)c23)/OC1=O] | 668.31 |
| 261 | 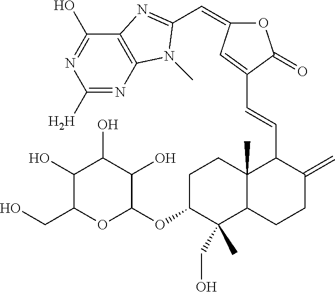 [SMILES: C=C1CCC2[C@](C)(CC[C@@H](OC3OC(CO)C(O)C(O)C3O)[C@@]2(C)CO)C1/C=C/C1=CC(=C\c2nc3c(O)ncnc3n2C)/OC1=O] | 669.30 |
| 262 | 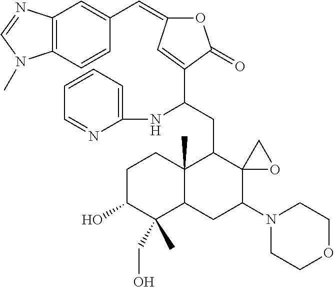 [SMILES: Cn1cnc2cc(/C=C3\C=C(C(CC4C5(CO5)C(N5CCOCC5)CC5[C@]4(C)CC[C@@H](O)[C@@]5(C)CO)Nc4ccccn4)C(=O)O3)ccc21] | 669.35 |
| 263 | 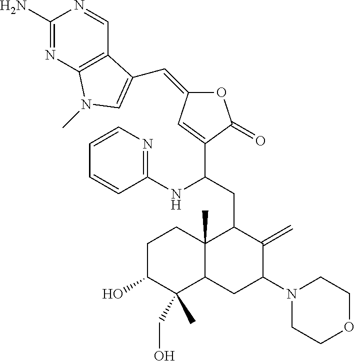 [SMILES: C=C1C(N2CCOCC2)CC2[C@](C)(CC[C@@H](O)[C@@]2(C)CO)C1CC(Nc1ccccn1)C1=C/C(=C\c2cn(C)c3nc(N)ncc23)OC1=O] | 669.36 |
| 264 | 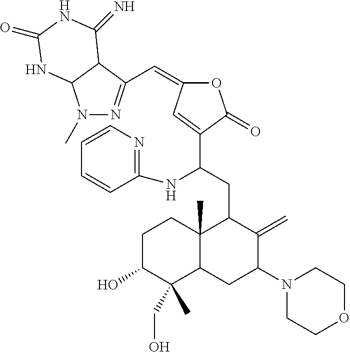 [SMILES: C=C1C(N2CCOCC2)CC2[C@](C)(CC[C@@H](O)[C@@]2(C)CO)C1CC(Nc1ccccn1)C1=C/C(=C\C2=NN(C)C3NC(=O)NC(=N)C23)OC1=O] | 670.36 |
| 265 | 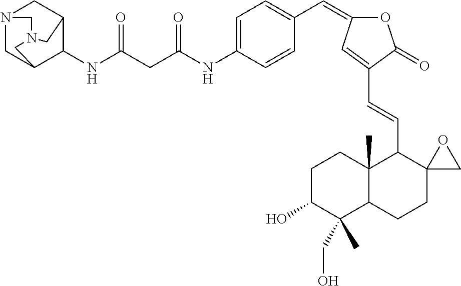 [SMILES: C[C@]12CC[C@@H](O)[C@@](C)(CO)C1CCC1(CO1)C2/C=C/C1=CC(=C\c2ccc(NC(=O)CC(=O)NC3C4CN5CC3CN(C4)C5)cc2)/OC1=O] | 672.35 |
| 266 | 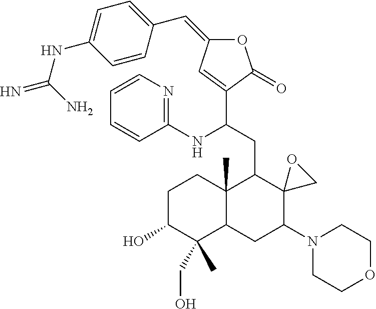 [SMILES: C[C@]12CC[C@@H](O)[C@@](C)(CO)C1CC(N1CCOCC1)C1(CO1)C2CC(Nc1ccccn1)C1=C/C(=C\c2ccc(NC(=N)N)cc2)OC1=O] | 672.36 |
| 267 | 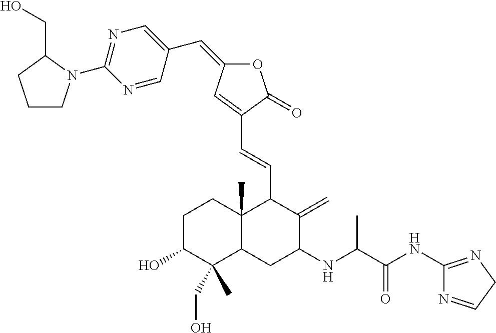 [SMILES: C=C1C(NC(C)C(=O)NC2=NCC=N2)CC2[C@](C)(CC[C@@H](O)[C@@]2(C)CO)C1/C=C/C1=CC(=C\c2cnc(N3CCCC3CO)nc2)/OC1=O] | 673.36 |
| 268 | 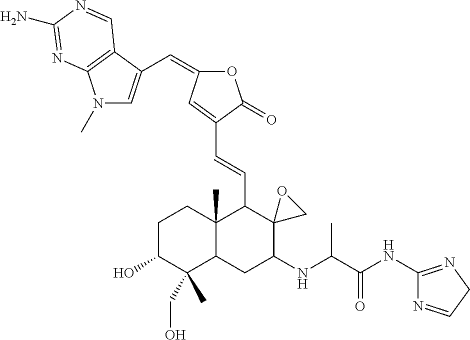 [SMILES: CC(NC1CC2[C@](C)(CC[C@@H](O)[C@@]2(C)CO)C(/C=C/C2=CC(=C\c3cn(C)c4nc(N)ncc34)/OC2=O)C12CO2)C(=O)NC1=NCC=N1] | 658.32 |
| 269 | 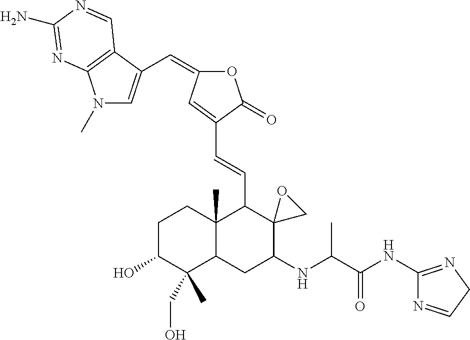 [SMILES: CC(NC1CC2[C@](C)(CC[C@@H](O)[C@@]2(C)CO)C(/C=C/C2=CC(=C\c3cn(C)c4nc(N)ncc34)/OC2=O)C12CO2)C(=O)NC1=NCC=N1] | 658.32 |
| 270 | 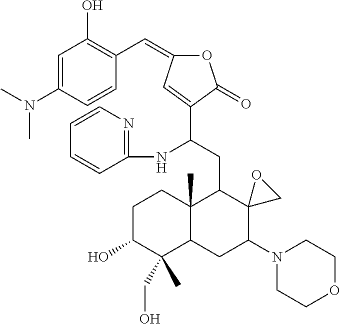 [SMILES: CN(C)c1ccc(/C=C2\C=C(C(CC3C4(CO4)C(N4CCOCC4)CC4[C@]3(C)CC[C@@H](O)[C@@]4(C)CO)Nc3ccccn3)C(=O)O2)c(O)c1] | 674.37 |
| 271 | 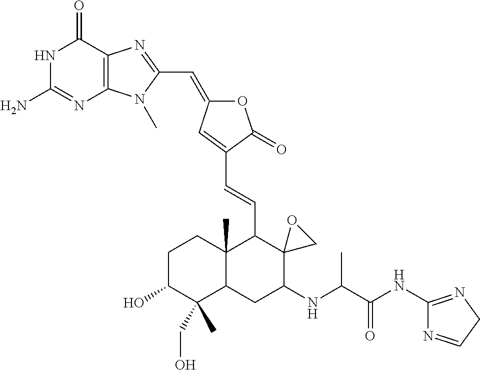 [SMILES: CC(NC1CC2[C@](C)(CC[C@@H](O)[C@@]2(C)CO)C(/C=C/C2=CC(=C\c3nc4c(=O)[nH]c(N)nc4n3C)/OC2=O)C12CO2)C(=O)NC1=NCC=N1] | 675.31 |
| 272 | 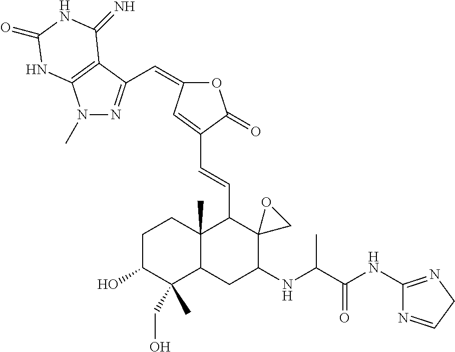 [SMILES: CC(NC1CC2[C@](C)(CC[C@@H](O)[C@@]2(C)CO)C(/C=C/C2=CC(=C\c3nn(C)c4[nH]c(=O)[nH]c(=N)c34)/OC2=O)C12CO2)C(=O)NC1=NCC=N1] | 675.31 |
| 273 | 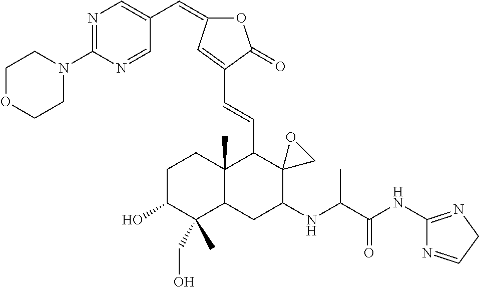 [SMILES: CC(NC1CC2[C@](C)(CC[C@@H](O)[C@@]2(C)CO)C(/C=C/C2=CC(=C\c3cnc(N4CCOCC4)nc3)/OC2=O)C12CO2)C(=O)NC1=NCC=N1] | 675.34 |
| 274 | 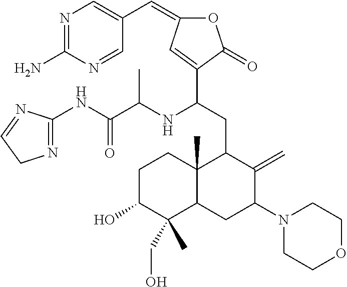 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2cnc(N)nc2)OC1=O] | 676.37 |
| 275 | 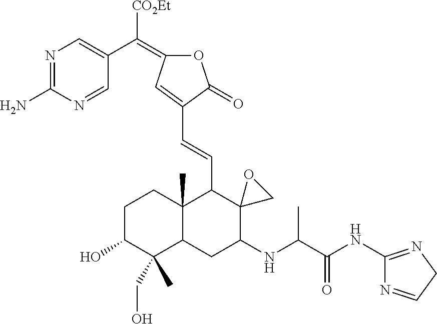 [SMILES: CCOC(=O)/C(=C1C=C(/C=C/C2C3(CO3)C(NC(C)C(=O)NC3=NCC=N3)CC3[C@]2(C)CC[C@@H](O)[C@@]3(C)CO)C(=O)O/1)c1cnc(N)nc1] | 677.32 |
| 276 | 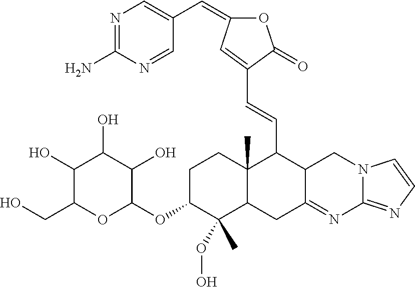 [SMILES: C[C@]12CC[C@@H](OC3OC(CO)C(O)C(O)C3O)[C@@](C)(OO)C1CC1=Nc3nccn3CC1C2/C=C/C1=CC(=C\c2cnc(N)nc2)/OC1=O] | 678.30 |
| 277 | 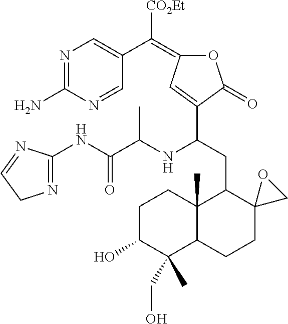 [SMILES: CCOC(=O)/C(=C1/C=C(C(CC2C3(CCC4[C@]2(C)CC[C@@H](O)[C@@]4(C)CO)CO3)NC(C)C(=O)NC2=NCC=N2)C(=O)O1)c1cnc(N)nc1] | 679.33 |
| 278 | 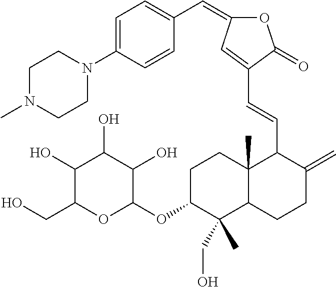 | 680.37 |
| 279 | 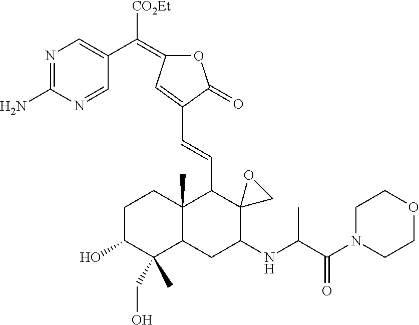 [SMILES: CCOC(=O)/C(=C1C=C(/C=C/C2C3(CO3)C(NC(C)C(=O)N3CCOCC3)CC3[C@]2(C)CC[C@@H](O)[C@@]3(C)CO)C(=O)O/1)c1cnc(N)nc1] | 681.34 |
| 280 | 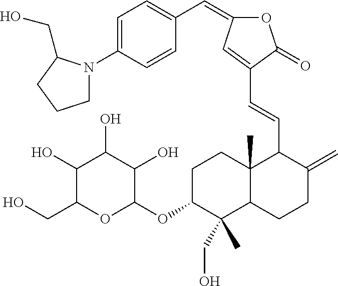 [SMILES: C=C1CCC2[C@](C)(CC[C@@H](OC3OC(CO)C(O)C(O)C3O)[C@@]2(C)CO)C1/C=C/C1=CC(=C\c2ccc(N3CCCC3CO)cc2)/OC1=O] | 681.35 |
| 281 | 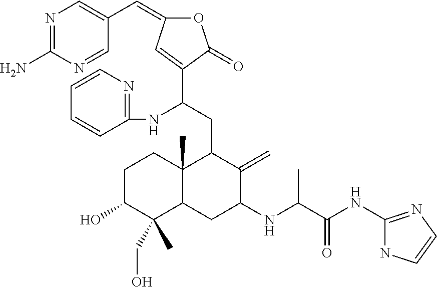 [SMILES: C=C1C(NC(C)C(=O)Nc2ncc[nH]2)CC2[C@](C)(CC[C@@H](O)[C@@]2(C)CO)C1CC(Nc1ccccn1)C1=C/C(=C\c2cnc(N)nc2)OC1=O] | 683.35 |
| 282 | 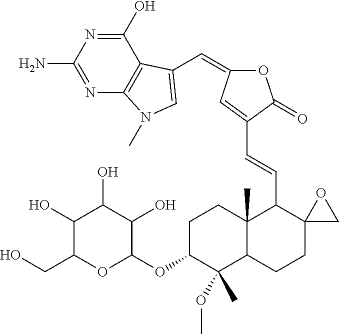 [SMILES: CO[C@@]1(C)C2CCC3(CO3)C(/C=C/C3=CC(=C\c4cn(C)c5nc(N)nc(O)c45)/OC3=O)[C@@]2(C)CC[C@H]1OC1OC(CO)C(O)C(O)C1O] | 684.30 |
| 283 | 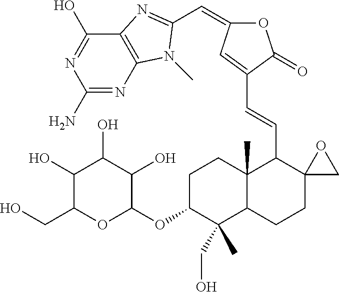 [SMILES: Cn1c(/C=C2C=C(/C=C/C3C4(CCC5[C@]3(C)CC[C@@H](OC3OC(CO)C(O)C(O)C3O)[C@@]5(C)CO)CO4)C(=O)O\2)nc2c(O)nc(N)nc21] | 685.30 |
| 284 | 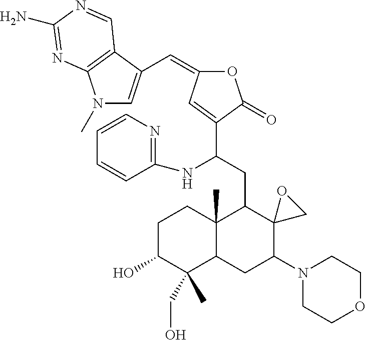 [SMILES: Cn1cc(/C=C2\C=C(C(CC3C4(CO4)C(N4CCOCC4)CC4[C@]3(C)CC[C@@H](O)[C@@]4(C)CO)Nc3ccccn3)C(=O)O2)c2cnc(N)nc21] | 685.36 |
| 285 | 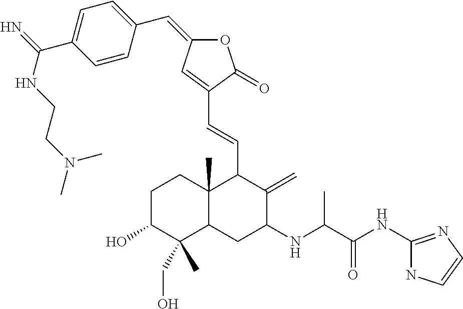 [SMILES: C=C1C(NC(C)C(=O)Nc2ncc[nH]2)CC2[C@](C)(CC[C@@H](O)[C@@]2(C)CO)C1/C=C/C1=CC(=C\c2ccc(C(=N)NCCN(C)C)cc2)/OC1=O] | 685.40 |
| 286 | 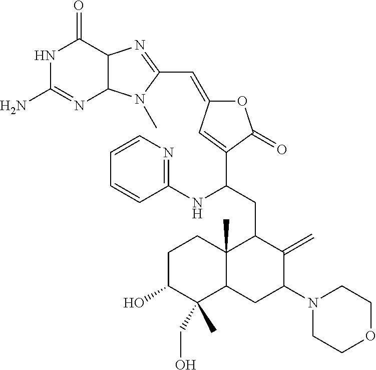 [SMILES: C=C1C(N2CCOCC2)CC2[C@](C)(CC[C@@H](O)[C@@]2(C)CO)C1CC(Nc1ccccn1)C1=C/C(=C\C2=NC3C(=O)NC(N)=NC3N2C)OC1=O] | 686.35 |
| 287 | 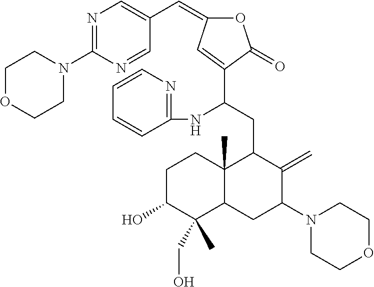 [SMILES: C=C1C(N2CCOCC2)CC2[C@](C)(CC[C@@H](O)[C@@]2(C)CO)C1CC(Nc1ccccn1)C1=C/C(=C\c2cnc(N3CCOCC3)nc2)OC1=O] | 686.38 |
| 288 | 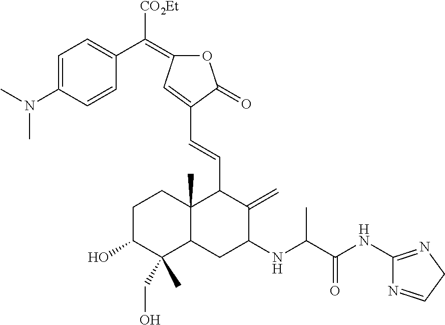 [SMILES: C=C1C(NC(C)C(=O)NC2=NCC=N2)CC2[C@](C)(CC[C@@H](O)[C@@]2(C)CO)C1/C=C/C1=CC(=C(/C(=O)OCC)c2ccc(N(C)C)cc2)/OC1=O] | 687.36 |
| 289 | 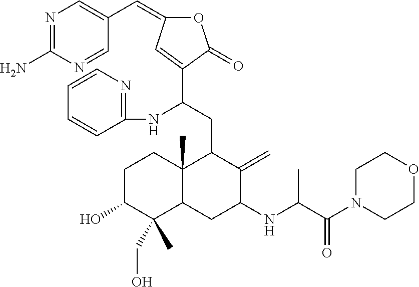 [SMILES: C=C1C(NC(C)C(=O)N2CCOCC2)CC2[C@](C)(CC[C@@H](O)[C@@]2(C)CO)C1CC(Nc1ccccn1)C1=C/C(=C\c2cnc(N)nc2)OC1=O] | 687.37 |
| 290 | 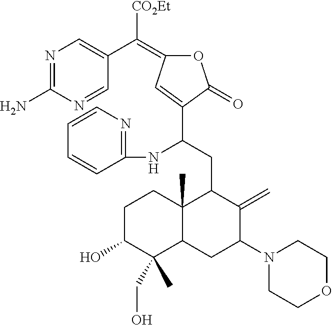 [SMILES: C=C1C(N2CCOCC2)CC2[C@](C)(CC[C@@H](O)[C@@]2(C)CO)C1CC(Nc1ccccn1)C1=C/C(=C(/C(=O)OCC)c2cnc(N)nc2)OC1=O] | 688.36 |
| 291 | 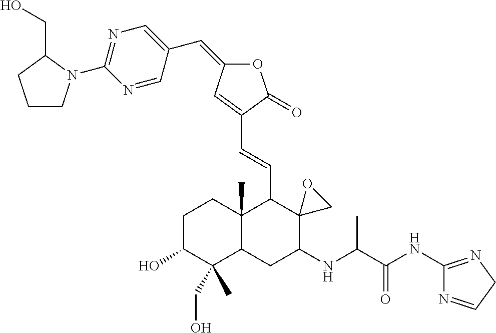 [SMILES: CC(NC1CC2[C@](C)(CC[C@@H](O)[C@@]2(C)CO)C(/C=C/C2=CC(=C\c3cnc(N4CCCC4CO)nc3)/OC2=O)C12CO2)C(=O)NC1=NCC=N1] | 689.35 |
| 292 | 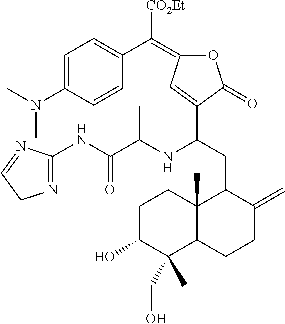 [SMILES: C=C1CCC2[C@](C)(CC[C@@H](O)[C@@]2(C)CO)C1CC(NC(C)C(=O)NC1=NCC=N1)C1=C/C(=C(/C(=O)OCC)c2ccc(N(C)C)cc2)OC1=O] | 689.38 |
| 293 | 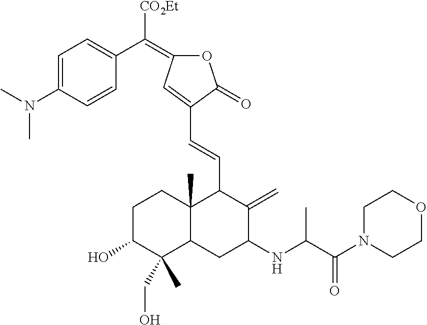 [SMILES: C=C1C(NC(C)C(=O)N2CCOCC2)CC2[C@](C)(CC[C@@H](O)[C@@]2(C)CO)C1/C=C/C1=CC(=C(/C(=O)OCC)c2ccc(N(C)C)cc2)/OC1=O] | 691.38 |
| 294 | 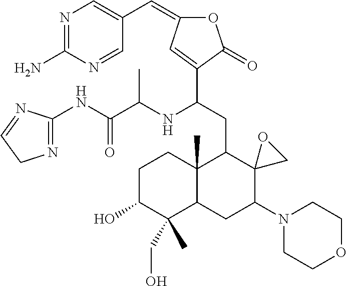 [SMILES: CC(NC(CC1C2(CO2)C(N2CCOCC2)CC2[C@]1(C)CC[C@@H](O)[C@@]2(C)CO)C1=C/C(=C\c2cnc(N)nc2)OC1=O)C(=O)NC1=NCC=N1] | 692.36 |
| 295 | 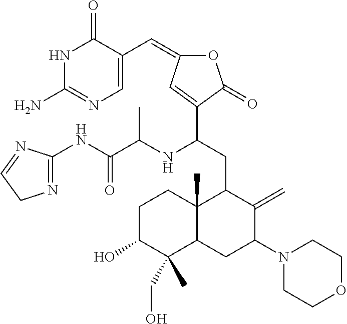 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2cnc(N)[nH]c2=O)OC1=O] | 692.36 |
| 296 | 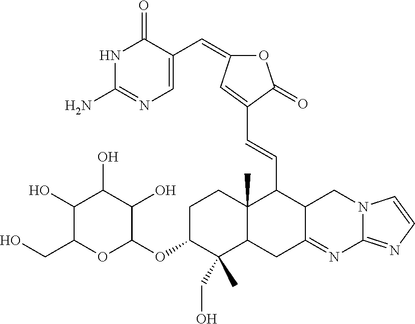 [SMILES: C[C@]12CC[C@@H](OC3OC(CO)C(O)C(O)C3O)[C@@](C)(CO)C1CC1=Nc3nccn3CC1C2/C=C/C1=CC(=C\c2cnc(N)[nH]c2=O)/OC1=O] | 694.30 |
| 297 | 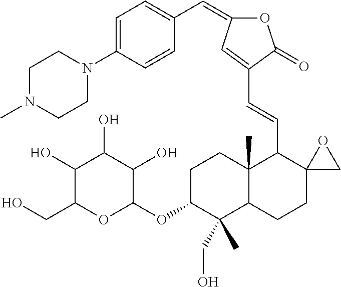 [SMILES: CN1CCN(c2ccc(/C=C3C=C(/C=C/C4C5(CCC6[C@]4(C)CC[C@@H](OC4OC(CO)C(O)C(O)C4O)[C@@]6(C)CO)CO5)C(=O)O\3)cc2)CC1] | 696.36 |
| 298 | 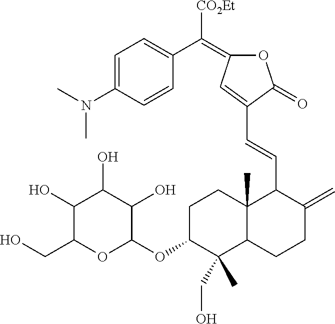 [SMILES: C=C1CCC2[C@](C)(CC[C@@H](OC3OC(CO)C(O)C(O)C3O)[C@@]2(C)CO)C1/C=C/C1=CC(=C(/C(=O)OCC)c2ccc(N(C)C)cc2)/OC1=O] | 697.35 |
| 299 | 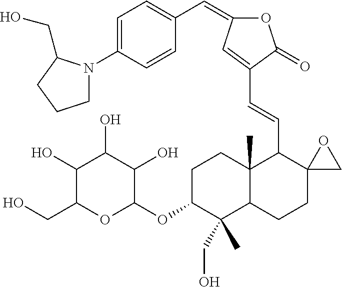 [SMILES: C[C@]12CC[C@@H](OC3OC(CO)C(O)C(O)C3O)[C@@](C)(CO)C1CCC1(CO1)C2/C=C/C1=CC(=C\c2ccc(N3CCCC3CO)cc2)/OC1=O] | 697.35 |
| 300 | 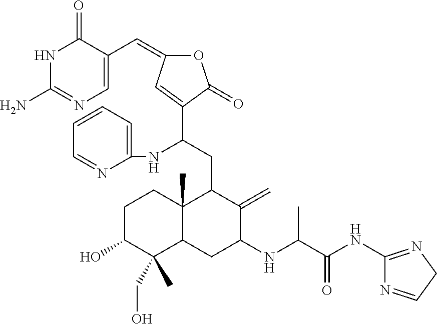 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2cnc(N)[nH]c2=O)OC1=O] | 699.35 |
| 301 | 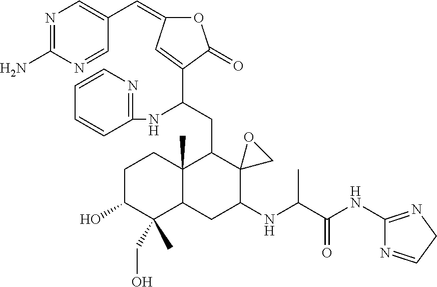 [SMILES: CC(NC1CC2[C@](C)(CC[C@@H](O)[C@@]2(C)CO)C(CC(Nc2ccccn2)C2=C/C(=C\c3cnc(N)nc3)OC2=O)C12CO2)C(=O)NC1=NCC=N1] | 699.35 |
| 302 | 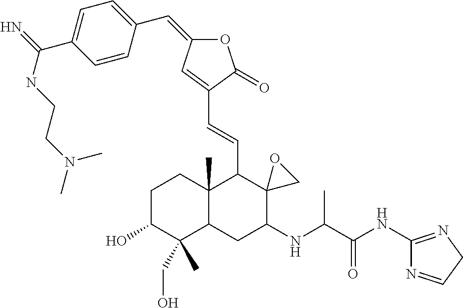 [SMILES: CC(NC1CC2[C@](C)(CC[C@@H](O)[C@@]2(C)CO)C(/C=C/C2=CC(=C\c3ccc(C(=N)NCCN(C)C)cc3)/OC2=O)C12CO2)C(=O)NC1=NCC=N1] | 700.38 |
| 303 | 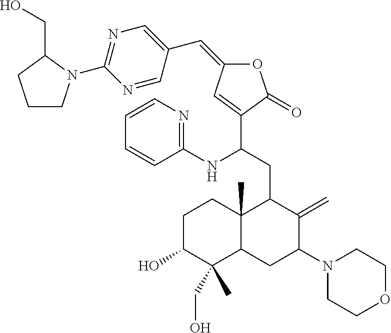 [SMILES: C=C1C(N2CCOCC2)CC2[C@](C)(CC[C@@H](O)[C@@]2(C)CO)C1CC(Nc1ccccn1)C1=C/C(=C\c2cnc(N3CCCC3CO)nc2)OC1=O] | 700.39 |
| 304 | 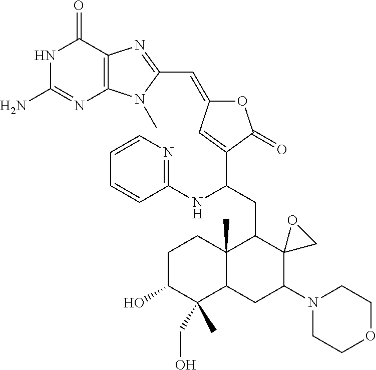 [SMILES: Cn1c(/C=C2\C=C(C(CC3C4(CO4)C(N4CCOCC4)CC4[C@]3(C)CC[C@@H](O)[C@@]4(C)CO)Nc3ccccn3)C(=O)O2)nc2c(=O)[nH]c(N)nc21] | 702.35 |
| 305 | 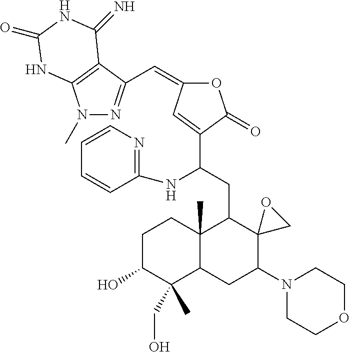 [SMILES: Cn1nc(/C=C2\C=C(C(CC3C4(CO4)C(N4CCOCC4)CC4[C@]3(C)CC[C@@H](O)[C@@]4(C)CO)Nc3ccccn3)C(=O)O2)c2c(=N)[nH]c(=O)[nH]c21] | 702.35 |
| 306 | 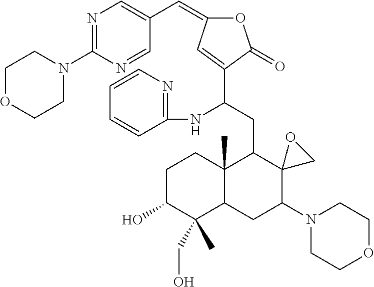 [SMILES: C[C@]12CC[C@@H](O)[C@@](C)(CO)C1CC(N1CCOCC1)C1(CO1)C2CC(Nc1ccccn1)C1=C/C(=C\c2cnc(N3CCOCC3)nc2)OC1=O] | 702.37 |
| 307 | 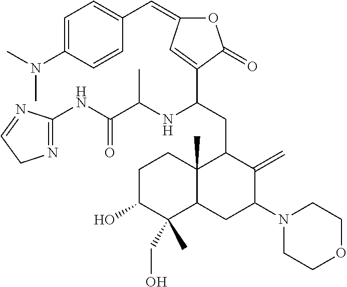 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2ccc(N(C)C)cc2)OC1=O] | 702.41 |
| 308 | 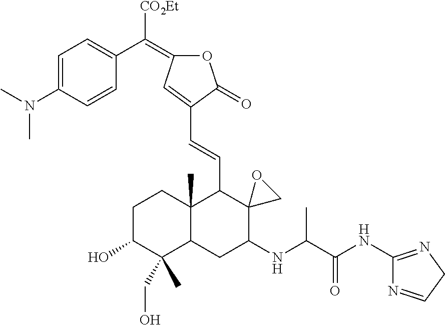 [SMILES: CCOC(=O)/C(=C1C=C(/C=C/C2C3(CO3)C(NC(C)C(=O)NC3=NCC=N3)CC3[C@]2(C)CC[C@@H](O)[C@@]3(C)CO)C(=O)O/1)c1ccc(N(C)C)cc1] | 703.36 |
| 309 | 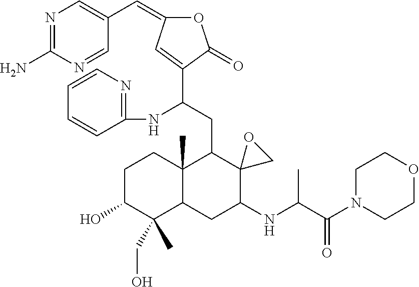 [SMILES: CC(NC1CC2[C@](C)(CC[C@@H](O)[C@@]2(C)CO)C(CC(Nc2ccccn2)C2=C/C(=C\c3cnc(N)nc3)OC2=O)C12CO2)C(=O)N1CCOCC1] | 703.37 |
| 310 | 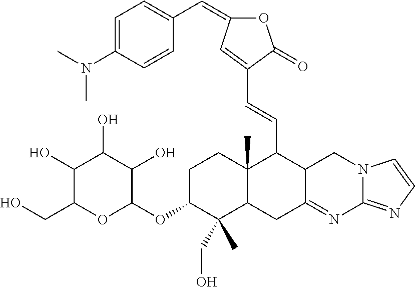 [SMILES: CN(C)c1ccc(/C=C2C=C(/C=C/C3C4Cn5ccnc5N=C4CC4[C@]3(C)CC[C@@H](OC3OC(CO)C(O)C(O)C3O)[C@@]4(C)CO)C(=O)O\2)cc1] | 704.34 |
| 311 | 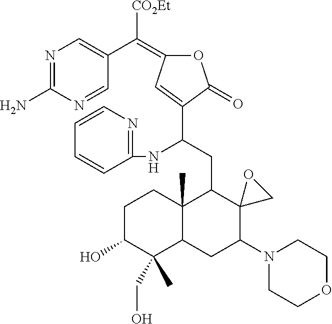 [SMILES: CCOC(=O)/C(=C1/C=C(C(CC2C3(CO3)C(N3CCOCC3)CC3[C@]2(C)CC[C@@H](O)[C@@]3(C)CO)Nc2ccccn2)C(=O)O1)c1cnc(N)nc1] | 704.35 |
| 312 | 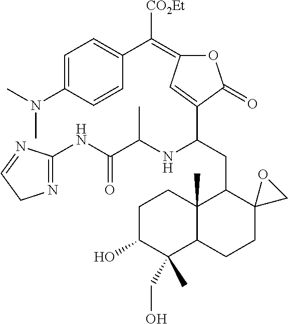 [SMILES: CCOC(=O)/C(=C1/C=C(C(CC2C3(CCC4[C@]2(C)CC[C@@H](O)[C@@]4(C)CO)CO3)NC(C)C(=O)NC2=NCC=N2)C(=O)O1)c1ccc(N(C)C)cc1] | 705.37 |
| 313 | 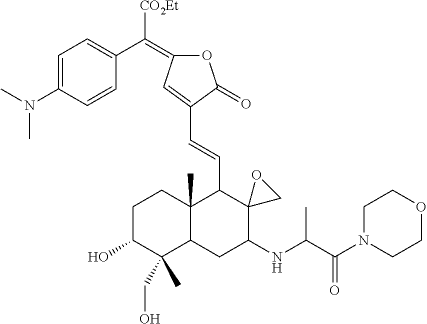 [SMILES: CCOC(=O)/C(=C1C=C(/C=C/C2C3(CO3)C(NC(C)C(=O)N3CCOCC3)CC3[C@]2(C)CC[C@@H](O)[C@@]3(C)CO)C(=O)O/1)c1ccc(N(C)C)cc1] | 707.38 |
| 314 | 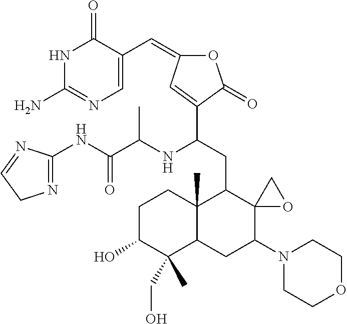 [SMILES: CC(NC(CC1C2(CO2)C(N2CCOCC2)CC2[C@]1(C)CC[C@@H](O)[C@@]2(C)CO)C1=C/C(=C\c2cnc(N)[nH]c2=O)OC1=O)C(=O)NC1=NCC=N1] | 708.36 |
| 315 | 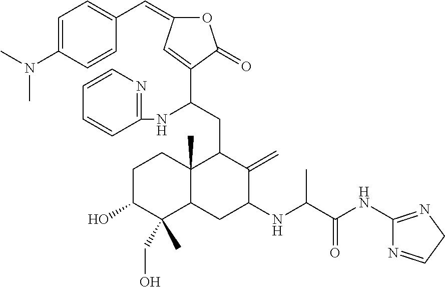 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2ccc(N(C)C)cc2)OC1=O] | 709.40 |
| 316 | 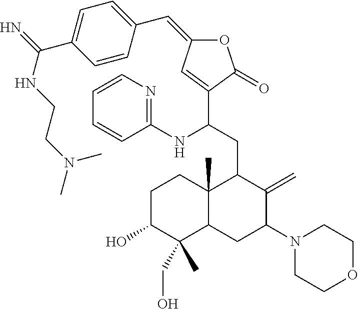 [SMILES: C=C1C(N2CCOCC2)CC2[C@](C)(CC[C@@H](O)[C@@]2(C)CO)C1CC(Nc1ccccn1)C1=C/C(=C\c2ccc(C(=N)NCCN(C)C)cc2)OC1=O] | 712.43 |
| 317 | 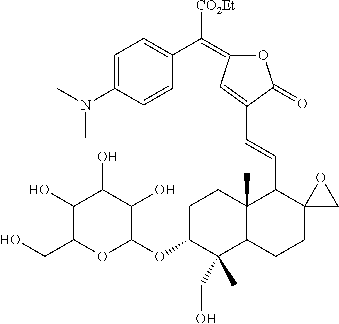 [SMILES: CCOC(=O)/C(=C1C=C(/C=C/C2C3(CCC4[C@]2(C)CC[C@@H](OC2OC(CO)C(O)C(O)C2O)[C@@]4(C)CO)CO3)C(=O)O/1)c1ccc(N(C)C)cc1] | 713.34 |
| 318 | 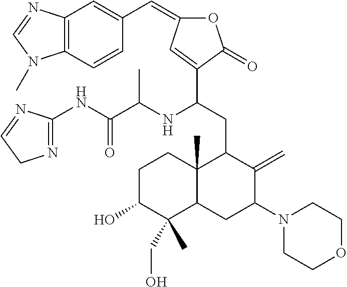 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2ccc3c(c2)ncn3C)OC1=O] | 713.39 |
| 319 | 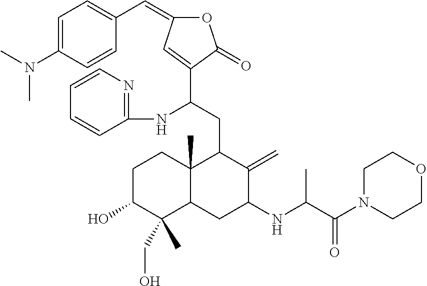 [SMILES: C=C1C(NC(C)C(=O)N2CCOCC2)CC2[C@](C)(CC[C@@H](O)[C@@]2(C)CO)C1CC(Nc1ccccn1)C1=C/C(=C\c2ccc(N(C)C)cc2)OC1=O] | 713.42 |
| 320 | 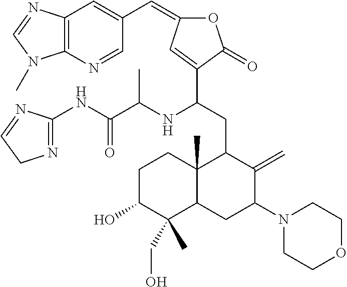 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2cnc3c(c2)ncn3C)OC1=O] | 714.39 |
| 321 | 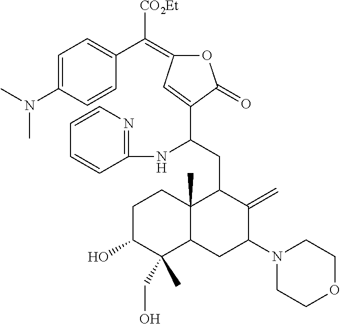 [SMILES: C=C1C(N2CCOCC2)CC2[C@](C)(CC[C@@H](O)[C@@]2(C)CO)C1CC(Nc1ccccn1)C1=C/C(=C(/C(=O)OCC)c2ccc(N(C)C)cc2)OC1=O] | 714.40 |
| 322 | 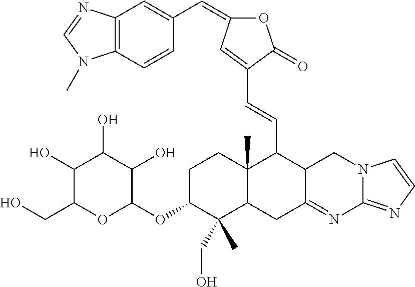 [SMILES: Cn1cnc2cc(/C=C3C=C(/C=C/C4C5Cn6ccnc6N=C5CC5[C@]4(C)CC[C@@H](OC4OC(CO)C(O)C(O)C4O)[C@@]5(C)CO)C(=O)O\3)ccc21] | 715.32 |
| 323 | 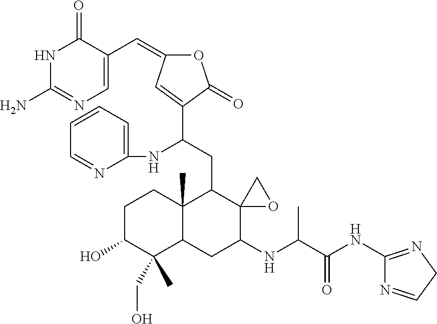 [SMILES: CC(NC1CC2[C@](C)(CC[C@@H](O)[C@@]2(C)CO)C(CC(Nc2ccccn2)C2=C/C(=C\c3cnc(N)[nH]c3=O)OC2=O)C12CO2)C(=O)NC1=NCC=N1] | 715.34 |
| 324 | 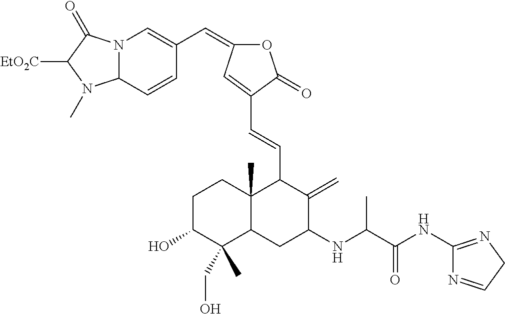 [SMILES: C=C1C(NC(C)C(=O)NC2=NCC=N2)CC2[C@](C)(CC[C@@H](O)[C@@]2(C)CO)C1/C=C/C1=CC(=C\C2=CN3C(=O)C(C(=O)OCC)N(C)C3C=C2)/OC1=O] | 716.35 |
| 325 | 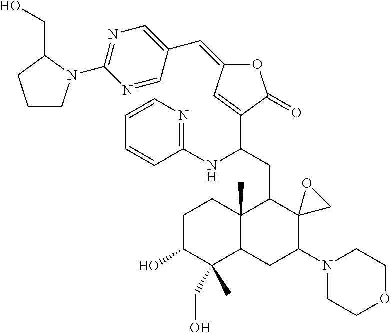 [SMILES: C[C@]12CC[C@@H](O)[C@@](C)(CO)C1CC(N1CCOCC1)C1(CO1)C2CC(Nc1ccccn1)C1=C/C(=C\c2cnc(N3CCCC3CO)nc2)OC1=O] | 716.39 |
| 326 | 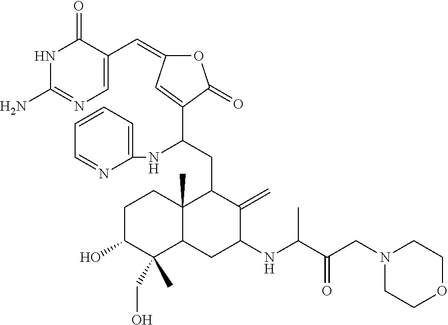 [SMILES: C=C1C(NC(C)C(=O)CN2CCOCC2)CC2[C@](C)(CC[C@@H](O)[C@@]2(C)CO)C1CC(Nc1ccccn1)C1=C/C(=C\c2cnc(N)[nH]c2=O)OC1=O] | 717.38 |
| 327 | 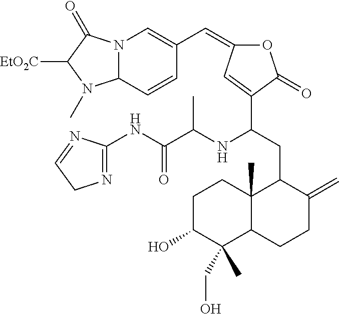 [SMILES: C=C1CCC2[C@](C)(CC[C@@H](O)[C@@]2(C)CO)C1CC(NC(C)C(=O)NC1=NCC=N1)C1=C/C(=C\C2=CN3C(=O)C(C(=O)OCC)N(C)C3C=C2)OC1=O] | 718.37 |
| 328 | 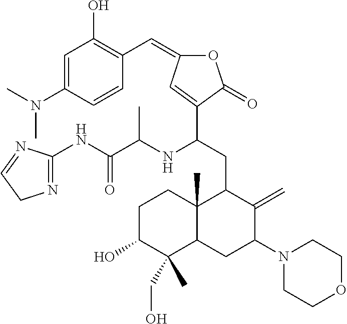 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\c2ccc(N(C)C)cc2O)OC1=O] | 718.41 |
| 329 | 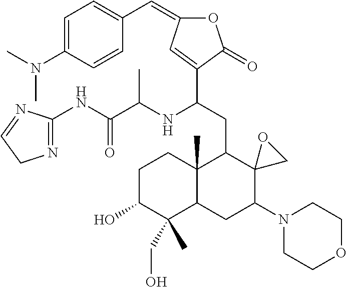 [SMILES: CC(NC(CC1C2(CO2)C(N2CCOCC2)CC2[C@]1(C)CC[C@@H](O)[C@@]2(C)CO)C1=C/C(=C\c2ccc(N(C)C)cc2)OC1=O)C(=O)NC1=NCC=N1] | 718.41 |
| 330 | 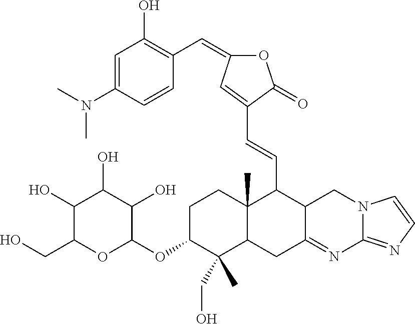 [SMILES: CN(C)c1ccc(/C=C2C=C(/C=C/C3C4Cn5ccnc5N=C4CC4[C@]3(C)CC[C@@H](OC3OC(CO)C(O)C(O)C3O)[C@@]4(C)CO)C(=O)O\2)c(O)c1] | 720.34 |
| 331 | 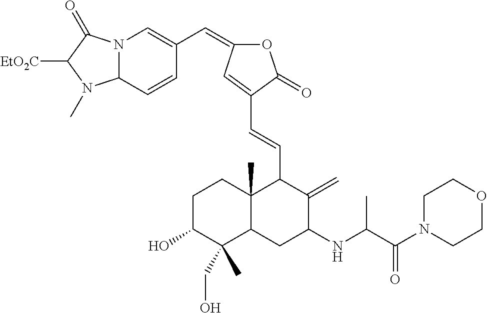 [SMILES: C=C1C(NC(C)C(=O)N2CCOCC2)CC2[C@](C)(CC[C@@H](O)[C@@]2(C)CO)C1/C=C/C1=CC(=C\C2=CN3C(=O)C(C(=O)OCC)N(C)C3C=C2)/OC1=O] | 720.37 |
| 332 | 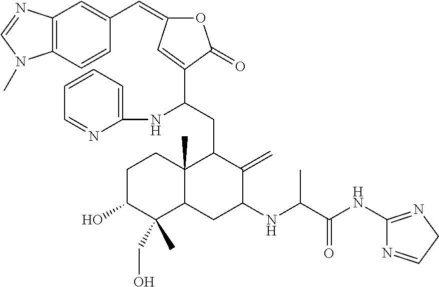 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2ccc3c(c2)ncn3C)OC1=O] | 720.37 |
| 333 | 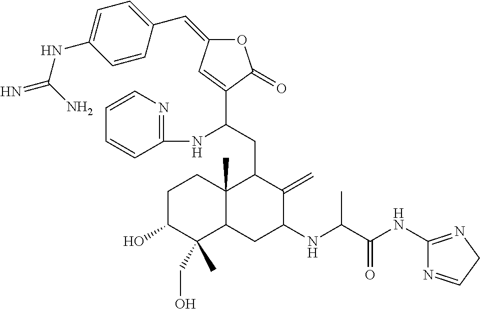 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2ccc(NC(=N)N)cc2)OC1=O] | 723.39 |
| 334 | 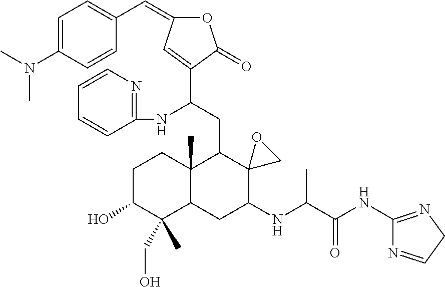 [SMILES: CC(NC1CC2[C@](C)(CC[C@@H](O)[C@@]2(C)CO)C(CC(Nc2ccccn2)C2=C/C(=C\c3ccc(N(C)C)cc3)OC2=O)C12CO2)C(=O)NC1=NCC=N1] | 725.39 |
| 335 | 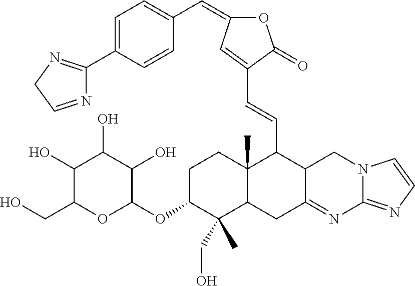 [SMILES: C[C@]12CC[C@@H](OC3OC(CO)C(O)C(O)C3O)[C@@](C)(CO)C1CC1=Nc3nccn3CC1C2/C=C/C1=CC(=C\c2ccc(C3=NCC=N3)cc2)/OC1=O] | 727.32 |
| 336 | 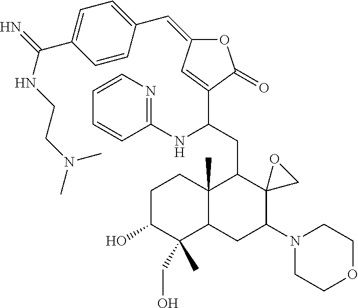 [SMILES: CN(C)CCNC(=N)c1ccc(/C=C2\C=C(C(CC3C4(CO4)C(N4CCOCC4)CC4[C@]3(C)CC[C@@H](O)[C@@]4(C)CO)Nc3ccccn3)C(=O)O2)cc1] | 728.43 |
| 337 | 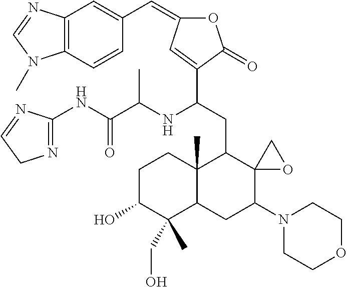 [SMILES: CC(NC(CC1C2(CO2)C(N2CCOCC2)CC2[C@]1(C)CC[C@@H](O)[C@@]2(C)CO)C1=C/C(=C\c2ccc3c(c2)ncn3C)OC1=O)C(=O)NC1=NCC=N1] | 729.38 |
| 338 | 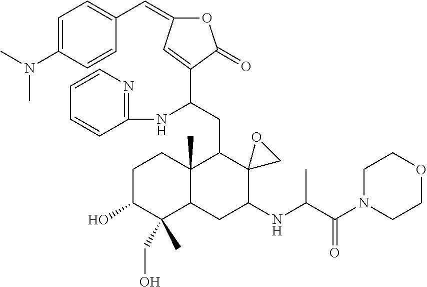 [SMILES: CC(NC1CC2[C@](C)(CC[C@@H](O)[C@@]2(C)CO)C(CC(Nc2ccccn2)C2=C/C(=C\c3ccc(N(C)C)cc3)OC2=O)C12CO2)C(=O)N1CCOCC1] | 729.41 |
| 339 | 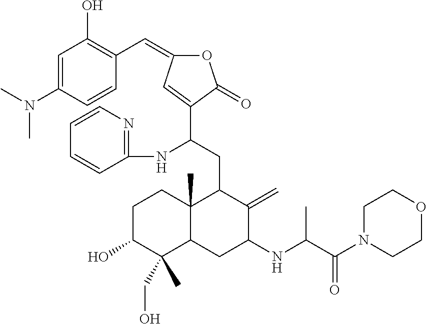 [SMILES: C=C1C(NC(C)C(=O)N2CCOCC2)CC2[C@](C)(CC[C@@H](O)[C@@]2(C)CO)C1CC(Nc1ccccn1)C1=C/C(=C\c2ccc(N(C)C)cc2O)OC1=O] | 729.41 |
| 340 | 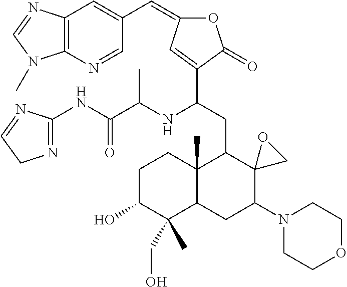 [SMILES: CC(NC(CC1C2(CO2)C(N2CCOCC2)CC2[C@]1(C)CC[C@@H](O)[C@@]2(C)CO)C1=C/C(=C\c2cnc3c(c2)ncn3C)OC1=O)C(=O)NC1=NCC=N1] | 730.38 |
| 341 | 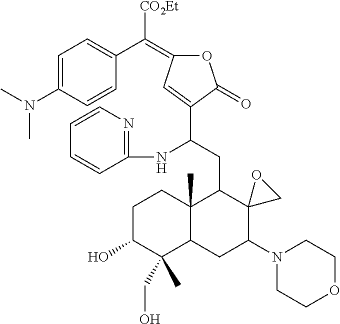 [SMILES: CCOC(=O)/C(=C1/C=C(C(CC2C3(CO3)C(N3CCOCC3)CC3[C@]2(C)CC[C@@H](O)[C@@]3(C)CO)Nc2ccccn2)C(=O)O1)c1ccc(N(C)C)cc1] | 730.39 |
| 342 | 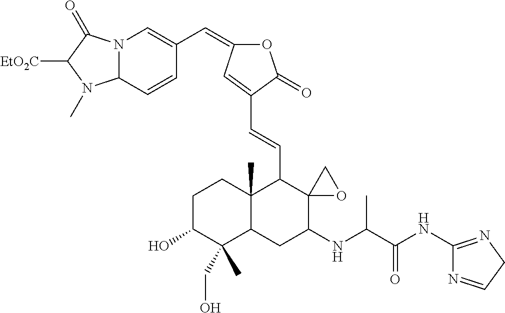 [SMILES: CCOC(=O)C1C(=O)N2C=C(/C=C3C=C(/C=C/C4C5(CO5)C(NC(C)C(=O)NC5=NCC=N5)CC5[C@]4(C)CC[C@@H](O)[C@@]5(C)CO)C(=O)O\3)C=CC2N1C] | 732.35 |
| 343 | 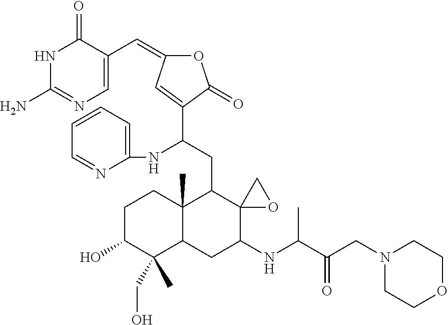 [SMILES: CC(NC1CC2[C@](C)(CC[C@@H](O)[C@@]2(C)CO)C(CC(Nc2ccccn2)C2=C/C(=C\c3cnc(N)[nH]c3=O)OC2=O)C12CO2)C(=O)CN1CCOCC1] | 733.38 |
| 344 | 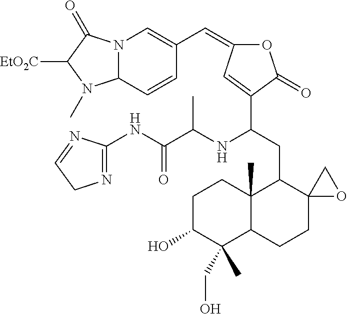 [SMILES: CCOC(=O)C1C(=O)N2C=C(/C=C3\C=C(C(CC4C5(CCC6[C@]4(C)CC[C@@H](O)[C@@]6(C)CO)CO5)NC(C)C(=O)NC4=NCC=N4)C(=O)O3)C=CC2N1C] | 734.36 |
| 345 | 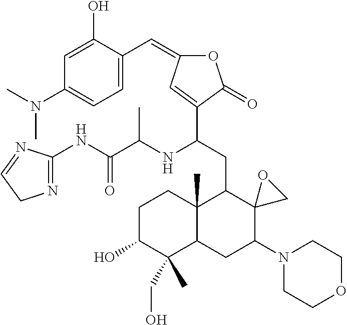 [SMILES: CC(NC(CC1C2(CO2)C(N2CCOCC2)CC2[C@]1(C)CC[C@@H](O)[C@@]2(C)CO)C1=C/C(=C\c2ccc(N(C)C)cc2O)OC1=O)C(=O)NC1=NCC=N1] | 734.40 |
| 346 | 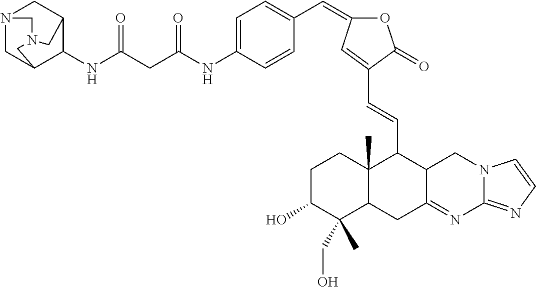 [SMILES: C[C@]12CC[C@@H](O)[C@@](C)(CO)C1CC1=Nc3nccn3CC1C2/C=C/C1=CC(=C\c2ccc(NC(=O)CC(=O)NC3C4CN5CC3CN(C4)C5)cc2)/OC1=O] | 735.37 |
| 347 | 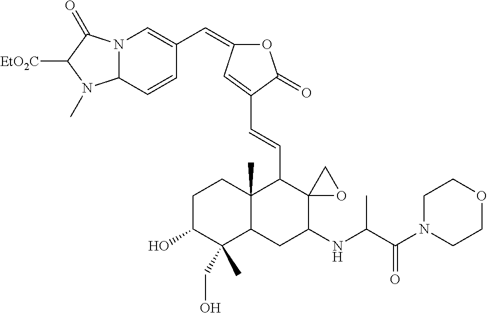 [SMILES: CCOC(=O)C1C(=O)N2C=C(/C=C3C=C(/C=C/C4C5(CO5)C(NC(C)C(=O)N5CCOCC5)CC5[C@]4(C)CC[C@@H](O)[C@@]5(C)CO)C(=O)O\3)C=CC2N1C] | 736.37 |
| 348 | 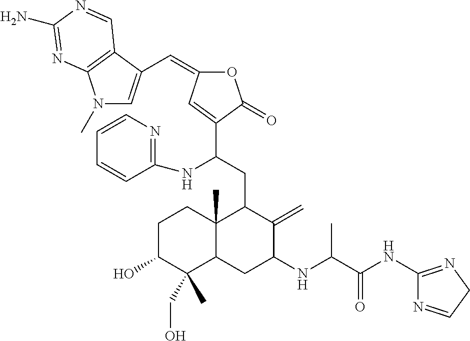 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2cn(C)c3nc(N)ncc23)OC1=O] | 736.38 |
| 349 | 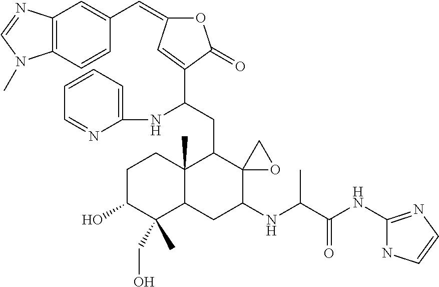 [SMILES: CC(NC1CC2[C@](C)(CC[C@@H](O)[C@@]2(C)CO)C(CC(Nc2ccccn2)C2=C/C(=C\c3ccc4c(c3)ncn4C)OC2=O)C12CO2)C(=O)Nc1ncc[nH]1] | 738.39 |
| 350 | 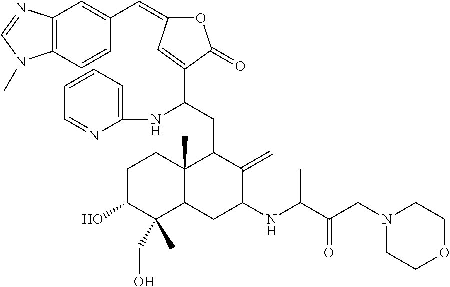 [SMILES: C=C1C(NC(C)C(=O)CN2CCOCC2)CC2[C@](C)(CC[C@@H](O)[C@@]2(C)CO)C1CC(Nc1ccccn1)C1=C/C(=C\c2ccc3c(c2)ncn3C)OC1=O] | 738.41 |
| 351 | 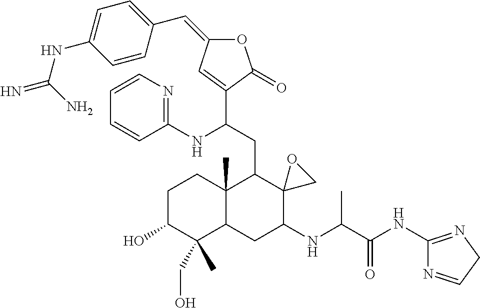 [SMILES: CC(NC1CC2[C@](C)(CC[C@@H](O)[C@@]2(C)CO)C(CC(Nc2ccccn2)C2=C/C(=C\c3ccc(NC(=N)N)cc3)OC2=O)C12CO2)C(=O)NC1=NCC=N1] | 739.38 |
| 352 | 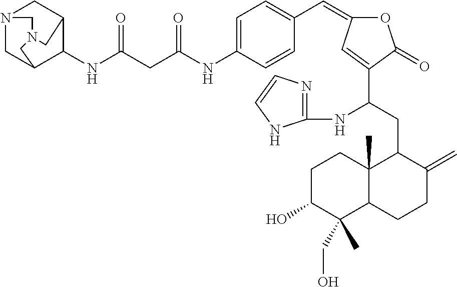 [SMILES: C=C1CCC2[C@](C)(CC[C@@H](O)[C@@]2(C)CO)C1CC(Nc1ncc[nH]1)C1=C/C(=C\c2ccc(NC(=O)CC(=O)NC3C4CN5CC3CN(C4)C5)cc2)OC1=O] | 739.41 |
| 353 | 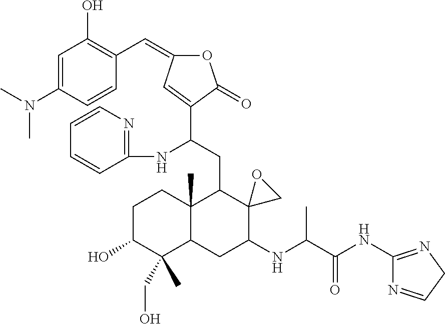 [SMILES: CC(NC1CC2[C@](C)(CC[C@@H](O)[C@@]2(C)CO)C(CC(Nc2ccccn2)C2=C/C(=C\c3ccc(N(C)C)cc3O)OC2=O)C12CO2)C(=O)NC1=NCC=N1] | 741.38 |
| 354 | 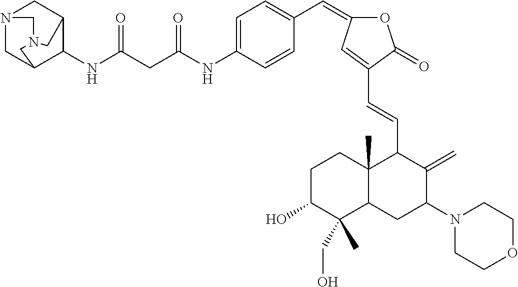 [SMILES: C=C1C(N2CCOCC2)CC2[C@](C)(CC[C@@H](O)[C@@]2(C)CO)C1/C=C/C1=CC(=C\c2ccc(NC(=O)CC(=O)NC3C4CN5CC3CN(C4)C5)cc2)/OC1=O] | 741.41 |
| 355 | 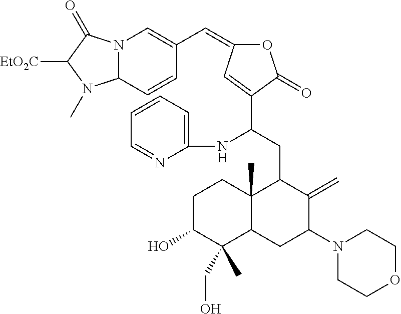 [SMILES: C=C1C(N2CCOCC2)CC2[C@](C)(CC[C@@H](O)[C@@]2(C)CO)C1CC(Nc1ccccn1)C1=C/C(=C\C2=CN3C(=O)C(C(=O)OCC)N(C)C3C=C2)OC1=O] | 743.39 |
| 356 | 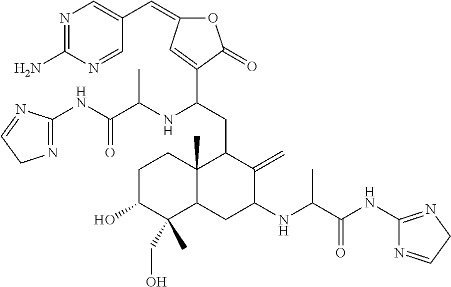 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\c2cnc(N)nc2)OC1=O] | 743.39 |
| 357 | 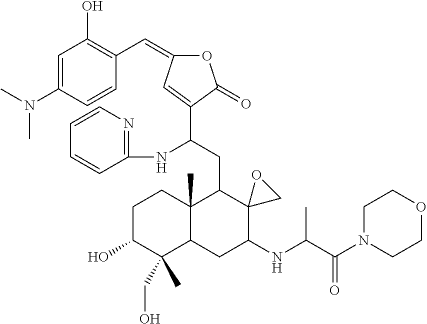 [SMILES: CC(NC1CC2[C@](C)(CC[C@@H](O)[C@@]2(C)CO)C(CC(Nc2ccccn2)C2=C/C(=C\c3ccc(N(C)C)cc3O)OC2=O)C12CO2)C(=O)N1CCOCC1] | 745.41 |
| 358 | 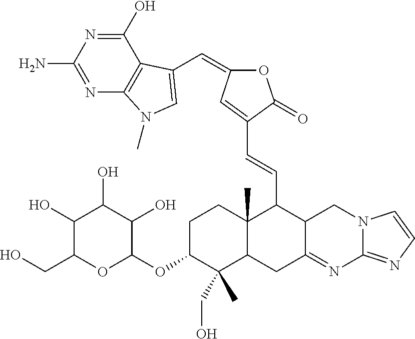 [SMILES: Cn1cc(/C=C2C=C(/C=C/C3C4Cn5ccnc5N=C4CC4[C@]3(C)CC[C@@H](OC3OC(CO)C(O)C(O)C3O)[C@@]4(C)CO)C(=O)O\2)c2c(O)nc(N)nc21] | 747.32 |
| 359 | 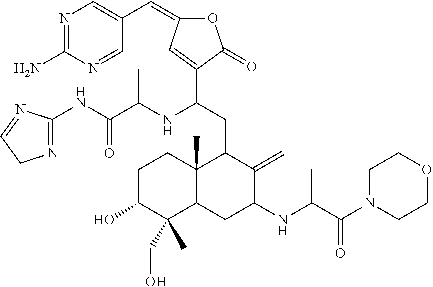 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2cnc(N)nc2)OC1=O] | 747.41 |
| 360 | 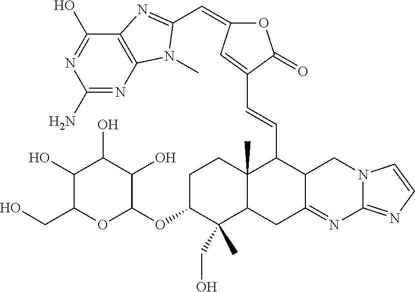 [SMILES: Cn1c(/C=C2C=C(/C=C/C3C4Cn5ccnc5N=C4CC4[C@]3(C)CC[C@@H](OC3OC(CO)C(O)C(O)C3O)[C@@]4(C)CO)C(=O)O\2)nc2c(O)nc(N)nc21] | 748.32 |
| 361 | 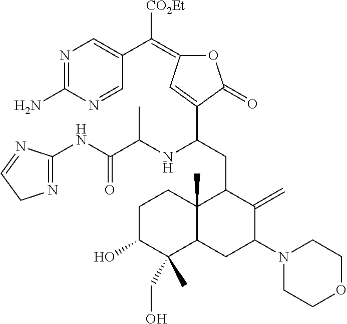 [SMILES: C=C1C(N2CCOCC2)CC2[C@](C)(CC[C@@H](O)[C@@]2(C)CO)C1CC(NC(C)C(=O)NC1=NCC=N1)C1=C/C(=C(/C(=O)OCC)c2cnc(N)nc2)OC1=O] | 748.39 |
| 362 | 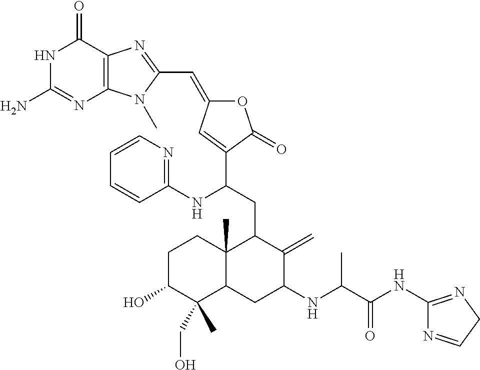 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2nc3c(=O)[nH]c(N)nc3n2C)OC1=O] | 753.37 |
| 363 | 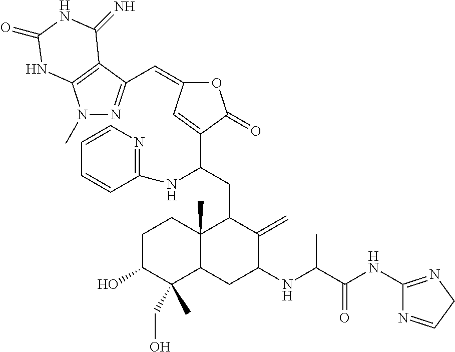 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2nn(C)c3[nH]c(=O)[nH]c(=N)c23)OC1=O] | 753.37 |
| 364 | 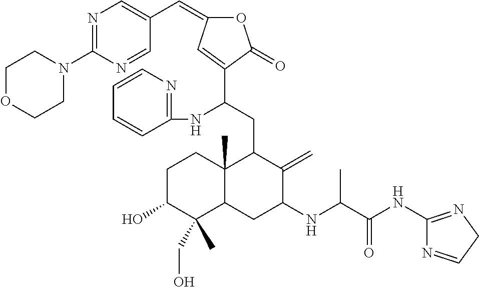 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2cnc(N3CCOCC3)nc2)OC1=O] | 753.40 |
| 365 | 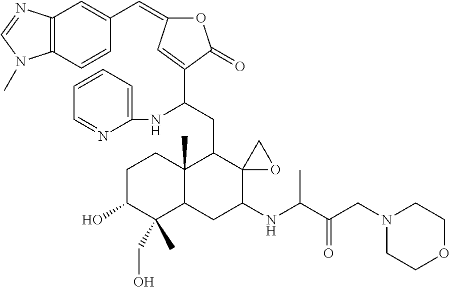 [SMILES: CC(NC1CC2[C@](C)(CC[C@@H](O)[C@@]2(C)CO)C(CC(Nc2ccccn2)C2=C/C(=C\c3ccc4c(c3)ncn4C)OC2=O)C12CO2)C(=O)CN1CCOCC1] | 754.41 |
| 366 | 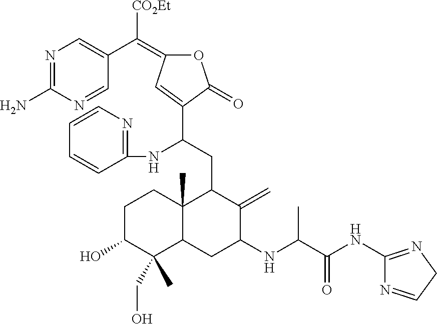 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C(/C(=O)OCC)c2cnc(N)nc2)OC1=O] | 755.38 |
| 367 | 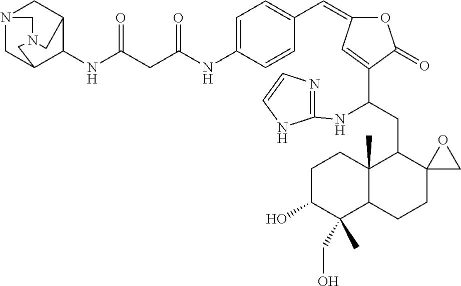 [SMILES: C[C@]12CC[C@@H](O)[C@@](C)(CO)C1CCC1(CO1)C2CC(Nc1ncc[nH]1)C1=C/C(=C\c2ccc(NC(=O)CC(=O)NC3C4CN5CC3CN(C4)C5)cc2)OC1=O] | 755.40 |
| 368 | 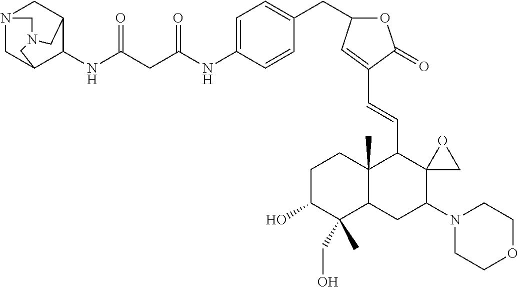 [SMILES: C[C@]12CC[C@@H](O)[C@@](C)(CO)C1CC(N1CCOCC1)C1(CO1)C2/C=C/C1=CC(Cc2ccc(NC(=O)CC(=O)NC3C4CN5CC3CN(C4)C5)cc2)OC1=O] | 757.41 |
| 369 | 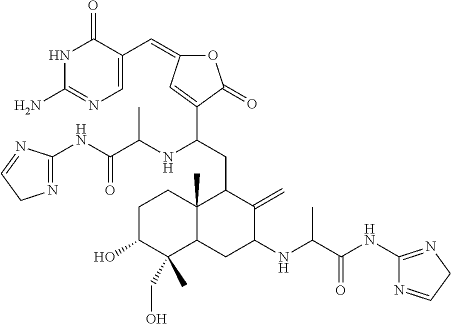 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\c2cnc(N)[nH]c2=O)OC1=O] | 759.38 |
| 370 | 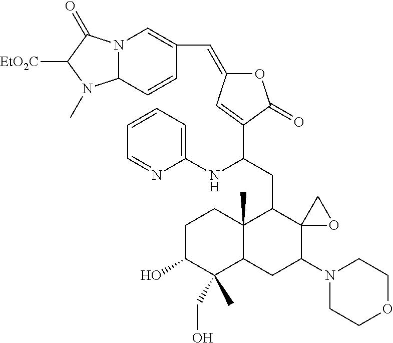 [SMILES: CCOC(=O)C1C(=O)N2C=C(/C=C3\C=C(C(CC4C5(CO5)C(N5CCOCC5)CC5[C@]4(C)CC[C@@H](O)[C@@]5(C)CO)Nc4ccccn4)C(=O)O3)C=CC2N1C] | 759.38 |
| 371 | 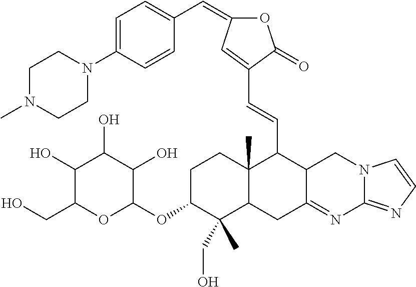 [SMILES: CN1CCN(c2ccc(/C=C3C=C(/C=C/C4C5Cn6ccnc6N=C5CC5[C@]4(C)CC[C@@H](OC4OC(CO)C(O)C(O)C4O)[C@@]5(C)CO)C(=O)O\3)cc2)CC1] | 759.38 |
| 372 | 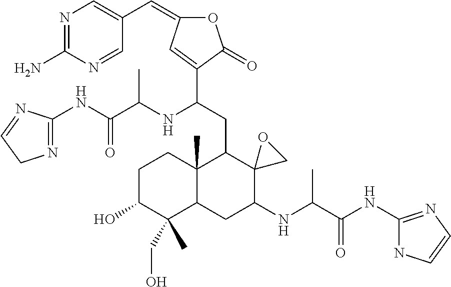 [SMILES: CC(NC(CC1C2(CO2)C(NC(C)C(=O)Nc2ncc[nH]2)CC2[C@]1(C)CC[C@@H](O)[C@@]2(C)CO)C1=C/C(=C\c2cnc(N)nc2)OC1=O)C(=O)NC1=NCC=N1] | 759.38 |
| 373 | 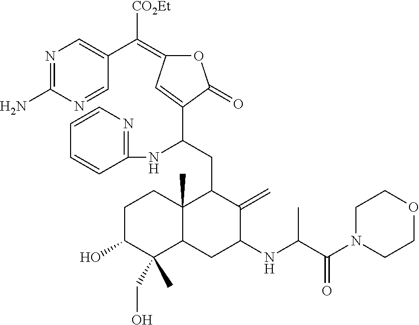 [SMILES: C=C1C(NC(C)C(=O)N2CCOCC2)CC2[C@](C)(CC[C@@H](O)[C@@]2(C)CO)C1CC(Nc1ccccn1)C1=C/C(=C(/C(=O)OCC)c2cnc(N)nc2)OC1=O] | 759.40 |
| 374 | 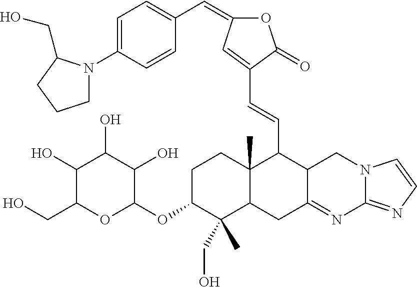 [SMILES: C[C@]12CC[C@@H](OC3OC(CO)C(O)C(O)C3O)[C@@](C)(CO)C1CC1=Nc3nccn3CC1C2/C=C/C1=CC(=C\c2ccc(N3CCCC3CO)cc2)/OC1=O] | 760.37 |
| 375 | 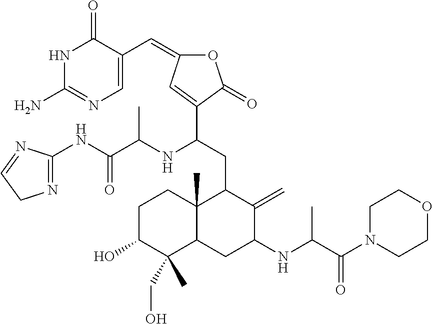 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2cnc(N)[nH]c2=O)OC1=O] | 763.40 |
| 376 | 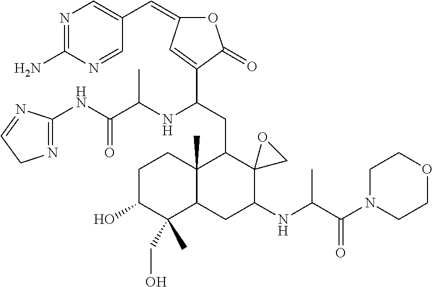 [SMILES: CC(NC(CC1C2(CO2)C(NC(C)C(=O)N2CCOCC2)CC2[C@]1(C)CC[C@@H](O)[C@@]2(C)CO)C1=C/C(=C\c2cnc(N)nc2)OC1=O)C(=O)NC1=NCC=N1] | 763.40 |
| 377 | 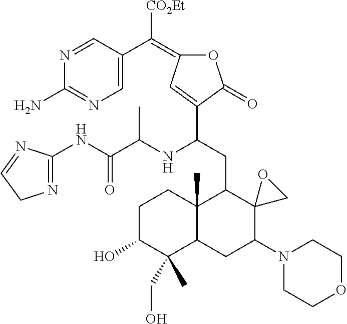 [SMILES: CCOC(=O)/C(=C1/C=C(C(CC2C3(CO3)C(N3CCOCC3)CC3[C@]2(C)CC[C@@H](O)[C@@]3(C)CO)NC(C)C(=O)NC2=NCC=N2)C(=O)O1)c1cnc(N)nc1] | 764.39 |
| 378 | 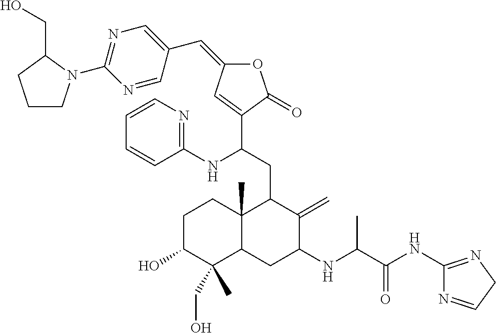 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2cnc(N3CCCC3CO)nc2)OC1=O] | 767.41 |
| 379 | 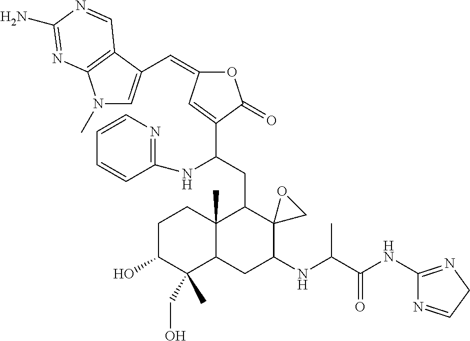 [SMILES: CC(NC1CC2[C@](C)(CC[C@@H](O)[C@@]2(C)CO)C(CC(Nc2ccccn2)C2=C/C(=C\c3cn(C)c4nc(N)ncc34)OC2=O)C12CO2)C(=O)NC1=NCC=N1] | 752.38 |
| 380 | 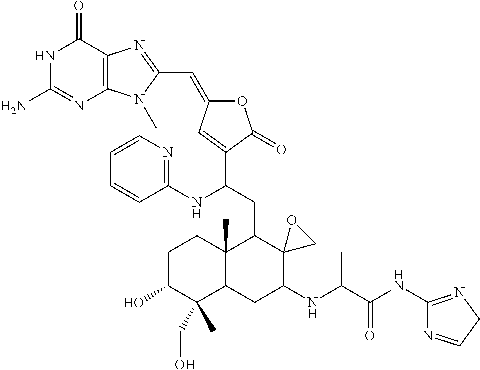 [SMILES: CC(NC1CC2[C@](C)(CC[C@@H](O)[C@@]2(C)CO)C(CC(Nc2ccccn2)C2=C/C(=C\c3nc4c(=O)[nH]c(N)nc4n3C)OC2=O)C12CO2)C(=O)NC1=NCC=N1] | 769.37 |
| 381 | 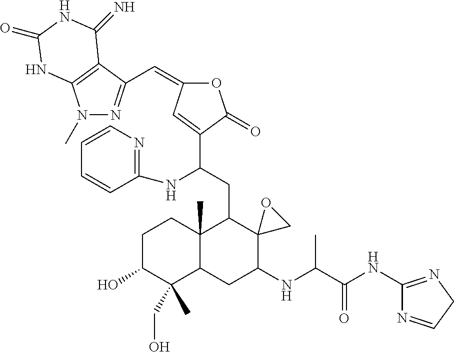 [SMILES: CC(NC1CC2[C@](C)(CC[C@@H](O)[C@@]2(C)CO)C(CC(Nc2ccccn2)C2=C/C(=C\c3nn(C)c4[nH]c(=O)[nH]c(=N)c34)OC2=O)C12CO2)C(=O)NC1=NCC=N1] | 769.37 |
| 382 | 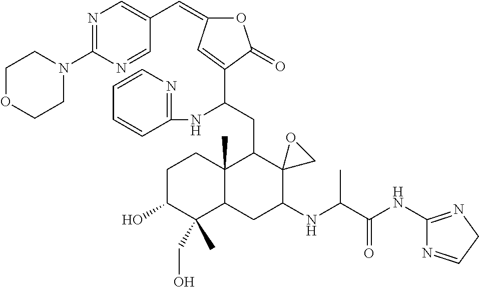 [SMILES: CC(NC1CC2[C@](C)(CC[C@@H](O)[C@@]2(C)CO)C(CC(Nc2ccccn2)C2=C/C(=C\c3cnc(N4CCOCC4)nc3)OC2=O)C12CO2)C(=O)NC1=NCC=N1] | 769.39 |
| 383 | 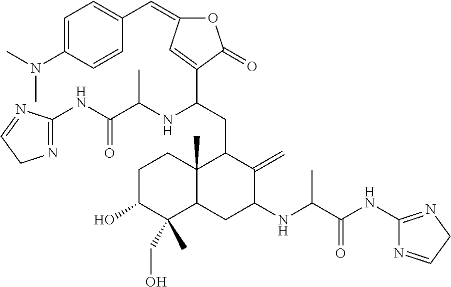 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\c2ccc(N(C)C)cc2)OC1=O] | 769.43 |
| 384 | 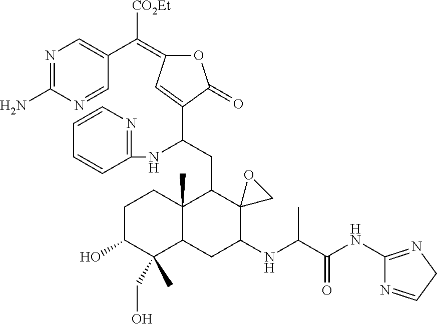 [SMILES: CCOC(=O)/C(=C1/C=C(C(CC2C3(CO3)C(NC(C)C(=O)NC3=NCC=N3)CC3[C@]2(C)CC[C@@H](O)[C@@]3(C)CO)Nc2ccccn2)C(=O)O1)c1cnc(N)nc1] | 771.37 |
| 385 | 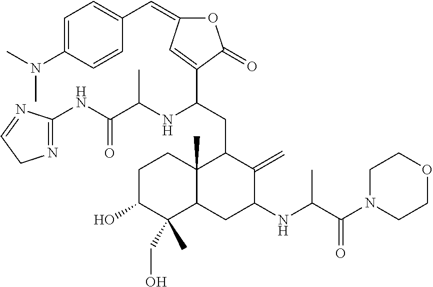 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2ccc(N(C)C)cc2)OC1=O] | 773.45 |
| 386 | 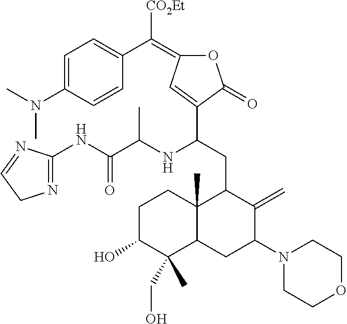 [SMILES: C=C1C(N2CCOCC2)CC2[C@](C)(CC[C@@H](O)[C@@]2(C)CO)C1CC(NC(C)C(=O)NC1=NCC=N1)C1=C/C(=C(/C(=O)OCC)c2ccc(N(C)C)cc2)OC1=O] | 774.43 |
| 387 | 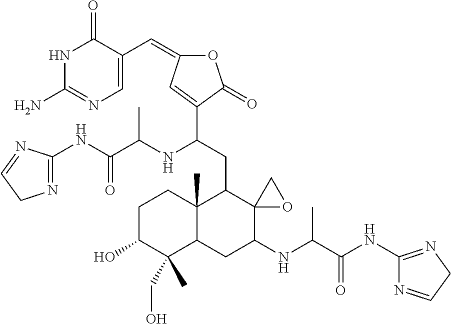 [SMILES: CC(NC(CC1C2(CO2)C(NC(C)C(=O)NC2=NCC=N2)CC2[C@]1(C)CC[C@@H](O)[C@@]2(C)CO)C1=C/C(=C\c2cnc(N)[nH]c2=O)OC1=O)C(=O)NC1=NCC=N1] | 775.38 |
| 388 | 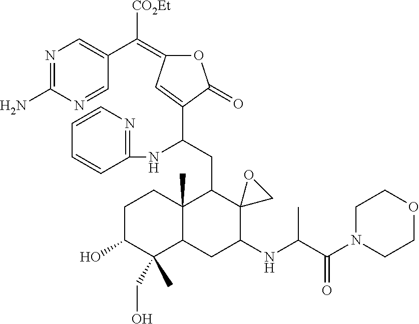 [SMILES: CCOC(=O)/C(=C1/C=C(C(CC2C3(CO3)C(NC(C)C(=O)N3CCOCC3)CC3[C@]2(C)CC[C@@H](O)[C@@]3(C)CO)Nc2ccccn2)C(=O)O1)c1cnc(N)nc1] | 775.39 |
| 389 | 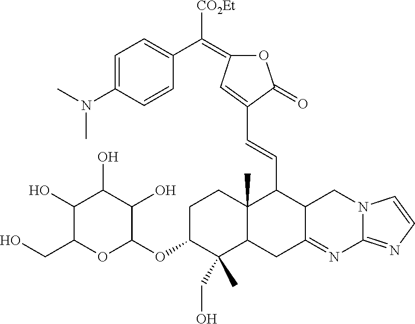 [SMILES: CCOC(=O)/C(=C1C=C(/C=C/C2C3Cn4ccnc4N=C3CC3[C@]2(C)CC[C@@H](OC2OC(CO)C(O)C(O)C2O)[C@@]3(C)CO)C(=O)O/1)c1ccc(N(C)C)cc1] | 776.36 |
| 390 | 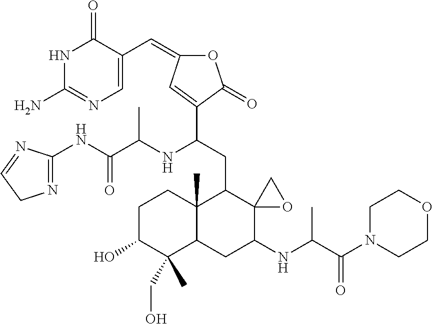 [SMILES: CC(NC(CC1C2(CO2)C(NC(C)C(=O)N2CCOCC2)CC2[C@]1(C)CC[C@@H](O)[C@@]2(C)CO)C1=C/C(=C\c2cnc(N)[nH]c2=O)OC1=O)C(=O)NC1=NCC=N1] | 779.40 |
| 391 | 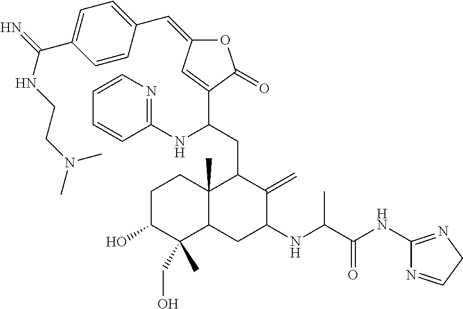 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\c2ccc(C(=N)NCCN(C)C)cc2)OC1=O] | 779.45 |
| 392 | 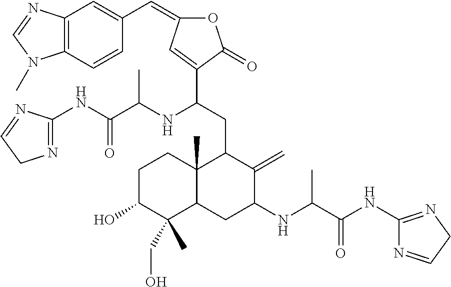 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\c2ccc3c(c2)ncn3C)OC1=O] | 780.41 |
| 393 | 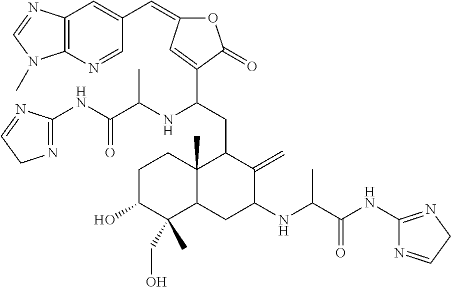 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\c2cnc3c(c2)ncn3C)OC1=O] | 781.40 |
| 394 | 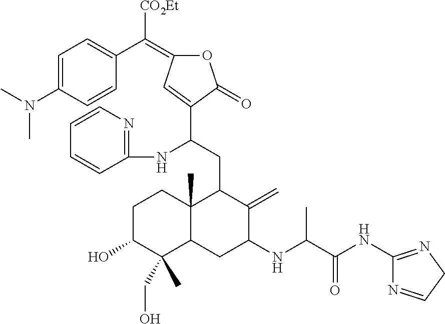 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C(/C(=O)OCC)c2ccc(N(C)C)cc2)OC1=O] | 781.42 |
| 395 | 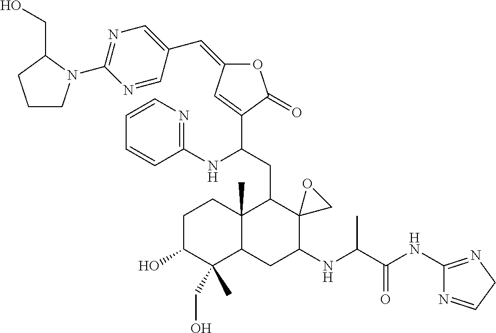 [SMILES: CC(NC1CC2[C@](C)(CC[C@@H](O)[C@@]2(C)CO)C(CC(Nc2ccccn2)C2=C/C(=C\c3cnc(N4CCCC4CO)nc3)OC2=O)C12CO2)C(=O)NC1=NCC=N1] | 783.41 |
| 396 | 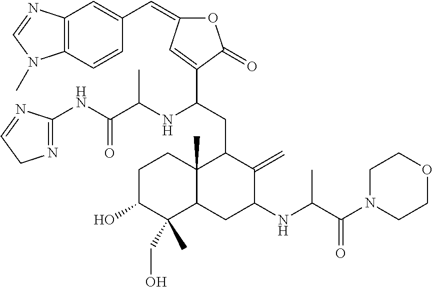 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2ccc3c(c2)ncn3C)OC1=O] | 784.43 |
| 397 | 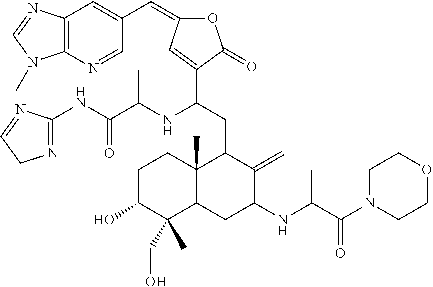 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2cnc3c(c2)ncn3C)OC1=O] | 785.42 |
| 398 | 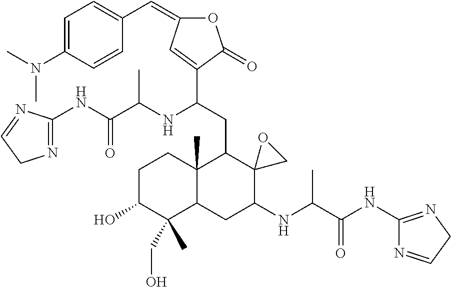 [SMILES: CC(NC(CC1C2(CO2)C(NC(C)C(=O)NC2=NCC=N2)CC2[C@]1(C)CC[C@@H](O)[C@@]2(C)CO)C1=C/C(=C\c2ccc(N(C)C)cc2)OC1=O)C(=O)NC1=NCC=N1] | 785.42 |
| 399 | 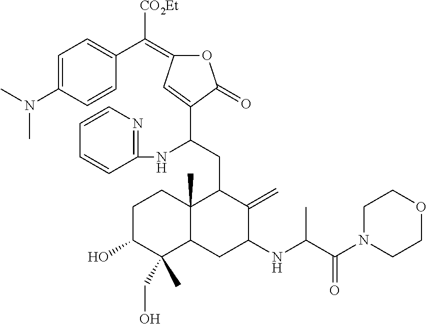 [SMILES: C=C1C(NC(C)C(=O)N2CCOCC2)CC2[C@](C)(CC[C@@H](O)[C@@]2(C)CO)C1CC(Nc1ccccn1)C1=C/C(=C(/C(=O)OCC)c2ccc(N(C)C)cc2)OC1=O] | 785.44 |
| 400 | 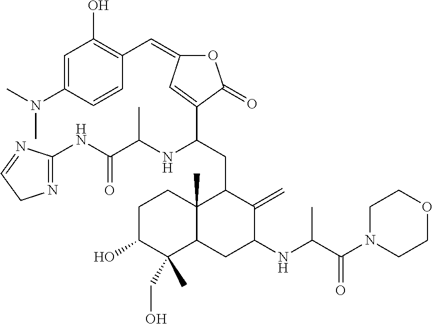 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\c2ccc(N(C)C)cc2O)OC1=O] | 789.44 |
| 401 | 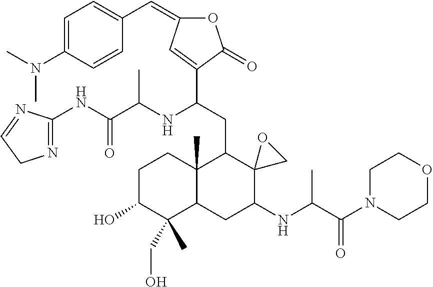 [SMILES: CC(NC(CC1C2(CO2)C(NC(C)C(=O)N2CCOCC2)CC2[C@]1(C)CC[C@@H](O)[C@@]2(C)CO)C1=C/C(=C\c2ccc(N(C)C)cc2)OC1=O)C(=O)NC1=NCC=N1] | 789.44 |
| 402 | 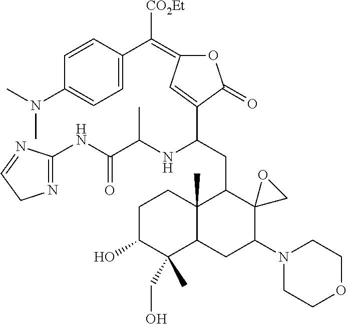 [SMILES: CCOC(=O)/C(=C1/C=C(C(CC2C3(CO3)C(N3CCOCC3)CC3[C@]2(C)CC[C@@H](O)[C@@]3(C)CO)NC(C)C(=O)NC2=NCC=N2)C(=O)O1)c1ccc(N(C)C)cc1] | 790.43 |
| 403 | 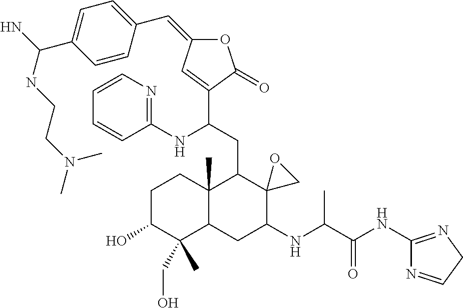 [SMILES: CC(NC1CC2[C@](C)(CC[C@@H](O)[C@@]2(C)CO)C(CC(Nc2ccccn2)C2=C/C(=C\c3ccc(C(N)NCCN(C)C)cc3)OC2=O)C12CO2)C(=O)NC1=NCC=N1] | 794.44 |
| 404 | 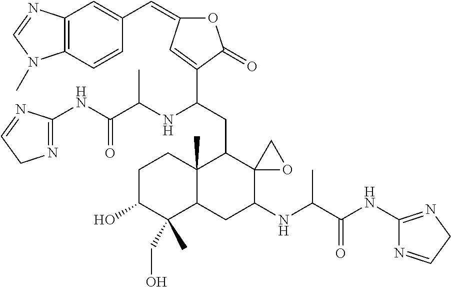 [SMILES: CC(NC(CC1C2(CO2)C(NC(C)C(=O)NC2=NCC=N2)CC2[C@]1(C)CC[C@@H](O)[C@@]2(C)CO)C1=C/C(=C\c2ccc3c(c2)ncn3C)OC1=O)C(=O)NC1=NCC=N1] | 796.40 |
| 405 | 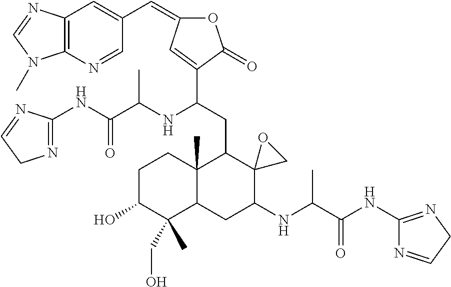 [SMILES: CC(NC(CC1C2(CO2)C(NC(C)C(=O)NC2=NCC=N2)CC2[C@]1(C)CC[C@@H](O)[C@@]2(C)CO)C1=C/C(=C\c2cnc3c(c2)ncn3C)OC1=O)C(=O)NC1=NCC=N1] | 797.40 |
| 406 | 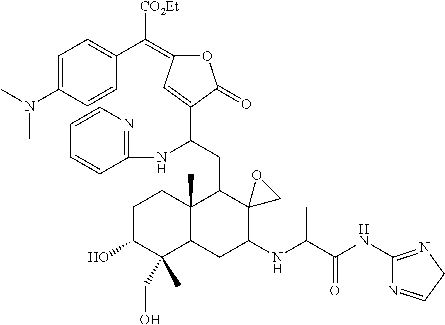 [SMILES: CCOC(=O)/C(=C1/C=C(C(CC2C3(CO3)C(NC(C)C(=O)NC3=NCC=N3)CC3[C@]2(C)CC[C@@H](O)[C@@]3(C)CO)Nc2ccccn2)C(=O)O1)c1ccc(N(C)C)cc1] | 797.41 |
| 407 | 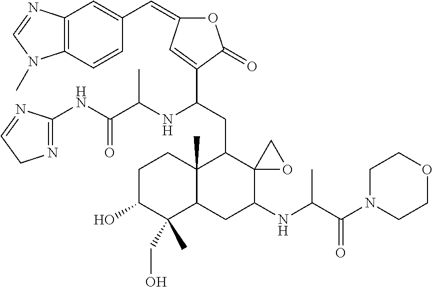 [SMILES: CC(NC(CC1C2(CO2)C(NC(C)C(=O)N2CCOCC2)CC2[C@]1(C)CC[C@@H](O)[C@@]2(C)CO)C1=C/C(=C\c2ccc3c(c2)ncn3C)OC1=O)C(=O)NC1=NCC=N1] | 800.42 |
| 408 | 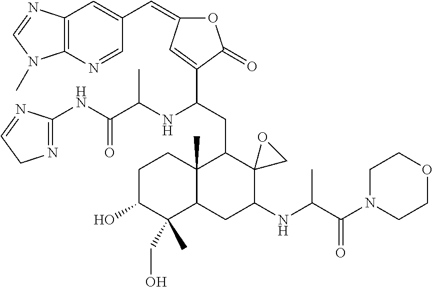 [SMILES: CC(NC(CC1C2(CO2)C(NC(C)C(=O)N2CCOCC2)CC2[C@]1(C)CC[C@@H](O)[C@@]2(C)CO)C1=C/C(=C\c2cnc3c(c2)ncn3C)OC1=O)C(=O)NC1=NCC=N1] | 801.42 |
| 409 | 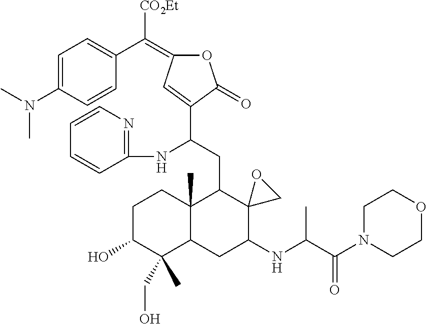 [SMILES: CCOC(=O)/C(=C1/C=C(C(CC2C3(CO3)C(NC(C)C(=O)N3CCOCC3)CC3[C@]2(C)CC[C@@H](O)[C@@]3(C)CO)Nc2ccccn2)C(=O)O1)c1ccc(N(C)C)cc1] | 801.43 |
| 410 | 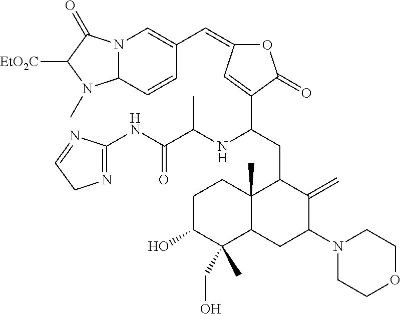 [SMILES: C=C1C(N2CCOCC2)CC2[C@](C)(CC[C@@H](O)[C@@]2(C)CO)C1CC(NC(C)C(=O)NC1=NCC=N1)C1=C/C(=C\C2=CN3C(=O)C(C(=O)OCC)N(C)C3C=C2)OC1=O] | 803.42 |
| 411 | 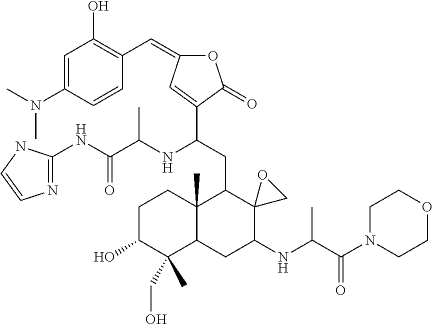 [SMILES: CC(NC(CC1C2(CO2)C(NC(C)C(=O)N2CCOCC2)CC2[C@]1(C)CC[C@@H](O)[C@@]2(C)CO)C1=C/C(=C\c2ccc(N(C)C)cc2O)OC1=O)C(=O)Nc1ncc[nH]1] | 805.44 |
| 412 | 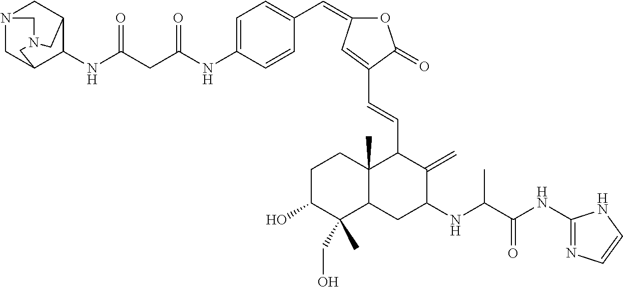 [SMILES: C=C1C(NC(C)C(=O)Nc2ncc[nH]2)CC2[C@](C)(CC[C@@H](O)[C@@]2(C)CO)C1/C=C/C1=CC(=C\c2ccc(NC(=O)CC(=O)NC3C4CN5CC3CN(C4)C5)cc2)/OC1=O] | 808.43 |
| 413 | 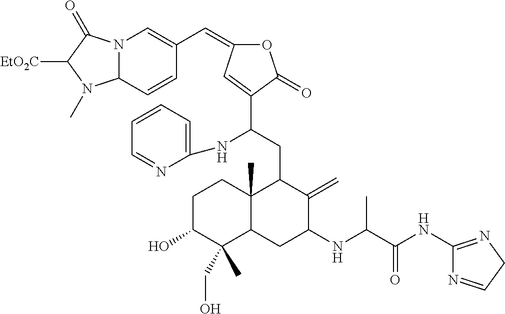 [SMILES: C=C1C(NC(C)C(=O)NC2=NCC=N2)CC2[C@](C)(CC[C@@H](O)[C@@]2(C)CO)C1CC(Nc1ccccn1)C1=C/C(=C\C2=CN3C(=O)C(C(=O)OCC)N(C)C3C=C2)OC1=O] | 810.41 |
| 414 | 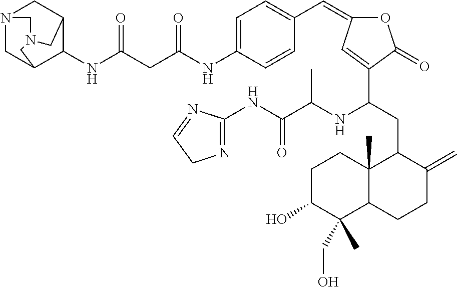 [SMILES: C=C1CCC2[C@](C)(CC[C@@H](O)[C@@]2(C)CO)C1CC(NC(C)C(=O)NC1=NCC=N1)C1=C/C(=C\c2ccc(NC(=O)CC(=O)NC3C4CN5CC3CN(C4)C5)cc2)OC1=O] | 810.44 |
| 415 | 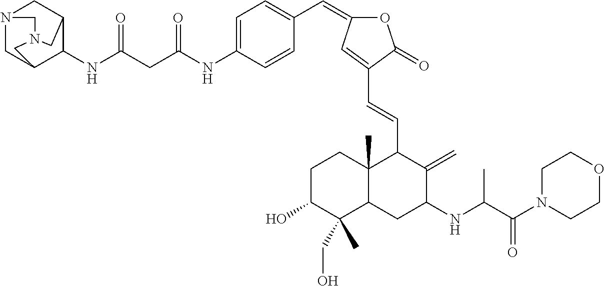 [SMILES: C=C1C(NC(C)C(=O)N2CCOCC2)CC2[C@](C)(CC[C@@H](O)[C@@]2(C)CO)C1/C=C/C1=CC(=C\c2ccc(NC(=O)CC(=O)NC3C4CN5CC3CN(C4)C5)cc2)/OC1=O] | 812.45 |
| 416 | 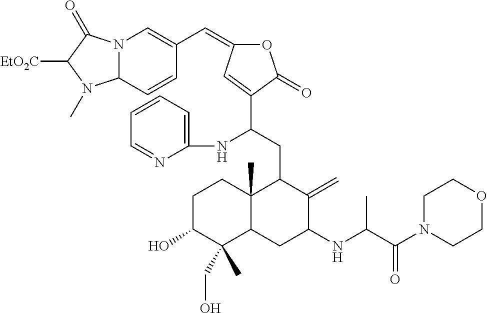 [SMILES: C=C1C(NC(C)C(=O)N2CCOCC2)CC2[C@](C)(CC[C@@H](O)[C@@]2(C)CO)C1CC(Nc1ccccn1)C1=C/C(=C\C2=CN3C(=O)C(C(=O)OCC)N(C)C3C=C2)OC1=O] | 814.43 |
| 417 | 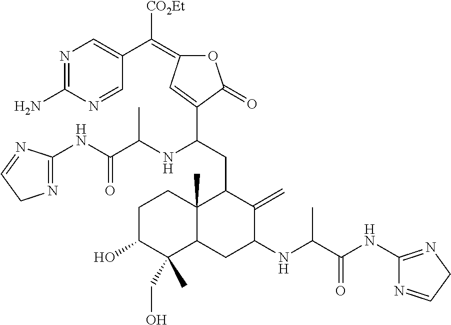 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C(/C(=O)OCC)c2cnc(N)nc2)OC1=O] | 815.41 |
| 418 | 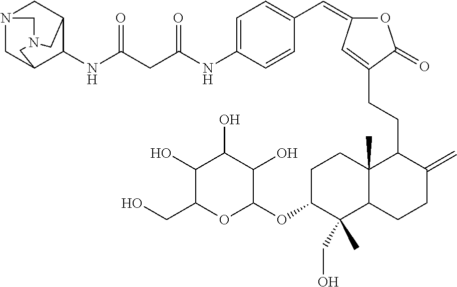 [SMILES: C=C1CCC2[C@](C)(CC[C@@H](OC3OC(CO)C(O)C(O)C3O)[C@@]2(C)CO)C1CCC1=C/C(=C\c2ccc(NC(=O)CC(=O)NC3C4CN5CC3CN(C4)C5)cc2)OC1=O] | 818.41 |
| 419 | 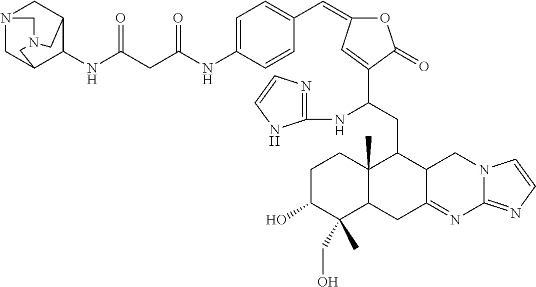 [SMILES: C[C@]12CC[C@@H](O)[C@@](C)(CO)C1CC1=Nc3nccn3CC1C2CC(Nc1ncc[nH]1)C1=C/C(=C\c2ccc(NC(=O)CC(=O)NC3C4CN5CC3CN(C4)C5)cc2)OC1=O] | 818.42 |
| 420 | 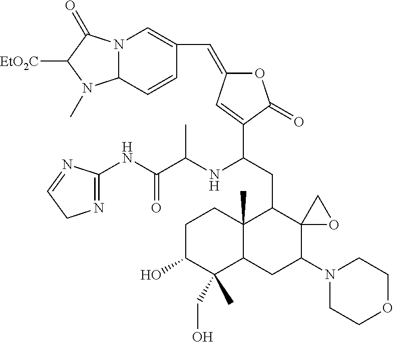 [SMILES: CCOC(=O)C1C(=O)N2C=C(/C=C3\C=C(C(CC4C5(CO5)C(N5CCOCC5)CC5[C@]4(C)CC[C@@H](O)[C@@]5(C)CO)NC(C)C(=O)NC4=NCC=N4)C(=O)O3)C=CC2N1C] | 819.42 |
| 421 | 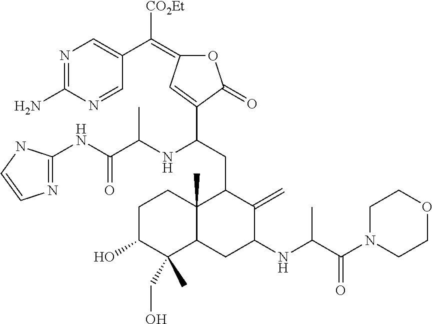 [SMILES: C=C1C(NC(C)C(=O)N2CCOCC2)CC2[C@](C)(CC[C@@H](O)[C@@]2(C)CO)C1CC(NC(C)C(=O)Nc1ncc[nH]1)C1=C/C(=C(/C(=O)OCC)c2cnc(N)nc2)OC1=O] | 819.43 |
| 422 | 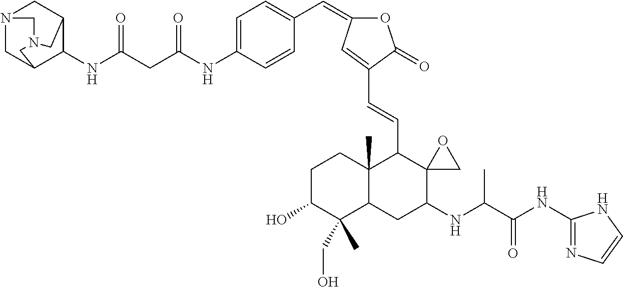 [SMILES: CC(NC1CC2[C@](C)(CC[C@@H](O)[C@@]2(C)CO)C(/C=C/C2=CC(=C\c3ccc(NC(=O)CC(=O)NC4C5CN6CC4CN(C5)C6)cc3)/OC2=O)C12CO2)C(=O)Nc1ncc[nH]1] | 824.42 |
| 423 | 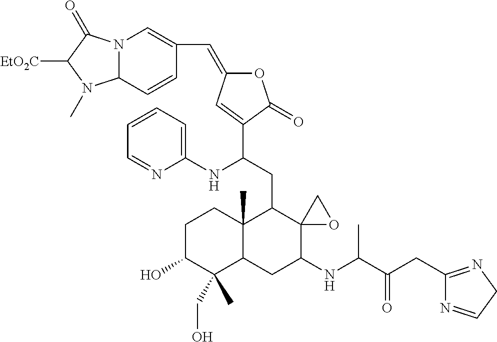 [SMILES: CCOC(=O)C1C(=O)N2C=C(/C=C3\C=C(C(CC4C5(CO5)C(NC(C)C(=O)CC5=NCC=N5)CC5[C@]4(C)CC[C@@H](O)[C@@]5(C)CO)Nc4ccccn4)C(=O)O3)C=CC2N1C] | 826.40 |
| 424 | 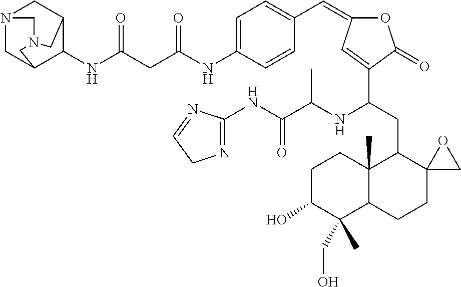 [SMILES: CC(NC(CC1C2(CCC3[C@]1(C)CC[C@@H](O)[C@@]3(C)CO)CO2)C1=C/C(=C\c2ccc(NC(=O)CC(=O)NC3C4CN5CC3CN(C4)C5)cc2)OC1=O)C(=O)NC1=NCC=N1] | 826.44 |
| 425 | 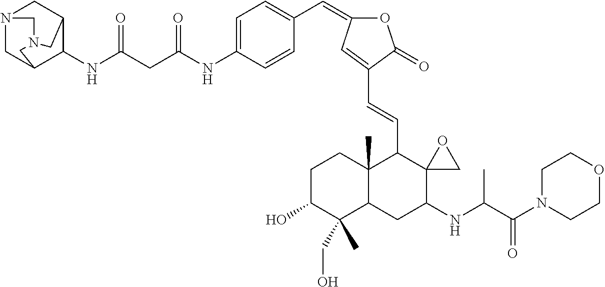 [SMILES: CC(NC1CC2[C@](C)(CC[C@@H](O)[C@@]2(C)CO)C(/C=C/C2=CC(=C\c3ccc(NC(=O)CC(=O)NC4C5CN6CC4CN(C5)C6)cc3)/OC2=O)C12CO2)C(=O)N1CCOCC1] | 828.44 |
| 426 | 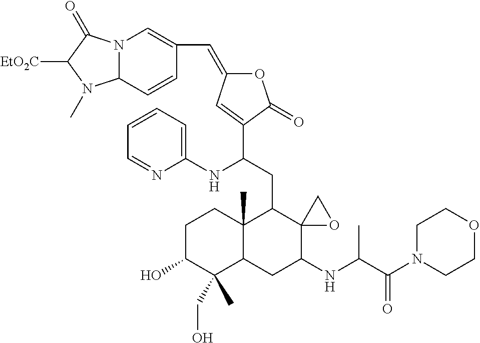 [SMILES: CCOC(=O)C1C(=O)N2C=C(/C=C3\C=C(C(CC4C5(CO5)C(NC(C)C(=O)N5CCOCC5)CC5[C@]4(C)CC[C@@H](O)[C@@]5(C)CO)Nc4ccccn4)C(=O)O3)C=CC2N1C] | 830.42 |
| 427 | 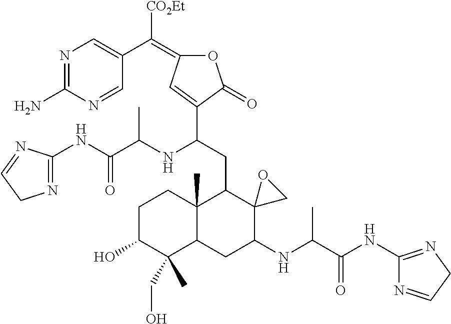 [SMILES: CCOC(=O)/C(=C1/C=C(C(CC2C3(CO3)C(NC(C)C(=O)NC3=NCC=N3)CC3[C@]2(C)CC[C@@H](O)[C@@]3(C)CO)NC(C)C(=O)NC2=NCC=N2)C(=O)O1)c1cnc(N)nc1] | 831.40 |
| 428 | 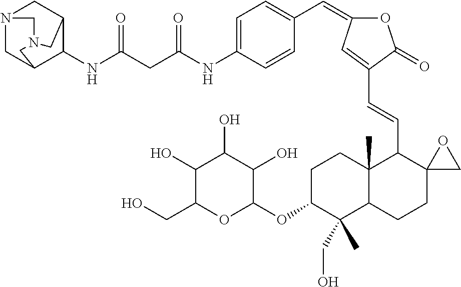 [SMILES: C[C@]12CC[C@@H](OC3OC(CO)C(O)C(O)C3O)[C@@](C)(CO)C1CCC1(CO1)C2/C=C/C1=CC(=C\c2ccc(NC(=O)CC(=O)NC3C4CN5CC3CN(C4)C5)cc2)/OC1=O] | 834.41 |
| 429 | 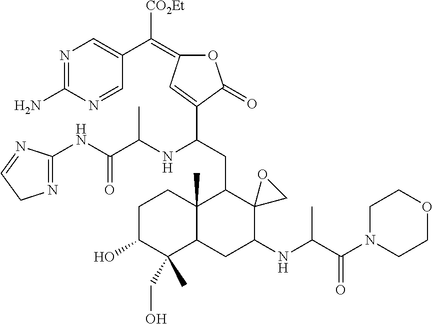 [SMILES: CCOC(=O)/C(=C1/C=C(C(CC2C3(CO3)C(NC(C)C(=O)N3CCOCC3)CC3[C@]2(C)CC[C@@H](O)[C@@]3(C)CO)NC(C)C(=O)NC2=NCC=N2)C(=O)O1)c1cnc(N)nc1] | 835.42 |
| 430 | 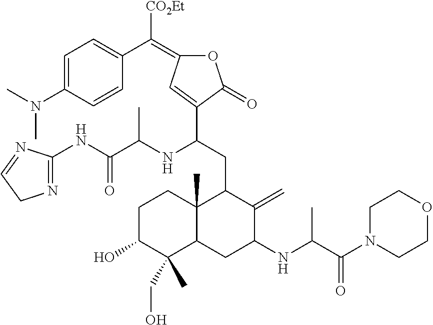 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C(/C(=O)OCC)c2ccc(N(C)C)cc2)OC1=O] | 845.47 |
| 431 | 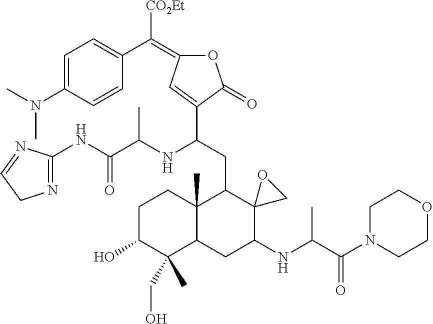 [SMILES: CCOC(=O)/C(=C1/C=C(C(CC2C3(CO3)C(NC(C)C(=O)N3CCOCC3)CC3[C@]2(C)CC[C@@H](O)[C@@]3(C)CO)NC(C)C(=O)NC2=NCC=N2)C(=O)O1)c1ccc(N(C)C)cc1] | 861.46 |
| 432 | 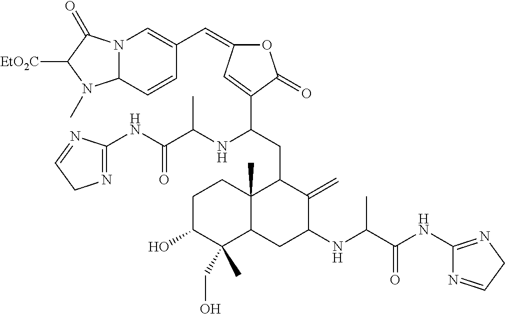 [SMILES: C=C1C(NC(C)C(=O)NC2=NCC=N2)CC2[C@](C)(CC[C@@H](O)[C@@]2(C)CO)C1CC(NC(C)C(=O)NC1=NCC=N1)C1=C/C(=C\C2=CN3C(=O)C(C(=O)OCC)N(C)C3C=C2)OC1=O] | 870.44 |
| 433 | 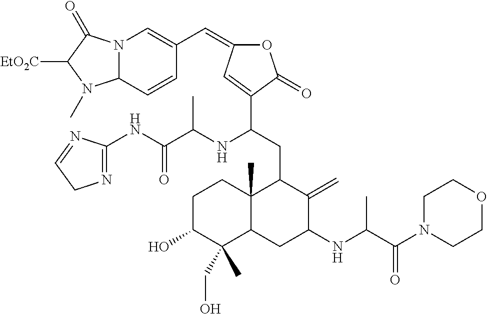 [SMILES: C=C1C(NC(C)C(=O)N2CCOCC2)CC2[C@](C)(CC[C@@H](O)[C@@]2(C)CO)C1CC(NC(C)C(=O)NC1=NCC=N1)C1=C/C(=C\C2=CN3C(=O)C(C(=O)OCC)N(C)C3C=C2)OC1=O] | 874.46 |
| 434 | 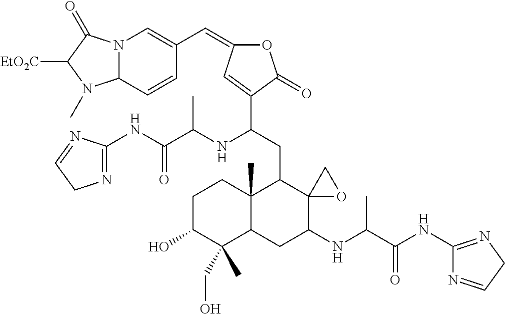 [SMILES: CCOC(=O)C1C(=O)N2C=C(/C=C3\C=C(C(CC4C5(CO5)C(NC(C)C(=O)NC5=NCC=N5)CC5[C@]4(C)CC[C@@H](O)[C@@]5(C)CO)NC(C)C(=O)NC4=NCC=N4)C(=O)O3)C=CC2N1C] | 886.43 |
| 435 | 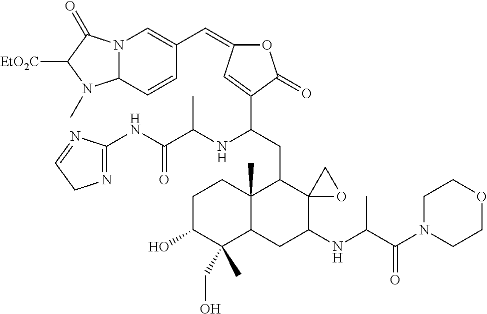 [SMILES: CCOC(=O)C1C(=O)N2C=C(/C=C3\C=C(C(CC4C5(CO5)C(NC(C)C(=O)N5CCOCC5)CC5[C@]4(C)CC[C@@H](O)[C@@]5(C)CO)NC(C)C(=O)NC4=NCC=N4)C(=O)O3)C=CC2N1C] | 890.45 |
| 436 | 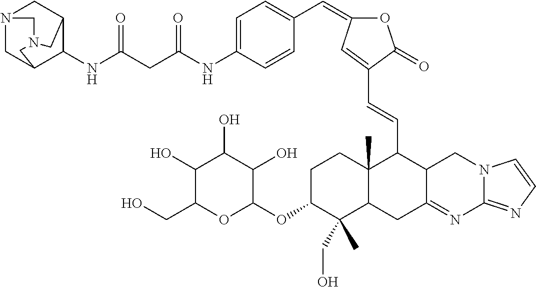 [SMILES: C[C@]12CC[C@@H](OC3OC(CO)C(O)C(O)C3O)[C@@](C)(CO)C1CC1=Nc3nccn3CC1C2/C=C/C1=CC(=C\c2ccc(NC(=O)CC(=O)NC3C4CN5CC3CN(C4)C5)cc2)/OC1=O] | 897.43 |

### 3. GENERAL METHOD OF INJECTION PREPARATION

### Example 437 Preparation of Injection I

The example compound 5.0 g, ethanol 600 mL (95%), 1,2-propanediol 600 mL and Tween (80) 100 mL were dissolved and the injection water was added up to total volume of 5000 mL. The solution was filtered with 0.22 μm membrane filter and sterilized for 30 min at 100° C. to obtain 1000 preparation of injection 5 mg/5 mL.

### Example 438

### Preparation of Injection II

The example compound 8.0 g, DMSO 50 mL, 1,2-propanediol 100 mL and Tween 80 100 mL were dissolved and the injection water was added up to total volume of 5000 mL. The solution was filtered with 0.22 μm membrane filter and sterilized 30 min at 100° C. to obtain 1000 preparation of injection 8 mg/5 mL.

### 4. ACTIVITY ASSAY

### Example 439 Antivirus Studies In Vitro

Test Samples:

examples of 23, 35, 37, 38, 40, 41, 44, 45, 46, 50, 53, 55, 57, 58, 59, 62, 63, 65, 66, 68, 69, 70, 71, 76, 77, 78, 79, 83, 95, 124 and 188.

Virus Strains:

from NIAID

Methods:

compounds were diluted to 20 mg/mL in DMSO then prepared in for log 10 dilutions of 100 μg/mL down to 0.1 μg/mL) in MEM solution with 50 μg/mL gentamicin and 2% FBS. Each dilution was added to 5 wells of a 96-well plate with 80-100% confluent cells. Three wells of each dilution were infected with virus, and two wells remained uninfected as toxicity controls. The multiplicity of infection (MOI) was as low as possible to achieve CPE within 3-5 days of incubation. A known active compound was tested in parallel as a control. After untreated virus control wells reached maximum cytopathic effect (CPE), each well was scored microscopically. Plates were then stained with neutral red dye for approximately 2 hours, then supernatant dye was removed from the wells and the incorporated dye was extracted in 50:50 Sorensen citrate buffer/ethanol, then the optical density was read on a spectrophotometer. Optical densities were converted to percent of cell controls and normalized to the virus control, then the concentration of test compound required to inhibit CPE by 50% (EC₅₀) was calculated by regression analysis.

Results:

the in-vitro antiviral screening result see Table 2

TABLE 2
In-Vitro Antiviral Screening Result (EC50)

| Example | | | | | | | | | | | | | | | |
|---|---|---|---|---|---|---|---|---|---|---|---|---|---|---|---|
| Compd. | A | B | C | D | E | F | G | H | I | J | K | L | M | N | O |
| 95 | − | ++ | − | + | − | ++ | + | ++ | ++ | ++ | − | − | + | ++ | ++ |
| 58 | ++ | ++ | ++ | ++ | ++ | ++ | ++ | ++ | ++ | ++ | + | ++ | + | ++ | − |
| 57 | ++ | ++ | ++ | ++ | ++ | ++ | ++ | ++ | ++ | ++ | ++ | + | ++ | − | ++ |
| 40 | + | − | + | − | − | ++ | − | − | ++ | ++ | − | + | − | + | − |
| 83 | ++ | +++ | ++ | ++ | ++ | ++ | ++ | ++ | ++ | ++ | ++ | ++ | ++ | ++ | ++ |
| 55 | ++ | ++ | ++ | ++ | ++ | ++ | ++ | ++ | ++ | ++ | − | ++ | ++ | − | ++ |
| 37 | ++ | +++ | ++ | ++ | ++ | ++ | ++ | ++ | ++ | ++ | ++ | ++ | ++ | ++ | ++ |
| 35 | + | ++ | ++ | ++ | − | ++ | ++ | ++ | ++ | ++ | ++ | − | ++ | + | ++ |
| 53 | ++ | ++ | ++ | ++ | +++ | ++ | ++ | ++ | ++ | ++ | − | ++ | − | ++ | − |
| 45 | ++ | ++ | ++ | − | ++ | ++ | ++ | − | ++ | ++ | − | + | + | − | ++ |
| 79 | ++ | ++ | ++ | ++ | ++ | ++ | ++ | ++ | − | ++ | ++ | ++ | − | ++ | + |
| 188 | − | ++ | + | − | + | ++ | + | − | + | ++ | + | + | − | ++ | + |
| 23 | ++ | ++ | ++ | − | ++ | ++ | ++ | − | − | ++ | ++ | − | ++ | ++ | ++ |
| 38 | − | ++ | + | + | − | ++ | + | + | − | ++ | + | + | − | ++ | + |
| 41 | ++ | ++ | − | + | ++ | + | − | + | ++ | ++ | − | + | ++ | ++ | − |
| 44 | − | ++ | − | + | − | ++ | − | + | − | + | − | + | − | + | − |
| 46 | ++ | ++ | + | − | ++ | ++ | + | + | ++ | ++ | + | − | ++ | ++ | + |
| 50 | − | ++ | ++ | − | + | + | ++ | ++ | + | ++ | ++ | − | − | ++ | ++ |
| 59 | ++ | ++ | − | + | ++ | ++ | − | + | ++ | ++ | − | + | ++ | + | − |
| 62 | − | ++ | ++ | + | − | + | ++ | + | − | ++ | ++ | + | − | ++ | ++ |
| 63 | ++ | ++ | + | + | ++ | ++ | + | ++ | ++ | + | + | + | ++ | ++ | + |
| 71 | − | ++ | ++ | + | − | + | ++ | + | − | ++ | ++ | + | − | + | ++ |
| 65 | ++ | ++ | − | + | ++ | ++ | + | − | + | ++ | − | − | ++ | ++ | − |
| 66 | + | ++ | ++ | − | + | + | ++ | + | + | − | ++ | ++ | + | + | ++ |
| 68 | − | ++ | ++ | − | − | ++ | ++ | − | − | ++ | ++ | − | − | ++ | ++ |
| 69 | ++ | ++ | − | + | ++ | ++ | − | + | ++ | ++ | − | + | ++ | + | − |
| 70 | − | ++ | + | + | − | + | + | + | − | − | + | + | − | ++ | + |
| 124 | ++ | ++ | ++ | + | ++ | ++ | ++ | + | ++ | ++ | ++ | + | ++ | ++ | ++ |
| 76 | − | ++ | − | + | + | ++ | − | + | − | + | − | + | + | + | − |
| 77 | ++ | ++ | + | − | ++ | + | − | + | + | ++ | − | + | ++ | ++ | − |
| 78 | + | ++ | + | + | + | ++ | + | − | + | ++ | + | − | + | ++ | + |

Note:
all vehicle: DMSO;
all compound concentration range: 0.1-100 μg/mL;
−represent >100 μg/mL,
+represent <100 μg/mL,
++represent <50 μg/mL,
+++represent <10 μg/mL;
A: Dengue virus, Virus Strain: type 2, New Guinea C;
B: .Entrovirus-71, Virus Strain: Tainan/4643/98,;
C: Japanese encepahalitis virus, Virus Strain: SA-14/V1;
D: respiratory syncytial virus, Virus Strain: A2;
E: Rift Valley fever virus, Virus Strain: MP-12;
F: SARS coronavirus, Virus Strain: Urbani;
G: Venezuelan equine encephalitis virus, Virus Strain: TC-83;
H: Influenza A virus H₁N₁, Virus Strain: Califomia/07/2009;
I: Hepatitis C virus, Virus Strain: CON1;
J: Hepatitis B virus, Virus Strain: 02094;
K: Hepatitis A virus, Virus Strain: pHM175;
L: herpes simplex virus, Virus Strain: type 2;
M: human papillomavirus, Virus Strain: type 8;
N: herpes simplex virus, Virus Strain: type 1;
O: HIV-1, Virus Strain: M;
P: Human rhinovirus A serotype 89, Virus Strain: 41467-Gallo;
Q: Human herpesvirus 1, Virus Strain: 17;
R: Rubella virus, Virus Strain: TO-336;
S: Human parainfluenza 1 virus, Strain: strain C39;
T: Human parainfluenza 1 virus Virus Strain: strain C39. Compound No. (see the Table 2. The Structures of Andrographolide Analogs).

Conclusion:

Table 2. showed the example compounds of the present invention possess excellent antiviral activity against variety virus. It is especially noteworthy that the example compounds of 37, 53, 57 and 83 are effective against virus of B, E and F with EC₅₀ less than 1 μg/ml; the example compounds of 23, 35, 38, 40, 41, 44, 45, 46, 50, 55, 58, 59, 62, 63, 65, 66, 68, 69, 70, 71, 76, 77, 78, 79, 95, 124 and 188 are effective against many virus with EC₅₀ less than 50 μg/ml.

### Example 440 Efficacy Studies of Anticancer In Vivo

Test Samples:

examples of 1, 6, 11, 14, 22, 25, 31, 35, 36, 37, 39, 40, 45, 46, 55, 57, 58, 71, 76, 95, 108, 124, 184, 188, 210 [symbol] 246.

Experimental Animals:

Kunming healthy mice provided by the Animal Center of Beijing Academy of Military Medical.

Methods:

xenografts cultured S₁₈₀ tumor cells were implanted subcutaneously into the flank region of mice and tumors were allowed to grow to the desired average size of 100 mg. The mice were randomized into control and treatment groups with 10 mice per group. The control group was injected with the vehicle used to dissolve the drug. Other groups received the test compounds (example compound 18, 24, 25, 26 and 27) and positive group, cyclophosphamide (CTX) and 5-fluorouracil (5-FU) at the dose and schedule as indicated in Table VI. Injections were I.V. via the tail vein. Tumor measurements were taken every other day 20% tumor growth inhibition which was not statistically significant.

Results: the in vivo experimental data showed anti-tumor efficacy of example compounds 71, 188, 210, 108, 39, 1, 11 and 57 are statistically significant.

TABLE 3
Growth Inhibition of S₁₈₀ sarcoma ( X̅ ± SD, n = 16)

| Example | adminis- | | Tumor | Inhibition | P value |
|---|---|---|---|---|---|
| Compound | tration | mg/kg | weight(g) | rate(%) | (<) |
| saline | iv | 50 | 1.25 ± 0.31 | — | — |
| CTX | iv | 15 | 0.48 ± 0.19 | 54.16 | 0.00** |
| 22 | iv | 12.5 | 34.29 ± 3.88 | 21.03 | 0.88 |
| 14 | iv | 50 | 34.27 ± 2.05 | 23.18 | 0.71 |
| 95 | ip | 100 | 25.52 ± 1.72 | 16.19 | 0.12 |
| 58 | ip | 25 | 24.67 ± 1.67 | 11.13 | 0.06 |
| 57 | ip | 200 | 23.58 ± 1.20 | 40.96 | 0.00** |
| 55 | ip | 200 | 25.56 ± 3.28 | 11.15 | 0.1 |
| 40 | ip | 400 | 26.87 ± 1.47 | 15 | 0.2 |
| 35 | ip | 100 | 27.74 ± 2.40 | 40.08 | 0.00** |
| 37 | ip | 300 | 28.37 ± 1.62 | 17.87 | 0.09 |
| 36 | ip | 200 | 28.18 ± 2.02 | 20.82 | 0.05 |
| 39 | ip | 100 | 25.98 ± 1.02 | 9.45 | 0.00** |
| 1 | iv | 15 | 0.64 ± 0.69 | 44.93 | 0.00** |
| 11 | iv | 20 | 0.49 ± 0.36 | 58.33 | 0.00** |
| 45 | iv | 15 | 1.36 ± 0.79 | −16.03 | — |
| 108 | ip | 200 | 0.97 ± 0.27 | 47.57 | 0.00** |
| 31 | ip | 400 | 1.36 ± 0.46 | 26.49 | 0.01* |
| 6 | iv | 100 | 1.36 ± 0.82 | 44.32 | 0.01* |
| 246 | iv | 30 | 1.25 ± 0.44 | 26.03 | 0.1 |
| 184 | iv | 200 | 0.81 ± 0.47 | 15.21 | 0.15 |
| 25 | iv | 50 | 0.83 ± 0.24 | 10.21 | 0.15 |
| 188 | ip | 50 | 0.72 ± 0.34 | 42.4 | 0.00** |
| 210 | ip | 100 | 0.72 ± 0.32 | 42.4 | 0.00** |
| 46 | ip | 70 | 0.92 ± 0.37 | 26.4 | 0.07 |
| 71 | ip | 150 | 0.54 ± 0.11 | 51.8 | 0.00** |
| 124 | ip | 100 | 0.84 ± 0.38 | 32.8 | 0.03 |
| 76 | ip | 100 | 0.81 ± 0.71 | 16.8 | 0.15 |

Note:
all vehicle: DMSO; dose range: 4-100 mg/kg;
— represent of Inhibition rate % < 10, + represent <20, ++ represent <40, +++ represent >40;
*P < 0.01: compared with the control group significantly difference;
**p < 0.001: compared with the control group was very significant difference. Inhibition rate more than 40% of the sample was statistically significant better than control group.

1. A compound of the formula I: 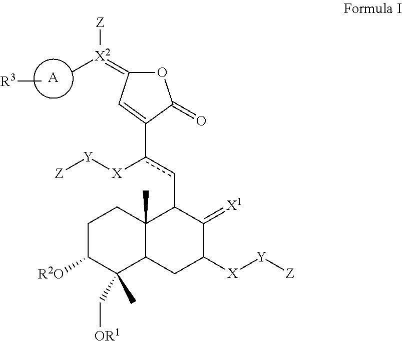
or stereoisomers, tautoers, prodrug, pharmaceutically acceptable salts, complex salts or solvates thereof, wherein: the dotted lines [symbol] are absent or selected but is not limited from: —C—C—, —C═C—, —C-heterobond, 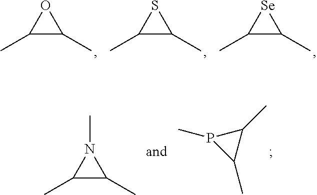
X, X¹ and X² is absent or independently selected but is not limited from: hydrogen, halogen, —C—, —S—, —O—, —NH—, —NR—, —NRR, —NHC(O)—, —NRC(O)—, —NHSO₂—, —NRSO₂—, —SO₂NH—, —SO₂NR—, —C(O)—, —C(O)NH—, —C(O)NR—, —C(O)R—, —CO₂—, —C(O)H, —C(O)NH₂—, —CO₂H, —C(NH)NH—, —C(NH)NR—, —C(S)—, —C(S)NH—, —C(S)NR—, —C(S)R—, —C(NH)NH—, —C(NH)NR—, ═CH—, ═CH₂, ═CH—O—, ═CH—S—, ═CH—Se—, ═CH—NR—, ═CH—NH—, ═CH—PR—, wherein R is selected but is not limited from: —C₁₋₆ alkyl, —CO₂H, —CO₂C₁₋₆alkyl, COC₁₋₆alkyl, phenyl, —CH₂phenyl, heteroalkyl and heteroaryl;
Y is absent or selected but is not limited from: —CH₂—, —CH₂—CH₂—, —CH₂—CH₂—CH₂—, —CHF—, and —CF₂—, wherein each CH₂ and CHF is unsubstituted or substituted with 1 or 2 substituents selected from R^{a};
Z is absent or selected but is not limited from: hydrogen, halogen, —C₁₋₆alkyl, —C₂₋₆alkenyl, —C₂₋₆alkynyl, —C₃₋₁₀cycloalkyl, —C₃₋₁₀cycloalkenyl, aryl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, guanidine, amidine —SC₁₋₆alkyl, —SOC₁₋₆alkyl, —SO₂C₁₋₆alkyl, —NHSO₂C₁₋₆alkyl, —NHC(O)C₁₋₆alkyl, —SO₂NHC₁₋₆alkyl, —C(O)OH, —C(O)OC₁₋₆alkyl, —C(O)NHC₁₋₆alkyl, P(O)(OH)2, P(O)(OR)2, —(CH₂)ₘP(O)(OH)₂, —(CH₂)ₘP(O)(OC₁₋₆alkyl)₂, —(CH₂)ₘP(O)(NR^{b}C(R^{c}))₂, C₃₋₈ amino acid, C₃₋₁₀(OH)₀₋₁₀polyhydroxide;
each R¹, R² and R³ is absent or independently selected but is not limited from: hydrogen, halogen, —C₁₋₁₀alkyl, —C₂₋₆alkenyl, —C₂₋₆lkynyl, —(CH₂)ₚC₃₋₁₀ cycloalkyl, —(CH₂)ₚC₃₋₇cycloalkylaryl, —(CH₂)ₚC₃₋₇cycloalkylheteroaryl, —(CH₂)ₚC₄₋₁₀cycloalkenyl, —(CH₂)ₚC₄₋₇ cycloalkenyl-aryl, —(CH₂)ₚC₄₋₇cycloalkenylheteroaryl, —(CH₂)ₚheteroaryl, —C₂₋₆alkenylalkyl, —C₂₋₆alkenylaryl, —C₂₋₆alkenyl-heteroaryl, —C₂₋₆alkenyl-C₃₋₇cycloalkyl, —C₂₋₆alkynylalkyl, —C₂₋₆alkynylaryl, —C₂₋₆alkynyheteroaryl, —C₂₋₆alkynyl-C₃₋₇cycloalkyl, —C₂₋₆alkynyl-C₃₋₇cycloalkenyl, —C₂₋₆alkynyl-C₂₋₇cycloheteroalkyl, —C₂₋₆alkynyl-C₂₋₇cycloheteroalkenyl, —C(O)(CH₂)₀₋₃phenyl, —(CH₂)ₚC(O)phenyl, —C(NH) (CH₂)₀₋₃phenyl, —(CH₂)ₘP(O)(NR^{b}C(R^{c}))₂, C₃₋₈ amino acid, C₃₋₁₀(OH)₀₋₁₀polyhydroxide, aryl, biphenyl, —C₃₋₁₀heterocycle, —C₃₋₁₀heteroaryl, —CN, —CF₃, —OH, —OC₁₋₆alkyl, —NH₂, —NHC₁₋₆alkyl, —N(C₁₋₆alkyl)₂, —NHC(O)C₁₋₆alkyl, guanidine, amidine —SC₁₋₆alkyl, —SOC₁₋₆alkyl, —SO₂C₁₋₆alkyl, —NHSO₂C₁₋₆alkyl, —SO₂NHC₁₋₆alkyl, —C(O)OH, —C(O)OC₁₋₆alkyl, —C(O)NHC₁₋₆alkyl, P(O)(OH)2, P(O)(OR)2, —(CH₂)ₘP(O)(OH)₂,-(CH₂)ₘP(O)(OC₁₋₆alkyl)₂, —(CH₂)ₘP(O)(NR^{b}C(R^{c}))₂, C₃₋₈ amino acid, C₃₋₁₀(OH)₀₋₁₀polyhydroxide;
wherein each CH₂ is unsubstituted or substituted with 1 or 2 substituents selected from: halogen, —CF₃, —OH, —NH₂, —C₁₋₆alkyl, —OC₁₋₅alkyl, —NHC₁₋₆alkyl and —N(C₁₋₆alkyl)₂, each alkyl, alkenyl and alkynyl is unsubstituted or substituted with 1, 2 or 3 substituents selected from: halogen, CF₃, —OH, —NH₂, —C₁₋₆alkyl, —OC₁₋₆alkyl, —NHC₁₋₆alkyl and —N(C₁₋₆alkyl)₂, and each cycloalkyl, cycloalkenyl, cycloheteroalkyl, cycloheteroalkenyl, phenyl, aryl and heteroaryl is unsubstituted or substituted with 1, 2, 3 or 4 substituents independently selected from R^{a};
A ring is selected from: —C₃₋₁₄alkylcycle, —C₃₋₁₄arylcycle, —C₃₋₁₄heterocycle and —C₃₋₁₄heteroaryl;
said —C₃₋₁₄alkylcycle, arylcycle, heterocycle and —C₃₋₁₄heteroaryl are selected but are not limited from acridinyl, azetidinyl, acridinyl, azocinyl, azepanyl, azepinyl, aziridinyl, azirinyl, azete, benzothiazole, benzofuranyl, benzimidazolyl, benzofuranyl, benzothranyl, benzothiofuranyl, benzthiazolyl, benztetrazolyl, benzisoxazolyl, benzisothiazolyl, benzimidazolinyl, benzopyrazolyl, benzotriazolyl, benzothiophenyl, benzoxazolyl, benz-1H-tetrazolyl, benz-2H-tetrazolyl, benz-3H-tetrazolyl, benz-4H-tetrazolyl, benz-5H-tetrazolyl, benzothienyl, benzofurazanyl, benzodiazepinyl, carbazolyl, carbolinyl, cinnolinyl, carbazolyl, carbolinyl, chromanyl, chromenyl, coumarinyl, decahydroquinolinyl, 4aH-carbazolyl, 1,4-dioxanyl, 2H, 6H-1,5,2-dithiazinyl, dihydrofuro[2,3-b]tetrahydrothran, furazanyl, hexahydroazepinyl, imidazole, imidazolyl, indolinyl, indolazinyl, indazolyl, isoindolyl isoquinolyl, imidazolidinyl, imidazolinyl, indolyl, indolazinyl, indazolyl, 1H-indazolyl, indolenyl, indolinyl, indolizinyl, 3H-indolyl, isobenzofuranyl, isoquinolyl, 1,2-Isoxazole, 1,3-Isoxazole, isoxazolyl, isochromanyl, isoindolinyl, isothiazolyl, isoxazoline, isoquinolinyl, methylenedioxybenzoyl, methylenedioxyphenyl, morpholinyl, naphthpyridinyl, N-oxides oxetanyl, oxadiazolyl, oxazolyl, oxazolinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxolanyl, oxirenyl, oxete, oxiranyl, oxanyl, oxetanyl, oxepanyl, oxepinyl, oxazolinyl, pyrazole, 2(1H)-pyrimidinone, piperidine, thiiranyl, phenanthridinyl, phenanthrolinyl, phenazinyl, phenothiazinyl, phenoxathiinyl, phenoxazinyl, phthalazinyl, piperidinyl, 2H-pyrrolyl, pyridin-2-one, piperazinyl, piperidonyl, 4-piperizinyl, piperonyl, pteridinyl, purinyl, pyrazinyl, pyrazolidinyl, pyridazine, pyrazolinyl, pyridazinyl, pyridoimidazole, pyridothiazole, pyridyl, pyrimidinyl, pyridopyridinyl, pyrrazolyl, pyrrolinyl, pyrazolyl, pyrrolyl, pyranyl, pyrazine, pyridinyl, pyrrolidinyl, quinazolinyl, quinolyl, quinoxalinyl, 4H-quinolizinyl, quinuclidinyl, quinoxaline-2(1H)-one, quinolinyl, thiadiazolyl, thranyl, 6H-1,2,5-thiadiazinyl, 1,2,3-thiadiazolyl, 1,2,4-thiadiazinyl, 1,2,5-thiadiazinyl, 1,3,4-trizzolyl, 1,3,4-thiadiazinyl, thiomorpholinyl, thienyl, thiomorpholinyl, triazolyl, thiirenyl, thietanyl, thiete, thiolanyl, thiophenyl, thianyl, thiopyranyl, thiepanyl, thiepinyl, thiazole, thionaphthenyl, purinyl, 2,4,6-trihydroxypurinyl, 1,2-thiazole, 1,3-thiazole, xanthenyl;
wherein A ring includes the above said heteroaryls, as well as dihydro and tetrathydro analogs and is, unsubstituted or optionally substituted with 1, 2 or 3 same or different substituents and each substituent selected but are not limited from: hydrogen, halogen, —OH, —OR, —SH—, —SR—, —O—, —NO₂, —NH₂, —NH—, —NR—, —NRR, —CF₃, —CN, —C(O)—, —NHC(O)—, —NRC(O)—, —C(O)NH—, —C(O)NR—, —NHSO₂—, —NRSO₂—, —SO₂NH—, —SO₂NR—, —C(O)R—, —CO₂—, —C(NH)NH—, —C(NH)NR—, wherein R is selected from: —C₁₋₆ alkyl, —CH₂F, —CHF₂, —CF₃, —CO₂H, —CO₂C₁₋₆alkyl, COC₁₋₆alkyl, phenyl, —CH₂phenyl, heteroalkyl and heteroaryl oxo, —(CH₂)₀₋₃OH, —CN, —NH₂, —NH(C₁₋₆alkyl), —N(C₁₋₆alkyl)2, —C₁₋₆alkyl, —OC₁₋₆alkyl, halogen, —CH₂F, —CHF₂, —CF₃, —CO₂H, —CO₂C₁₋₆alkyl, —C₃₋₇cycloalkyl, phenyl, CH₂phenyl, heteroaryl and CH₂ heteroaryl;
R^{a}, R^{b} and R^{c} are same or different selected but is not limited from hydrogen, halogen, —C—, —S—, —O—, —NH—, —NR—, —NRR, —NHC(O)—, —NRC(O)—, —NHSO₂—, —NRSO₂—, —SO₂NH—, —SO₂NR—, —C(O)—, —C(O)NH—, —C(O)NR—, —C(O)R—, —CO₂—, —C(O)H, —C(O)NH₂—, —CO₂H, —C(NH)NH—, —C(NH)NR—, —C(S)—, —C(S)NH—, —C(S)NR—, —C(S)R—, —C(NH)NH—, —C(NH)NR—, wherein R is selected from: —C₁ alkyl, —CO₂H, —CO₂C₁₋₆alkyl, COC₁₋₆alkyl, phenyl, —CH₂phenyl, heteroalkyl and heteroaryl; m is 0, 1, 2, 3 or 4; p is 0, 1, 2, or 3;

2. According to the claim 1, wherein: a compound of andrographolide derivatives and analogs is selected but is not limited from the exemplified examples or stereoisomers, tautomers, pharmaceutically acceptable salts, inorganic acid salt, organic acid salt, organic basic salt, organic basic salt, complex salt, prodrug or solvates thereof in association with a pharmaceutically acceptable excipient or carrier; in addition, an acid or a base may be incorporated into the composition to facilitate processing, to enhance stability, or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl)aminomethane and the like; bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, parabromophenylsulfonic acid, propionic acid, p-toluenesulfonic acid, salicylic acid, stearic acid, succinic acid, taimic acid, tartaric acid, thioglycolic acid, toluenesulfonic acid, uric acid, and the like; salts of polyprotic acids, such as sodium phosphate, disodium hydrogen phosphate, and sodium dihydrogen phosphate can also be used; when the base is a salt, the cation can be any pharmaceutically acceptable cation, such as ammonium, alkali metals, alkaline earth metals, and the like, example may include sodium, potassium, lithium, magnesium, calcium and ammonium; suitable acids are pharmaceutically acceptable organic or inorganic acids, examples of inorganic acids include hydrochloric acid, hydrobromic acid, hydriodic acid, sulfuric acid, nitric acid, boric acid, phosphoric acid, and the like.

3. A compound according to the claim 1, wherein: a process for the manufacture of a compound of formula I to form andrographolide derivatives and analogs is obtained by modification at 7-, 12- or 15-position with a bond of C—C, C—O, C—S, C—N or C—P under catalysis at −78° C. to 90° C., by a solvent selected from THF, 1,4-dioxane, N, N-dimethylformamide, N, N-dimethylacetamide, toluene, ethanol, or methanol, at room temperature to 180° C., and a catalyst selected from organic base, inorganic base, molecular sieves or alumina; the introduction of unsubstituted or substituted alicyclic, arylring, aliheterocyclic or arylheterocyclic base is prepared into andrographolide derivative or analogue; in addition, the present invention is composed of the acid or base to increases the solubility, to enhance the stability or for other reasons; examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, disopropylethylamine, ethanolamine, ethylenediamine, triethanolamine, triethylamine, trisopropanolamine, trimethylamine, tris(hydroxymethyl) aminomethane; examples of pharmaceutically acceptable bases include acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, parabromophenylsulfonic acid, propionic acid, p-toluenesulfonic acid, salicylic acid, stearic acid, succinic acid, taimic acid, tartaric acid, thioglycolic acid, toluenesulfonic acid, uric acid;

4. A method according to the claim 1, wherein: the amount of one or more of the compounds of general formula I, or a pharmaceutically acceptable salt thereof, present in the composition for treating, preventing or slowing the progression of virus, cancer, bacteria, fungi and other infections, including inflammation, inflammatory diseases and immune system disease associated with viruses, cancer, bacterial and fungi, alone or with the following drugs known to be used in conjunction dose in a range of about 0.02-2.0 g/kg; means of various methods of treatment and therapy, where the virus are selected but are not limited from adenovirus, herpes simplex type 1, herpes simplex type 2, varicella-zoster virus, epstein-barn virus, human cytomegalovirus, human herpesvirus, type 8 human papillomavirus, BK virus, JC virus, smallpox, human bocavirus, parvovirus, B19 human astrovirus, norwalk virus, coxsackievirus, hepatitis A virus, hepatitis B virus, hepatitis C virus, poliovirus, rhinovirus, severe acute respiratory syndrome virus, yellow fever virus, dengue virus, west nile virus, rubella virus Hepatitis E virus, human immunodeficiency virus, influenza virus, guanarito virus, junin virus, lassa virus, machupo virus, Sabia virus, Crimean-Congo hemorrhagic fever virus, ebola virus, marburg virus, measles virus, mumps virus, parainfluenza virus, respiratory syncytial virus, human metapneumovirus, rabies virus, hepatitis D rotavirus; In addition, the compositions of the invention are useful for inhibiting viral replication, the treatment of viral infections, the treatment of infections which are caused by DNA viruses, such as e.g. herpes simplex virus, the cytomegalovirus, papovavirus, the varicella zoster virus or Epstein-Barr virus, the treatment of infections which are caused by RNA viruses, such as togaviruses or retroviruses, the treatment of infections which are caused by HTLV-I and II, the treatment of infections which are caused by lentiviruses; In some embodiments, and the treatment of infections are caused by HIV-1 and 2.

5. A method according to the claim 1, wherein: a compound of andrographolide derivatives and analogs for treating, preventing or slowing the progression of virus, bacteria, fungi and other infections associated with inflammation and inflammatory diseases, immune system complications of viral infection by respiratory tract, urinary tract, skin and soft tissue, sepsis, bone and joint, abdominal, pelvic viridans, endocarditis, anaerobic, anthrax, syphilis or gonorrhea comprising: aids, cutaneous lesion ssociated with AIDS, AIDS related malignant tumours, alphaviruses causing encephalitis, arenavirus, arthropod-borne viral encephalitis, avian influenza, bolivian hemorrhagic fever, coxsackievirus infection, crimean-congo hemorrhagic fever, cytomegalovirus infection, dengue, eastern equine encephalitis, ebola virus infection, echovirus infection, epstein-barn virus infection, epstein-barn virusrelated malignant tumours, fifth disease, filovirus, flavivirus, german measles, hemorrhagic fever with renal syndrome, herpes virus, herpes simplex virus infection, herpes zoster virus, human papilloma virus associated epidermal lesions, human papilloma virus in cervical cancer, Japanese encephalitis, kaposi sarcoma, korean hemorrhagic fever, kyasanur forest disease, lassa fever, lymphocytic choriomeningitis, molluscum contagiosum, murray valley encephalitis, norwalk virus related diarrhea, omsk hemorrhagic fever, orthomyxoviruses, parainfluenza virus infection, paramyxovirus, parvovirus B19 infection, picornavirus, rotavirus diarrhea, poxviruses, rabies, respiratory syncytial virus infection, rubeola, smallpox, St. Louis encephalitis, tick-borne encephalitis, variola, venezuelan equine encephalitis, viral hemorrhagic fevers, viruses in leukemia and lymphoma, western equine encephalitis, west Nile virus disease septicemia, endocarditis infection, adenovirus serotype 14, T-cell leukemia/lymphoma, alastrim, andes viral infection, argentine hemorrhagic fever, astrovirus infection, avian encephalomyelitis viral infection, avian nephritis viral infection, avian orthoreoviral infection, avian pneumoviral infection, borna disease, bornholm disease, bovine adenoviral infection, bovine coronaviral infection, bovine ephemeral fever, bovine herpesviral infection 4, bovine parvoviral infection, bovine viral diarrhea, brazilian hemorrhagic fever, bronchiolitis, bundibugyo ebolaviral infection, bundibugyo viral infection, cat flu, cervical intraepithelial neoplasia, chandipura viral infection, channel catfish viral infection, chicken anaemia viral infection, chickenpox, chikungunya outbreaks, common cold, cowpox, coxsackie viral infection, cricket paralysis viral infection, cuevaviral infection, cytomegaloviral infection, cytomegalovirus colitis, cytomegalovirus retinitis, derzsy's disease, downie bodies, dukes' disease, ebola viral infection, ebolaviral infection, elephant endotheliotropic herpesviral infection, epidemic polyarthritis, epidermodysplasia verruciformis, epsteinbarr virus infection, feline leukemia viral infection, filoviridae, foot-and-mouth disease, genital wart, hantaviral infection, henipaviral infection, hepatitis A, hepatitis B, hepatitis C, hepatoviral infection, herpes genitalis, herpes simplex, herpes zoster, herpesviral encephalitis, herpesviral meningitis, herpetic keratoconjunctivitis, HPV-positive oropharyngeal cancer, human bocaviral infection, human cytomegaloviral infection, human respiratory syncytial viral infection, body rhinitis, infectious mononucleosis, infectious pancreatic necrosis, koi herpes viral infection, kunjin viral infection, labrea fever, laryngeal papillomatosis, leucosis, liebermeister's rule, lloviu cuevaviral infection, lloviu viral infection, lujo viral infection, marburg marburgviral infection, marburg virus disease, mayaro virus disease, menangle viral infection, monkeypox, mononegavirales, mononucleosis, mumps, encephalitis viral infection, myxomatosis, nephropathia epidemica, oropouche fever, parvoviral B19, phytoreoviral infection, plantar wart, pogosta disease, porcine adenoviral infection, central nervous system lymphoma, retinal necrosis, rubella panencephalitis, qalyub viral infection, rabbit haemorrhagic disease, ramsay hunt syndrome type II, ravn viral infection, reston ebolaviral infection, reston viral infection, rhinoviral infection, rocio viral encephalitis, roseoloviral infection, ross river fever, rotaviral infection, shope papilloma viral infection, simian foamy viral infection, sudan ebolaviral infection, sudan viral infection, swine vesicular disease, tai forest ebolaviral infection, tropical spastic paraparesis, turkey coronaviral infection, turkey viral Hepatitis, turkeypox, varicella zoster viral infection, venezuelan hemorrhagic fever, verruca plana, viral arthritis, viral arthritis, viral hemorrhagic septicemia, template:viral systemic infection, virus sin nombre, woodchuck hepatitis viral infection, yellow fever, zika fever, template, zoonotic viral infection induced by adenovirus, herpes simplex type 1, herpes simplex type 2, varicella-zoster virus, epstein-barn virus, human cytomegalovirus, human herpesvirus, type 8 human papillomavirus, BK virus, JC virus, smallpox, human bocavirus, parvovirus, B19 human astrovirus, norwalk virus, coxsackievirus, hepatitis A virus, hepatitis B virus, hepatitis C virus, poliovirus, rhinovirus, severe acute respiratory syndrome virus, yellow fever virus, dengue virus, west nile virus, rubella virus Hepatitis E virus, human immunodeficiency virus (HW), influenza virus, guanarito virus, junin virus, lassa virus, machupo virus, Sabiá virus, Crimean-Congo hemorrhagic fever virus, ebola virus, marburg virus, measles virus, mumps virus, parainfluenza virus, respiratory syncytial virus, human metapneumovirus, rabies virus and hepatitis D rotavirus.

6. According to claim 1, wherein: a compound of andrographolide derivatives and analogs is administered together with at least one known antiviral agents, antifungal agents or anti-inflammatory agents selected but is not limited from a cytidine analog, an uridine analog, an adenosine analog, a guanosine analog, a thymidine analog or an inosine analog comprising: deoxycytidine; 2′,3′-dideoxycytidine; 2′,3′-didehydrocytidine carbocyclic, 2′,3′-didehydro-2′,3′-dide-oxycy-tidine, 2′,3′-didehydro-2′,3′-dideoxy-5-methylcytidine, fluoro-2′,3′-dideoxycytidine, 3-(4-hydroxy-1′,2′-butadi-enyl)cytosine, 3′-azido-2′,3′-dideoxy-5-methyl-cytosine, 3′-azido-2′,3′-dideoxy-5-methyl-cytosine, 3′-azido-2′,3′-dide-oxy-5-methyl-cytosine-N4-OH,3′-azido-2′,3′-dideoxy-5-methylcytosine-N4Me, 3′-azido-2′,3′-dideoxycytosine, 3′-azido-2′,3′-dideoxy-5-fluorocytosine, 2′,3′-dideoxy-2′,3′-didehydrocytidine, beta-L-5-fluoro-2′,3′-dideoxy-2′,3′-didehydrolamivudine, racivir, elvucitabine, apricitabine, emtricitabine, apricit abine, deoxyuridine, 5-Methyluridine, 3′-azido-2′,3′-dideoxy-5-chlorouridine, 3′-azido-2′,3′-dideoxy-5-ethyluridine, 3′-azido-2′,3′-dideoxyuridine, 3′-fluoro-2′,3′-dideoxy-5-bromouridine, 3′-fluoro-2′,3′-dideoxy-5-ethyluridine, 3′-azido-2′,3′-dideoxy-5-bromouridine, 3′-azido-2′,3′-dide-oxyuridine, 3′-fluoro-2′,3′-dideoxy-5-chlorouridine, 3′-fluoro-2′,3′-dideox-yuridine, 2′,3′-dideoxy-3′-azidouridine, 2,3′-dideoxy-3′-3′-fluoro-5-chiorouridine, deoxyadenosine, 2,3′-dideoxyadenosine, 2′,3′-dideoxy-2′-fluoroaraadenosine, 2-chiorodeoxyadenosine, 9-(4-hydroxy-1′,2′-butadienyl)adenine, 9-(2-phosphonomethoxyethyl) adenine, 2′,3′-didehydro-2′,3′-dideoxyadenosine, dideoxyadenosine, 5-methyl-2′,3′-dideoxyadenosine, 3′-fluoro-2′,3′-dideoxyarabinothrano-syladenine, 3′-fluoro-2′,3′-dideoxyadenosine, 2,3′-dideoxy-2′,3′-didehydro-N6-(O-methyl-benzyl)adenosine, 2′,3′-di-deoxy-2′,3′-didehydro-N6-(2-methylpropyl)adenoma, 2′,3′-dideoxy-3′-fluo-roadenosine, 2,3′-dideoxyguanosine, 2′,3′-didehydroguanosine; 3′-azido-3′-deoxyguanosine, 3′-fluoro-2′,3′-dideoxy-guanosine, dideoxyguanosine, 3′-azideo-2′,3′-dideoxyguanosine, 3′-fluoro-2′,3′-dideoxyguanosine, 2′,3′-dideoxy-3′-azidoguano-sine, 3′-deoxythymidine; 2′,3′-dideoxythymidine, 2′,3′-didehydrothymidine, 3′-azido-3′-deoxythymidine; 3′-fluoro-3′-deoxythymidine, 3′-fluoro-2′,3′-dideoxythy-midine, 3′-deoxy-2′,3′-didehydrothymidine, 2′,3′-didehydro-2′,3′-dideoxy-thymidine, 2′,3′-dideoxyinosine,2,6-diaminopurine-2′,3′-dideoxyriboside; 2,6-diaminopurine-3′-azido-2′,3′-dideoxyri-boside; 2,6-diaminopurine-3′-fluoro-2′,3‘-dideoxyriboside, 3-phosphonomethoxyethyl-2,6-diaminopurine, 2,6-di-aminopurine-2’, 3′-dideoxyriboside, 3′-azido-2′,3′-dideoxy-diaminopurine, 3′-fluoro-2′,3′-dideoxy diaminopurine, 2′,3′-di-deoxy-3′-fluoro-2,6-diaminopurineriboside, abacavir, acyclovir aciclovir, adefovir, alovudine, amantadine, ampligen, amprenavir, arbidol, atazanavir, atripla, boceprevir, cidofovir, combivir, cytarabine, darunavir, delavirdine, didanosine, desciclovir, didanosine, disoproxil, docosanol, edoxudine, efavirenz, enfuvirtide, entecavir, entry inhibitors, elvucitabine, emtricitabine, famciclovir, fomivirsen, fosamprenavir, foscarnet, fosfonet, fusion inhibitor, ganciclovir, ibacitabine, imunovir, idoxuridine, imiquimod, indinavir, inosine, oseltamivir, penciclovir, peramivir, rimantadine, ribavirin, ritonavir, saquinavir, stavudine, tenofovir, tenofovir, fiacitabine, Fialuridine, doxuridine, Foscamet, Lobucavir, Sorivudine, trifluridine, tromantadine, ribavirine, stavudine, tipranavir, trizivir, truvada, valaciclovir, valganciclovir, vicriviroc, idarabine, viramidine, zalcitabine, zan amivir, zidovudine, synergistic enhancer, integrase inhibitor, interferon type III, interferon type II, interferon type I, interferon, lopinavir, loviride, maraviroc, moroxydine, methisazone, nelfinavir, nevirapine, nexavir, peginterferon alfa-2a, pleconaril, protease inhibitor, raltegravir, reverse transcriptase inhibitor.

7. A method according to the claim 1, wherein: a method for treating cancer, comprising:
administration to a compound of the andrographolide, derivatives and analogs,
a pharmaceutically acceptable salt or prodrug from thereof; a cancer is selected but is not limited from the multiple myeloma, leukemia, lymphoma, acute leukemia, chronic leukemia, acute lymphocytic leukemia, acute nonlym phocytic leukemia, chronic lymphocytic leukemia, chronic myelogenous leukemia, acute myeloid leukemia, hairy cell leukemia, Hodgkin's lymphoma, non-Hodgkin's lymphoma, multiple myeloma, hematologic cancer is of low, intermediate, or high grade, brain cancer, cancers of the head and neck, lung cancer, breast cancer, cancers of the reproductive system, cancers of the digestive system, pancreatic cancer, and cancers of the urinary system, cancer of the upper digestive tract or colorectal cancer, bladder cancer, renal cell carcinoma, prostate cancer, cancers of oral cavity and pharynx, cancers of the respiratory system, cancers of bones and joints, cancers of soft tissue, skin cancers, cancers of the genital system, cancers of the eye and orbit, cancers of the nervous system, cancers of the lymphatic system, and cancers of the endocrine system. In certain embodiments, these cancer s may be selected from the group consisting of: cancer of the tongue, mouth, pharynx, or other oral cavity; esophageal cancer, stomach cancer, or cancer of the small intestine; colon cancer or rectal, anal, or anorectal cancer; cancer of the liver, intrahepatic bile duct, gallbladder, pancreas, or other biliary or digestive organs; laryngeal, bron chial, and other cancers of the respiratory organs; heart cancer, melanoma, basal cell carcinoma, squamous cell carcinoma, other non-epithelial skin cancer; uterine or cervical cancer; uterine corpus cancer; ovarian, vulvar, vaginal, or other female genital cancer; prostate, testicular, penile or other male genital cancer; urinary bladder cancer; cancer of the kidney; renal, pelvic, or urethral cancer or other cancer of the genitourinary organs; thyroid cancer or other endocrine cancer; and cutaneous T-cell lymphoma, both granulocytic and monocytic, adenocarcinoma, angiosarcoma, astrocy toma, acoustic neuroma, anaplastic astrocytoma, basal cell carcinoma, blastoglioma, chondrosarcoma, choriocarcinoma, chordoma, craniopharyngioma, cutaneous melanoma, cystadenocarcinoma, endotheliosarcoma, embryonal carcinoma, ependymoma, Ewing's tumor, epithelial carcinoma, fibrosarcoma, gastric cancer, genitourinary tract cancers, glioblastoma multiforme, hemangioblastoma, hepatocellular carcinoma, hepatoma, Kaposi's sarcoma, large cell carcinoma, leiomyosarcoma, liposarcoma, lymphangiosarcoma, lymphangioendo theliosarcoma, medullary thyroid carcinoma, medulloblastoma, meningioma mesothelioma, myelomas, myxosarcoma neuroblastoma, neurofibrosarcoma, ohgodendroglioma, osteogenic sarcoma, epithelial ovarian cancer, papillary carcinoma, papillary adenocarcinomas, parathyroid tumors, pheochromocytoma, pinealoma, plasmacy tomas, retinoblastoma, rhabdomyosarcoma, sebaceous gland carcinoma, seminoma, skin cancers, melanoma, small cell lung carcinoma, squamous cell carcinoma, sweat gland carcinoma, synovioma, thyroid cancer, uveal melanoma, Wilm's tumor, ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pan creatic duct; bladder cancer such as a transitional cell carcinoma inurinary bladder, urothelial carcinomas, tumors in the urothelial cells that line the bladder, squamous cell carcinomas, adenocarcinomas, small cell cancers; myelodysplasia, myeloproliferative disorders; bone cancer; lung cancer such as non-small cell lung cancer, squamous cell carcinomas, adenocarcinomas, large cell undifferentiated carcinomas, small cell lung cancer; skin cancer, basal cell carcinoma, melanoma, squamous cell carcinoma actinic keratosis, eye retinoblastoma; cutaneous or intraocular melanoma; primary liver cancer; kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus, hepatitis C virus, and hepa tocellular carcinoma; human lymphotropic virus-type 1 and adult T-cell leukemia/lymphoma; and human papilloma virus and cervical cancer; central nervous system cancers, primary brain tumors, gliomas, Oligodendroglioma, Ependymoma, Meningioma, Lymphoma, Schannoma, medulloblastoma; peripheral nervous system cancers, acoustic neuromas, malignant peripheral nerve sheath tumor including neurofi bromas and schwannomas, malignant fibrous cytoma, malig nant fibrous histiocytoma, malignant meningioma, malignant mesothelioma, and malignant mixed Müllerian tumor; oral cavity and oropharyngeal cancer such as, hypopharyngeal cancer, laryngeal cancer, nasopharyngeal cancer, and oropha ryngeal cancer; stomach cancer such as lymphomas, gastric stromal tumors, and carcinoid tumors; testicular cancer such as germ cell tumors, which include seminomas and nonseminomas, and gonadal stromal tumors, which include Leydig cell tumors and Sertoli cell tumors; thymus cancer such as to thymomas, thymic carcinomas, carcinoids or carcinoid tumors; rectal cancer; and colon cancer.

8. The method according to claims 1, wherein said compounds is administered together with at least one known cancer, chemotherapeutic and immune agent selected but is not limited from cyclophosphamide, vincristine, busulfan, vinblastine, cisplatin, carboplatin, mitomycin C, doxorubicin, colchicine, etoposide, paclitaxel, docetaxel, camptothecin, topotecan, arsenic trioxide, 5-azacytidine, 5-fluorouracil, methotrexate, 5-fluoro-2-deoxyuridine, hydroxyurea, thioguanine, melphalan, chlorambucil, ifosfamide, mitoguazone, epirubicin, aclarubicin, bleomycin, mitoxantrone, elliptinium acetate, fludarabine, octreotide, retinoic acid, tamoxifen, doxazosin, terazosin tamsulosin, tamsulosin, fluorine pyridinoline, lovastatin, simvastatin, pravastatin, fluvastatin, atorvastatin, atorvastatin, amprenavir, abacavir, flavonoids pyridinoline, ritonavir, saquinavir, rofecoxib, alanosine, retinal, tretinoin tocoferil, 13-cis-retinoic acid, 9-cis-retinoic acid, α-difluoro-methyl ornithine, fenretinide, N-4-carboxyphenyl retinamide, genistein, ara-C, CB-64D, CB-184, 1LX23-7553, lactacystin, MG-132, PS-341, Glcevec, ZD1839 (IRessa), SH268, Herceptin, Rituxan, Gamcitabine, ABT-378, AG1776, BMS-232, 632, CEP2563, SU6668, EMD121974, R115777, SCH66336, L-778, 123, BAL9611, TAN-1813, UCN-01, Roscovitine, Olonoucine, Valecoxib.

9. A compound according to the claim 1, wherein: a compound of andrographolide derivatives and analogs is, independently at each occurrence, selected but is not limited from the example 1 to 440 and below list: 7-(4-morpholino)-11,12-dedihydro-14-deoxyandrographolide, 7-(4-morpholino)-12-(2-(pyridine-2-yl)amino)-14-deoxyandrographolide, 7-(N-(1-morpholino-1-oxopropan-2-yl)amino)-11,12-dedihydro-14-deoxyandrographolide, 7-(4-morpholino)-12-((2-((4H-imidazol-2-yl)amino)-1-oxopropan-2-yl)amino)-14-deoxyandrographolide, 7-(N-(1-morpholino-1-oxopropan-2-yl)amino)-12-(2-(pyridine-2-yl)amino)-14-deoxyandrographolide, 7-((2-((4H-imidazol-2-yl)amino)-1-oxopropan-2-yl)amino)-11,12-dedihydro-14-deoxyandrographolide, 7-((2-((4H-imidazol-2-yl)amino)-1-oxopropan-2-yl)amino)-12-(2-(pyridine-2-yl)amino)-14-deoxyandrographolide, 7-(N-(1-morpholino-1-oxopropan-2-yl)amino)-12-((2-((4H-imidazol-2-yl)amino)-1-oxopropan-2-yl)amino)-14-deoxyandrographolide, 7,12-dis((2((4H-imidazol-2-yl)amino)-1-oxopropan-2-yl)amino)-14-deoxyandrographolide, 11,12-dedihydro-14-deoxy-15-(2-amino-4-oxygen-3H-pyrimidine-5-yl)methylene)andrographolide, 7-(4-morpholino)-11,12-dedihydro-14-deoxy-15-(2-amino-5-oxygen-3H-pyrimidine-5-ylmethylene)andrographolide, 7-((2-((4H-imidazol-2-yl)amino)-1-oxopropan-2-yl)amino)-11,12-dedihydro-14-deoxy-15-(2-amino-5-oxygen-3H-pyrimidine-5-ylmethylene)andrographolide, 7-(N-(1-morpholino-1-oxopropan-2-yl)amino)-11,12-dedihydro-14-deoxy-15-(2-amino-5-oxygen-3H-pyrimidine-5-ylmethylene)andrographolide, 12-(2-(pyridine-2-yl)amino)-14-deoxy-15-(2-amino-5-oxygen 3H-pyrimidine-5-ylmethylene)andrographolide, 7-(4-morpholino)-12-(2-(pyridine-2-yl)amino)-14-deoxy-15-(2-amino-5-oxygen-3H-pyrimidine-5-ylmethylene)andrographolide, 7-(N-(1-morpholino-1-oxopropan-2-yl)amino)-12-(2-(pyridine-2-yl)amino)-14-deoxy-15-(2-amino-5-oxygen-3H-pyrimidine-5-ylmethylene)andrographolide, 7-((2-((4H-imidazol-2-yl)amino)-1-oxopropan-2-yl)amino)-12-(2-(pyridine-2-yl)amino)-14-deoxy-15-(2-amino-5-oxygen-3H-pyrimidine-5-ylmethylene)andrographolide, 7-(4-morpholino)-12((2-((4H-imidazol-2-yl)amino)-1-oxopropan-2-yl)amino)-14-deoxy-15-(2-amino-5-oxygen-3H-pyrimidine-5-ylmethylene)andrographolide, 7,12-bis((2-((4H-imidazol-2-yl)amino)-1-oxopropan-2-yl)amino)-14-deoxy-15-(2-amino-5-oxygen-3H-pyrimidine-5-ylmethylene) andrographolide, 7-(N-(1-morpholino-1-oxopropan-2-yl)amino)-12-((2-((4H-imidazol-2-yl)amino)-1-oxopropan-2-yl)amino)-14-deoxy-15-(2-amino-5-oxygen-3H-pyrimidine-5-ylmethylene)andrographolide, 11,12-dedihydro-14-deoxy-15-(2-amino-5-oxygen-3H-pyrimidine-5-ylmethylene)-8,17-epoxyandrographolide, 7-(4-morpholino)-11,12-dedihydro-15-(2-amino-5-oxygen-3H-pyrimidine-5-ylmethylene)-8,17-epoxyandrographolide, 7-((2-((4H-imidazol-2-yl)amino)-1-oxopropan-2-yl)amino)-11,12-dedihydro-14-deoxy-15-(2-amino-5-oxygen-3H-pyrimidine-5-ylmethylene)-8,17-epoxyandrographolide, 7-(N-(1-morpholino-1-oxopropan-2-yl) amino)-11,12-dedihydro-14-deoxy-15-(2-amino-5-oxygen-3H-pyrimidine-5-ylmethylene)-8,17-epoxyandrographolide, 12-(2-(pyridine-2-yl)amino)-14-deoxy-15-(2-amino-5-oxygen-3H-pyrimidine-5-ylmethylene)-8,17-epoxyandrographolide, 7-(4-morpholino)-12-(2-(pyridine-2-yl)amino)-14-deoxy-15-(2-amino-5-oxygen-3H-pyrimidine-5-ylmethylene)-8,17-epoxyandrographolide, 7-(N-(1-morpholino-1-oxopropan-2-yl)amino)-12-(2-(pyridine-2-yl)amino)-14-deoxy-15-(2-amino-5-oxygen-3H-pyrimidine-5-ylmethylene)-8,17-epoxyandrographolide, 7-((2-((4H-imidazol-2-yl)amino)-1-oxopropan-2-yl)amino)-12-(2-(pyridine-2-yl)amino)-14-deoxy-15-(2-amino-5-oxygen-3H-pyrimidine-5-ylmethylene)-8,17-epoxyandrographolide, 12-((2-((4H-imidazol-2-yl)amino)-1-oxopropan-2-yl)amino)-14-deoxy-15-(2-amino-5-oxygen-3H-pyrimidine-5-ylmethylene)-8,17-epoxyandrographolide, 7-(4-morpholino)-12-((2-((4H-imidazol-2-yl)amino)-1-oxopropan-2-yl)amino)-14-deoxy-15-(2-amino-5-oxygen-3H-pyrimidine-5-ylmethylene)-8,17-epoxyandrographolide, 7,12-bis((2-((4H-imidazol-2-yl)amino)-1-oxopropan-2-yl)amino)-14-deoxy-15-(2-amino-5-oxygen-3H-pyrimidine-5-ylmethylene)-8,17-epoxyandro-grapholide, 7-(N-(1-morpholino-1-oxopropan-2-yl)amino)-12-((2-((4H-imidazol-2-yl)amino)-1-oxopropan-2-yl)amino)-14-deoxy-15-(2-amino-5-oxygen-3H-pyrimidine-5-ylmethylene)-8,17-epoxyandrographolide11,12-dedihydro-14-deoxy-15-(4-(dimethylamino)benzylidene)andrographolide, 7-(4-morpholino)-11,12-dedihydro-14-deoxy-15-(4-(dimethylamino)benzylidene) andrographolide12-(2-(pyridine-2-yl)amino)-14-deoxy-15-(4-(dimethylamino) benzylidene)andrographolide, 7-(4-morpholino)-12-(2-(pyridine-2-yl)amino)-14-deoxy-15-(4-(dimethylamino)benzylidene)andrographolide, 7-(N-(1-morpholino-1-oxopropan-2-yl)amino)-11,12-dedihydro-14-deoxy-15-(4-(dimethylamino)benzylidene)andrographolide, 7-(4-morpholino)-12-(2-(pyridine-2-yl)amino)-14-deoxy-15-(4-(dimethylamino)benzylidene)andrographolide, 7-(N-(1-morpholino-1-oxopropan-2-yl)amino)-12-(2-(pyridine-2-yl)amino)-14-deoxy-15-(4-(dimethylamino)benzylidene)andrographolide7-((2-((4H-imidazol-2-yl)amino)-1-oxopropan-2-yl)amino)-11,12-dedihydro-14-deoxy-15-(4-(dimethylamino)benzylidene) andrographolide7-((2-((4H-imidazol-2-yl)amino)-1-oxopropan-2-yl)amino)-12-(2-(pyridine-2-yl)amino)-14-deoxy-15-(4-(dimethylamino)benzylidene)andrographolide, 12-((2-((4H-imi-dazol-2-yl)amino)-1-oxopropan-2-yl)amino))-14-deoxy-15-(4-(dimethylamino)benzylidene)andrographolide, 7-(4-morpholino)-12-((2-((4H-imidazol-2-yl)amino)-1-oxopropan-2-yl)amino))-14-deoxy-15-(4-(dimethyl-amino)benzylidene)andrographolide, 7-(N-(1-morpholino-1-oxopropan-2-yl)amino)-12-((2-((4H-imidazol-2-yl)amino)-1-oxopropan-2-yl)amino))-14-deoxy-15-(4-(dimethylamino)benzylidene)andrographolide, 7,12-dis((2((4H-imidazol-2-yl)amino)-1-oxopropan-2-yl)amino))-14-deoxy-15-(4-(dimethylamino)benzylidene) andrographolide, 11,12-dedihydro-14-deoxy-15-((2-aminopyrimidin-5-yl)methylene) andrographolide, 7-(4-mor-pholino)-11,12-dedihydro-14-deoxy-15-((2-aminopyrimidin-5-yl)methylene)andrographolide, 12-(2-(pyridine-2-yl)amino)-14-deoxy-15-((2-aminopyrimidin-5-yl)methylene)andrographolide, 7-(4-morpholino)-12-(2-(pyridine-2-yl)amino)-14-deoxy-15-((2-aminopyrimidin-5-yl)methylene) andrographolide, 7-((24(4H-imida-zol-2-yl)amino)-1-oxopropan-2-yl)amino)-11,12-dedihydro-14-deoxy-15-((2-aminopyrimidin-5-yl)methylene)andrographolide, 74(2-((4H-imidazol-2-yl)amino)-1-oxopropan-2-yl)amino)-12-(2-(pyridine-2-yl)amino)-14-deoxy-15-((2-aminopyrimidin-5-yl)methylene)andrographolide, 7-(N-(1-morpholino-1-oxopropan-2-yl)amino)-12-(2-(pyridine-2-yl)amino)-14-deoxy-15-((2-aminopyrimidin-5-yl)methylene)andrographolide, 7-(N-(1-morpholino-1-oxopropan-2-yl)amino)-11,12-dedihydro-14-deoxy-15-((2-aminopyrimidin-5-yl)methylene) andrographolide, 12-((2-((4H-imidazol-2-yl)amino)-1-oxopropan-2-yl)amino)-14-deoxy-15-((2-aminopyrimidin-5-yl)methylene)andrographolide, 7-(4-morpholino)-12-((2-((4H-imidazol-2-yl)amino)-1-oxopropan-2-yl)amino)-14-deoxy-15-((2-aminopyrimidin-5-yl)methylene)andrographolide, 7-(N-(1-morpholino-1-oxopropan-2-yl)amino)-12-((2-((4H-imidazol-2-yl)amino)-1-oxopropan-2-yl)amino)-14-deoxy-15-((2-aminopyrimidin-5-yl)methylene)andrographolide, 11,12-dedihydro-14-deoxy-15-(2-morpholinopyrimidin-5-ylmethylene)andrographolide, 7-(4-morpholino)-12-(2-(pyridine-2-yl)amino)-14-deoxy-15-(2-morpholinopyrimi-din-5-ylmethylene) andrographolide, 7-((2-((4H-imidazol-2-yl)amino)-1-oxopropan-2-yl)amino)-11,12-dedihydro-14-deoxy-15-(2-morpholinopyrimidin-5-ylmethylene)andrographolide, 7-(4-morpholino)-11,12-dedihydro-14-deoxy-(Z)-15-(1-(2-aminopyrimidin-5-yl)-2-ethoxy-2-oxoethylidene)andrographolide, 7-(4-morpholino)-11,12-dedihydro-14-deoxy-15-(2-morpholinopyrimidin-5-ylmethylene)andrographolide, 12-(2-(pyridine-2-yl)amino)-14-deoxy-15-(2-morpholinopyrimidin-5-ylmethylene)andrographolide, 7-((2-((4H-imidazol-2-yl)amino)-1-oxopropan-2-yl)amino)-12-(2-(pyridine-2-yl)amino)-14-deoxy-15-(2-morpholinopyrimidin-5-ylmethylene)andrographolide, 11,12-dedihydro-14-deoxy-(Z)-15-(1-(2-aminopyrimidin-5-yl)-2-ethoxy-2-oxoethylidene)andrographolide, 7-(N-(1-morpholino-1-oxopropan-2-yl)amino)-11,12-dedihydro-14-deoxy-(Z)-15-(1-(2-aminopyrimidin-5-yl)-2-ethoxy-2-oxoethylidene) andrographolide, 7-((2-((4H-imidazol-2-yl)amino)-1-oxopropan-2-yl)amino)-11,12-dedihydro-14-deoxy-(Z)-15-(1-(2-aminopyrimidin-5-yl)-2-ethoxy-2-oxoethylidene) andrographolide, 12-(2-(pyridine-2-yl)amino)-14-deoxy-(Z)-15-(1-(2-aminopyrimidin-5-yl)-2-ethoxy-2-oxoethylidene)andrographolide, 7-(4-morpholino)-12-(2-(pyridine-2-yl)amino)-14-deoxy-(Z)-15-(1-(2-aminopyrimidin-5-yl)-2-ethoxy-2-oxoethylidene)andrographolide, 7-((2-((4H-imidazol-2-yl)amino)-1-oxopropan-2-yl)amino)-12-(2-(pyridine-2-yl)amino)-14-deoxy-(Z)-15-(1-(2-aminopyrimidin-5-yl)-2-ethoxy-2-oxoethylidene)andrographolide, 7-(N-(1-morpholino-1-oxopropan-2-yl)amino)-12-(2-(pyridine-2-yl)amino)-14-deoxy-(Z)-15-(1-(2-aminopyrimidin-5-yl)-2-ethoxy-2-oxoethylidene)andrographolide, 12-((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)andrographolide, 7-(4-morpholino)-12-((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)andrographolide, 7-(N-(1-morpholino-1-oxopropan-2-yl)amino)-12-((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)andrographolide, 7,12-dis((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)andrographolide, 12-(2-(pyridine-2-yl)amino)-14-deoxy-(Z)-15-(1-(4-(dimethylamino)phenyl)-2-ethoxy-2-oxoethylidene) andrographolide, 7-(4-morpholino)-11,12-dedihydro-14-deoxy-(Z)-15-(1-(4-(dimethylamino)phenyl)-2-ethoxy-2-oxoethylidene)andrographolide, 12-(2-(pyridine-2-yl)amino)-14-deoxy-(Z)-15-(1-(4-(dimethylamino)phenyl)-2-ethoxy-2-oxoethylidene)-8,17-epoxyandrographolide, 7-(4-morpholino)-12-(2-(pyridine-2-yl)amino)-14-deoxy-(Z)-15-(1-(4-(dimethylamino)phenyl)-2-ethoxy-2-oxoethylidene) andrographolide, 7-(N-(1-morpholino-1-oxopropan-2-yl)amino)-11,12-dedihydro-14-deoxy-(Z)-15-(1-(4-(dimethylamino)phenyl)-2-ethoxy-2-oxoethylidene) andrographolide, 7-(N-(1-morpholino-1-oxopropan-2-yl)amino)-12-(2-(pyridine-2-yl)amino)-14-deoxy-(Z)-15-(1-(4-(dimethylamino)phenyl)-2-ethoxy-2-oxoethylidene) andrographolide, 7-((2-((4H-imidazol-2-yl)amino)-1-oxopropan-2-yl)amino)-11,12-dedihydro-14-deoxy-(Z)-15-(1-(4-(dimethylamino)phenyl)-2-ethoxy-2-oxoethylidene) andrographolide, 7-((2-((4H-imidazol-2-yl)amino)-1-oxopropan-2-yl)amino)-12-(2-(pyridine-2-yl)amino)-14-deoxy-(Z)-15-(1-(4-(dimethylamino)phenyl)-2-ethoxy-2-oxoethylidene)andrographolide, 12-((2-((4H-imidazol-2-yl)amino)-1-oxopropan-2-yl)amino)-14-deoxy-(Z)-15-(1-(4-(dimethylamino)phenyl)-2-ethoxy-2-oxoethylidene) andrographolide, 7-(4-morpholino)-12-((2-((4H-imidazol-2-yl)amino)-1-oxopropan-2-yl)amino)-14-deoxy-(Z)-15-(1-(4-(dimethylamino)phenyl)-2-ethoxy-2-oxoethylidene)andrographolide, 7-(N-(1-morpholino-1-oxopropan-2-yl)amino)-12-((2-((4H-imidazol-2-yl)amino)-1-oxopropan-2-yl)amino)-14-deoxy-(Z)-15-(1-(4-(dimethyl-amino)phenyl)-2-ethoxy-2-oxoethylidene)andrographolide, 11,12-dedihydro-14-deoxy-15-(4-(dimethyl-amino)-2-hydroxybenzylidene)) andrographolide, 7-(N-(1-morpholino-1-oxopropan-2-yl)amino)-12-(2-(pyridine-2-yl)amino)-14-deoxy-15-(4-(dimethylamino)-2-hydroxybenzylidene))andrographolide, 7-((2-((4H-imidazol-2-yl)amino)-1-oxopropan-2-yl)amino)-11,12-dedihydro-14-deoxy-15-(4-(dimethylamino)-2-hydro-xybenzylidene))andrographolide, 7-((2-((4H-imidazol-2-yl)amino)-1-oxopropan-2-yl)amino)-12-(2-(pyridine-2-yl)amino)-14-deoxy-15-(4-(dimethylamino)-2-hydroxybenzylidene))andrographolide, 12-(2-(pyridine-2-yl)amino)-14-deoxy-15-(4-(dimethylamino)-2-hydroxybenzylidene)) andrographolide, 7-(4-morpholino)-11,12-dedihydro-14-deoxy-15-(4-(dimethylamino)-2-hydroxybenzylidene))andrographolide, 7-(4-morpholino)-12-(2-(pyridine-2-yl)amino)-14-deoxy-15-(4-(dimethylamino)-2-hydroxybenzyl-idene))andrographolide, 7-(N-(1-morpholino-1-oxopropan-2-yl)amino)-11,12-dedihydro-14-deoxy-15-(4-(dimethylamino)-2-hydroxybenzylidene))andrographolide, 12((2-((4H-imidazol-2-yl)amino)-1-oxopropan-2-yl)amino)-14-deoxy-15-(4-(dimethylamino)-2-hydroxybenzylidene))andrographolide, 7-(4-morpholino)-12((2-((4H-imidazol-2-yl)amino)-1-oxopropan-2-yl)amino)-14-deoxy-15-(4-(dimethylamino)-2-hydroxybenzylidene))andrographolide, 7-(N-(1-morpholino-1-oxopropan-2-yl)amino)-12-((2-((4H-imidazol-2-yl)amino)-1-oxopropan-2-yl)amino)-14-deoxy-15-(4-(dimethylamino)-2-hydroxybenzylidene))andrographolide, 11,12-dedihydro-14-deoxy-15-(4-(dimethylamino)benzylidene)-8,17-epoxyandrographolide, 7-(4-morpholino)-11,12-dedihydro-14-deoxy-15-(4-(dimethylamino)benzylidene)-8,17-epoxyandrographolide, 7-(4-morpholino)-12-(2-(pyridine-2-yl-amino)-14-deoxy-15-(4-(dimethylamino)benzylidene)-8,17-epoxyandrographolide, 7-((2-((4H-imidazol-2-yl)amino)-1-oxopropan-2-yl)amino))-11,12-dedihydro-14-deoxy-15-(4-(dimethylamino)benzylidene)-8,17-epoxyandro-grapholide, 7((2-((4H-imidazol-2-yl)amino)-1-oxopropan-2-yl)amino)-12-(2-(pyridine-2-yl)amino)-14-deoxy-15-(4-(dimethylamino)benzylidene)-8,17-epoxyandrogra-pholide7-(N-(1-morpholino-1-oxopropan-2-yl)amino)-11,12-dedihydro-14-deoxy-15-(4-(dimethylamino)benzylidene)-8,17-epoxyandrographolide, 7-(N-(1-morpholino-1-oxopropan-2-yl)amino)-12-(2-(pyridine-2-yl)amino)-14-deoxy-15-(4-(dimethylamino)benzylidene)-8,17-epoxyandrographolide, 12((2-((4H-imidazol-2-yl)amino)-1-oxopropan-2-yl)amino))-14-deoxy-15-(4-(dimethylamino)benzylidene)-8,17-epoxyandrographolide, 7-(4-morpholino)-12-((2-((4H-imidazol-2-yl)amino)-1-oxopropan-2-yl)amino))-14-deoxy-15-(4-(dimethylamino)benzylidene)-8,17-epoxyandrographolide, 7-(N-(1-morpholino-1-oxopropan-2-yl)amino)-12-((2-((4H-imidazol-2-yl)amino)-1-oxopropan-2-yl)amino))-14-deoxy-15-(4-(dimethylamino)benzylidene)-8,17-epoxyandrographolide, 7,12-dis((2((4H-imidazol-2-yl)amino)-1-oxopropan-2-yl)amino))-14-deoxy-15-(4-(dimethylamino)benzyl-idene)-8,17-epoxyandrographolide, 11,12-dedihydro-14-deoxy-15-((2-aminopyrimidin-5-yl)methylene)-8,17-epoxyandrographolide, 7-(4-morpholino)-11,12-dedihydro-14-deoxy-15-((2-aminopyrimidin-5-yl)methyl-ene)-8,17-epoxyandrographolide, 12-(2-(pyridine-2-yl)amino)-14-deoxy-15-((2-aminopyrimidin-5-yl)methyl-ene)-8,17-epoxyandrographolide, 7-(4-morpholino)-12-(2-(pyridine-2-yl)amino)-14-deoxy-15-((2-amino-pyrimidin-5-yl)methylene)-8,17-epoxyandrographolide, 1-morpholino-1-oxopropan-2-yl)amino)-11,12-dedihydro-14-deoxy-15-((2-aminopyrimidin-5-yl)methylene)-8,17-epoxyandrograp-holide 7-(N-(1-morpholino-1-oxopropan-2-yl)amino)-12-(2-(pyridine-2-yl)amino)-14-deoxy-15-((2-aminopyrimidin-5-yl)methylene)-8,17-epoxyandrographolide, 12-((2-((4H-imidazol-2-yl)amino)-1-oxopropan-2-yl)amino)-14-deoxy-15-((2-aminopyrimidin-5-yl)methylene)-8,17-epoxyandrographolide, 7-(4-morpholino)-12-((2-((4H-imidazol-2-yl)amino)-1-oxopropan-2-yl)amino)-14-deoxy-15-((2-aminopyrimidin-5-yl)methylene)-8,17-epoxyandrogra-pholide, 7-(N-(1-morpholino-1-oxopropan-2-yl)amino)-12-((2-((4H-imidazol-2-yl)amino)-1-oxopropan-2-yl)amino)-14-deoxy-15-((2-aminopyrimidin-5-yl)methylene)-8,17-epoxyandrographolide, 7,12-dis((2((4H-imidazol-2-yl)amino)-1-oxopropan-2-yl)amino)-14-deoxy-15-((2-aminopyrimidin-5-yl)methylene)-8,17-epoxyandrographolide, 11,12-dedihydro-14-deoxy-(Z)-15-(1-(2-aminopyrimidin-5-yl)-2-ethoxy-2-oxoethylidene)-8,17-epoxyandrographolide, 7-(4-morpholino)-11,12-dedihydro-14-deoxy-(Z)-15-(1-(2-aminopyrimidin-5-yl)-2-ethoxy-2-oxoethylidene)-8,17-epoxyandrographolide12-(2-(pyridine-2-yl)amino)-14-deoxy-(Z)-15-(1-(2-aminopyrimidin-5-yl)-2-ethoxy-2-oxoethylidene)-8,17-epoxyandrographolide, 7-(4-morpholino)-12-(2-(pyridine-2-yl)amino)-14-deoxy-(Z)-15-(1-(2-aminopyrimidin-5-yl)-2-ethoxy-2-oxoethylidene)-8,17-epoxyandrographolide, 7-((2-((4H-imidazol-2-yl)amino)-1-oxopropan-2-yl)amino)-11,12-dedihydro-14-deoxy-(Z)-15-(1-(2-aminopyrimidin-5-yl)-2-ethoxy-2-oxoethylidene)-8,17-epoxyandrographolide, 7-((2-((4H-imidazol-2-yl)amino)-1-oxopropan-2-yl)amino)-12-(2-(pyridine-2-yl)amino)-14-deoxy-(Z)-15-(1-(2-aminopyrimidin-5-yl)-2-ethoxy-2-oxoethylidene)-8,17-epoxyandrographolide, 7-(N-(1-morpholino-1-oxopropan-2-yl)amino)-11,12-dedihydro-14-deoxy-(Z)-15-(1-(2-aminopyrimidin-5-yl)-2-ethoxy-2-oxoethylidene)-8,17-epoxyandrographolide, 7-(N-(1-morpholino-1-oxopropan-2-yl)amino)-12-(2-(pyridine-2-yl)amino)-14-deoxy-(Z)-15-(1-(2-aminopyrimidin-5-yl)-2-ethoxy-2-oxoethylidene)-8,17-epoxyandrographolide, 12((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)-8,17-epoxyandrographolide, 7-(4-morpholino)-12-((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)-8,17-epoxyandrographolide, 7-(N-(1-morpholino-1-oxopropan-2-yl)amino)-12-((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)-8,17-epoxyandrographolide, 7,12-dis((2-((4H-imidazol-2-yl)amino)-1-oxopropan-2-yl)amino)-14-deoxy-(Z)-15-(1-(2-aminopyrimidin-5-yl)-2-ethoxy-2-oxoethylidene)-8,17-epoxyandrographolide, 12-(2-(pyridine-2-yl)amino)-14-deoxy-(Z)-15-(1-(4-(dimethylamino)phenyl)-2-ethoxy-2-oxoethylidene)-8,17-epoxyandrographolide, 7-(4-morpholino)-11,12-dedihydro-14-deoxy-(Z)-15-(1-(4-(dimethylamino)phenyl)-2-ethoxy-2-oxoethylidene)-8,17-epoxyandrographolide, 12-(2-(pyridine-2-yl)amino)-14-deoxy-(Z)-15-(1-(4-(dimethylamino)phenyl)-2-ethoxy-2-oxoethylidene)-8,17-epoxyandrographolide, 7-(4-morpholino)-12-(2-(pyridine-2-yl)amino)-14-deoxy-(Z)-15-(1-(4-(dimethylamino)phenyl)-2-ethoxy-2-oxoethylidene)-8,17-epoxyandrographolide, 7-(N-(1-morpholino-1-oxopropan-2-yl)amino)-11,12-dedihydro-14-deoxy-(Z)-15-(1-(4-(dimethylamino)phenyl)-2-ethoxy-2-oxoethylidene)-8,17-epoxyandrographolide, 7-(N-(1-morpholino-1-oxopropan-2-yl)amino)-12-(2-(pyridine-2-yl)amino)-14-deoxy-(Z)-15-(1-(4-(dimethylamino)phenyl)-2-ethoxy-2-oxoethylidene)-8,17-epoxyandrographolide, 7((2-((4H-imidazol-2-yl)amino)-1-oxopropan-2-yl)amino)-11,12-dedihydro-14-deoxy-(Z)-15-(1-(4-(dimethylamino)phenyl)-2-ethoxy-2-oxoethylidene)-8,17-epoxyandrographolide, 7-((2-((4H-imidazol-2-yl)amino)-1-oxopropan-2-yl)amino)-12-(2-(pyridine-2-yl)amino)-14-deoxy-(Z)-15-(1-(4-(dimethylamino)phenyl)-2-ethoxy-2-oxoethylidene)-8,17-epoxyandrographolide, 12((2-((4H-imidazol-2-yl)amino)-1-oxopropan-2-yl)amino)-14-deoxy-(Z)-15-(1-(4-(dimethylamino)phenyl)-2-ethoxy-2-oxoethylidene)-8,17-epoxyandrographolide, 7-(4-morpholino)-12-((2-((4H-imidazol-2-yl)amino)-1-oxopropan-2-yl)amino)-14-deoxy-(Z)-15-(1-(4-(dimethylamino)phenyl)-2-ethoxy-2-oxoethylidene)-8,17-epoxyandrographolide, 7-(N-(1-morpholino-1-oxopropan-2-yl)amino)-12-((2-((4H-imidazol-2-yl)amino)-1-oxopropan-2-yl)amino)-14-deoxy-(Z)-15-(1-(4-(dimethylamino)phenyl)-2-ethoxy-2-oxoethylidene)-8,17-epoxyandrographolide, 11,12-dedihydro-14-deoxy-15-(4-(dimethylamino)-2-hydroxybenzylidene))-8,17-epoxyandrographolide, 7-(4-morpholino)-11,12-dedihydro-14-deoxy-15-(4-(dimethylamino)-2-hydroxybenzylidene))-8,17-epoxyandrographolide, 12-(2-(pyridine-2-yl)amino)-14-deoxy-15-(4-(dimethylamino)-2-hydroxybenzylidene))-8,17-epoxyandrographolide, 7-(4-morpholino)-12-(2-(pyridine-2-yl)amino)-14-deoxy-15-(4-(dimethylamino)-2-hydroxybenzylidene))-8,17-epoxyandrographolide, 7-(N-(1-morpholino-1-oxopropan-2-yl)amino)-11,12-dedihydro-14-deoxy-15-(4-(dimethylamino)-2-hydroxybenzylidene))-8,17-epoxyandrographolide, 7-(N-(1-morpholino-1-oxopropan-2-yl)amino)-12-(2-(pyridine-2-yl)amino)-14-deoxy-15-(4-(dimethylamino)-2-hydroxybenzylidene))-8,17-epoxyandrographolide, 7-((2-((4H-imidazol-2-yl)amino)-1-oxopropan-2-yl)amino)-11,12-dedihydro-14-deoxy-15-(4-(dimethylamino)-2-hydroxybenzylidene))-8,17-epoxyandrographolide, 7-((2-((4H-imidazol-2-yl)amino)-1-oxopropan-2-yl)amino)-12-(2-(pyridine-2-yl)amino)-14-deoxy-15-(4-(dimethylamino)-2-hydroxybenzylidene))-8,17-epoxyandrographolide, 12-((2-((4H-imidazol-2-yl)amino)-1-oxopropan-2-yl)amino)-14-deoxy-15-(4-(dimethylamino)-2-hydroxybenzylidene))-8,17-epoxyandrographolide, 7-(4-morpholino)-12-((2-((4H-imidazol-2-yl)amino)-1-oxopropan-2-yl)amino)-14-deoxy-15-(4-(dimethylamino)-2-hydroxybenzylidene))-8,17-epoxyandrographolide, 7-(N-(1-morpholino-1-oxopropan-2-yl)amino)-12-((2-((4H-imidazol-2-yl)amino)-1-oxopropan-2-yl)amino)-14-deoxy-15-(4-(dimethylamino)-2-hydroxybenzylidene))-8,17-epoxyandrographolide, 11,12-dedihydro-14-deoxy-8,17-epoxyandrographolide, 7-(4-morpholino)-11,12-dedihydro-14-deoxy-8,17-epoxyandrographolide, 7-(N-(1-morpholino-1-oxopropan-2-yl)amino)-11,12-dedihydro-14-deoxy-8,17-epoxyandrographolide, 7((2-((4H-imidazol-2-yl)amino)-1-oxopropan-2-yl)amino)-11,12-dedihydro-14-deoxy-8,17-epoxyandrographolide, 7-(4-morpholino)-12-(2-(pyridine-2-yl)amino)-14-deoxy-8,17-epoxyandrographolide, 7-(N-(1-morpholino-1-oxopropan-2-yl)amino)-12-(2-(pyridine-2-yl)amino)-14-deoxy-8,17-epoxyandrographolide, 7-((2-((4H-imidazol-2-yl)amino)-1-oxopropan-2-yl)amino)-12-(2-(pyridine-2-yl)amino)-14-deoxy-8,17-epoxyandrographolide, 7-(4-morpholino)-12((2-((4H-imidazol-2-yl)amino)-1-oxopropan-2-yl)amino))-14-deoxy-8,17-epoxyandrographolide, 7-(N-(1-morpholino-1-oxopropan-2-yl)amino)-12-((2-((4H-imidazol-2-yl)amino)-1-oxopropan-2-yl)amino)-14-deoxy-8,17-epoxyandrographolide, 7,12-dis((2-((4H-imidazol-2-yl)amino)-1-oxopropan-2-yl)amino)-14-deoxy-8,17-epoxyandrographolide, 11,12-dedihydro-14-deoxy-15-(1-methyl-2-(ethoxycarbonyl)-3-oxo-1,2,3,8a-tetrahydroimidazo[1,2-a]pyridine) andrographolide, 7-(4-morpholino)-11,12-dedihydro-14-deoxy-15-(1-methyl-2-(ethoxycarbonyl)-3-oxo-1,2,3,8a-tetrahydroimidazo[1,2-a]pyridine) andrographolide, 7-((2-((4H-imidazol-2-yl)amino)-1-oxopropan-2-yl)amino)-11,12-dedihydro-14-deoxy-15-(1-methyl-2-(ethoxycarbonyl)-3-oxo-1,2,3,8a-tetrahydroimidazo[1,2-a]pyridine) andrographolide, 7-(N-(1-morpholino-1-oxopropan-2-yl)amino)-11,12-dedihydro-14-deoxy-15-(1-methyl-2-(ethoxycarbonyl)-3-oxo-1,2,3,8a-tetrahydroimidazo[1,2-a]pyridine) andrographolide, 12-(2-(pyridine-2-yl)amino)-14-deoxy-15-(1-methyl-2-(ethoxycarbonyl)-3-oxo-1,2,3,8a-tetrahydroimidazo[1,2-a]pyridine) andrographolide, 7-(4-morpholino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)andrographolide, 7-(N-(1-morpholino-1-oxopropan-2-yl)amino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)andrographolide, 7-((2-((4H-imidazol-2-yl)amino)-1-oxopropan-2-yl)amino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)andrographolide, 12-((2-((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine)andrographolide, 7-(4-morpholino)-12-((2((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine)andrographolide, 7-(N-(1-morpholino-1-oxopropan-2-yl)amino)-12-((2-((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine) andrographolide, 7,12-dis((2-((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine)andrographolide, 11,12-dedihydro-14-deoxy-(E)-15-((1-methyl-1H-benzo[d]imidazol-5-yl)methylene)andrographolide, 7-(4-morpholino)-11,12-dedihydro-14-deoxy-(E)-15-((1-methyl-1H-benzo[d]imidazol-5-yl)methylene)andrographolide, 7-(N-(1-morpholino-1-oxopropan-2-yl)amino-11,12-dedihydro-14-deoxy-((E)-15-((1-methyl-1H-benzo[d]imidazol-5-yl)methylene)andrographolide, 7-((2-((4H-imidazol-2-yl)amino)-1-oxopropan-2-yl)amino)-11,12-dedihydro-14-deoxy-(E)-15-((1-methyl-1H-benzo[d]imidazol-5-yl)methylene)andrographolide, 12-(2-(pyridine-2-yl)amino)-14-deoxy-(E)-15-((1-methyl-1H-benzo[d]imidazol-5-yl)methylene)andrographolide, 7-(4-morpholino)-12-(2-(pyridine-2-yl)amino)-14-deoxy-(E)-15-((1-methyl-1H-benzo[d]imidazol-5-yl)methylene)andrographolide, 7-(N-(1-morpholino-1-oxopropan-2-yl)amino)-12-(2-(pyridine-2-yl)amino)-14-deoxy-(E)-15-((1-methyl-1H-benzo[d]imidazol-5-yl)methylene)andrographolide, 7-((2-((4H-imidazol-2-yl)amino)-1-oxopropan-2-yl)amino)-12-(2-(pyridine-2-yl)amino)-14-deoxy-(E)-15-((1-methyl-1H-benzo[d]imidazol-5-yl)methylene)andrographolide, 12-((2-((4H-imidazol-2-yl)amino)-1-oxopropan-2-yl)amino)-14-deoxy-(E)-15-((1-methyl-1H-benzo[d]imidazol-5-yl)methylene)andrographolide, 7-(4-morpholino)-12-(2-(pyridine-2-yl)amino)-14-deoxy-(E)-15-((1-methyl-1H-benzo[d]imidazol-5-yl)methylene)andrographolide, 7-(N-(1-morpholino-1-oxopropan-2-yl)amino)-12-((2-((4H-imidazol-2-yl)amino)-1-oxopropan-2-yl)amino)-14-deoxy-(E)-15-((1-methyl-1H-benzo[d]imidazol-5-yl)methylene)andrographolide, 7,12-dis((2((4H-imidazol-2-yl)amino)-1-oxopropan-2-yl)amino)-14-deoxy-(E)-15-((1-methyl-1H-benzo[d]imidazol-5-yl)methylene) andrographolide, 12-((2-((4H-imidazol-2-yl)amino)-1-oxopropan-2-yl)amino)-14-deoxy-(E)-15-((3-methyl-3H-imidazo[4,5-b]pyridin-6-yl)methylene)andrographolide, 7-(4-morpholino)-12-((2-((4H-imidazol-2-yl)amino)-1-oxopropan-2-yl)amino)-14-deoxy-(E)-15-((3-methyl-3H-imidazo[4,5-b]pyridin-6-yl)methylene)andrographolide, 7-(N-(1-morpholino-1-oxopropan-2-yl)amino)-12-((2-((4H-imidazol-2-yl)amino)-1-oxopropan-2-yl)amino)-14-deoxy-(E)-15-((3-methyl-3H-imidazo[4,5-b]pyridin-6-yl)methylene)andrographolide, 7,12-dis((2-((4H-imidazol-2-yl)amino)-1-oxopropan-2-yl)amino)-14-deoxy-(E)-15-((3-methyl-3H-imidazo[4,5-b]pyridin-6-yl)methylene) andrographolide, 11,12-dedihydro-14-deoxy-(E)-15-((2-amino-9-methyl-6-oxo-6,9-dihydro-1H-purin-8-yl)methylene)andrographolide, 7-(4-morpholino)-11,12-dedihydro-14-deoxy-(E)-15-((2-amino-9-methyl-6-oxo-6,9-dihydro-1H-purin-8-yl)methylene)andrographolide, 7-((2-((4H-imidazol-2-yl)amino)-1-oxopropan-2-yl)amino)-11,12-dedihydro-14-deoxy-(E)-15-((2-amino-9-methyl-6-oxo-6,9-dihydro-1H-purin-8-yl)methylene)andrographolide, 12-(2-(pyridine-2-yl)amino)-14-deoxy-(E)-15-((2-amino-9-methyl-6-oxo-6,9-dihydro-1H-purin-8-yl)methylene) andrographolide, 7-(4-morpholino)-12-(2-(pyridine-2-yl)amino)-14-deoxy-(E)-15-((2-amino-9-methyl-6-oxo-6,9-dihydro-1H-purin-8-yl)methylene)andrographolide, 7-((2-((4H-imidazol-2-yl)amino)-1-oxopropan-2-yl)amino)-12-(2-(pyridine-2-yl)amino)-14-deoxy-(E)-15-((2-amino-9-methyl-6-oxo-6,9-dihydro-1H-purin-8-yl)methylene)andrographolide, 11,12-dedihydro-14-deoxy-(E)-15-((4-imino-1-methyl-6-oxo-4,5,6,7-tetrahydro-1H-pyrazolo[3,4-d]pyrimidin-3-yl)methylene) andrographolide, 7-(4-morpholino)-11,12-dedihydro-14-deoxy-(E)-15-((4-imino-1-methyl-6-oxo-4,5,6,7-tetrahydro-1H-pyrazolo[3,4-d]pyrimidin-3-yl)methylene) andrographolide, 7-((2-((4H-imidazol-2-yl)amino)-1-oxopropan-2-yl)amino)-11,12-dedihydro-14-deoxy-(E)-15-((4-imino-1-methyl-6-oxo-4,5,6,7-tetrahydro-1H-pyrazolo[3,4-d]pyrimidin-3-yl)methylene)andrographolide, 12-(2-(pyridine-2-yl)amino)-14-deoxy-(E)-15-((4-imino-1-methyl-6-oxo-4,5,6,7-tetrahydro-1H-pyrazolo[3,4-d]pyrimidin-3-yl)methylene)andrographolide, 7-(4-morpholino)-12-(2-(pyridine-2-yl)amino)-14-deoxy-(E)-15-((4-imino-1-methyl-6-oxo-4,5,6,7-tetrahydro-1H-pyrazolo[3,4-d]pyrimidin-3-yl)methylene)andrographolide, 7-((2-((4H-imidazol-2-yl)amino)-1-oxopropan-2-yl)amino)-12-(2-(pyridine-2-yl)amino)-14-deoxy-(E)-15-((4-imino-1-methyl-6-oxo-4,5,6,7-tetrahydro-1H-pyrazolo[3,4-d]pyrimidin-3-yl)methylene)andrographolide, 11,12-dedihydro-14-deoxy-(E)-15-((2-amino-7-methyl-7H-pyrrolo[2,3-d]pyrimidin-5-yl)methylene)andrographolide, 7-(4-morpholino)-11,12-dedihydro-14-deoxy-(E)-15-((2-amino-7-methyl-7H-pyrrolo[2,3-d]pyrimidin-5-yl)methylene)andrographolide, 7((2-((4H-imidazol-2-yl)amino)-1-oxopropan-2-yl)amino)-11,12-dedihydro-14-deoxy-(E)-15-((2-amino-7-methyl-7H-pyrrolo[2,3-d]pyrimidin-5-yl)methylene)andrographolide, 12-(2-(pyridine-2-yl)amino)-14-deoxy-(E)-15-((2-amino-7-methyl-7H-pyrrolo[2,3-d]pyrimidin-5-yl)methylene) andrographolide, 7-(4-morpholino)-12-(2-(pyridine-2-yl)amino)-14-deoxy-(E)-15-((2-amino-7-methyl-7H-pyrrolo[2,3-d]pyrimidin-5-yl)methylene)andrographolide, 74(2-((4H-imidazol-2-yl)amino)-1-oxopropan-2-yl)amino)-12-(2-(pyridine-2-yl)amino)-14-deoxy-(E)-15-((2-amino-7-methyl-7H-pyrrolo[2,3-d]pyrimidin-5-yl)methylene) andrographolide, 11,12-dedihydro-14-deoxy-(E)-15-(4-(N-(2-(dimethylamino)ethyl) carbamimidoyl)benzylidene)andrographolide, 7-(4-morpholino)-11,12-dedihydro-14-deoxy-(E)-15-(4-(N-(2-(dimethylamino)ethyl)carbamimidoyl)benzylidene) andrographolide, 7-((2-((4H-imidazol-2-yl)amino)-1-oxopropan-2-yl)amino)-11,12-dedihydro-14-deoxy-(E)-15-(4-(N-(2-(dimethylamino)ethyl)carbamimidoyl)benzylidene)andrographolide, 12-(2-(pyridine-2-yl)amino)-14-deoxy-(E)-15-(4-(N-(2-(dimethylamino)ethyl)carbamimidoyl)benzylidene)andrographolide, 7-(4-morpholino)-12-(2-(pyridine-2-yl)amino)-14-deoxy-(E)-15-(4-(N-(2-(dimethylamino)ethyl)carbamimidoyl)benzylidene)andrographolide, 7-((2-((4H-imidazol-2-yl)amino)-1-oxopropan-2-yl)amino)-12-(2-(pyridine-2-yl)amino)-14-deoxy-(E)-15-(4-(N-(2-(dimethylamino)ethyl)carbamimidoyl)benzylidene) andrographolide, 11,12-dedihydro-14-deoxy-(E)-15-(4-guanidinobenzylidene) andrographolide, 7-(4-morpholino)-11,12-dedihydro-14-deoxy-(E)-15-(4-guanidinobenzylidene)andrographolide, 7-((2-((4H-imidazol-2-yl)amino)-1-oxopropan-2-yl)amino)-11,12-dedihydro-14-deoxy-(E)-15-(4-guanidinobenzylidene) andrographolide, 12-(2-(pyridine-2-yl)amino)-14-deoxy-(E)-15-(4-guanidinobenzylidene)andrographolide, 7-(4-morpholino)-12-(2-(pyridine-2-yl)amino)-14-deoxy-(E)-15-(4-guanidinobenzylidene)andrographolide, 7-((2-((4H-imidazol-2-yl)amino)-1-oxopropan-2-yl)amino)-12-(2-(pyridine-2-yl)amino)-14-deoxy-(E)-15-(4-guanidinobenzylidene)andrographolide, 11,12-dedihydro-14-deoxy-(E)-15-((2-(2-(hydroxymethyl)pyrrolidin-1-yl)pyrimidin-5-yl)methylene)-andrographolide, 7-(4-morpholino)-11,12-dedihydro-14-deoxy-(E)-15-((2-(2-(hydroxymethyl)pyrrolidin-1-yl)pyrimidin-5-yl)methylene)andrographolide, 7-((2-((4H-imidazol-2-yl)amino)-1-oxopropan-2-yl)amino)-11,12-dedihydro-14-deoxy-(E)-15-((2-(2-(hydroxymethyl)pyrrolidin-1-yl)pyrimidin-5-yl)methylene)andrographolide, 12-(2-(pyridine-2-yl)amino)-14-deoxy-(E)-15-((2-(2-(hydroxymethyl)pyrrolidin-1-yl) pyrimidin-5-yl)methylene)andrographolide, 7-(4-morpholino)-12-(2-(pyridine-2-yl)amino)-14-deoxy-(E)-15-((2-(2-(hydroxymethyl)pyrrolidin-1-yl)pyrimidin-5-yl)methylene)andrographolide, 7-((2-((4H-imidazol-2-yl)amino)-1-oxopropan-2-yl)amino)-12-(2-(pyridine-2-yl)amino)-14-deoxy-(E)-15-((2-(2-(hydroxymethyl) pyrrolidin-1-yl)pyrimidin-5-yl)methylene)andrographolide, 11,12-dedihydro-14-deoxy-15-(1-methyl-2-(ethoxycarbonyl)-3-oxo-1,2,3,8a-tetrahydroimidazo[1,2-a]pyridine)-8,17-epoxyandrographolide, 7-(4-morpholino)-11,12-dedihydro-14-deoxy-15-(1-methyl-2-(ethoxycarbonyl)-3-oxo-1,2,3,8a-tetrahydroimidazo[1,2-a]pyridine)-8,17-epoxyandrographolide, 7-(N-(1-morpholino-1-oxopropan-2-yl)amino)-11,12-dedihydro-14-deoxy-15-(1-methyl-2-(ethoxycarbonyl)-3-oxo-1,2,3,8^{a}-tetrahydroimidazo[1,2-a]pyridine)-8,17-epoxyandrographolide, 7-((2-((4H-imidazol-2-yl)amino)-1-oxopropan-2-yl)amino)-11,12-dedihydro-14-deoxy-15-(1-methyl-2-(ethoxycarbonyl)-3-oxo-1,2,3,8a-tetrahydroimidazo[1,2-a]pyridine)-8,17-epoxyandrographolide, 12-(2-(pyridine-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 7-(4-morpholino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 7-(N-(1-morpholino-1-oxopropan-2-yl)amino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 7-((2-((4H-imidazol-2-yl)amino)-1-oxopropan-2-yl)amino)-12-(2-(pyridine-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 12-((2-((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 7-(4-morpholino)-12-((2-((4H-imidazol-2-yl)amino)-1-oxopropan-2-yl)amino)-14-deoxy-15-(1-methyl-2-(ethoxycarbonyl)-3-oxo-1,2,3,8a-tetrahydroimidazo[1,2-a]pyridine)-8,17-epoxyandrographolide, 11,12-dedihydro-14-deoxy-(E)-15-((1-methyl-1H-benzo[d]imidazol-5-yl)methylene)-8,17-epoxyandrographolide, 7-(4-morpholino)-11,12-dedihydro-14-deoxy-(E)-15-((1-methyl-1H-benzo[d]imidazol-5-yl)methylene)-8,17-epoxyandrographolide, 7-(N-(1-morpholino-1-oxopropan-2-yl)amino-11,12-dedihydro-14-deoxy-((E)-15-((1-methyl-1H-benzo[d]imidazol-5-yl)methylene)-8,17-epoxyandrographolide, 7-((2-((4H-imidazol-2-yl)amino)-1-oxopropan-2-yl)amino)-11,12-dedihydro-14-deoxy-(E)-15-((1-methyl-1H-benzo[d]imidazol-5-yl)methylene)-8,17-epoxyandrographolide, 12-(2-(pyridine-2-yl)amino)-14-deoxy-(E)-15-((1-methyl-1H-benzo[d]imidazol-5-yl)methylene)-8,17-epoxyandrographolide, 7-(4-morpholino)-12-(2-(pyridine-2-yl)amino)-14-deoxy-(E)-15-((1-methyl-1H-benzo[d]imidazol-5-yl)methylene)-8,17-epoxyandrographolide, 7-(N-(1-morpholino-1-oxopropan-2-yl)amino)-12-(2-(pyridine-2-yl)amino)-14-deoxy-(E)-15-((1-methyl-1H-benzo[d]imidazol-5-yl)methylene)-8,17-epoxyandrographolide, 7-((2-((4H-imidazol-2-yl)amino)-1-oxopropan-2-yl)amino)-12-(2-(pyridine-2-yl)amino)-14-deoxy-(E)-15-((1-methyl-1H-benzo[d]imidazol-5-yl)methylene)-8,17-epoxyandrographolide, 12-((2-((4H-imidazol-2-yl)amino)-1-oxopropan-2-yl)amino)-14-deoxy-(E)-15-((1-methyl-1H-benzo[d]imidazol-5-yl)methylene)-8,17-epoxyandrographolide, 7-(4-morpholino)-12-(2-(pyridine-2-yl)amino)-14-deoxy-(E)-15-((1-methyl-1H-benzo[d]imidazol-5-yl)methylene)-8,17-epoxyandrographolide, 12-((2-((4H-imidazol-2-yl)amino)-1-oxopropan-2-yl)amino)-14-deoxy-(E)-15-((3-methyl-3H-imidazo[4,5-b]pyridin-6-yl)methylene)-8,17-epoxyandrographolide, 7-(4-morpholino)-12((2-((4H-imidazol-2-yl)amino)-1-oxopropan-2-yl)amino)-14-deoxy-(E)-15-((3-methyl-3H-imidazo[4,5-b]pyridin-6-yl)methylene)-8,17-epoxyandrographolide, 7-(N-(1-morpholino-1-oxopropan-2-yl)amino)-12-((2-((4H-imidazol-2-yl)amino)-1-oxopropan-2-yl)amino)-14-deoxy-(E)-15-((3-methyl-3H-imidazo[4,5-b]pyridin-6-yl)methylene)-8,17-epoxyandrographolide, 7,12-dis((2-((4H-imidazol-2-yl)amino)-1-oxopropan-2-yl)amino)-14-deoxy-(E)-15((3-methyl-3H-imidazo[4, 5-b]pyridin-6-yl)methylene)-8,17-epoxyandrographolide, 11,12-dedihydro-14-deoxy-(E)-15-((2-amino-9-methyl-6-oxo-6,9-dihydro-1H-purin-8-yl)methylene)-8,17-epoxyandrographolide, 7-(4-morpholino)-11,12-dedihydro-14-deoxy-(E)-15-((2-amino-9-methyl-6-oxo-6,9-dihydro-1H-purin-8-yl)methylene)-8,17-epoxyandrographolide, 7-((2-((4H-imidazol-2-yl)amino)-1-oxopropan-2-yl)amino)-11,12-dedihydro-14-deoxy-(E)-15-((2-amino-9-methyl-6-oxo-6,9-dihydro-1H-purin-8-yl)methylene)-8,17-epoxyandrographolide, 12-(2-(pyridine-2-yl)amino)-14-deoxy-(E)-15-((2-amino-9-methyl-6-oxo-6,9-dihydro-1H-purin-8-yl)methylene)-8,17-epoxyandrographolide, 7-(4-morpholino)-12-(2-(pyridine-2-yl)amino)-14-deoxy-(E)-15-((2-amino-9-methyl-6-oxo-6,9-dihydro-1H-urin-8-yl)methylene)-8,17-epoxyandrographolide, 7((2-((4H-imidazol-2-yl)amino)-1-xopropan-2-yl)amino)-12-(2-(pyridine-2-yl)amino)-14-deoxy-(E)-15-((2-amino-9-ethyl-6-oxo-6,9-dihydro-1H-purin-8-yl)methylene)-8,17-epoxyandrographolide, 11,12-edihydro-14-deoxy-(E)-15-((4-imino-1-methyl-6-oxo-4,5,6,7-tetrahydro-1H-pyrazolo[3,4-d]pyrimidin-3-yl)methylene)-8,17-epoxyandrographolide, 7-(4-morpholino)-11,12-dedihydro-14-deoxy-(E)-15-((4-imino-1-methyl-6-oxo-4,5,6,7-tetrahydro-1H-pyrazolo[3,4-d]pyrimidin-3-yl)methylene)-8,17-epoxyandrographolide, 7-((2-((4H-imidazol-2-yl)amino)-1-oxopropan-2-yl)amino)-11,12-dedihydro-14-deoxy-(E)-15-((4-imino-1-methyl-6-oxo-4,5,6,7-tetrahydro-1H-pyrazolo[3,4-d]pyrimidin-3-yl)methylene)-8,17-epoxyandrographolide, 12-(2-(pyridine-2-yl)amino)-14-deoxy-(E)-15-((4-imino-1-methyl-6-oxo-4,5,6,7-tetrahydro-1H-pyrazolo[3,4-d]pyrimidin-3-yl)methylene)-8,17-epoxyandrographolide, 7-(4-morpholino)-12-(2-(pyridine-2-yl)amino)-14-deoxy-(E)-15-((4-imino-1-methyl-6-oxo-4,5,6,7-tetrahydro-1H-pyrazolo[3, 4-d]pyrimidin-3-yl)methylene)-8,17-epoxyandrographolide, 7-((2-((4H-imidazol-2-yl)amino)-1-oxopropan-2-yl)amino)-12-(2-(pyridine-2-yl)amino)-14-deoxy-(E)-15-((4-imino-1-methyl-6-oxo-4,5,6,7-tetrahydro-1H-pyrazolo[3,4-d]pyrimidin-3-yl)methylene)-8,17-epoxyandrographolide, 11,12-dedihydro-14-deoxy-(E)-15-((2-amino-7-methyl-7H-pyrrolo[2,3-d]pyrimidin-5-yl)methylene)-8,17-epoxyandrographolide, 7-(4-morpholino)-11,12-dedihydro-14-deoxy-(E)-15-((2-amino-7-methyl-7H-pyrrolo[2,3-d]pyrimidin-5-yl)methylene)-8,17-epoxyandrographolide, 7-((2-((4H-imidazol-2-yl)amino)-1-oxopropan-2-yl)amino)-11,12-dedihydro-14-deoxy-(E)-15-((2-amino-7-methyl-7H-pyrrolo[2,3-d]pyrimidin-5-yl)methylene)-8,17-epoxyandrographolide, 12-(2-(pyridine-2-yl)amino)-14-deoxy-(E)-15-((2-amino-7-methyl-7H-pyrrolo[2,3-d]pyrimidin-5-yl)methylene)-8,17-epoxyandrographolide, 7-(4-morpholino)-12-(2-(pyridine-2-yl)amino)-14-deoxy-(E)-15-((2-amino-7-methyl-7H-pyrrolo[2,3-d]pyrimidin-5-yl)methylene)-8,17-epoxyandrographolide, 7((2-((4H-imidazol-2-yl)amino)-1-oxopropan-2-yl)amino)-12-(2-(pyridine-2-yl)amino)-14-deoxy-(E)-15-((2-amino-7-methyl-7H-pyrrolo[2,3-d]pyrimidin-5-yl)methylene)-8,17-epoxyandrographolide, 11,12-dedihydro-14-deoxy-(E)-15-(4-(N-(2-(dimethylamino)ethyl)carbamimidoyl)benzylidene)-8,17-epoxyandro-grapholide, 7-(4-morpholino)-11,12-dedihydro-14-deoxy-(E)-15-(4-(N-(2-(dimethylamino)ethyl)carbamimidoyl)benzylidene)-8,17-epoxyandrographolide, 7-((2-((4H-imidazol-2-yl)amino)-1-oxopropan-2-yl)amino)-11,12-dedihydro-14-deoxy-(E)-15-(4-(N-(2-(dimethylamino)ethyl)carbamimidoyl)benzylidene)-8,17-epoxyandrographolide, 12-(2-(pyridine-2-yl)amino)-14-deoxy-(E)-15-(4-(N-(2-(dimethylamino)ethyl)carbamimidoyl)benzylidene)-8,17-epoxyandrographolide, 7-(4-morpholino)-12-(2-(pyridine-2-yl)amino)-14-deoxy-(E)-15-(4-(N-(2-(dimethylamino)ethyl)carbamimidoyl)benzylidene)-8,17-epoxyandrographolide, 7-((2-((4H-imidazol-2-yl)amino)-1-oxopropan-2-yl)amino)-12-(2-(pyridine-2-yl)amino)-14-deoxy-(E)-15-(4-(N-(2-(dimethylamino)ethyl)carbamimidoyl)benzylidene)-8,17-epoxyandrographolide, 11,12-dedihydro-14-deoxy-(E)-15-(4-guanidinobenzylidene)-8,17-epoxyandrographolide, 7-(4-morpholino)-11,12-dedihydro-14-deoxy-(E)-15-(4-guanidinobenzylidene)-8,17-epoxyandrographolide, 7-((2-((4H-imidazol-2-yl)amino)-1-oxopropan-2-yl)amino)-11,12-dedihydro-14-deoxy-(E)-15-(4-guanidinobenzylidene)-8,17-epoxyandrographolide, 12-(2-(pyridine-2-yl)amino)-14-deoxy-(E)-15-(4-guanidinobenzylidene)-8,17-epoxyandrographolide, 7-(4-morpholino)-12-(2-(pyridine-2-yl)amino)-14-deoxy-(E)-15-(4-guanidinobenzylidene)-8,17-epoxyandrographolide, 7-((2-((4H-imidazol-2-yl)amino)-1-oxopropan-2-yl)amino)-12-(2-(pyridine-2-yl)amino)-14-deoxy-(E)-15-(4-guanidinobenzylidene)-8,17-epoxyandrographolide, 11,12-dedihydro-14-deoxy-(E)-15-((2-(2-(hydroxymethyl)pyrrolidin-1-yl) pyrimidin-5-yl)methylene)-8,17-epoxyandrographolide, 7-(4-morpholino)-11,12-dedihydro-14-deoxy-(E)-15-((2-(2-(hydroxymethyl)pyrrolidin-1-yl)pyrimidin-5-yl)methylene)-8,17-epoxyandrographolide, 7-((2-((4H-imidazol-2-yl)amino)-1-oxopropan-2-yl)amino)-11,12-dedihydro-14-deoxy-(E)-15-((2-(2-(hydroxymethyl) pyrrolidin-1-yl)pyrimidin-5-yl)methylene)-8,17-epoxyandrographolide, 12-(2-(pyridine-2-yl)amino)-14-deoxy-(E)-15-((2-(2-(hydroxymethyl)pyrrolidin-1-yl)pyrimidin-5-yl)methylene)-8,17-epoxyandrographolide, 7-(4-morpholino)-12-(2-(pyridine-2-yl)amino)-14-deoxy-(E)-15-((2-(2-(hydroxymethyl)pyrrolidin-1-yl)pyrimidin-5-yl)methylene)-8,17-epoxyandrographolide, 7-((2-((4H-imidazol-2-yl)amino)-1-oxopropan-2-yl)amino)-12-(2-(pyridine-2-yl)amino)-14-deoxy-(E)-15-((2-(2-(hydroxymethyl)pyrrolidin-1-yl)pyrimidin-5-yl)methylene)-8,17-epoxyandrographolide, 3,14, 19-triacetylandrographolide, 3,19-diacetyl-11,12-dedihydro-14-deoxyandrographolide, 3,19-diacetyl-7-hydroxyl-11,12-dedihydro-14-deoxyandrographolide, 3,19-diacetyl-7-chloro-11,12-dedihydro-14-deoxyandrographolide, 3,19-diacetyl-7-oxo-11,12-dedihydro-14-deoxyandrographolide, 3,19-diacetyl-7-(4-morpholino)-11,12-dedihydro-14-deoxyandrographolide, 3-(4-(4-methylpiperazin-1-yl)-4-oxobutanoyl)-12-((2-aminophenyl)thio)-14-deoxyandrographolide, 3-(4-(4-methylpiperazin-1-yl)-4-oxobutanoyl)-12-((2-aminophenyl)thio)andrographolide, 3-(4-(4-methylpiperazin-1-yl)-4-oxobutanoyl)-12-nitromethyl-14-deoxyandrographolide, 12-((5-amino-4H-1,2,4-triazol-3-yl)thio)-14-deoxyandrographolide, 12-((4,6-dimethylpyrimidin-2-yl)thio)-14-deoxyandrographolide, 12-((2-aminophenyl)thio)-14-deoxyandrographolide, 12-((2-aminophenyl)thio)andrographolide, 11,12-dedihydro-14-deoxy-15-(propan-2-ylidene)andrographolide, 15,15-dimethylol-11,12-dedihydro-14-deoxyandrographolide, 11,12-dedihydro-14-deoxy-(E)-15-(1,3-dihydroxypropan-2-ylidene)andrographolide, 11,12-dedihydro-14-deoxy-(E)-15-(2,4-dichlorobenzylidene)andrographolide, 11,12-dedihydro-14-deoxy-(E)-5-(2,4-difluorobenzylidene)andrographolide, 11,12-dedihydro-14-deoxy-(E)-15-(furan-3-ylmethylene)andrographolide, 11,12-dedihydro-14-deoxy-15-(4-((3,4,5-trihydroxy-6-(hydroxymethyl)tetrahydro-2H-pyran-2-yl)oxy)benzylidene)andrographolide, 12-((2-((4H-imidazol-2-yl)amino)-1-oxopropan-2-yl)amino)-14-deoxy-(E)-15-(4-(3-((1S,5S,7S)-1,3,5-triazaadamantan-6-ylamino)-3-oxopropanamido)benzylidene) andrographolide, 12-nitromethyl-14-deoxyandrographolide, 12-(4-morpholino)-14-deoxyandrographolide, 12-((3-chlorophenyl)amino)-14-deoxyandrographolide, 12-((1r,3s,5R,7S)-3-hydroxyadamantan-1-yl)amino)-14-deoxyandrographolide, 12-((1s,3s,5s)-1,3,5-triazaadamantan-7-ylamino)-14-deoxyandrographolide, 12-((1-(((1r,3s,5R,7S)-3-hydroxyadamantan-1-yl)amino)-1-oxopropan-2-yl)amino)-14-deoxyandrographolide, 12-(diethoxyphosphoryl)-14-deoxyandrographolide, 3,19-diformyl-11,12-dedihydro-14-deoxyandrographolide, 3,14,19-triformyl-11,12-dedihydro-14-deoxyandrographolide, 3-(4-((3,4,5-trihydroxy-6-(hydroxymethyl)tetrahydro-2H-pyran-2-yl)oxy)benzoyl)-11,12-dedihydro-14-deoxyandrographolide, 3-(4-((3,4,5-trihydroxy-6-(hydroxymethyl)tetrahydro-2H-pyran-2-yl)oxy)benzoyl)-12-nitromethyl-14-deoxyandrographolide, 3-(4-((3,4,5-trihydroxy-6-(hydroxymethyl)tetrahydro-2H-pyran-2-yl)oxy)benzoyl)-12-(4-morpholino)-14-deoxyandrographolide, 3-(4-((3,4,5-trihydroxy-6-(hydroxymethyl)tetrahydro-2H-pyran-2-yl)oxy)benzoyl)-12-((2-aminophenyl)thio)-14-deoxyandrographolide, (E)-3-(2-(3,3,6a,10b-tetramethyl-8-methylenedecahydro-1H-naphtho[2,1-d][1,3]dioxin-7-yl)vinyl)furan-2(5H)-one, (E)-4-hydroxy-3-(2-(3,3,6a,10b-tetramethyl-8-methylenedecahydro-1H-naphtho[2,1-d][1,3]dioxin-7-yl)ethylidene)dihydrofuran-2(3H)-one, (E)-5-oxo-4-(2-(3,3,6a,10b-tetramethyl-8-methylenedecahydro-1H-naphtho[2,1-d][1,3]dioxin-7-yl)ethylidene)tetrahydrofuran-3-yl methanesulfonate, (E)-4-hydroxy-3-(2-(9-hydroxy-3,3,6a,10b-tetramethyl-8-methylenedecahydro-1H-naphtho[2,1-d][1,3]dioxin-7-yl)ethylidene)dihydrofuran-2(3H)-one, (E)-4-hydroxy-3-(2-(3,3,6a,10b-tetramethyldecahydrospiro[naphtho[2,1-d][1,3]dioxine-8,2′-oxiran]-7-yl)ethylidene)dihydrofuran-2(3H)one, (E)-3-(2-(6a,10b-dimethyl-8-methylene-3-(3-nitrophenyl)decahydro-1H-naphtho[2,1-d][1,3]dioxin-7-yl)ethylidene)-4-hydroxydihydrofuran-2(3H)-one, (E)-3-(2-(6a,10b-dimethyl-8-methylene-3-(4-((3,4,5-trihydroxy-6-(hydroxymethyl)tetrahydro-2H-pyran-2-yl)oxy)phenyl)decahydro-1H-naphtho[2,1-d][1,3]dioxin-7-yl)ethylidene)-4-hydroxydihydrofuran-2(3H)-one, (E)-(7-acetoxy-6,9a-dimethyl-10-(2-(2-oxo-2,5-dihydrofuran-3-yl)vinyl)-5a,6,7,8,9,9a,10,10a-octahydro-5H-imidazo[1,5-a]naphtho[2, 3-d]imidazol-6-yl)methyl acetate, (E)-dimethyl(4-(2-(6-hydroxy-5-(hydroxymethyl)-5,8a-dimethyl-2-methylenedecahydronaphthalen-1-yl)vinyl)-5-oxotetrahydrofuran-3-yl)phosphonate, (E)-2-(1,2-dihydroxyethyl)-4-(6-hydroxy-5-(hydroxymethyl)-5,8a-dimethyl-2-methylenedecahydronaphthalen-1-yl)but-2-enehydrazide, 3-(7-hydroxy-6-(hydroxymethyl)-6,9a-dimethyl-3a,4,5,5a,6,7,8,9,9a,9b-decahydro-1H-cyclopenta[a]naphthalen-2-yl)furan-2(5H)one, 6-(hydroxymethyl)-6,9a-dimethyl-2-(2-oxo-2,5-dihydrofuran-3-yl)-2,4,5,5a,6,7,8,9,9a,9b-decahydro-1H-cyclopenta[a]naphthalen-7-yl-4-morpholino-4-oxobutanoate, 6-(hydroxymethyl)-6,9a-dimethyl-2-(2-oxo-2,5-dihydrofuran-3-yl)-2,4,5,5a,6,7, 8,9,9a,9b-decahydro-1H-cyclopenta[a]naphthalen-7-yl-4-(4-methylpiperazin-1-yl)-4-oxobutanoate, 6-(hydroxymethyl)-6,9a-dimethyl-2-(2-oxo-2,5-dihydrofuran-3-yl)-2,4,5,5a,6,7, 8,9,9a,9b-decahydro-1H-cyclopenta[a]naphthalen-7-yl-4-((3,4,5-trihydroxy-6-(hydroxymethyl)tetrahydro-2H-pyran-2-yl)oxy)benzoate,

10. A compound according to the claim 1, wherein: the administration of a compound of andrographolide derivatives and analogs may be by oral route, parenteral, subcutaneous, intravenous, intramuscular, intra-peritoneal, transdermal, buccal, intrathecal, intracranial, intranasal or topical routes.
